(19) United States
(12) Patent Application Publication
(10) Pub. No.: US 2024/0033427 A1
(43) Pub. Date: Feb. 1, 2024

(54) SYSTEMS AND METHODS FOR DIABETES MANAGEMENT

(71) Applicant: ABBOTT DIABETES CARE INC., Alameda, CA (US)

(72) Inventors: Erwin S. Budiman, Fremont, CA (US); Matthew T. Novak, Oakland, CA (US); Gary A. Hayter, Oakland, CA (US)

(21) Appl. No.: 18/226,550

(22) Filed: Jul. 26, 2023

Related U.S. Application Data
(60) Provisional application No. 63/392,753, filed on Jul. 27, 2022.

(51) Int. Cl. A61M 5/172

(52) U.S. Cl. CPC A61M 5/1723; A61M 2205/52; A61M 2230/005; A61M 2230/201; A61M 2205/502; A61M 2205/18

(57) ABSTRACT

Systems, devices and methods are provided for incorporating a medication delivery device into an integrated management system. The integrated management system may be an integrated diabetes management system and may include a glucose monitor, a connected insulin pen, and software. The integrated management system may produce a plurality of reports that may include data related to analyte levels (e.g., glucose levels) and medication delivered (e.g., insulin delivered). The medication delivery device may also provide feedback to the user.

### CROSS-REFERENCE TO RELATED APPLICATIONS

This application claims priority to U.S. Provisional Application No. 63/392,753, filed Jul. 27, 2022, which is herein expressly incorporated by reference in its entirety for all purposes. This application is also related to U.S. Provisional Application No. 63/177,706, filed Apr. 21, 2021, and U.S. Provisional Application No. 63/236,910, filed Aug. 25, 2021, both of which are expressly incorporated herein by reference in their entireties for all purposes.

### FIELD

The subject matter described herein relates generally to systems, devices, and methods relating to integrated systems for diabetes management such as, for example, an integrated platform that connects insulin pens with a common viewing platform such that data can be shared among many parties.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including inconvenience, testing discretion, pain associated with glucose testing, and cost.

For patients that rely on the administration of medications (e.g., insulin) to treat or manage diabetes, it is desirable to have systems, devices, or methods that can integrate glucose data with insulin dosing data and provide more actionable insights to both patients, caregivers, and HCPs. Manual logging of insulin doses is not only a huge time commitment but often results in inaccurate dosing logs. Moreover, sharing these manual logs with the HCP is a big hassle for patients and fraught with workflow issues.

Therapeutic management of diabetes often requires the use of multiple drugs that have different delivery frequencies (i.e., daily, weekly) as well as routes of administration (oral or injection). The combination of drug types, delivery frequencies and routes of administration can be difficult to manage, creating significant cognitive burden for a user that often translates to poor dose regimen concordance. Studies have shown that amongst insulin-dependent people with Type 1 diabetes, up to 24% of mealtime rapid acting insulin boluses and 36% of long acting once daily basal doses are missed, leading to poor glucose control and diabetes management. Existing dose logbooks are only as useful as a user wants to make them. Their efficacy is directly correlated to a user's willingness to log doses and reference past loggings. The dawn of connected dosing technology, such as BLUETOOTH®-enabled insulin pens, has enabled doses to be logged automatically into companion mobile phone applications as they are delivered with no user action required. While useful in day-to-day management, this method suffers from two notable shortcomings: (1) it is not able to correlate directly with glycemic outcomes; and (2) insulin doses are often presented in tabular form that is difficult to read and interpret over a large time window.

For these and other reasons, needs exist for improved systems, methods, and devices relating to integrated systems for diabetes management.

### SUMMARY

Provided herein are example embodiments of systems, devices and methods relating to management of diabetes, including integrated managements systems that include an analyte monitoring device, a medication delivery device, a reader device, monitoring software, and reporting software. In many embodiments, the analyte monitoring device (e.g., a continuous or flash glucose monitor) and the medication delivery device (e.g., an insulin pen) are communicatively coupled with the reader device to enable an easy transfer of analyte data, dose logs, and other information to computing devices that include monitoring and/or reporting software. The integrated system can include GUI displays that instruct and assist the user in connecting a medication delivery device to the monitoring software on, e.g., the reader device, such that dosing logs and other information can be transferred from the medication delivery device. The integrated system also includes reporting software that can produce a plurality of reports that incorporate data regarding analyte levels and metrics and medication dosing amounts and metrics.

This method addresses both of those concerns by utilizing glucose data as an indicator of poor dose concordance and presenting the data in a way that will allow a trained health care professional to quickly identify situations where poor medication dose concordance results in poor glycemic control. In some embodiments, the glucose data may be the sole indicator of poor dose concordance.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are block diagrams of example embodiments of a integrated management system.

FIG. 2A is a schematic diagram depicting an example embodiment of a sensor control device.

FIG. 2B is a block diagram depicting an example embodiment of a sensor control device.

FIG. 3A is a schematic diagram depicting an example embodiment of a medication delivery device.

FIG. 3B is a block diagram depicting an example embodiment of a medication delivery device.

FIG. 3C is a schematic diagram depicting an example embodiment of a medication delivery device with a smart button.

FIG. 4A is a schematic diagram depicting an example embodiment of a display device.

FIG. 4B is a block diagram depicting an example embodiment of a display device.

FIG. 5 is a block diagram depicting an example embodiment of a user interface device.

FIG. 6 is a flow diagram of a user experience of an example embodiment of the integrated management system.

FIG. 7 is a flow diagram of a user experience of adding a medication delivery device to the integrated management system.

FIG. 8A is a block diagram depicting an example embodiment of a Snapshot report.

FIGS. 8B-8C are example embodiments of Snapshot reports.

FIG. 9A is a block diagram depicting an example embodiment of a Weekly Summary report.

FIGS. 9B-9G are example embodiments of Weekly Summary reports.

FIG. 10A is a block diagram depicting an example embodiment of a Daily Log report.

FIGS. 10B-10C are example embodiments of Daily Log reports.

FIG. 11A is a block diagram depicting an example embodiment of a Daily Pattern report.

FIGS. 11B-11C are example embodiments of Daily Pattern reports.

FIGS. 11D-11E are exemplary embodiments of Daily Pattern report GUIs that may be displayed on the display device.

FIG. 12A is a block diagram depicting an example embodiment of a Mealtime Patterns report.

FIGS. 12B-12C are example embodiments of a Mealtime Patterns report.

FIG. 13A is a block diagram depicting an example embodiment of a Device Details report.

FIG. 13B is an example embodiment of a Device Details report.

FIG. 14 is a block diagram depicting an example embodiment of an AGP report.

FIG. 15A is a block diagram depicting an example embodiment of a GUI from a Patient Dashboard.

FIG. 15B is an example embodiment of a GUI from a Patient Dashboard.

FIG. 16A is a block diagram depicting an example embodiment of a GUI related to a Data Sources Modal with Connected Pen.

FIG. 16B is an example embodiment of a GUI related to a Data Sources Modal with Connected Pen.

FIG. 17A is a block diagram depicting an example embodiment of an alert related to a connected insulin pen.

FIG. 17B is an example embodiment of an alert related to a connected insulin pen.

FIGS. 18A-18B are block diagrams depicting example embodiments of comparative AGP reports.

FIG. 18C is an exemplary AGP illustrating all data.

FIG. 18D is an exemplary AGP illustrating data excluding missed doses.

FIG. 19 is an example embodiment of a Comparison Report.

FIGS. 20A-20D are example embodiments of GUIs for use with connecting a medical delivery device with a monitoring application.

FIGS. 20E-20F are example embodiments of GUIs for use with managing the user's connected medication delivery device.

FIGS. 21A-21B are example embodiments of GUIs for use with downloading and reviewing dosing records.

FIG. 21C are example embodiments of GUIs for use with editing medication dosage notes.

FIG. 21D is an example embodiment of a GUI for use with prompting a user to learn about connecting a medication delivery device.

FIG. 22 are example embodiments of GUIs for use with customizing a name for a medication delivery device.

FIGS. 23A-23C are example embodiments of GUIs for use concerning errors in transferred data.

FIGS. 24A-24C are example embodiments of Insulin Summary Report GUIs.

FIG. 25 is an exemplary method for displaying a missed meal dose alert or message.

FIG. 26 is an exemplary method for displaying a correction dose alert or message.

FIG. 27 is an exemplary method for displaying a congratulatory alert or message.

FIGS. 28A-β are exemplary embodiments of GUIs of Daily View Report GUIs.

FIGS. 29A-D are exemplary methods for displaying analyte profiles based on filtered data.

FIGS. 30A-C are exemplary methods for displaying analyte metrics based on filtered data.

FIGS. 31A-B are exemplary methods for providing feedback on a medication delivery device.

FIG. 32A is an exemplary method for providing haptic feedback on a medication delivery device.

FIG. 32B is an exemplary method for providing a safeguard against a higher than recommended dose being administered.

FIG. 32C is an exemplary method of adjusting alert parameters in response to a medication dose.

FIGS. 32D-32G are exemplary methods for providing safeguards against late dose administration.

FIG. 32H is an exemplary method of providing a safeguard when a zero dose is recommended.

FIG. 32I is an exemplary method of how actions on the medication delivery device may affect a display of a reader device.

FIG. 32J is an exemplary method of providing shortcuts to doses on the medication delivery device.

FIG. 33A is a side view of a connected pen.

FIG. 33B is an end view of the connected pen of FIG. 33A.

FIGS. 34A-34D depict a medication pen and a smart button.

FIG. 35 is a depiction of an exemplary medication pump.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

Generally, embodiments of the present disclosure include systems, devices, and methods related to integrated diabetes management. The integrated diabetes management system can include smart delivery systems, such as connected, smart insulin pens, glucose sensors, software to receive and process data from the glucose sensors and smart delivery systems, and a viewing platform capable of determining and visualizing dose analytics. The integrated management system (IMS) can further include reports that include insights as to the effects of various inulin doses and treatment advice, including dosing recommendations.

The integrated diabetes management can be implemented as software and/or firmware instructions stored in a memory of a computing device for execution by at least one processor or processing circuitry thereof. The computing device can be in the possession of a user or healthcare professional (HCP), and the user or HCP can interface with the computing device through a user interface. According to some embodiments, the computing device can be a server or trusted computer system that is accessible through a network, and the integrated management software can be presented to the user in the form of an interactive web page by way of a browser executed on a local display device (having the user interface) in communication with the server or trusted computer system through the network. In this and other embodiments, the integrated management software can be executed across multiple devices, or executed, in part, on processing circuitry of a local display device and, in part, on processing circuitry of a server or trusted computer system. It will be understood by those of skill in the art that when the IMS is described as performing an action, such action is performed according to instructions stored in a computer memory (including instructions hardcoded in read only memory) that, when executed by at least one processor of at least one computing device, causes the IMS to perform the described action. In all cases the action can alternatively be performed by hardware that is hardwired to implement the action (e.g., dedicated circuitry) as opposed to performance by way of instructions stored in memory.

Furthermore, as used herein, a system on which the IMS is implemented can be referred to as an integrated management system. The integrated management system can be configured for the sole purpose of providing integrated management or can be a multifunctional system of which integrated management is only one aspect. For example, in some embodiments the integrated management system can also be capable of monitoring analyte levels of a user. In some embodiments the integrated management system can also be capable of delivering medication to the user, such as with an injection or infusion device. In some embodiments, the integrated management system is capable of both monitoring analytes and delivering medication.

These embodiments and others described herein represent improvements in the field of computer-based dose determination, analyte monitoring, and medication delivery systems. The specific features and potential advantages of the disclosed embodiments are further discussed below.

Before describing the integrated management embodiments in detail, it is first desirable to describe examples of integrated management systems on or through which the integrated management application can be implemented.

Example Embodiments of Integrated Systems

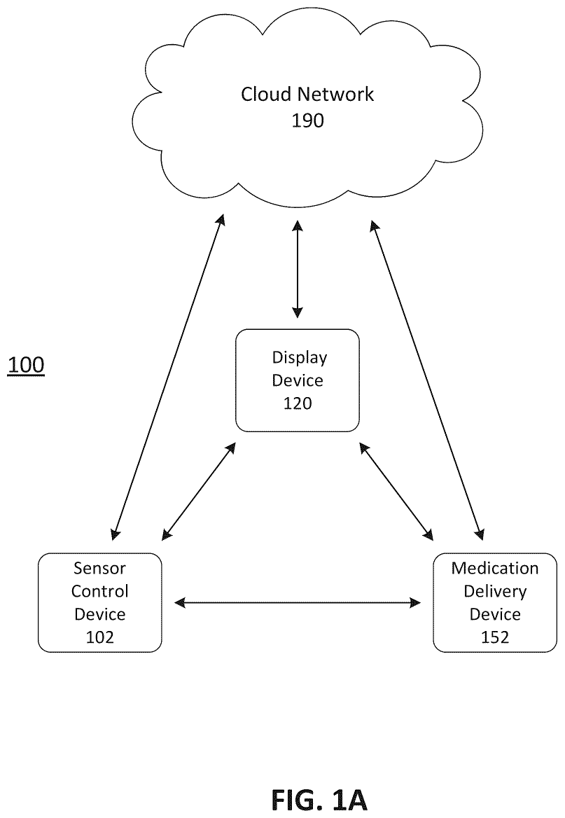

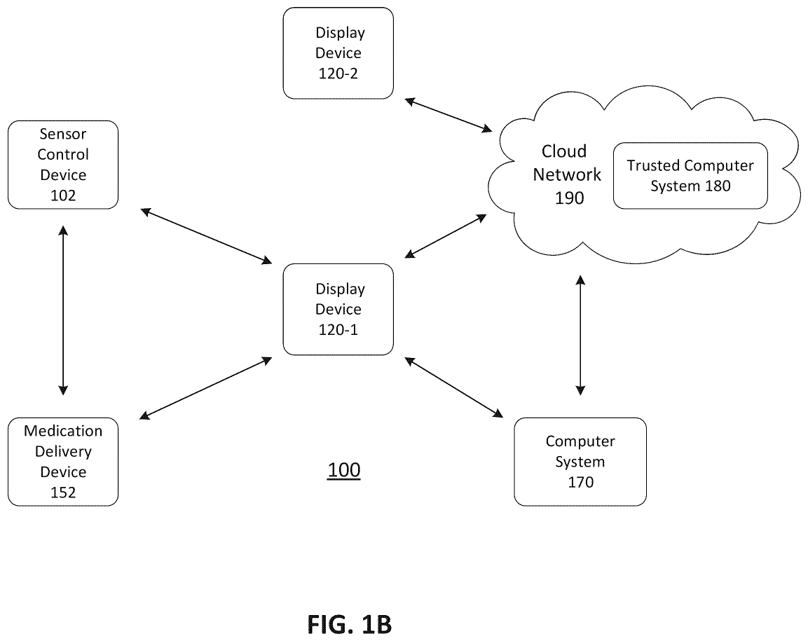

FIG. 1A is a block diagram depicting an example embodiment of integrated system 100. In this embodiment, integrated management system 100 is capable of delivering one or more medications, logging medication doses, monitoring one or more analytes, and determining and viewing analytics, and providing treatment advice. This multifunctional example is used to illustrate the high degree of interconnectivity and performance obtainable by system 100.

Here, system 100 includes a sensor control device (SCD) 102 configured to collect analyte level information from a user, a medication delivery device (MDD) 152 configured to deliver medication to the user, and a display device 120 configured to present information to the user and receive input or information from the user. The structure and function of each device will be described in detail herein.

System 100 is configured for highly interconnected and highly flexible communication between devices. Each of the three devices 102, 120, and 152, can communicate directly with each other (without passing through an intermediate electronic device) or indirectly with each other (such as through cloud network 190, or through another device and then through network 190). Bidirectional communication capability between devices, as well as between devices and network 190, is shown in FIG. 1A with a double-sided arrow. However, those of skill in the art will appreciate that any of the one or more devices (e.g., SCD) can be capable of unidirectional communication such as, for example, broadcasting, multicasting, or advertising communications. In each instance, whether bidirectional or unidirectional, the communication can be wired or wireless. The protocols that govern communication over each path can be the same or different, and can be either proprietary or standardized. For example, wireless communication between devices 102, 120, and 152 can be performed according to a BLUETOOTH® (including BLUETOOTH® Low Energy) standard, a Near Field Communication (NFC) standard, a Wi-Fi (802.11x) standard, a mobile telephony standard, or others. All communications over the various paths can be encrypted, and each device of FIG. 1A can be configured to encrypt and decrypt those communications sent and received. In each instance the communication pathways of FIG. 1A can be direct (e.g., BLUETOOTH® or NFC) or indirect (e.g., Wi-Fi, mobile telephony, or other internet protocol). Embodiments of system 100 do not need to have the capability to communicate across all of the pathways indicated in FIG. 1A.

In addition, although FIG. 1A depicts a single display device 120, a single SCD 102, and a single MDD 152, those of skill in the art will appreciate that system 100 can comprise a plurality of any of the aforementioned devices. By way of example only, system 100 can comprise a single SCD 102 in communication with multiple (e.g., two, three, four, etc.) display devices 120 and/or multiple MDDs 152. Alternatively, system 100 can comprise a plurality of SCDs 102 in communication with a single display device 120 and/or a single MDD 152. Furthermore, each of the plurality of devices can be of the same or different device types. For example, system 100 can comprise multiple display devices 120, including a smart phone, a handheld receiver, and/or a smart watch, each of which can be in communication with SCD 102 and/or MDD 152, as well in communication with each other.

Analyte data can be transferred between each device within system 100 in an autonomous fashion (e.g., transmitting automatically according to a schedule), or in response to a request for analyte data (e.g., sending a request from a first device to a second device for analyte data, followed by transmission of the analyte data from the second device to the first device). Other techniques for communicating data can also be employed to accommodate more complex systems like cloud network 190.

FIG. 1B is a block diagram depicting another example embodiment of integrated management system 100. Here, system 100 includes SCD 102, MDD 152, a first display device 120-1, a second display device 120-2, local computer system 170, and trusted computer system 180 that is accessible by cloud network 190. SCD 102 and MDD 152 are capable of communication with each other and with display device 120-1, which can act as a communication hub for aggregating information from SCD 102 and MDD 152, processing and displaying that information where desired, and transferring some or all of the information to cloud network 190 and/or computer system 170. Conversely, display device 120-1 can receive information from cloud network 190 and/or computer system 170 and communicate some or all of the received information to SCD 102, MDD 152, or both. Computer system 170 may be a personal computer, a server terminal, a laptop computer, a tablet, or other suitable data processing device. Computer system 170 can include or present software for data management and analysis and communication with the components in system 100. Computer system 170 can be used by the user or a medical professional to display and/or analyze analyte data measured by SCD 102. Furthermore, although FIG. 1B depicts a single SCD 102, a single MDD 152, and two display devices 120-1 and 120-2, those of skill in the art will appreciate that system 100 can include a plurality of any of the aforementioned devices, wherein each plurality of devices can comprise the same or different types of devices.

Referring still to FIG. 1B, according to some embodiments, trusted computer system 180 can be within the possession of a manufacturer or distributor of a component of system 100, either physically or virtually through a secured connection, and can be used to perform authentication of the devices of system 100 (e.g., devices 102, 120-n, 152), for secure storage of the user's data, and/or as a server that serves a data analytics program (e.g., accessible via a web browser) for performing analysis on the user's measured analyte data and medication history. Trusted computer system 180 can also act as a data hub for routing and exchanging data between all devices in communication with system 180 through cloud network 190. In other words, all devices of system 100 that are capable of communicating with cloud network 190 (e.g., either directly with an internet connection or indirectly via another device), are also capable of communicating with all of the other devices of system 100 that are capable of communicating with cloud network 190, either directly or indirectly.

Display device 120-2 is depicted in communication with cloud network 190. In this example, device 120-2 can be in the possession of another user that is granted access to the analyte and medication data of the person wearing SCD 102. For example, the person in possession of display device 120-2 can be a parent of a child wearing SCD 102, as one example, or a caregiver of an elderly patient wearing SCD 102, as another example. System 100 can be configured to communicate analyte and medication data about the wearer through cloud network 190 (e.g., via trusted computer system 180) to another user with granted access to the data.

### Example Embodiments of Analyte Monitoring Devices

The analyte monitoring functionality of integrated management system 100 can be realized through inclusion of one or more devices capable of collecting, processing, and displaying analyte data of the user. Example embodiments of such devices and their methods of use are described in Int'l Publ. No. WO 2018/152241 and U.S. Patent Publ. No. 2011/0213225, both of which are incorporated by reference herein in their entireties for all purposes.

Analyte monitoring can be performed in numerous different ways. “Continuous Analyte Monitoring” devices (e.g., “Continuous Glucose Monitoring” devices), for example, can transmit data from a sensor control device to a display device continuously or repeatedly with or without prompting, e.g., automatically according to a schedule. “Flash Analyte Monitoring” devices (e.g., “Flash Glucose Monitoring” devices or simply “Flash” devices), as another example, can transfer data from a sensor control device in response to a user-initiated request for data by a display device (e.g., a scan), such as with a Near Field Communication (NFC) or Radio Frequency Identification (RFID) protocol.

Analyte monitoring devices that utilize a sensor configured to be placed partially or wholly within a user's body can be referred to as in vivo analyte monitoring devices. For example, an in vivo sensor can be placed in the user's body such that at least a portion of the sensor is in contact with a bodily fluid (e.g., interstitial (ISF) fluid such as dermal fluid in the dermal layer or subcutaneous fluid beneath the dermal layer, blood, or others) and can measure an analyte concentration in that bodily fluid. In vivo sensors can use various types of sensing techniques (e.g., chemical, electrochemical, or optical). Some systems utilizing in vivo analyte sensors can also operate without the need for finger stick calibration.

“In vitro” devices are those where a sensor is brought into contact with a biological sample outside of the body (or rather “ex vivo”). These devices typically include a port for receiving an analyte test strip carrying bodily fluid of the user, which can be analyzed to determine the user's blood glucose level. Other ex vivo devices have been proposed that attempt to measure the user's internal analyte level non-invasively, such as by using an optical technique that can measure an internal body analyte level without mechanically penetrating the user's body or skin. In vivo and ex vivo devices often include in vitro capability (e.g., an in vivo display device that also includes a test strip port).

The present subject matter will be described with respect to sensors capable of measuring a glucose concentration, although detection and measurement of concentrations of other analytes are within the scope of the present disclosure. These other analytes can include, for example, ketones, lactate, oxygen, hemoglobin A1C, acetyl choline, amylase, bilirubin, cholesterol, chorionic gonadotropin, creatine kinase (e.g., CK-MB), creatine, DNA, fructosamine, glutamine, growth hormones, hormones, peroxide, prostate-specific antigen, prothrombin, RNA, thyroid stimulating hormone, troponin and others. The concentration of drugs, such as, for example, antibiotics (e.g., gentamicin, vancomycin, and the like), digitoxin, digoxin, drugs of abuse, theophylline, and warfarin, may also be monitored. The sensor can be configured to measure two or more different analytes at the same or different times. In some embodiments, the sensor control device can be coupled with two or more sensors, where one sensor is configured to measure a first analyte (e.g., glucose) and the other one or more sensors are configured to measure one or more different analytes (e.g., any of those described herein). In other embodiments, a user can wear two or more sensor control devices, each of which is capable of measuring a different analyte.

The embodiments described herein can be used with all types of in vivo, in vitro, and ex vivo devices capable of monitoring the aforementioned analytes and others.

In many embodiments, the sensor operation can be controlled by SCD 102. The sensor can be mechanically and communicatively coupled with SCD 102, or can be just communicatively coupled with SCD 102 using a wireless communication technique. SCD 102 can include the electronics and power supply that enable and control analyte sensing performed by the sensor. In some embodiments the sensor or SCD 102 can be self-powered such that a battery is not required. SCD 102 can also include communication circuitry for communicating with another device that may or may not be local to the user's body (e.g., a display device). SCD 102 can reside on the body of the user (e.g., attached to or otherwise placed on the user's skin, or carried in the user's clothes, etc.). SCD 102 can also be implanted within the body of the user along with the sensor. Functionality of SCD 102 can be divided between a first component implanted within the body (e.g., a component that controls the sensor) and a second component that resides on or otherwise outside the body (e.g., a relay component that communicates with the first component and also with an external device like a computer or smartphone). In other embodiments, SCD 102 can be external to the body and configured to non-invasively measure the user's analyte levels. The sensor control device, depending on the actual implementation or embodiment, can also be referred to as a “sensor control unit,” an “on-body electronics” device or unit, an “on-body” device or unit, an “in body electronics” device or unit, an “in-body” device or unit, or a “sensor data communication” device or unit, to name a few.

In some embodiments, SCD 102 may include a user interface (e.g., a touchscreen) and be capable of processing the analyte data and displaying the resultant calculated analyte levels to the user. In such cases, the integrated management embodiments described herein can be implemented directly by SCD 102, in whole or in part. In many embodiments, the physical form factor of SCD 102 is minimized (e.g., to minimize the appearance on the user's body) or the sensor control device may be inaccessible to the user (e.g., if wholly implanted), or other factors may make it desirable to have a display device usable by the user to read analyte levels and interface with the sensor control device.

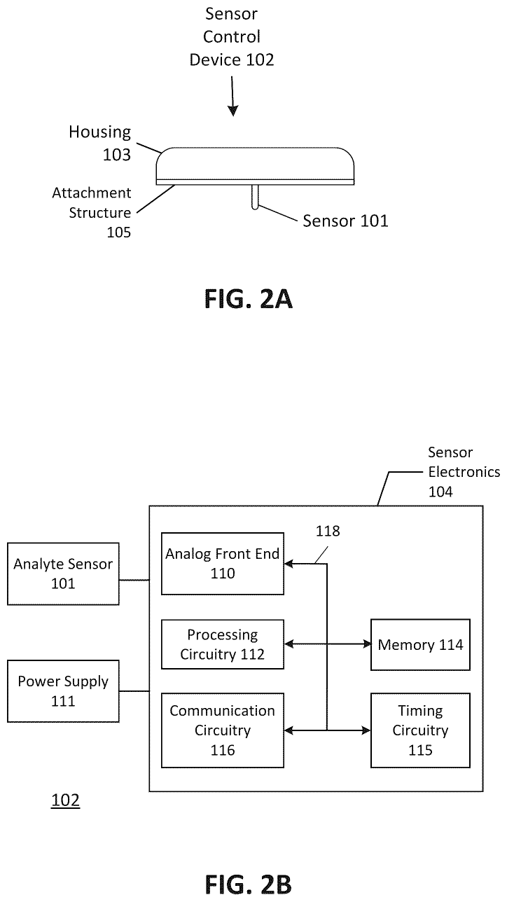

FIG. 2A is a side view of an example embodiment of SCD 102. SCD 102 can include a housing or mount 103 for sensor electronics (FIG. 2B), which can be electrically coupled with an analyte sensor 101, which is configured here as an electrochemical sensor. According to some embodiments, sensor 101 can be configured to reside partially within a user's body (e.g., through an exterior-most surface of the skin) where it can make fluid contact with a user's bodily fluid and be used, along with the sensor electronics, to measure analyte-related data of the user. A structure for attachment 105, such as an adhesive patch, can be used to secure housing 103 to a user's skin. Sensor 101 can extend through attachment structure 105 and project away from housing 103. Those of skill in the art will appreciate that other forms of attachment to the body and/or housing 103 may be used, in addition to or instead of adhesive, and are fully within the scope of the present disclosure.

SCD 102 can be applied to the body in any desired manner. For example, an insertion device (not shown), sometimes referred to as an applicator, can be used to position all or a portion of analyte sensor 101 through an external surface of the user's skin and into contact with the user's bodily fluid. In doing so, the insertion device can also position SCD 102 onto the skin. In other embodiments, the insertion device can position sensor 101 first, and then accompanying electronics (e.g., wireless transmission circuitry and/or data processing circuitry, and the like) can be coupled with sensor 101 afterwards (e.g., inserted into a mount), either manually or with the aid of a mechanical device. Examples of insertion devices are described in U.S. Patent Publication Nos. 2008/0009692, 2011/0319729, 2015/0018639, 2015/0025345, and 2015/0173661, 2018/0235520, all which are incorporated by reference herein in their entireties for all purposes.

FIG. 2B is a block diagram depicting an example embodiment of SCD 102 having analyte sensor 101 and sensor electronics 104. Sensor electronics 104 can be implemented in one or more semiconductor chips (e.g., an application specific integrated circuit (ASIC), processor or controller, memory, programmable gate array, and others). In the embodiment of FIG. 1B, sensor electronics 104 includes high-level functional units, including an analog front end (AFE) 110 configured to interface in an analog manner with sensor 101 and convert analog signals to and/or from digital form (e.g., with an A/D converter), a power supply 111 configured to supply power to the components of SCD 102, processing circuitry 112, memory 114, timing circuitry 115 (e.g., such as an oscillator and phase locked loop for providing a clock or other timing to components of SCD 102), and communication circuitry 116 configured to communicate in wired and/or wireless fashion with one or more devices external to SCD 102, such as display device 120 and/or MDD 152.

SCD 102 can be implemented in a highly interconnected fashion, where power supply 111 is coupled with each component shown in FIG. 2B and where those components that communicate or receive data, information, or commands (e.g., AFE 110, processing circuitry 112, memory 114, timing circuitry 115, and communication circuitry 116), can be communicatively coupled with every other such component over, for example, one or more communication connections or buses 118.

Processing circuitry 112 can include one or more processors, microprocessors, controllers, and/or microcontrollers, each of which can be a discrete chip or distributed amongst (and a portion of) a number of different chips. Processing circuitry 112 can include on-board memory. Processing circuitry 112 can interface with communication circuitry 116 and perform analog-to-digital conversions, encoding and decoding, digital signal processing and other functions that facilitate the conversion of data signals into a format (e.g., in-phase and quadrature) suitable for wireless or wired transmission. Processing circuitry 112 can also interface with communication circuitry 116 to perform the reverse functions necessary to receive a wireless transmission and convert it into digital data or information.

Processing circuitry 112 can execute instructions stored in memory 114. These instructions can cause processing circuitry 112 to process raw analyte data (or pre-processed analyte data) and arrive at a final calculated analyte level. In some embodiments, instructions stored in memory 114, when executed, can cause processing circuitry 112 to process raw analyte data to determine one or more of: a calculated analyte level, an average calculated analyte level within a predetermined time window, a calculated rate-of-change of an analyte level within a predetermined time window, and/or whether a calculated analyte metric exceeds a predetermined threshold condition. These instructions can also cause processing circuitry 112 to read and act on received transmissions, to adjust the timing of timing circuitry 115, to process data or information received from other devices (e.g., calibration information, encryption or authentication information received from display device 120, and others), to perform tasks to establish and maintain communication with display device 120, to interpret voice commands from a user, to cause communication circuitry 116 to transmit, and others. In embodiments where SCD 102 includes a user interface, then the instructions can cause processing circuitry 112 to control the user interface, read user input from the user interface, cause the display of information on the user interface, format data for display, and others. The functions described here that are coded in the instructions can instead be implemented by SCD 102 with the use of a hardware or firmware design that does not rely on the execution of stored software instructions to accomplish the functions.

Memory 114 can be shared by one or more of the various functional units present within SCD 102, or can be distributed amongst two or more of them (e.g., as separate memories present within different chips). Memory 114 can also be a separate chip of its own. Memory 114 is non-transitory, and can be volatile (e.g., RAM, etc.) and/or non-volatile memory (e.g., ROM, flash memory, F-RAM, etc.).

Communication circuitry 116 can be implemented as one or more components (e.g., transmitter, receiver, transceiver, passive circuit, encoder, decoder, and/or other communication circuitry) that perform the functions for communications over the respective communications paths or links. Communication circuitry 116 can include or be coupled to one or more antenna for wireless communication.

Power supply 111 can include one or more batteries, which can be rechargeable or single-use disposable batteries. Power management circuitry can also be included to regulate battery charging and monitor usage of power supply 111, boost power, perform DC conversions, and the like.

Additionally, an on-skin or sensor temperature reading or measurement can be collected by an optional temperature sensor (not shown). Those readings or measurements can be communicated (either individually or as an aggregated measurement over time) from SCD 102 to another device (e.g., display device 120). The temperature reading or measurement, however, can be used in conjunction with a software routine executed by SCD 102 or display device 120 to correct or compensate the analyte measurement output to the user, instead of or in addition to, actually outputting the temperature measurement to the user.

### Example Embodiments of Medication Delivery Devices

Insulin dosing systems can be categorized by whether the delivery is done in an automated or manual fashion. At one end of the spectrum, a fully automated insulin dosing system, such as a fully closed loop insulin dosing system, determines and delivers insulin doses via an insulin pump at any time based on information primarily from a glucose monitoring module such as a CGM system. At the other end of the spectrum, a fully manual insulin dosing system relies on the user to determine the proper dose based on known information (e.g., their state of health, meal consumption, and CGM system) and an insulin dosing calculation method. The user may then initiate insulin delivery on an insulin delivery module or device such as a traditional insulin syringe, an insulin pen, a smart insulin pen, or an insulin pump. Somewhere in between is a decision support system (DSS) that may receive information primarily from a glucose monitoring module (such as a CGM system) and determine an insulin dose recommendation, but requires the user to approve/deliver the dose via the corresponding insulin delivery module or device (MDD 152) (such as traditional insulin syringe, insulin pen, smart insulin pen, or insulin pump). Systems that determine dose recommendation guidance are described in U.S. Patent Publ. No. 2021/0050085 and U.S. application Ser. No. 17/591,229, which are hereby expressly incorporated by reference in their entirety for all purposes.

The medication delivery functionality of integrated management system 100 can be realized through inclusion of one or more medication delivery devices (MDDs) 152. MDD 152 can be any device configured to deliver a specific dose of medication. The MDD 152 can also include devices that transmit data regarding doses to the IMS, e.g., pen caps, even though the device itself may not deliver the medication. The MDD 152 can be configured as a portable injection device (PID) that can deliver a single dose per one injection, such as a bolus. The PID can be a basic manually-operated syringe, where the medication is either preloaded in the syringe or must be drawn into the syringe from a container prior to injection. In most embodiments, however, the PID includes electronics for interfacing with the user and performing the delivery of the medication. PIDs are often referred to as medication pens, although a pen-like appearance is not required. PIDs having user interface electronics are often referred to as smart pens. PIDs can be used to deliver one dose and then disposed of, or can be durable and used repeatedly to deliver many doses over the course of a day, week, or month. PIDs are often relied upon by users that practice a multiple daily injection (MDI) therapy regimen.

The MDD can also comprise a pump and infusion set. The infusion set includes a tubular cannula that resides at least partially within the recipient's body. The tubular cannula is in fluid communication with a pump, which can deliver medication through the cannula and into the recipient's body in small increments repeatedly over time. The infusion set can be applied to the recipient's body using an infusion set applicator, and the infusion set often stays implanted for 2 to 3 days or longer. A pump device includes electronics for interfacing with the user and for controlling the slow infusion of the medication. Both a PID and a pump can store the medication in a medication reservoir.

MDD 152 can function as part of a closed-loop system (e.g., an artificial pancreas system requiring no user intervention to operate), semi-closed loop system (e.g., an insulin loop system requiring seldom user intervention to operate, such as to confirm changes in dose), or an open loop system. For example, the diabetic's analyte level can be monitored in a repeated automatic fashion by SCD 102, and that information can be transmitted to the application and incorporated in various analytics and reports.

In many embodiments, the integrated management system may include data for different types of insulin (e.g., rapid-acting (RA), short-acting insulin, intermediate-acting insulin (e.g., NPH insulin), long-acting (LA), ultra long-acting insulin, and mixed insulin), and will be the same medication delivered by MDD 152. The type of insulin includes human insulin and synthetic insulin analogs. The insulin can also include premixed formulations. However, the integrated management embodiments set forth herein and the medication delivery capabilities of MDD 152 can be applied to other non-insulin medications. Such medications can include, but are not limited to exenatide, exenatide extended release, liraglutide, lixisenatide, semaglutide, pramlintide, metformin, SLGT1-i inhibitors, SLGT2-i inhibitors, and DPP4 inhibitors. The integrated management embodiments can also include combination therapies. Combination therapies can include, but are not limited to, insulin and glucagon-like peptide-1 receptor agonists (GLP-1 RA), insulin and pramlintide.

For ease of description of the integrated management embodiments herein, MDD 152 will often be described in the form of a PID, specifically a smart pen. However, those of skill in the art will readily understand that MDD 152 can alternatively be configured as a pen cap, a pump, or any other type of medication delivery device.

In some embodiments, the IMS may include a connected pen cap. After the connected pen cap is attached to an insulin pen and is paired with the display device, every time a dose of insulin is delivered, the connected pen cap may automatically transmit dose data to the display device via e.g., BLUETOOTH®.

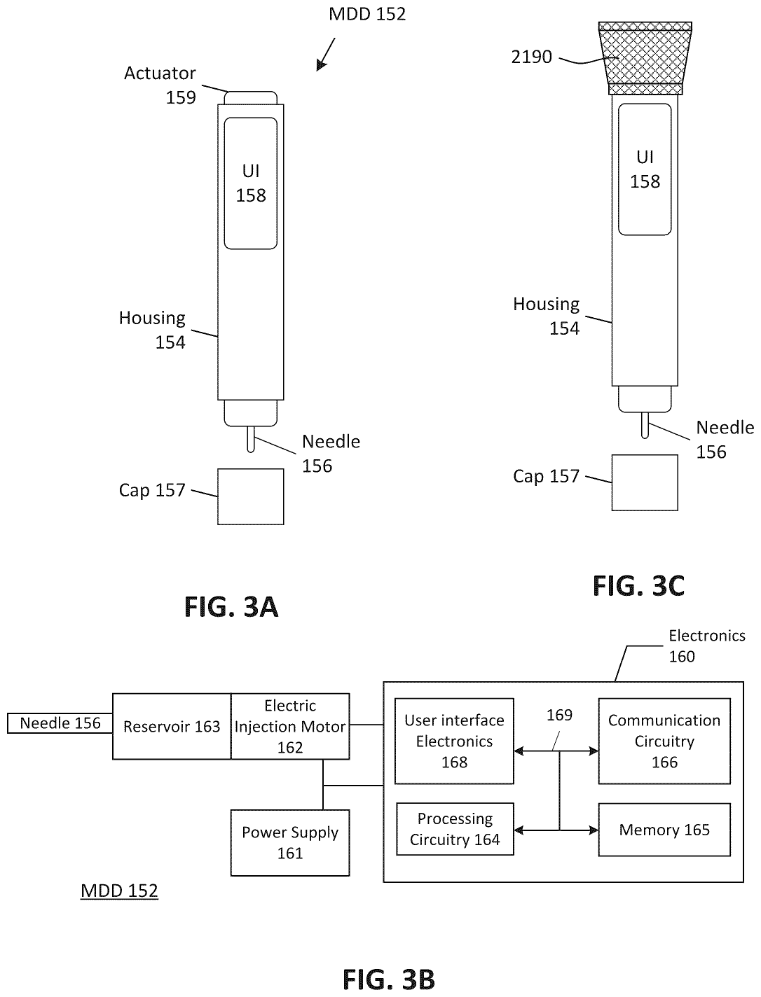

FIG. 3A is schematic diagram depicting an example embodiment of an MDD 152 configured as a PID, specifically a smart pen. MDD 152 can include a housing 154 for electronics, an injection motor, and a medication reservoir (see FIG. 3B), from which medication can be delivered through needle 156. Housing 154 can include a removable or detachable cap or cover 157 that, when attached, can shield needle 156 when not in use, and then be detached for injection. MDD 152 can also include a user interface 158 which can be implemented as a single component (e.g., a touchscreen for outputting information to the user and receiving input from the user) or as multiple components (e.g., a touchscreen or display in combination with one or more buttons, switches, or the like). MDD 152 can also include an actuator 159 that can be moved, depressed, touched or otherwise activated to initiate delivery of the medication from an internal reservoir through needle 156 and into the recipient's body. According to some embodiments, cap 157 and actuator 159 can also include one or more safety mechanisms to prevent removal and/or actuation to mitigate risk of a harmful medication injection. Details of these safety mechanisms and others are described in U.S. Patent Publ. No. 2019/0343385 (the '385 publication), which is hereby incorporated in its entirety for all purposes. FIG. 3C is a schematic diagram of an MDD 152 and a smart button configured to fit over the actuator of the delivery pen. The MDD 152, which may be a single use medication delivery pen, may include a display 158 and a dosing selector such as a rotatable dial to select the dose. A smart button 2190 may fit over the dosing selector and actuator 159 and snap together with the pen 2180. After smart button 2190 is paired with a reader device, the smart button 2190 may track the dose, store the dosing data, and transmit it to the reader device. Medication may be delivered by pushing straight down on the smart button 2190 to inject the dose. The smart button 2190 may also contain a display that can display the recommended dose and/or the dose administered. The terms “recommended dose” and “dose recommendation” may be used interchangeably herein.

FIG. 3B is a block diagram depicting an example embodiment of MDD 152 having electronics 160, coupled with a power supply 161 and an electric injection motor 162, which in turn is coupled with power supply 161 and a medication reservoir 163. Needle 156 is shown in fluid communication with reservoir 163, and a valve (not shown) may be present between reservoir 163 and needle 156. Reservoir 163 can be permanent or can be removable and replaced with another reservoir containing the same or different medication. Electronics 160 can be implemented in one or more semiconductor chips (e.g., an application specific integrated circuit (ASIC), processor or controller, memory, programmable gate array, and others). In the embodiment of FIG. 3B, electronics 160 can include high-level functional units, including processing circuitry 164, memory 165, communication circuitry 166 configured to communicate in wired and/or wireless fashion with one or more devices external to MDD 152 (such as display device 120), and user interface electronics 168.

MDD 152 can be implemented in a highly interconnected fashion, where power supply 161 is coupled with each component shown in FIG. 3B and where those components that communicate or receive data, information, or commands (e.g., processing circuitry 164, memory 165, and communication circuitry 166), can be communicatively coupled with every other such component over, for example, one or more communication connections or buses 169.

Processing circuitry 164 can include one or more processors, microprocessors, controllers, and/or microcontrollers, each of which can be a discrete chip or distributed amongst (and a portion of) a number of different chips. Processing circuitry 164 can include on-board memory. Processing circuitry 164 can interface with communication circuitry 166 and perform analog-to-digital conversions, encoding and decoding, digital signal processing and other functions that facilitate the conversion of data signals into a format (e.g., in-phase and quadrature) suitable for wireless or wired transmission. Processing circuitry 164 can also interface with communication circuitry 166 to perform the reverse functions necessary to receive a wireless transmission and convert it into digital data or information.

Processing circuitry 164 can execute software instructions stored in memory 165. These instructions can cause processing circuitry 164 to receive a selection or provision of a specified dose from a user (e.g., entered via user interface 158 or received from another device), process a command to deliver a specified dose (such as a signal from actuator 159), and control motor 162 to cause delivery of the specified dose. These instructions can also cause processing circuitry 164 to read and act on received transmissions, to process data or information received from other devices (e.g., calibration information, encryption or authentication information received from display device 120, and others), to perform tasks to establish and maintain communication with display device 120, to interpret voice commands from a user, to cause communication circuitry 166 to transmit, and others. In embodiments where MDD 152 includes user interface 158, then the instructions can cause processing circuitry 164 to control the user interface, read user input from the user interface (e.g., entry of a medication dose for administration or entry of confirmation of a recommended medication dose), cause the display of information on the user interface, format data for display, and others. The functions described here that are coded in the instructions can instead be implemented by MDD 152 with the use of a hardware or firmware design that does not rely on the execution of stored software instructions to accomplish the functions.

Memory 165 can be shared by one or more of the various functional units present within MDD 152, or can be distributed amongst two or more of them (e.g., as separate memories present within different chips). Memory 165 can also be a separate chip of its own. Memory 165 is non-transitory, and can be volatile (e.g., RAM, etc.) and/or non-volatile memory (e.g., ROM, flash memory, F-RAM, etc.).

Communication circuitry 166 can be implemented as one or more components (e.g., transmitter, receiver, transceiver, passive circuit, encoder, decoder, and/or other communication circuitry) that perform the functions for communications over the respective communications paths or links. Communication circuitry 166 can include or be coupled to one or more antenna for wireless communication. Details of exemplary antenna can be found in the '385 publication, which is hereby incorporated in its entirety for all purposes.

Power supply 161 can include one or more batteries, which can be rechargeable or single-use disposable batteries. Power management circuitry can also be included to regulate battery charging and monitor usage of power supply 161, boost power, perform DC conversions, and the like.

MDD 152 may also include an integrated or attachable in vitro glucose meter, including an in vitro test strip port (not shown) to receive an in vitro glucose test strip for performing in vitro blood glucose measurements.

### Example Embodiments of Display Devices

Display device 120 can be configured to display information pertaining to system 100 to the user and accept or receive input from the user also pertaining to system 100. Display device 120 can display recent measured analyte levels, in any number of forms, to the user. The display device can display historical analyte levels of the user as well as other metrics that describe the user's analyte information (e.g., time in range, ambulatory glucose profile (AGP), hypoglycemia risk levels, etc.). Display device 120 can display medication delivery information, such as historical dose information and the times and dates of administration. Display device 120 can display alarms, alerts, or other notifications pertaining to analyte levels and/or medication delivery.

Display device 120 can be dedicated for use with system 100 (e.g., an electronic device designed and manufactured for the primary purpose of interfacing with an analyte sensor and/or a medication delivery device), as well as devices that are multifunctional, general purpose computing devices such as a handheld or portable mobile communication device (e.g., a smartphone or tablet), or a laptop, personal computer, or other computing device. Display device 120 can be configured as a mobile smart wearable electronics assembly, such as a smart glass or smart glasses, or a smart watch or wristband. Display devices, and variations thereof, can be referred to as “reader devices,” “readers,” “handheld electronics” (or handhelds), “portable data processing” devices or units, “information receivers,” “receiver” devices or units (or simply receivers), “relay” devices or units, or “remote” devices or units, to name a few.

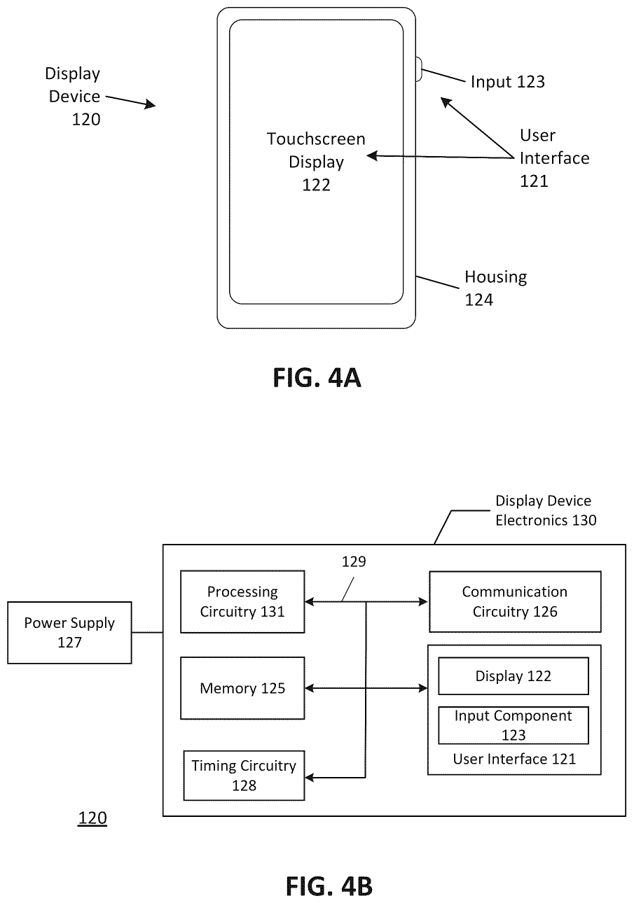

FIG. 4A is a schematic view depicting an example embodiment of display device 120. Here, display device 120 includes a user interface 121 and a housing 124 in which display device electronics 130 (FIG. 4B) are held. User interface 121 can be implemented as a single component (e.g., a touchscreen capable of input and output) or multiple components (e.g., a display and one or more devices configured to receive user input). In this embodiment, user interface 121 includes a touchscreen display 122 (configured to display information and graphics and accept user input by touch) and an input button 123, both of which are coupled with housing 124.

Display device 120 can have software stored thereon (e.g., by the manufacturer or downloaded by the user in the form of one or more “apps” or other software packages) that interface with SCD 102, MDD 152, and/or the user. In addition, or alternatively, the user interface can be affected by a web page displayed on a browser or other internet interfacing software executable on display device 120.

FIG. 4B is a block diagram of an example embodiment of a display device 120 with display device electronics 130. Here, display device 120 includes user interface 121 including display 122 and an input component 123 (e.g., a button, actuator, touch sensitive switch, capacitive switch, pressure sensitive switch, jog wheel, microphone, speaker, or the like), processing circuitry 131, memory 125, communication circuitry 126 configured to communicate to and/or from one or more other devices external to display device 120), a power supply 127, and timing circuitry 128 (e.g., such as an oscillator and phase locked loop for providing a clock or other timing to components of SCD 102). Each of the aforementioned components can be implemented as one or more different devices or can be combined into a multifunctional device (e.g., integration of processing circuitry 131, memory 125, and communication circuitry 126 on a single semiconductor chip). Display device 120 can be implemented in a highly interconnected fashion, where power supply 127 is coupled with each component shown in FIG. 4B and where those components that communicate or receive data, information, or commands (e.g., user interface 121, processing circuitry 131, memory 125, communication circuitry 126, and timing circuitry 128), can be communicatively coupled with every other such component over, for example, one or more communication connections or buses 129. FIG. 4B is an abbreviated representation of the typical hardware and functionality that resides within a display device and those of ordinary skill in the art will readily recognize that other hardware and functionality (e.g., codecs, drivers, glue logic) can also be included.

Processing circuitry 131 can include one or more processors, microprocessors, controllers, and/or microcontrollers, each of which can be a discrete chip or distributed amongst (and a portion of) a number of different chips. Processing circuitry 131 can include on-board memory. Processing circuitry 131 can interface with communication circuitry 126 and perform analog-to-digital conversions, encoding and decoding, digital signal processing and other functions that facilitate the conversion of data signals into a format (e.g., in-phase and quadrature) suitable for wireless or wired transmission. Processing circuitry 131 can also interface with communication circuitry 126 to perform the reverse functions necessary to receive a wireless transmission and convert it into digital data or information.

Processing circuitry 131 can execute software instructions stored in memory 125. These instructions can cause processing circuitry 131 to process raw analyte data (or pre-processed analyte data) and arrive at a corresponding analyte level suitable for display to the user. These instructions can cause processing circuitry 131 to read, process, and/or store a dose instruction from the user, and because the dose instruction to be communicated to MDD 152. These instructions can cause processing circuitry 131 to execute user interface software adapted to present an interactive group of graphical user interface screens to the user for the purposes of configuring system parameters (e.g., alarm thresholds, notification settings, display preferences, and the like), presenting current and historical analyte level information to the user, presenting current and historical medication delivery information to the user, collecting other non-analyte information from the user (e.g., information about meals consumed, activities performed, medication administered, and the like), and presenting notifications and alarms to the user. These instructions can also cause processing circuitry 131 to cause communication circuitry 126 to transmit, can cause processing circuitry 131 to read and act on received transmissions, to read input from user interface 121 (e.g., entry of a medication dose to be administered or confirmation of a recommended medication dose), to display data or information on user interface 121, to adjust the timing of timing circuitry 128, to process data or information received from other devices (e.g., analyte data, calibration information, encryption or authentication information received from SCD 102, and others), to perform tasks to establish and maintain communication with SCD 102, to interpret voice commands from a user, and others. The functions described here that are coded in the instructions can instead be implemented by display device 120 with the use of a hardware or firmware design that does not rely on the execution of stored software instructions to accomplish the functions.

Memory 125 can be shared by one or more of the various functional units present within display device 120, or can be distributed amongst two or more of them (e.g., as separate memories present within different chips). Memory 125 can also be a separate chip of its own. Memory 125 is non-transitory, and can be volatile (e.g., RAM, etc.) and/or non-volatile memory (e.g., ROM, flash memory, F-RAM, etc.).

Communication circuitry 126 can be implemented as one or more components (e.g., transmitter, receiver, transceiver, passive circuit, encoder, decoder, and/or other communication circuitry) that perform the functions for communications over the respective communications paths or links. Communication circuitry 126 can include or be coupled to one or more antenna for wireless communication.

Power supply 127 can include one or more batteries, which can be rechargeable or single-use disposable batteries. Power management circuitry can also be included to regulate battery charging and monitor usage of power supply 127, boost power, perform DC conversions, and the like.

Display device 120 can also include one or more data communication ports (not shown) for wired data communication with external devices such as computer system 170, SCD 102, or MDD 152. Display device 120 may also include an integrated or attachable in vitro glucose meter, including an in vitro test strip port (not shown) to receive an in vitro glucose test strip for performing in vitro blood glucose measurements.

Display device 120 can display the measured analyte data received from SCD 102 and can also be configured to output alarms, alert notifications, glucose values, etc., which may be visual, audible, tactile, or any combination thereof. In some embodiments, SCD 102 and/or MDD 152 can also be configured to output alarms, or alert notifications in visible, audible, tactile forms or combination thereof. Further details and other display embodiments can be found in, e.g., U.S. Patent Publ. No. 2011/0193704, which is incorporated herein by reference in its entirety for all purposes.

### Example Embodiments Related to Integrated Management

The following example embodiments relate to an integrated management system (IMS) that will, in many embodiments, be implemented as a set of software instructions stored and/or executed on one or more electronic devices. In some embodiments, the IMS is stored, executed, and presented to the user on the same single electronic device. In other embodiments, the IMS can be stored and executed on one device, and presented to the user on a different electronic device. For example, the IMS can be stored and executed on trusted computer system 180 and presented to the user by way of a webpage displayed through an internet browser executed on display device 120.

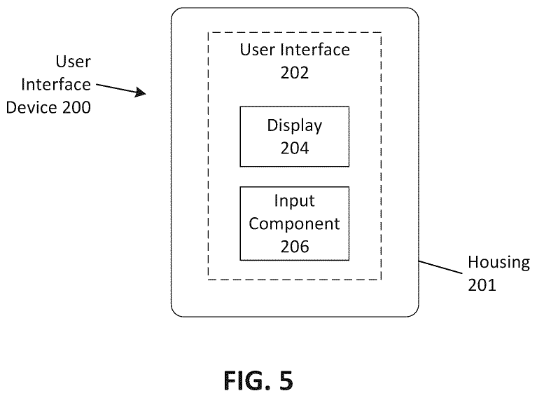

Thus, there are many different embodiments pertaining to the number and type of electronic devices that are used in storing, executing, and presenting the IMS or portions thereof to a user. With respect to presentation to the user, the device that is configured to implement this capability will be referred to herein as a user interface device (UID) 200. FIG. 5 is a block diagram depicting an example embodiment of UID 200. In this embodiment, UID 200 includes a housing 201 that is coupled with a user interface 202. The user interface 202 is capable of outputting information to the user and receiving input or information from the user. In some embodiments, the user interface 202 is a touchscreen. As shown here, the user interface 202 includes a display 204, that may be a touchscreen, and an input component 206 (e.g., a button, actuator, touch sensitive switch, capacitive switch, pressure sensitive switch, jog wheel, microphone, touch pad, soft keys, keyboard, or the like).

Many of the devices described herein can be implemented as UID 200. For example, display device 120 will, in many embodiments, be used as UID 200. In some embodiments, MDD 152 can be implemented as UID 200. In embodiments where SCD 102 includes a user interface, then SCD 102 can be implemented as UID 200. Computer system 170 can also be implemented as UID 200.

### Example Embodiments of the User Experience of the Integrated Management System

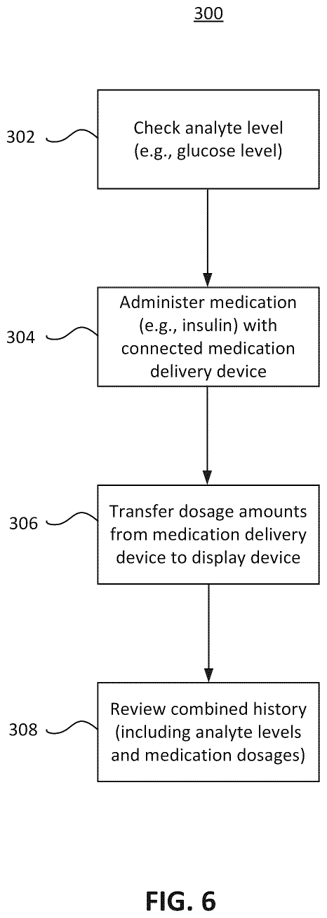

As seen in FIG. 6, in an exemplary method 300 of a user experience, a user can check an analyte level, such as a glucose level, in step 302. As described previously, a user may scan an SCD 102, or otherwise effect the transfer of an analyte level or data indicative of an analyte level to the display device 120. In step 304, the user administers the medication (e.g., insulin) from a connected medication delivery device 152. The medication delivery device 152 may be a smart, connected insulin pen, a connected pen cap, a smart button, or an automated insulin delivery (AID) device in a closed loop system, as described previously. In step 306, the user transfers the dosage information from the medication delivery device 152 to the display device 120. The transfer can be accomplished by methods known in the art. For example, the display device 120 (e.g., a smart phone) may scan the connected medication delivery device 152 and the information may be transferred via NFC. Alternatively, the dosage information may be transferred automatically without any further action required from the user. For instance, no scan may be necessary, and the dose information may be transferred automatically via BLUETOOTH® or another wireless communication protocol. For example, in the case of a connected pen cap, a connected insulin pen, or an AID device, the data may automatically transfer to the display device after the detection of an administered dose. The transfer of the medication information can be referred to in many ways, including scanning, transferring, downloading, uploading, exporting, importing, connecting, syncing, pairing, and other equivalent terminology. The data may also be automatically transferred via wireless communication such as BLUETOOTH®. The data being transferred can include data relating to medication (e.g., insulin) injections, shots, events, notes, log entries, or other equivalent terminology. The data may be referred to as insulin data, insulin records, insulin logs, insulin doses, or other equivalent terminology. After the dose information has been transferred, optionally, a notification may appear on the display device, e.g., a lockscreen or banner notification, which includes a message that a logbook or other report or application has been updated and may also include an amount of medication delivered and a delivery time. In step 308, the user and others (e.g., caretakers, HCPs) may then view the combined history, which may include the analyte levels and medication dosages, in a variety of displays and reports to visualize and assist the user in treating their diabetes.

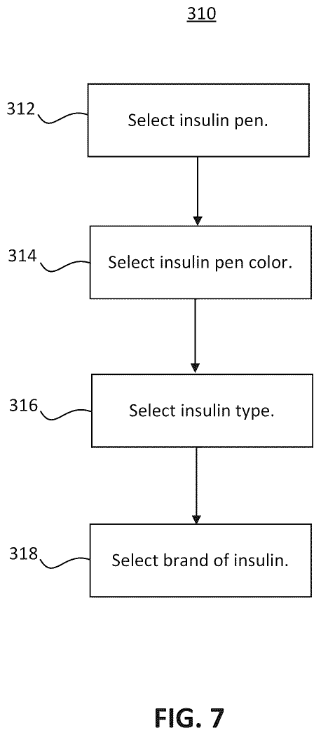

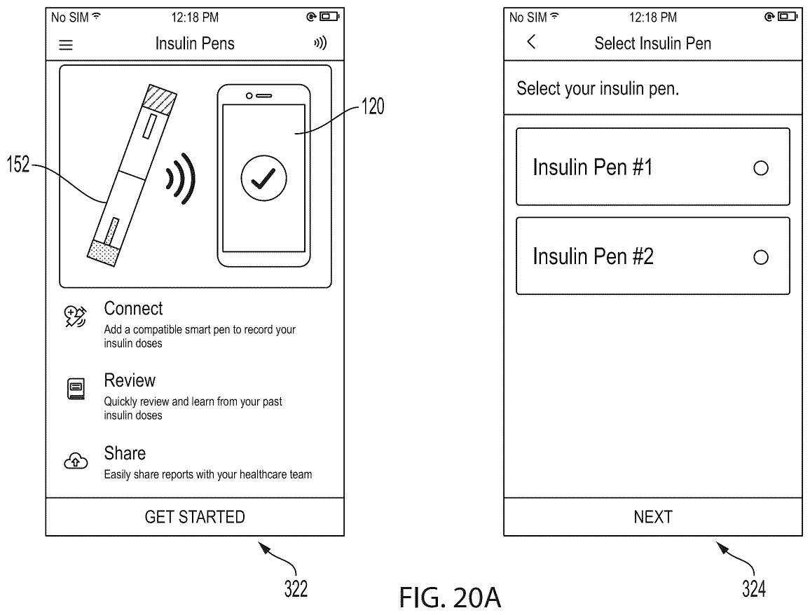

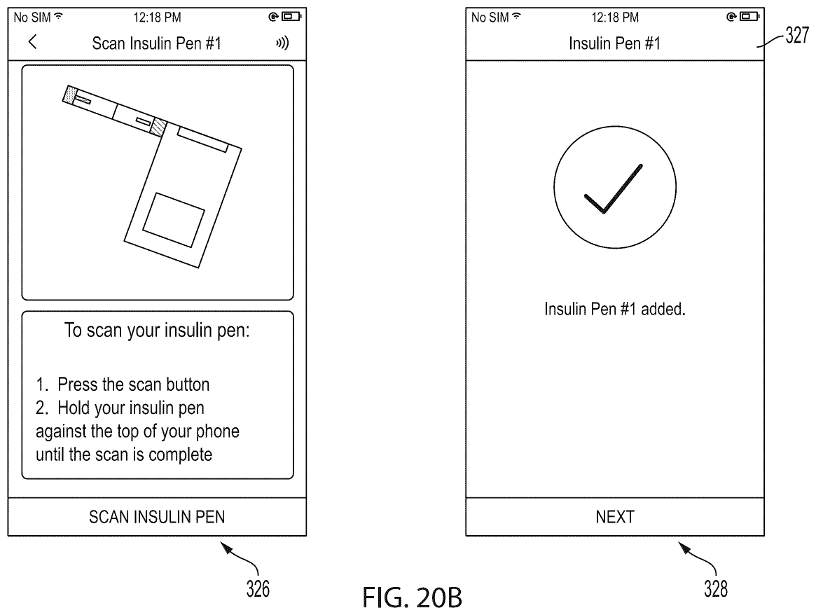

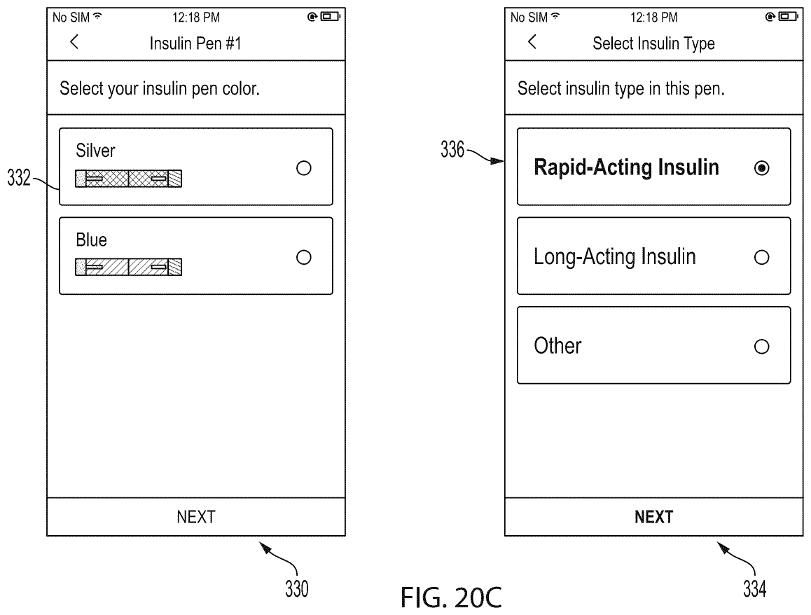

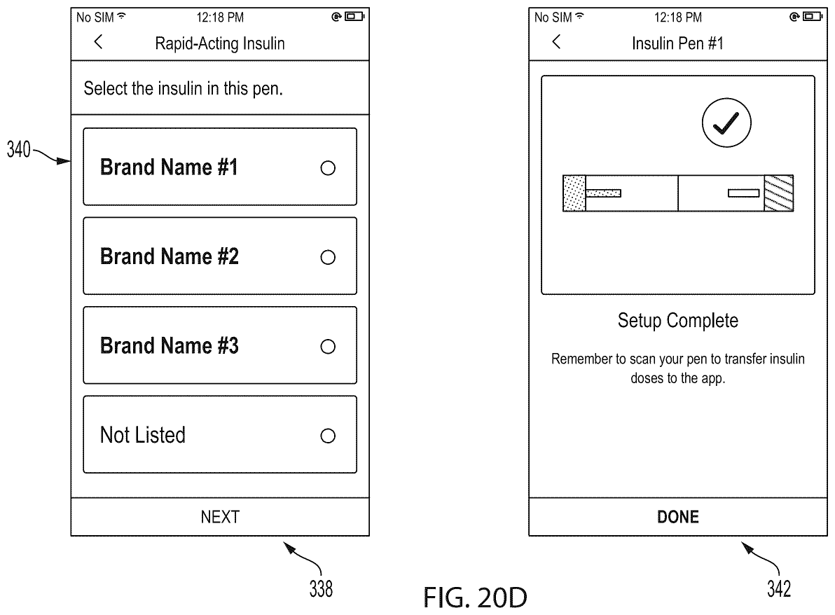

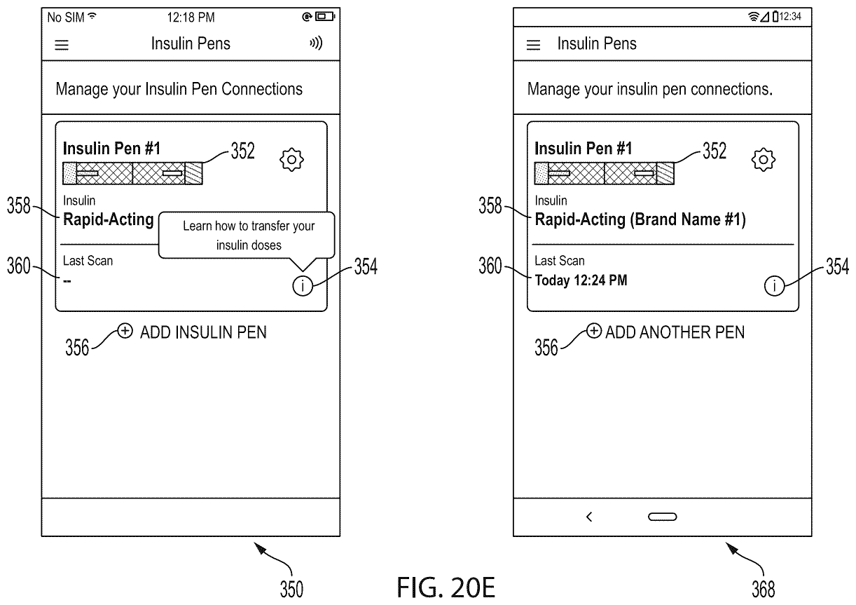

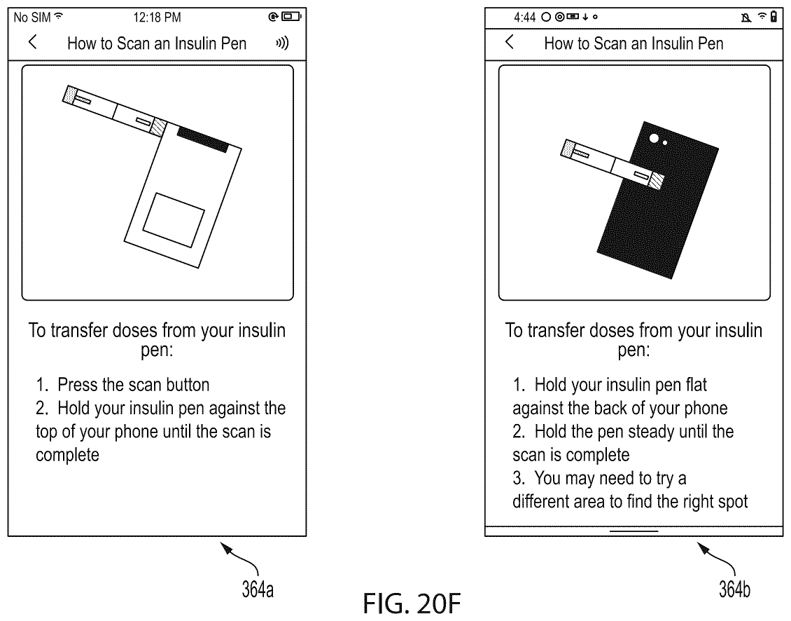

In order to be able to transfer data from the medication delivery device 152 to the IMS, the medication delivery device 152 must first be added to the IMS. FIG. 7 describes an exemplary method 320 for adding a medication delivery device 152 to the IMS and managing the connected medication delivery device 152. In method 310, a user can add an insulin pen by selecting “insulin pens” from a menu, such as a pull down menu. As seen in GUI 322 of FIG. a “Get Started” screen may contain a cartoon depiction of the medication delivery device 152 communicating with a display device 120, and also list different tasks or capabilities of the IMS after the medication delivery device 152 is connected to the IMS. These include connecting a compatible smart pen to record insulin doses, reviewing and learning from past doses, and Sharing reports with the user's healthcare team. After selecting “get started,” as seen in GUI 324 of FIG. 20A, the insulin pen settings may be opened, and the user may be prompted to select the medication delivery device 152 that they wish to connect in step 312. The medication delivery device 152, e.g., an insulin pen, may be listed by their brand names. After the user selects the appropriate medication delivery device and selects “next,” as seen in GUI 326 in FIG. 20B, a graphic or an animation may be presented that illustrates how to scan the medication delivery device 152 (e.g., insulin pen) with the display device 120 (e.g., smart phone). Additionally, GUI 326 may also contain text that instructs to scan by holding the insulin pen display flat against the back of the phone. The GUI 326 may also provide text that indicates that the user may need to move the insulin pen around slowly to find the correct spot to affect a connection/communication between the insulin pen and the phone. A link may also be provided to a user manual for the insulin pen that contains more information about how to connect the insulin pen to the phone. After a successful connection has been affected, a GUI 328 as seen in FIG. 20B may appear, which indicates that a new medication delivery device 152 has been added to the IMS. The name of the newly added medication delivery device may also appear on the GUI 328. After selecting “next,” in step 314, the user may be presented with a prompt to select a pen color for the newly connected medication delivery device 152. Selectable options with different colors 332 may appear on GUI 330, as seen in FIG. 20C. After selecting “next,” in GUI 334 in FIG. 20C, the user may be prompted to select the type of medication contained in the newly connected medication delivery device 152. Where the medication delivery device 152 is an insulin pen, in step 316, the user may be prompted to select between different types of insulin, e.g., rapid-acting insulin, long-acting insulin, or other, from a list 336. After selecting “next,” in step 318, the user may then be prompted to select the brand of the selected type of inulin in GUI 338 of FIG. 20D. A list 340 of available brands may be provided. Alternatively, the user may be able to add a new brand that is not included in the list provided. After selecting “next,” in GUI 342 of FIG. 20D, the user may see an indication that the setup of the new medication delivery device 152 is complete. The GUI 342 may also include a reminder to scan the connected medication delivery device 152 to transfer insulin doses to the software of the IMS. The GUI 342 may also include an animation that illustrates how to scan the medication delivery device 152 (e.g., insulin pen) with the display device 120 (e.g., smart phone). Additionally, the GUI may also contain text that instructs to scan by holding the insulin pen display flat against the back of the phone. The GUI may also provide text that indicates that the user may need to move the insulin pen around slowly to find the correct spot to affect a connection/communication between the insulin pen and the phone.

Additional medication delivery devices may be connected in the same manner described above. The additional medication delivery devices may be assigned a different color or other distinguishing feature or name from the previously connected medication delivery devices. For example, a medication delivery device 152 that delivers rapid acting insulin may be assigned a blue pen color, while a medication delivery device 152 that delivers long-acting insulin may be assigned a different color, e.g., red, silver, or black. Additional medication delivery devices may be added under the insulin pens menu, by selecting an option to add another medication delivery device, e.g., a plus symbol labeled “add another pen” (see 356 of FIG. 20E).

The user may select an option to “manage your own insulin pen connection” under the insulin pens menu. As seen in GUIs 350 and 368 of FIG. 20E, the user may be presented with a window depicting the connected medication delivery device 152, along with descriptions indicating the type and/or brand name of insulin delivered 358 by the medication delivery device 152, and details of the last scan 360, which may include the time of the scan and/or the amount of medication delivered. As described previously with respect to GUI 326, if a user selects an information i icon, a GUI 364a, b, as seen in FIG. 20F, may appear that includes information about how to transfer insulin logs and dosing information. The GUI 364a, b may include an animation of how to scan the medication delivery device 152 with the smart phone. Different GUIs may appear for instructing how to scan on an IOS system 364a and an Android system 364b. Additionally, the GUI 364a,b may also contain text that instructs to scan by holding the insulin pen display flat against the back of the phone. The GUI 364a,b may also provide text that indicates that the user may need to move the insulin pen around slowly to find the correct spot to result in a connection/communication between the insulin pen and the phone.

In some embodiments, the medication delivery device 152 may be a pen cap, such as Dialog®, that may transfer the dose data wirelessly via BLUETOOTH®. The medication delivery device 152 may need to be paired with the monitoring application using BLUETOOTH® rather than NFC. The monitoring application may remind the user to make sure that BLUETOOTH® is enabled on the display device 120. The monitoring application may display a GUI that prompts the user the enter and/or confirm a code on the medication delivery device 152. The GUI may also include a picture, diagram, or animation to show where the code is located on the medication delivery device 152. After the code is entered, the monitoring application may display a GUI that contains instructions to press and release a button on the medication delivery device 152 to pair the device with the display device 120. The instructions may include a textual description, diagram, picture, or animation to assist the user with the pairing. A window may appear on the display device 120 indicating that the medication delivery device 152 wants to pair with the display device 120. The user may need to select “Pair” before the pairing is completed. After the medication delivery device has been paired with the display device 120, a GUI that displays a confirmation that the pairing and/or setup is complete may be displayed.

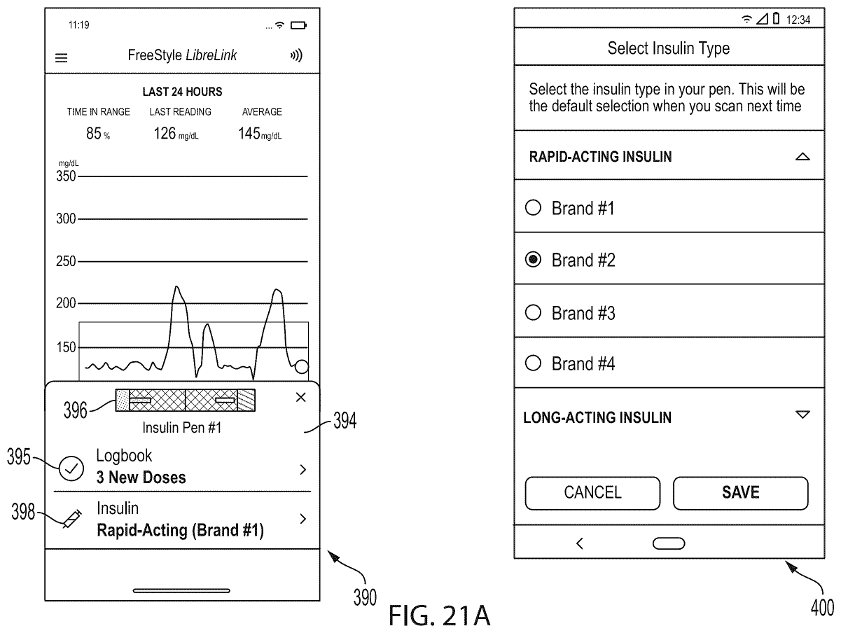

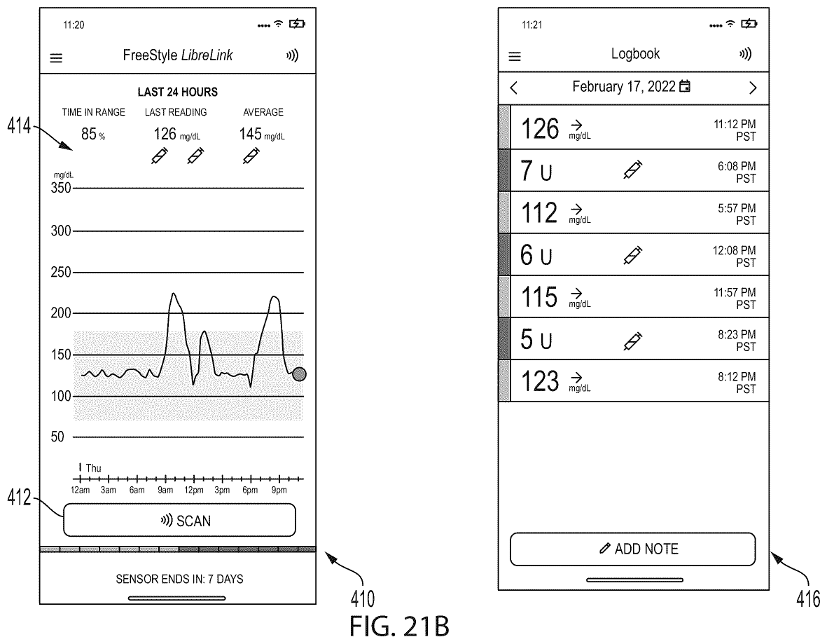

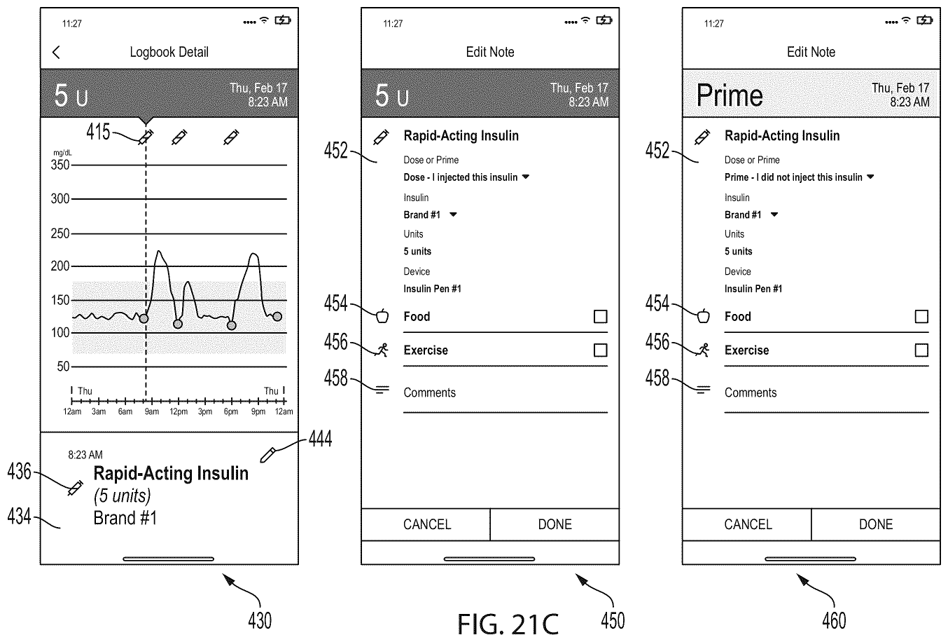

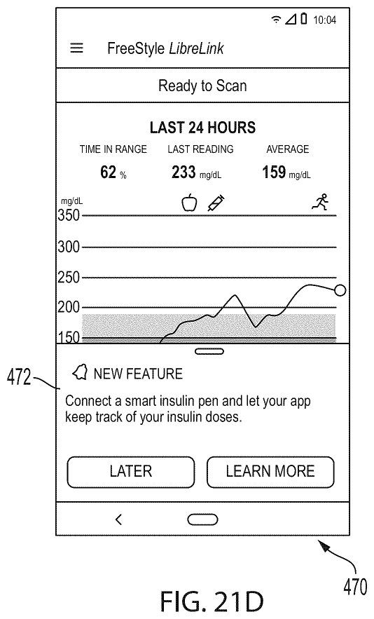

Once the medication delivery device 152 has been connected to the monitoring software on the display device 120, the dosing information may be downloaded or otherwise transferred (e.g., via NFC or BLUETOOTH®) from the medication delivery device 120 to the software. As seen in FIGS. 21A-21C, in GUI 390, the monitoring software may indicate that it can accept a download from a connected medication delivery device 152 (e.g., there may be a message indicating it is “ready to scan” in e.g., a banner). After the user has scanned the medication delivery device 152 in step 316, a window 394 may open indicating that a new dose(s) has appeared in a logbook. For example, the window 394 in GUI 390 indicates that 3 doses transferred from the medication delivery device to the monitoring software. The window 394 may also indicate which medication delivery device was scanned 396 and the type of insulin that was administered 398. Moreover, after the first time that the medication delivery device 152 is scanned, a GUI 400, as seen in FIG. 21A, may appear that allows the user to select a default selection for the insulin type, which will be used as the default the next time the user scans the insulin pen.

As seen in FIG. 21B, a home screen 410 may include a button or link 412 to select to scan a medication delivery device 152. The home screen 410 may also include an analyte profile, a time in range statistic, a last reading analyte level, an average analyte level, a graph of analyte readings for a time period (e.g., the current day), and an indication of the current sensor life. The home screen 410 may also include icons (e.g., syringe icons) 414 along the graph of the analyte readings that indicate when various medications were administered. The icons 414 may be positioned along the graph of analyte readings to correspond with/indicate the time that the medication was administered. In some embodiments, users may view and edit insulin notes by tapping the syringe icon 414 in the home screen 410. The user may tap the syringe icon to view the dose and then further tap a pencil icon to edit the entry, if needed. In some embodiments, a syringe icon with a question mark may mark a dose in which no additional comments or notes have been added. In some embodiments, a syringe icon with a notepad may indicate a dosage in which additional comments and notes have been added. In some embodiments, icons of syringes with no other annotations may mark a dose that was transferred automatically from a connected medication delivery device 152. Icons indicating food (e.g., an apple), exercise (e.g., a runner), or comments (e.g., a notepad) may also be indicated along or above the graph of the analyte reading, placed along the timeline (x-axis) according to when the food was consumed or the note was added.

A logbook GUI 416, as seen in FIG. 21B, may also be accessed from the insulin pen menu. The logbook GUI 416 may contain a listing of various events logged along with the time that the event occurred, including doses of medication administered. For the entries imported from the medication delivery device 152, a syringe icon may appear next to the amount of medication administered (e.g., in units). Instances in which the medication delivery device 152 was primed may also be listed in the logbook. Priming doses (or “prime”) refers to a medication (e.g., insulin) shot to clear air from the needle tip. Priming doses may also be referred to as an airshot, flow check, squirt, or other equivalent term. Notes that have been manually added may also be included in the logbook.

The monitoring application may allow a user to edit the notes that are associated with a particular medication dosing event. If the user wants to edit details about the insulin dose, the user may tap the line entry in the logbook GUI 416, which links to a logbook detail GUI 430, as seen in FIG. 21C. The analyte graph on the logbook detail GUI 430 may include a vertical line or other highlight to indicate the dose being viewed. If the user wants to edit the details about the selected insulin dose, the user may tap the edit (pencil) icon 444, which links to the Edit Note GUI 450, in which the user can edit the information associated with various fields. The Edit Note GUI 450 includes a banner that prominently indicates the dose amount and the date and time taken. It may also include various fields, such as, the type of dose (insulin administered or prime) 452, brand of insulin, number of units, name of the delivery device, food 454, exercise 456, and comments 458. The user may edit the type of insulin delivered by tapping the drop-down caret and selecting the insulin brand. The user may edit the dose from a therapy dose to a flow check (or prime) dose by tapping the drop-down caret icon to select the prime/flow check option. In some embodiments, after the user indicates that it is a prime dose, the banner at the top of the Edit Note screen will indicate that it is a prime dose, as seen in GUI 460 or FIG. 21C.

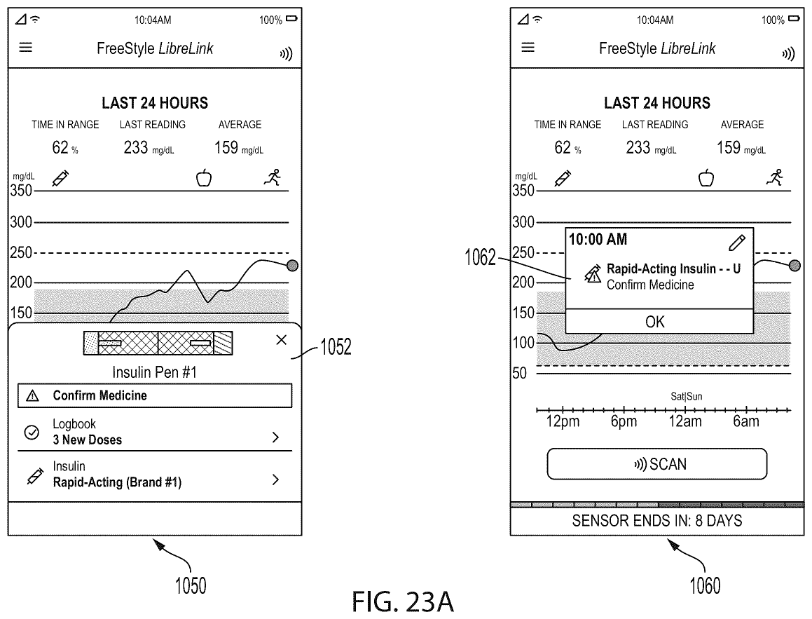

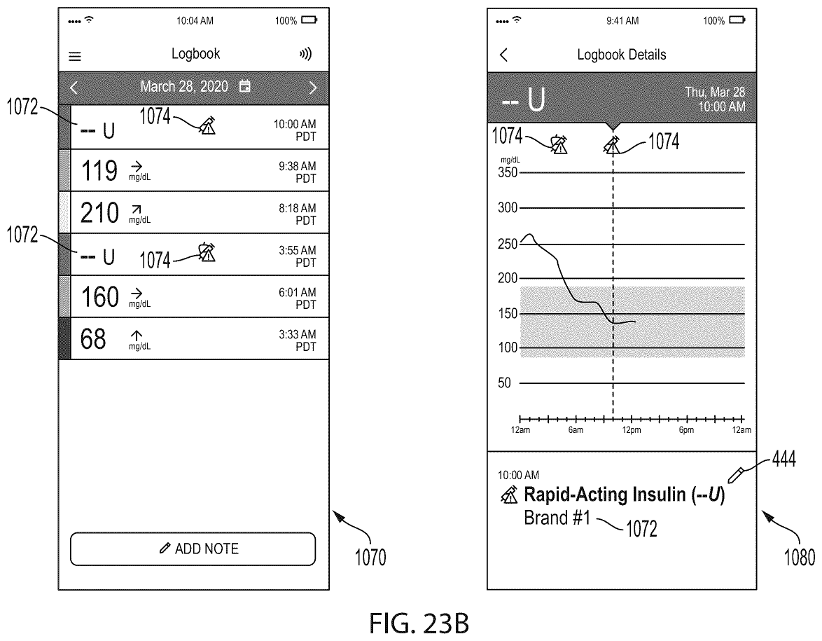

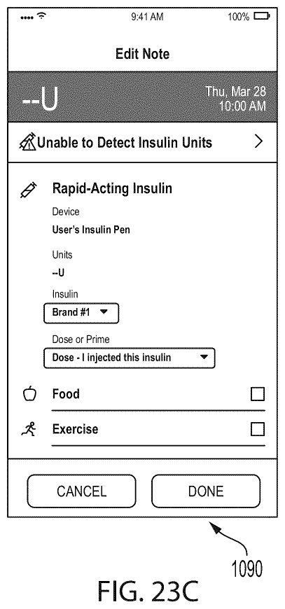

If there is an error, the monitoring application may request that the user confirm details of the dose administered. The details may include the dose amount, the type of medication, and the time it was delivered. As seen in FIGS. 23A-23C, window 1052 in GUI 1050 or window 1062 in GUI 1060 may appear to prompt the user to confirm details regarding the newly transferred dose(s) if an error was detected in the transferred dose data. Alternatively, errors may be noted in the Logbook GUI 1070 and/or Logbook Details GUI 1080. In some embodiments the Logbook GUI 1070 and/or Logbook Details GUI 1080 may include an error indicator associated with certain dosages. The error indicator may be a missing dosage amount 1072 and/or a different syringe icon (e.g., a syringe with an exclamation point or a question mark near it) 1074. The user can edit the dose entry with the error by tapping on the edit (pencil) icon 444, which can link to Edit Note GUI 1090 where individual fields can be edited as needed.

As seen in GUI 470 of FIG. 21D, the monitoring application may also prompt the user to learn more about a new feature of the monitoring application with a pop-up window 472. The prompt may inform the user of a new feature available by connecting a medication delivery device 152 (e.g., a smart insulin pen) and letting the application keep track of the user's insulin doses. A similar window may also pop up when an update is available for the monitoring application.

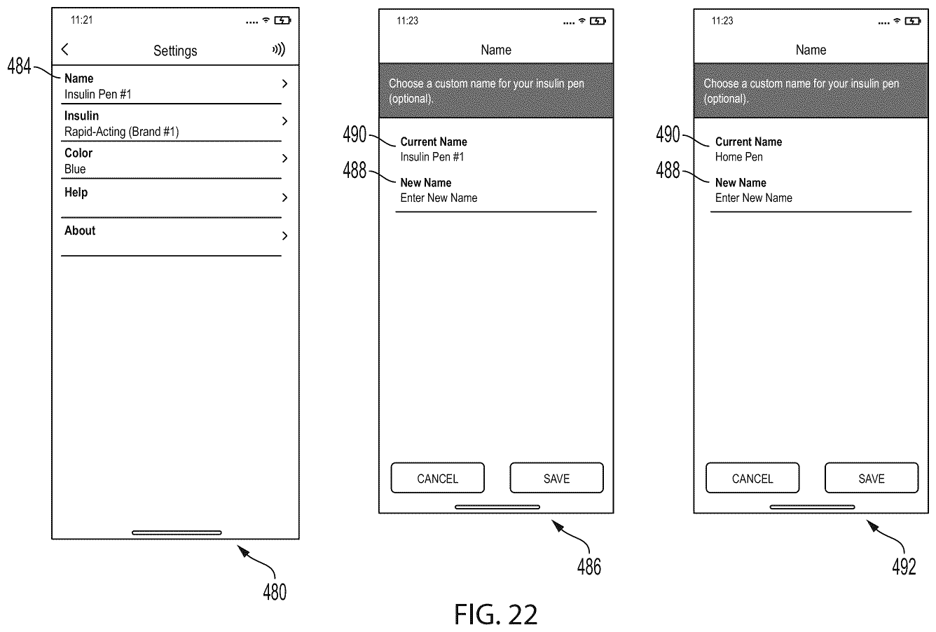

The monitoring application may also allow the user to customize the name of the connected medication delivery device 152. As seen in FIG. 22, the user may navigate to the insulin settings GUI 480, where the user may tap on or select the “name” field 482, which will open up the Name GUI window 486. The user may tap on or select the “new name” field 488 and proceed to type in the new chosen name for the device. In FIG. 22, the user entered “Home Pen” as their custom name for that device. After the user selects “save,” the new name will be displayed in the “current name” field 490 in updated GUI 492.

### Insights and Alerts

Missed Meal Dose Hints

The monitoring application may display an alert, hint, or prompt if a dose appears to have been missed. In some embodiments, the dose data is automatically transferred via BLUETOOTH® to the monitoring application. For example, based on glucose levels rising and no dose logged within a period of time, e.g., 1 hour, alternatively 2 hours, alternatively 3 hours, the monitoring application may display an alert that asks the user—“Did you miss a meal dose?” Furthermore, the alert may indicate that the user's glucose is rising and the last logged dose was X hours ago. The alert may also suggest that the user sync the medication delivery device 152 to update the dosing data. The alert may also suggest that, if the user did miss a dose, that the user should follow their HCP's advice on what to do. In some embodiments, where the alert relates to a specific meal, the alert may ask if the user missed their [breakfast/lunch/dinner] dose and then indicate that the user usually takes this dose before X:XX (e.g., the user usually takes their lunch dose before 1:30 pm).

The user may set the conditions under which the missed meal dose alerts or hints are given in order to minimize the number of alerts popping up on their device. For each of a breakfast, lunch, dinner, and long-acting dose, the user may specify that a missed dose hint should only be displayed after a certain time of day. For example, the user may set the breakfast dose hint to be displayed after 10:00 am, the lunch dose to be displayed after 1:30 pm, and the dinner dose to be displayed after 8:00 pm if no dose was detected during a period of time before the indicated time.

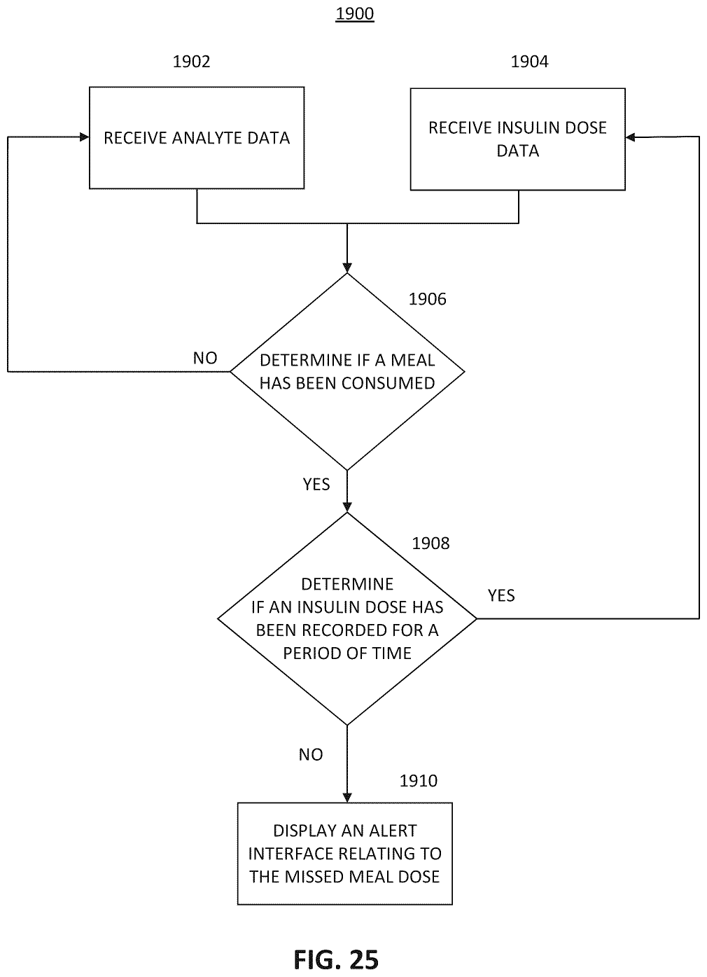

As seen in FIG. 25, in exemplary method 1900, beginning with step 1902, the system or application may receive analyte data. The data may be received from a server, the cloud, or from a sensor control device 102. In step 1904, the system may receive insulin data of the subject (e.g., from MDD 152). For example, the system or application can automatically receive the insulin data wirelessly, e.g., via BLUETOOTH®, from a connected pen, connected pen cap, or other medication delivery device 152, without requesting the data. In other embodiments, the system or application can check for the latest insulin dose data by requesting delivery information from different sources, including, but not limited to, the MDD 152, the MDD-associated application, or the interface that stores the latest insulin delivery information (such as the MDD application web server), or by checking the memory of the various applications for the latest insulin delivery information.

In step 1906, the system or application may determine if a meal has been consumed. This determination may be made by analyzing the received analyte data and determining if the analyte levels are rising above a high threshold or if a rate of change of the analyte level is greater than a minimum rate of change threshold. Meal dose detection is described in greater detail in U.S. Patent Publ. No. 2021/0030323, U.S. Patent Publ. No. 2021/0050085, and U.S. application Ser. No. 17/591,229, all of which are hereby expressly incorporated by reference in their entirety for all purposes. The high threshold analyte level may be 175 mg/dL, alternatively 180 mg/dL, alternatively 180 mg/dL, alternatively 190 mg/dL, alternatively 200 mg/dL, alternatively 210 mg/dL, alternatively 220 mg/dL, alternatively 230 mg/dL, alternatively 240 mg/dL, alternatively 250 mg/dL, alternatively 260 mg/dL, alternatively 270 mg/dL. The high threshold analyte level may be set by the user. If the system determines that a meal has not been consumed, then the system returns to step 1902 and receives additional analyte data.

If the system or application determines that a meal has been consumed, in step 1908, the system may analyze the insulin dose data received to determine if an insulin dose has been recorded or received for a period of time. The period of time may be at least 1 hour, for example about 1 hour, alternatively about 2 hours, alternatively about 3 hours. The period of time may optionally be set by the user instead of a default time set by the system. If an insulin dose has been recorded in the period of time, then the system determines that there has not been a missed dose and the system returns to step 1904 and receives additional insulin dose data.

In some embodiments, the period of time may be set for each meal by setting a time at which the meal dose should have been taken by the user. The system may also check to see if an insulin dose has been recorded or received before a set time for each meal. For example, if a breakfast dose was not recorded or received by 10:30 am, the system may determine that the user had missed their breakfast dose. Similarly, a lunchtime dose may have a time of 2:30 pm if the user will normally have taken a lunch dose by then. And a dinner dose may have a time of 7:30 pm if the user will normally have taken a dinner dose by that time. These times may be set by the user to align with their personal eating habits.

If the system or application determines that an insulin dose has not been recorded or received for the time period or by a set time for a particular meal, in step 1910, the system or application may display an alert interface relating to the missed meal dose. In some embodiments, the text of the alert interface may be customizable by the user.

Correction Dose Hints

The monitoring application may display an alert, hint, or prompt to the user to consider a correction dose if a period of time has passed since their last insulin dose and their glucose levels are still elevated. In some embodiments, the dose data is automatically transferred via BLUETOOTH® to the monitoring application. The user may set the conditions under which the correction dose alerts or hints are given in order to minimize the number of alerts popping up on their device. The user may configure the alerts to only be displayed when their glucose levels are above a high threshold, e.g., above about 250 mg/dL, and the period of time since the last insulin dose is longer than a minimum time threshold, e.g., about 2 hours.

The user may also customize the message that appears in the hint. For instance, the message may indicate that the dose they took X hours (e.g., 2 hours) ago isn't bringing down their glucose enough and that now is a good time to take an additional X units of insulin or take a walk around the park. A default message may be to follow their HCP's recommendations for correcting high glucose with treatment or exercise.

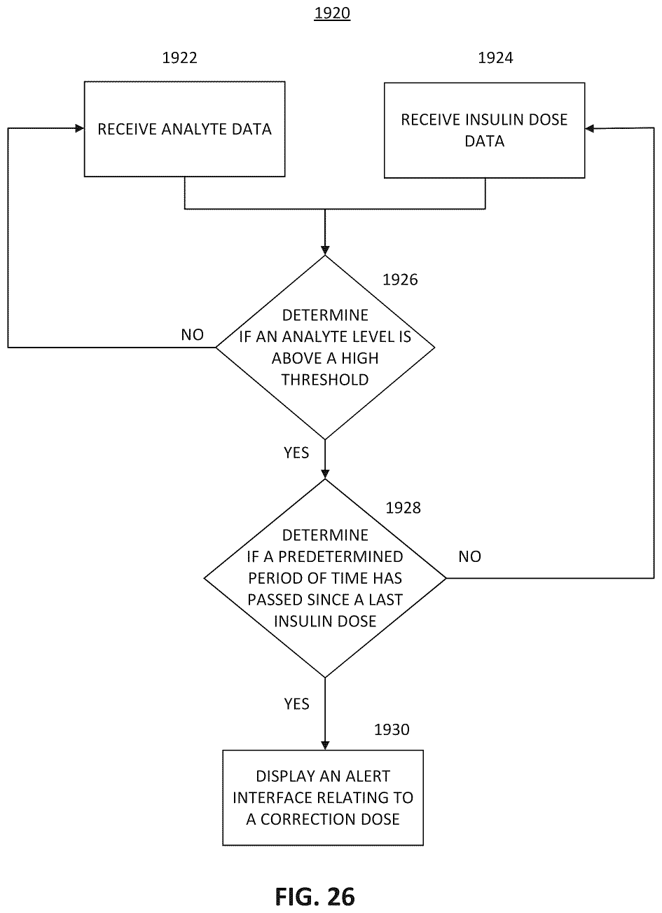

As seen in FIG. 26, in exemplary method 1920, beginning with step 1922, the system or application may receive analyte data. The data may be received from a server, the cloud, or from a sensor control device 102. In step 1924, the system or application may receive insulin data of the subject (e.g., from MDD 152). For example, the system or application can automatically receive the insulin data wirelessly, e.g., via BLUETOOTH®, from a connected pen, connected pen cap, or other medication delivery device 152, without requesting the data. In other embodiments, the system or application can check for the latest insulin dose data by requesting delivery information from different sources, including, but not limited to, the MDD 152, the MDD-associated application, or the interface that stores the latest insulin delivery information (such as the MDD application web server), or by checking the memory of the various applications for the latest insulin delivery information.

In step 1926, the system or application may determine if a received analyte level is above a high threshold, which may indicate that the user's glucose is outside of a target range. The high threshold analyte level may be at least 175 mg/dL, for example the high threshold analyte level may be 175 mg/dL, alternatively 180 mg/dL, alternatively 180 mg/dL, alternatively 190 mg/dL, alternatively 200 mg/dL, alternatively 210 mg/dL, alternatively 220 mg/dL, alternatively 230 mg/dL, alternatively 240 mg/dL, alternatively 250 mg/dL, alternatively 260 mg/dL, alternatively 270 mg/dL. The high threshold analyte level may be set by the user. If the system determines that an analyte level is not above a high threshold, then the system returns to step 1922 and receives additional analyte data.

If the system or application determines that an analyte level is above a high threshold, in step 1928, the system or application may analyze the insulin dose data received to determine if a period of time has passed since an insulin dose has been recorded or received. The period of time may be at least 2 hours, for example the period of time may be about 2 hours, alternatively about 2.5 hours, alternatively about 3 hours. The period of time may optionally be set by the user instead of a default time set by the system. If the period of time since the last insulin dose has been recorded has not passed yet, then the system returns to step 1924 and receives additional insulin dose data.

If the system or application determines that the period of time has passed since a last insulin dose, in step 1930, the system or application may display an alert interface relating to a correction dose. In some embodiments, the text of the alert interface may be customizable by the user.

Congratulatory Message

The monitoring application may also display congratulatory alerts or messages when an insulin dose resulted in glucose levels going back in a target range within a designated time period. For example, the alert or window may say “You got a glucose win! You got back in range 2 hours after your last insulin dose.” The user may have the option to add a note (either by typing or talk to text) with details about what they did that may have helped them reach their target range.

The user may set the conditions under which the alert or window is displayed by setting a maximum glucose level and a time limit to return to the target range. For example, the user may set the target range as under 180 mg/dL and the time after the dose to get to the target as 2 hours.

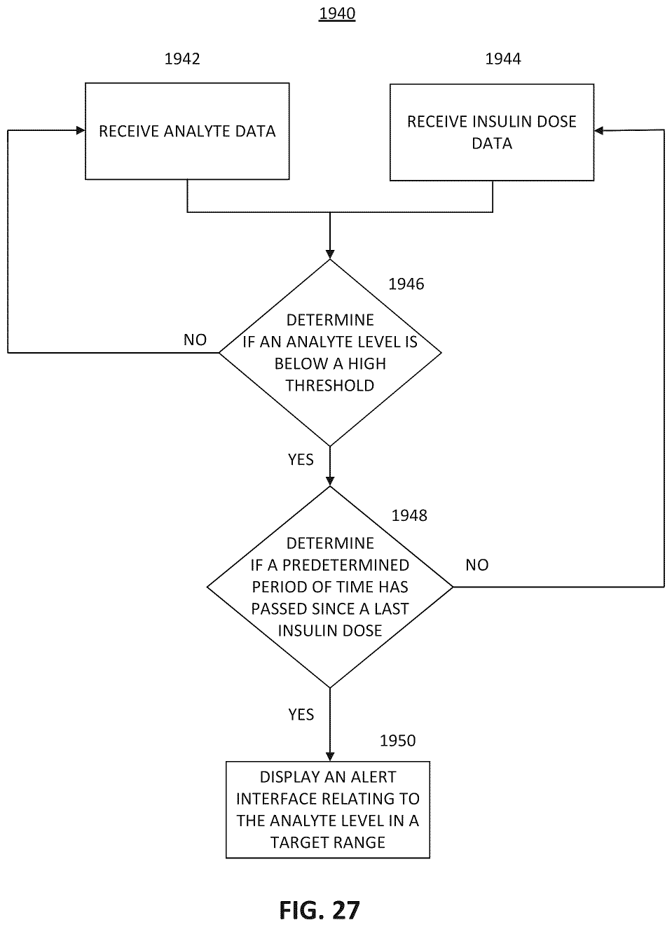

As seen in FIG. 27, in exemplary method 1940, beginning with step 1942, the system or application may receive analyte data. The data may be received from a server, the cloud, or from a sensor control device 102. In step 1944, the system may receive insulin data of the subject (e.g., from MDD 152). For example, the system can automatically receive the insulin data wirelessly, e.g., via BLUETOOTH®, from a connected pen, connected pen cap, or other medication delivery device 152, without requesting the data. In other embodiments, the system or application can check for the latest insulin dose data by requesting delivery information from different sources, including, but not limited to, the MDD 152, the MDD-associated application, or the interface that stores the latest insulin delivery information (such as the MDD application web server), or by checking the memory of the various applications for the latest insulin delivery information.

In step 1946, the system or application may determine if a received analyte level is below a high threshold, which may indicate that the user's glucose is inside a target range. The high threshold analyte level may be 190 mg/dL, alternatively 185 mg/dL, alternatively 180 mg/dL, alternatively 175 mg/dL, alternatively 170 mg/dL. The high threshold analyte level may be set by the user. If the system or application determines that an analyte level is not below a high threshold, then the system or application may return to step 1942 and receives additional analyte data.

If the system determines that an analyte level is below a high threshold, in step 1948, the system may analyze the insulin dose data received to determine if a period of time has passed since an insulin dose has been recorded or received. The period of time may be at least 2 hours, for example about 2 hours, alternatively about 2.5 hours, alternatively about 3 hours. The period of time may optionally be set by the user instead of a default time set by the system. If the period of time since the last insulin dose has been recorded has not passed yet, then the system returns to step 1944 and receives additional insulin dose data.

If the system determines that the period of time has passed since a last insulin dose, in step 1950, the system may display an alert interface relating to the analyte level being within a target range. In some embodiments, the text of the alert interface may be customizable by the user.

### Reports

The monitoring application may transfer data, including analyte levels from the SCD 102 and dosing data/logs from the medication delivery device 152, to a reporting application. The reporting application may be able to generate a plurality of reports that summarize and highlight various aspects of the analyte and medication data and history. The reporting application may be run on the display device or on a separate computing device. In some embodiments, the monitoring application may include instructions that, when executed by one or more processors, generates and display reports that include the insulin data in the monitoring application.

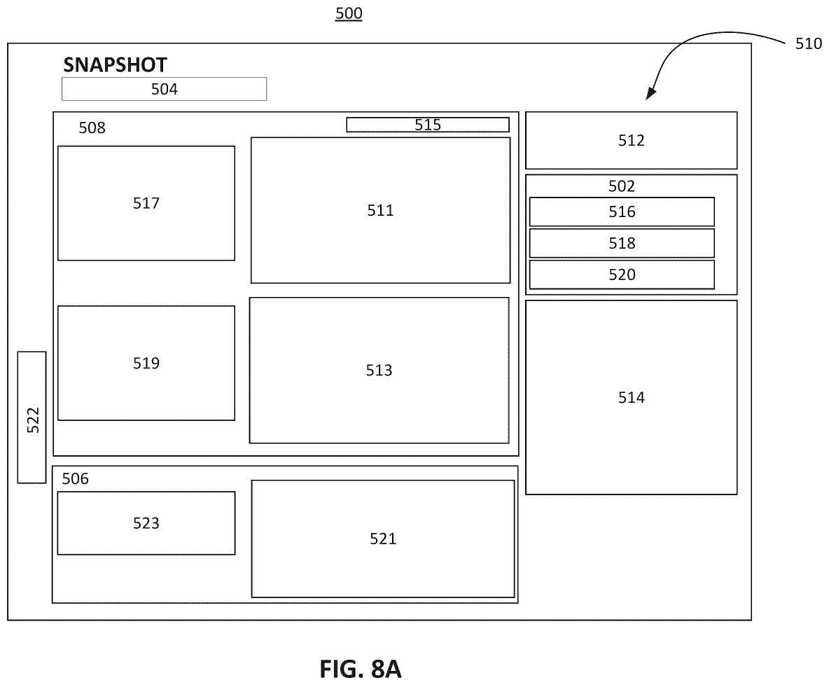

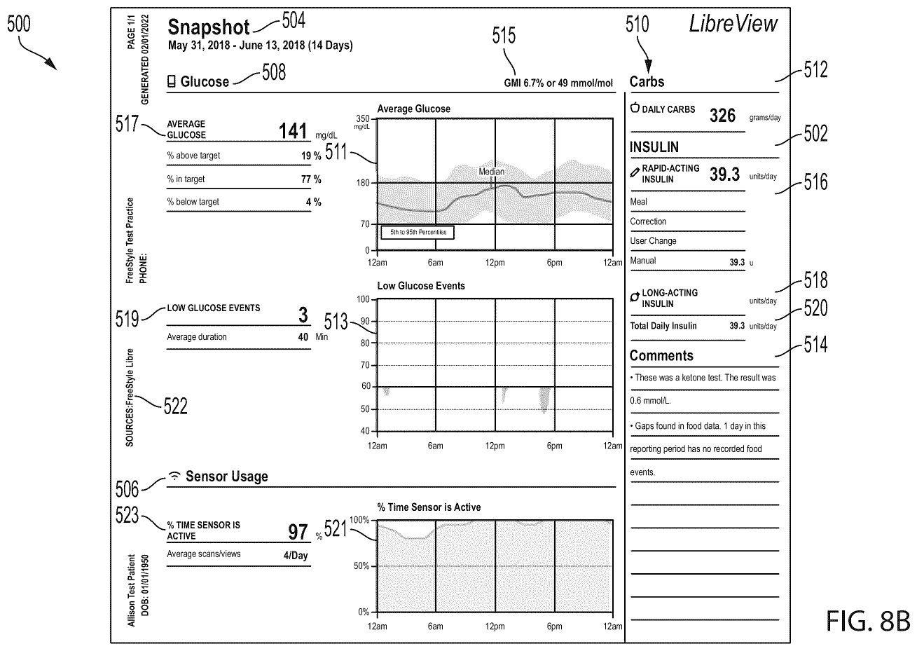

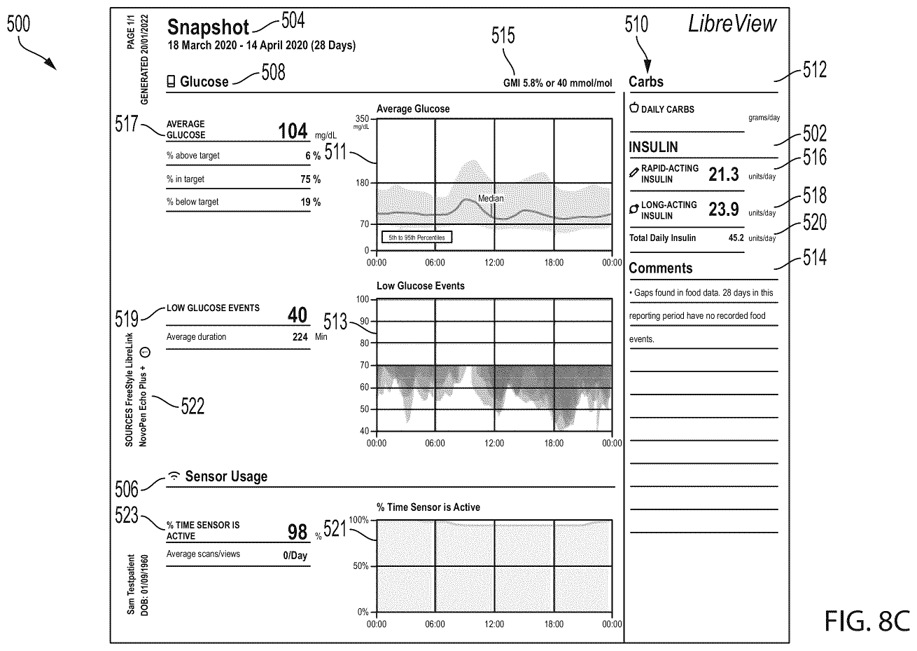

FIGS. 8A-8C depict example embodiments of an insulin dosage interface 502, as part of analyte monitoring system report GUI 500. According to one aspect of the embodiments, GUI 500 is a snapshot report covering a predetermined time period 504 (e.g., 14 days), and comprising a plurality of report portions on a single report GUI, including: a sensor usage interface portion 506, a glucose trend interface 508, which can include an AGP graph 511, a low glucose events graph 513, and other related glucose metrics (e.g., Glucose Management Indicator 515); a health information interface 510.

The AGP graph 511 may display the hourly 5^{th}, 25^{th}, 50^{th} (median), 75^{th}, and 95^{th} percentiles of glucose readings, presented over the “typical” day based on all days within the selected timeframe. The AGP graph 511 may also include two horizontal lines, which indicate the boundaries of the target range defined in the glucose statistics and targets portion and the Time-in-Ranges portion. The report 500 may also include a section 517 that lists metrics and/or statistics related to the average glucose. Metrics section 517 may list the average glucose, the percent of time above target range, the percent of time in a target range, and the percent of time below a target range. The low glucose events graph 513 may include a graph of the events in which the subject's glucose levels dropped below a low threshold, e.g., 72 mg/dL. The graph may show the glucose concentration (mg/dL) vs. time, such that the times of the low event episodes during the day are readily apparent. The report 500 may also include a section 519 that lists metrics and/or statistics related to the low glucose events. The low glucose events metrics section 519 may list the number of low glucose events and the average duration of the low glucose events.

The sensor usage interface 506 may include a Percentage Time Sensor Active graph 521 and a metrics section 523. The metric section 523 may include a percentage that the time sensor has been active and an Average Scans/Views metric (e.g., indicative of an average sum of a number of scans and a number of views). The Percentage Time Sensor Active graph 521 may include a graph of the percent of time that the sensor is active vs. a time of day (e.g., from 12 am to 12 am). An axis of the Percentage Time Sensor Active graph 521 may be aligned with a corresponding axis of one or more other graphs (e.g., average glucose trend graph 511, low glucose events graph 513), such that the user can visually correlate data between multiple graphs from two or more portions of the report GUI by the common units (e.g., time of day) from the aligned axes.

The health information interface 510 may include a carbohydrates section 512, insulin administered section 502, and a comments section 514. The carbohydrates section 512 may include information logged by the user about the user's average daily carbohydrate intake and may include the average total number of carbohydrates consumed by the user during the time period 504.

The insulin administered section 502 may include a list of medication dosages (e.g., insulin dosages) that were administered during the time period 504. The list may include separate entries for rapid acting insulin 516, long acting insulin 518, and a total daily amount of insulin 520. The entries may include an icon indicating of the insulin type, and/or the type of insulin (e.g., rapid acting, long acting, basal, etc.), and/or the brand name of the insulin, and/or the average amount of insulin administered. Where multiple insulin pens are connected, the medication dosages portion of the display 502 may include different icons for the different types of insulin pens, e.g., different colors and/or icons. For example, a rapid-acting pen may have a light green icon 516 while a long-acting insulin pen may have a dark green icon 518.

In some embodiments, the insulin dose amounts may have been manually entered by the user and/or determined by a dosage calculator, rather than transferred directly from a connected medication delivery device 152. The insulin dose amounts may have been entered into either the monitoring application or the reporting application. Where the insulin dose amounts were manually entered, the report 500 may note the amount of rapid-acting insulin 516 and long-acting insulin 518 delivered, but may not list the brand names of the insulins. The units/day that were administered for each type of insulin and the average total daily insulin (units/day) may also be displayed in the insulin dosage interface 502. For the rapid-acting insulin dose 516, the dose may be listed as the total dose (in units/day) that was administered, but may also be broken down into the different components, which may include a meal amount, a correction amount, a user change, and a manual input.

The comments interface 514 may can include additional information about the user's analyte and medication patterns presented in a narrative format. For example, the comments section 514 may include an indication of the trend of the number of tests per day as compared to a previous reporting period, detected fluctuations in the reporting period, and a ratio of the correction insulin of the average daily dose.

The report 500 may also include a listing of the sources 522 of the information provided. The sources may include the name of the glucose monitoring device, and also include the name of the insulin delivery device (e.g., brand name). If more than one insulin delivery device is being used by the patient, the sources section 525 may list all of the medication delivery devices or may contain the name of the first connected pen and the number (numerical value) of the additional medical device(s), but may not include the names of any specific insulin pens.

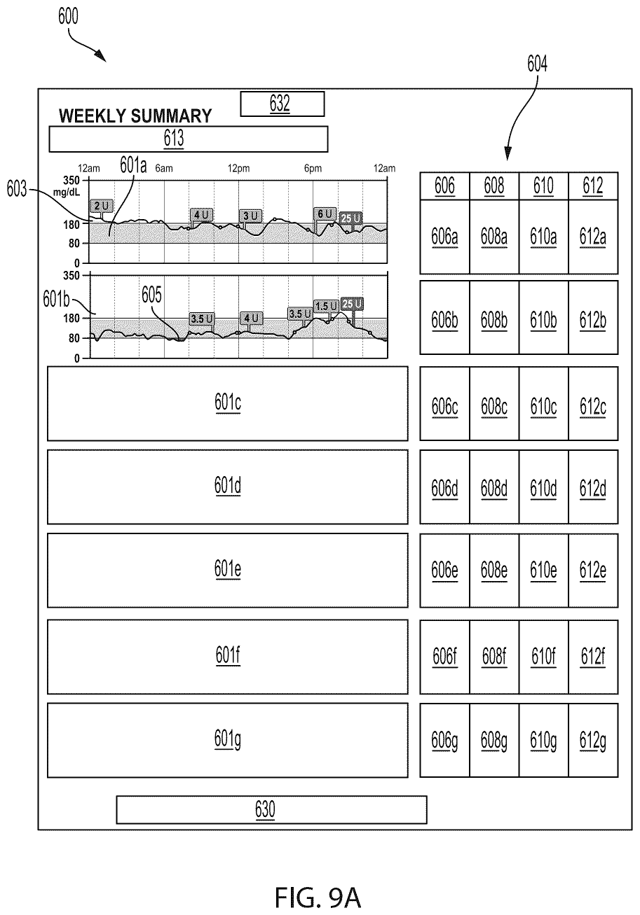

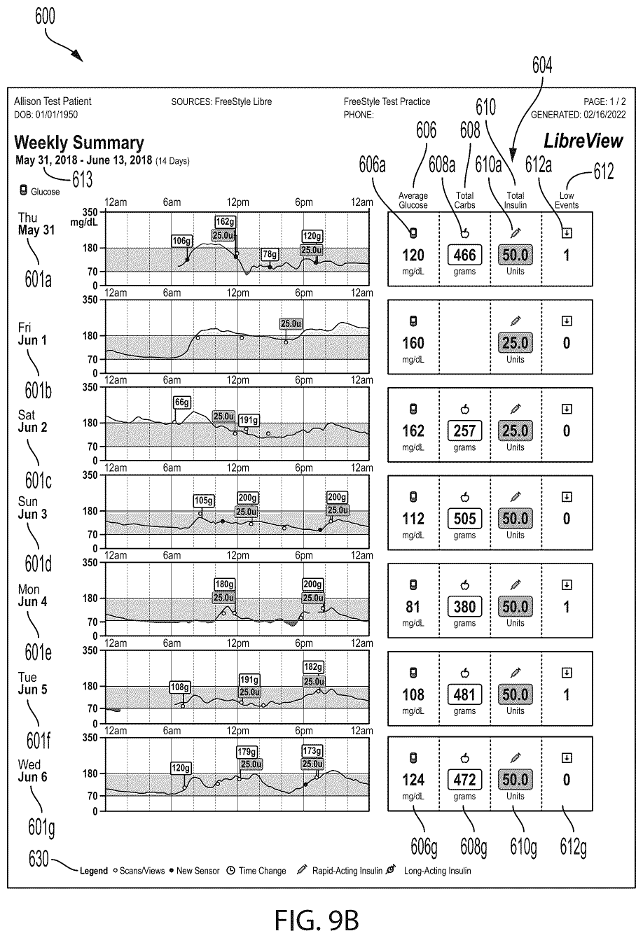

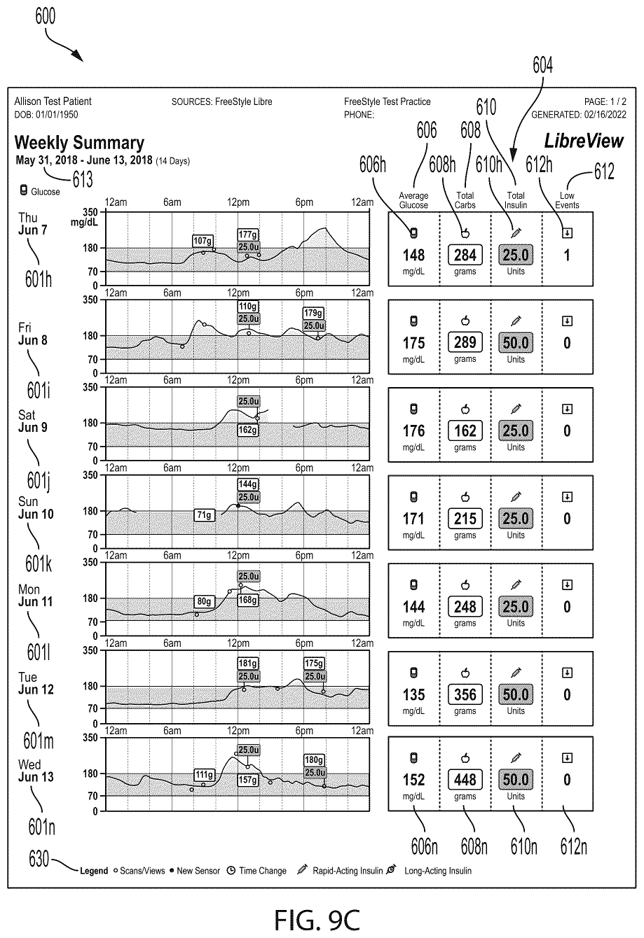

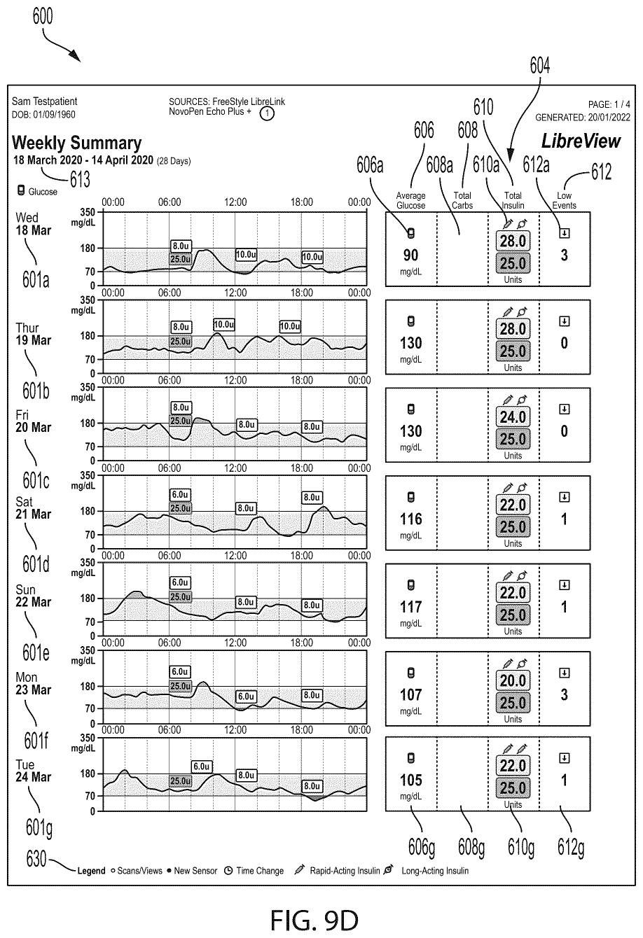

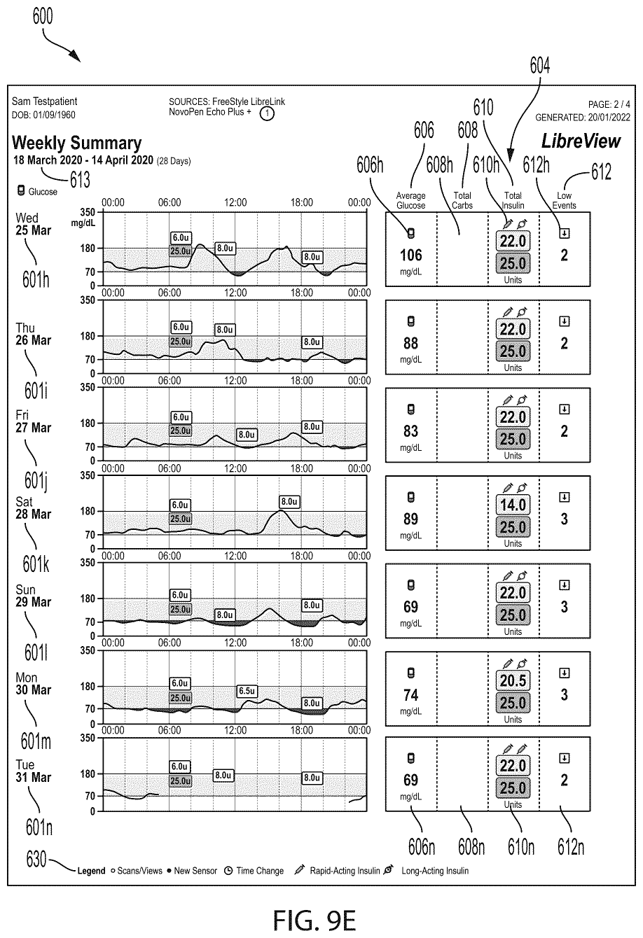

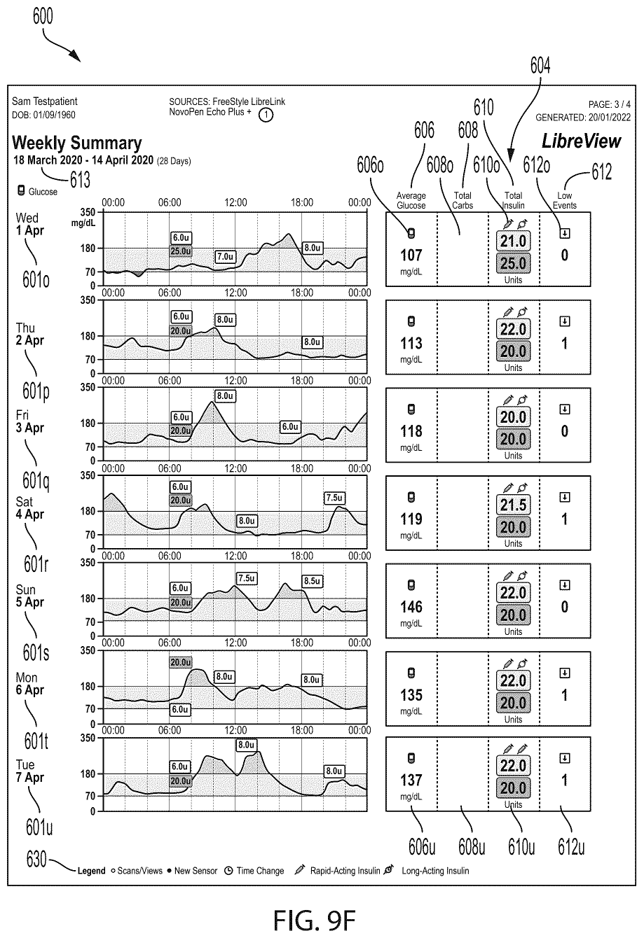

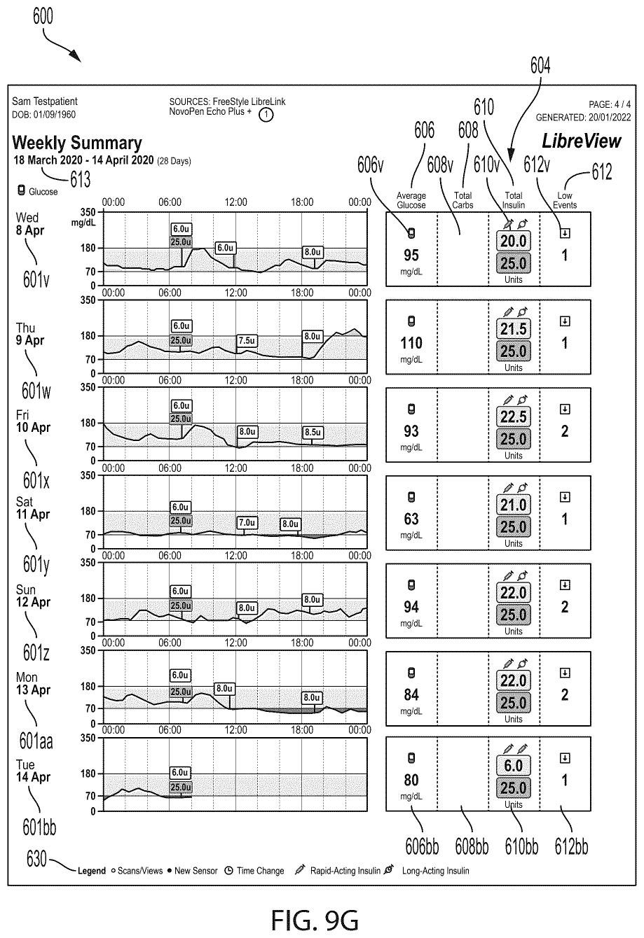

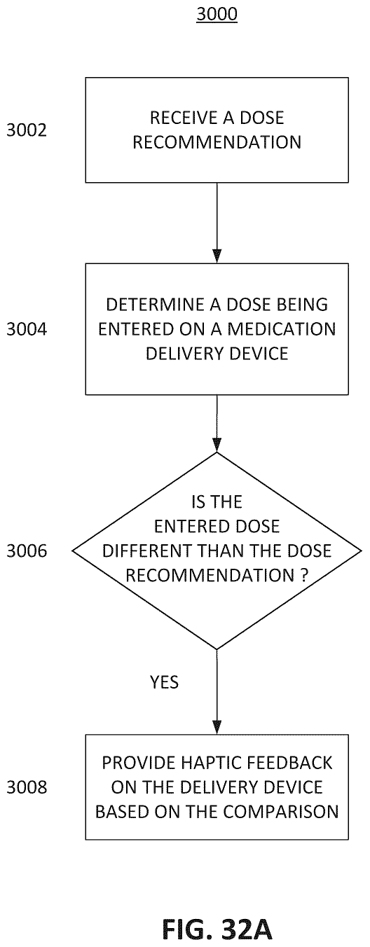

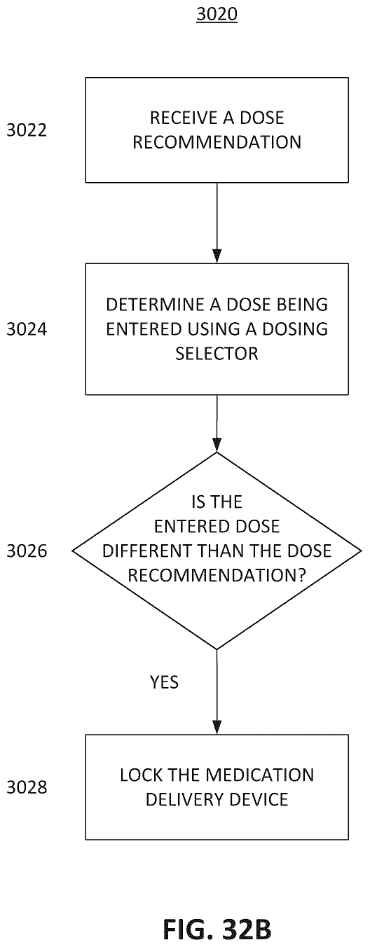

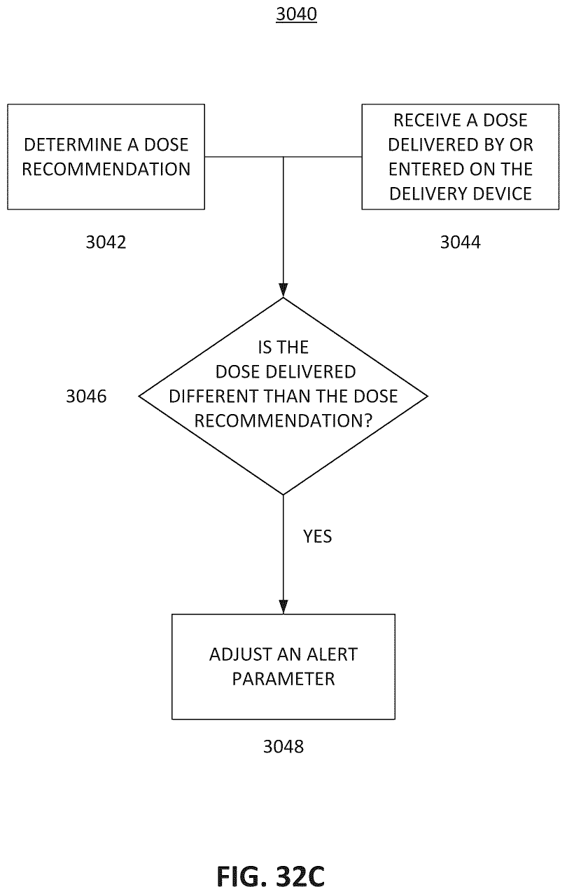

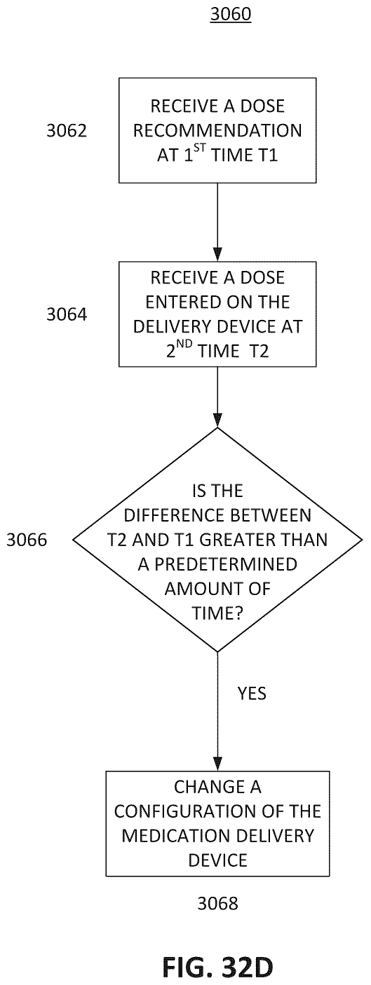

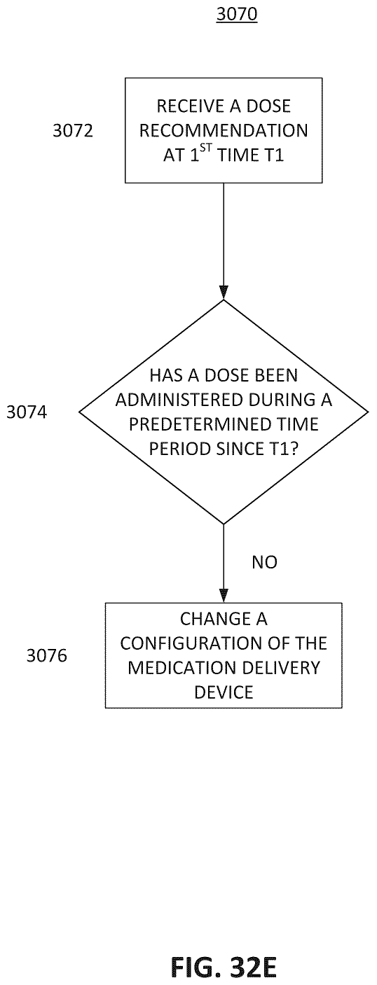

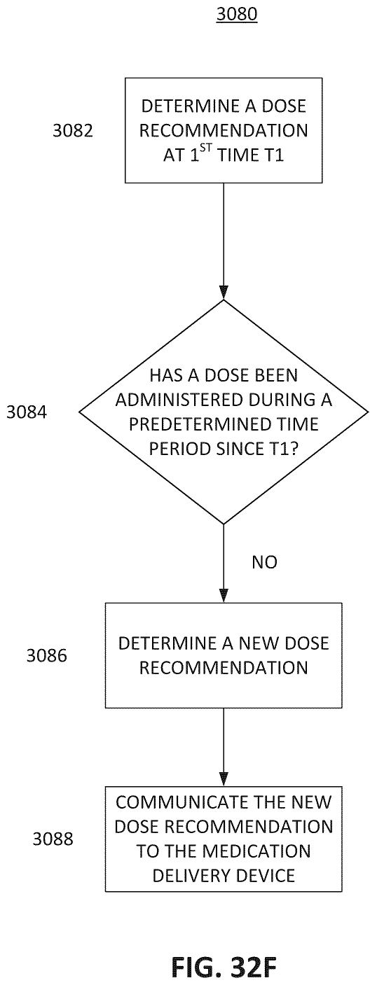

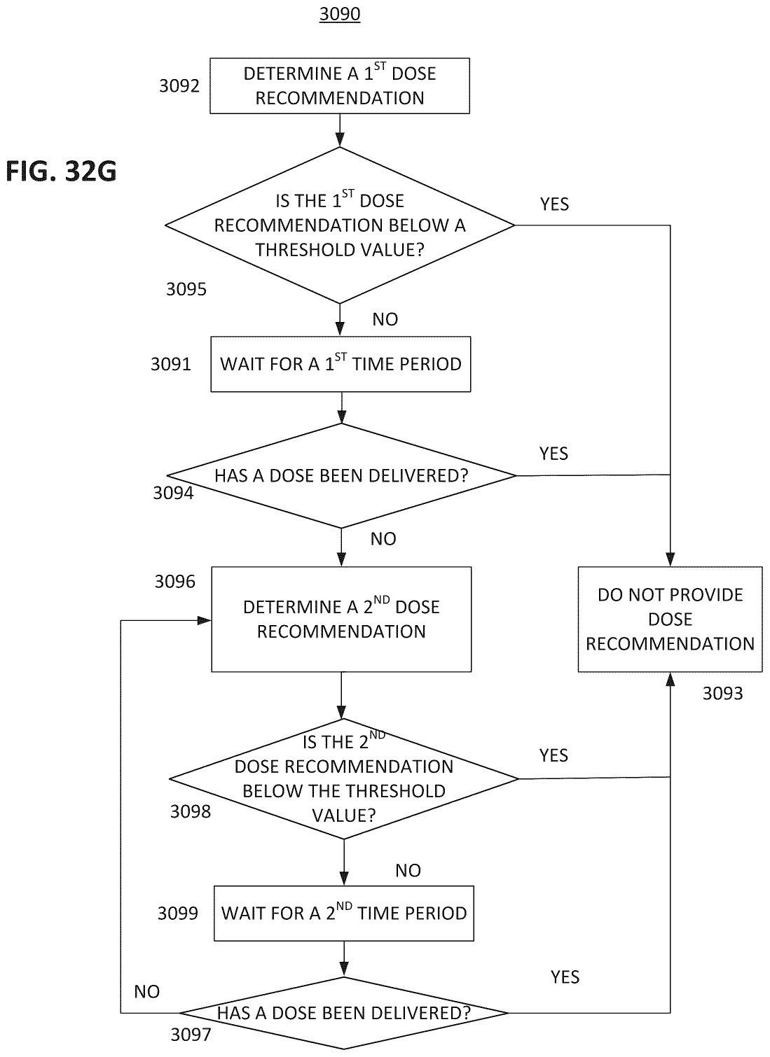

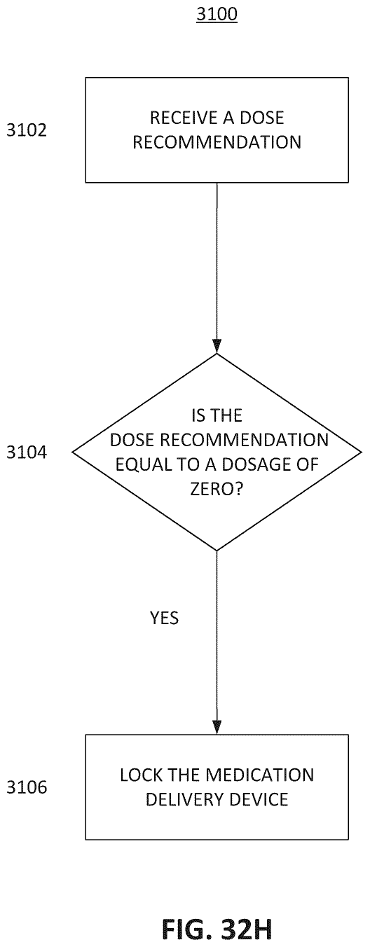

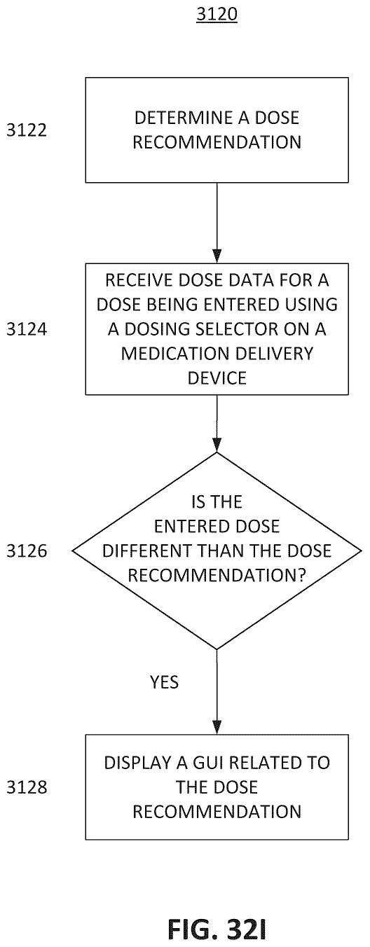

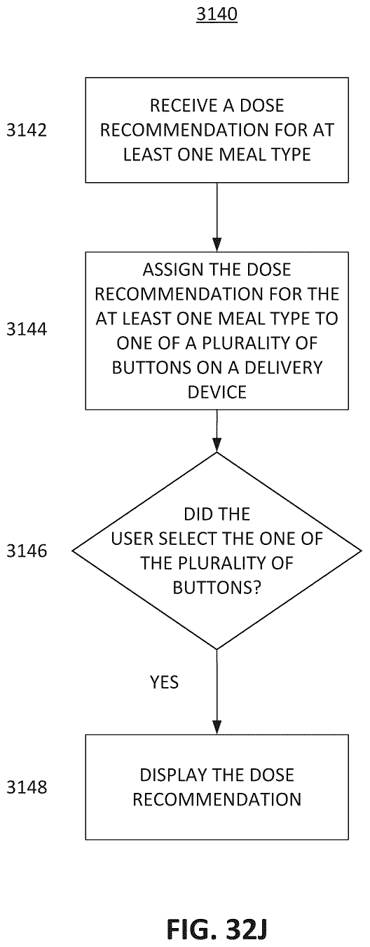

FIGS. 9A-9G depict example embodiments of another analyte monitoring system report GUI 600 including information regarding insulin doses administered. According to one aspect of the embodiments, GUI 600 is a Weekly Summary report for a time period 613 that includes a plurality of report portions, wherein each report portion is representative of a different day of the week. The time period may be for a single week or a plurality of weeks, e.g., 7 days, 14 days (see, e.g., FIGS. 9B-9C), 21 days, 28 days (see, e.g., FIGS. 9D-9G), 32 days, 36 days, etc. Each report portion may include a glucose trend graph 601a-601n for each day of the time period 613, which can include the user's measured glucose levels over a twenty-four hour period, and a health information interface 606, 608, 610, 612. For each day of the time period 613, the health information interface may include information about the user's average daily glucose 606a-606bb, carbohydrate intake 608a-608bb, insulin dosages 610a-610bb, and hypoglycemic (“low”) events 612a-612bb.

In some embodiments, the glucose trend graphs 601a-601bb may include sensor usage markers to indicate that a scan, a view, or both had occurred at a particular time during the twenty-four hour period. The glucose trend graphs 601a-601bb may also include color-coding to highlight portions of the trend graph that are outside of the target range. For example, the trend graphs 601a-601bb may include coloring of the area under the curve a certain color or changing the portion of the graph to be a certain color, or otherwise highlighting the region with the corresponding color for the portion outside of the target range. The area under the curve of the portion above the target range 603 may be colored, e.g., yellow or orange, and the area under the curve of the portion below the target range 605 may be colored, e.g., red.

The “low” events column 612a-612bb may list the number of low events that were detected where the user's analyte levels went below a lower threshold (e.g., 80 mg/dL for glucose, alternatively 75 mg/dL). The low events may also be highlighted in the glucose trend graph by shading an area under the curve of the low event in a different color, e.g., red.

Similarly, the total carbohydrate amount 608a-608bb ingested may be listed for each day in the health information interface 604. These carbohydrate entries may be distinguishable from the insulin dosage entries by appearing in a different color, e.g., orange, and/or with a different icon, e.g., an apple. The total carbohydrate entry 608a-608bb may be blank, a dash, or a plurality of dashes if no value for the amount of carbohydrates is available. Moreover, the amounts of carbohydrates ingested may also be included in the glucose trend graphs 602a-602bb. Similar to the insulin doses, the amount of carbohydrates in different meals or snacks can be noted at or near the time of ingestion, and the amount may be listed in an orange box. If insulin was administered at or around the same time as a meal (ingestion of carbohydrates), the amount of insulin may appear below the amount of carbohydrate ingested on the glucose trend graph.

The insulin dosages column 610a-610bb interface may include the total amount of insulin administered that day for each of the different types of insulin. The total dose amounts for the different insulin types may be differentiated by different colors. For instance, the total rapid-acting insulin administered may be denoted in a light green box and the total long-acting insulin administered may be denoted in a dark green box. The entry for the amount of insulin administered may appear as a dash or a plurality of dashes if no value is available. The specific doses of the different insulins may also be noted in the glucose trend graph 601a-601bb. For example, a box containing the dosage amount may appear in the glucose trend graph at or near the time at which the dose was administered. The boxes may be color-coded, similar to the total insulin amounts, in order to allow the user to quickly determine which insulin was administered. For example, the rapid-acting insulin doses administered may be in light green boxes and the total long-acting insulin doses administered may be in dark green boxes. The long-acting insulin doses may or may not be displayed on the glucose trend graphs. Moreover, when a rapid-acting dose was administered at or around the same time as a long-acting dose, the long-acting dose may appear below the box entry for the rapid-acting dose on the glucose trend graph. The insulin brand names for the different types of insulin administered may also be displayed in a legend 630, which may be located at the bottom of the weekly summary report 600.

In some embodiments, the dosing information may have been manually entered by the user rather than transferred directly from a connected medication delivery device 152. The insulin dose amounts may have been entered into either the monitoring application or the reporting application. Where the insulin dose amounts were manually entered, the report 600 may note the amount of rapid-acting insulin delivered, but may not list the brand name of the insulin in the legend 630, although the legend 630 may include the icon corresponding to the particular type of insulin administered. In some embodiments, the dosing information may be automatically transferred from the connected delivery device. The sources 632 of the data may include the name of the device providing the analyte data levels and the number of the additional merged glucose devices, but may not include the names of any specific insulin pens. The units/day that were administered for each type of insulin and the average total daily insulin (units/day) may also be displayed in the insulin dosage interface 610. The insulin doses administered on the glucose trend graph may be displayed below a carbohydrate entry if they are close in time. In other embodiments, the brand name of the insulin may be included in the weekly summary report 600, in, e.g., the legend 630.

The report may also list the sources 632 of the data, including the name of the device providing the analyte data levels and the name of the primary medication delivery device. When data from multiple connected insulin pens have been included in the weekly summary report, the sources 632 may contain the name of the first connected pen and the number (numerical value) of the additional medical devices.

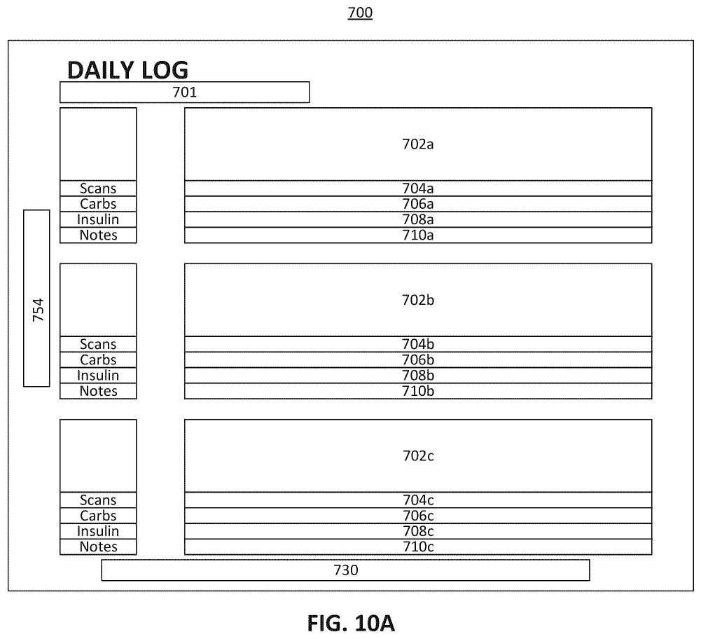

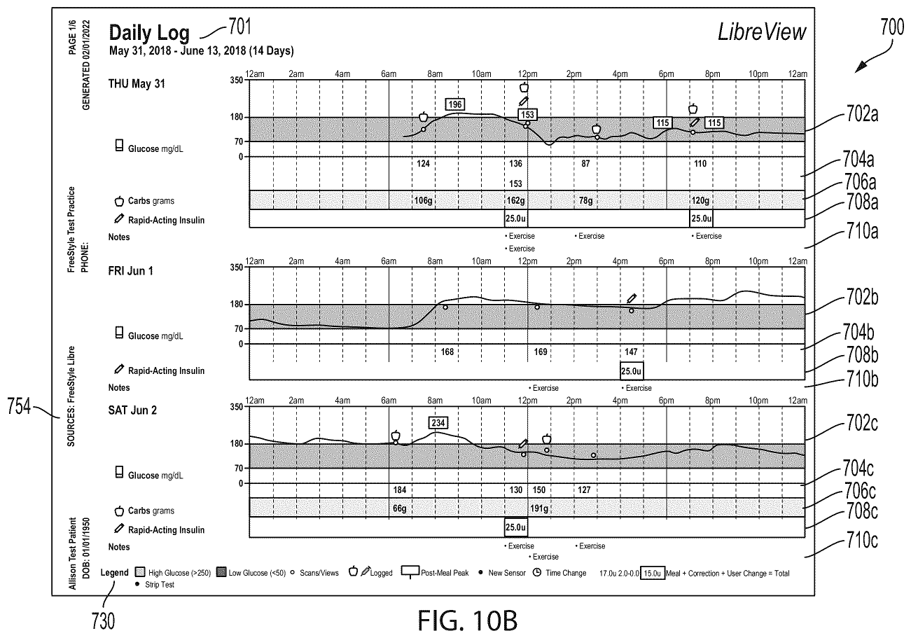

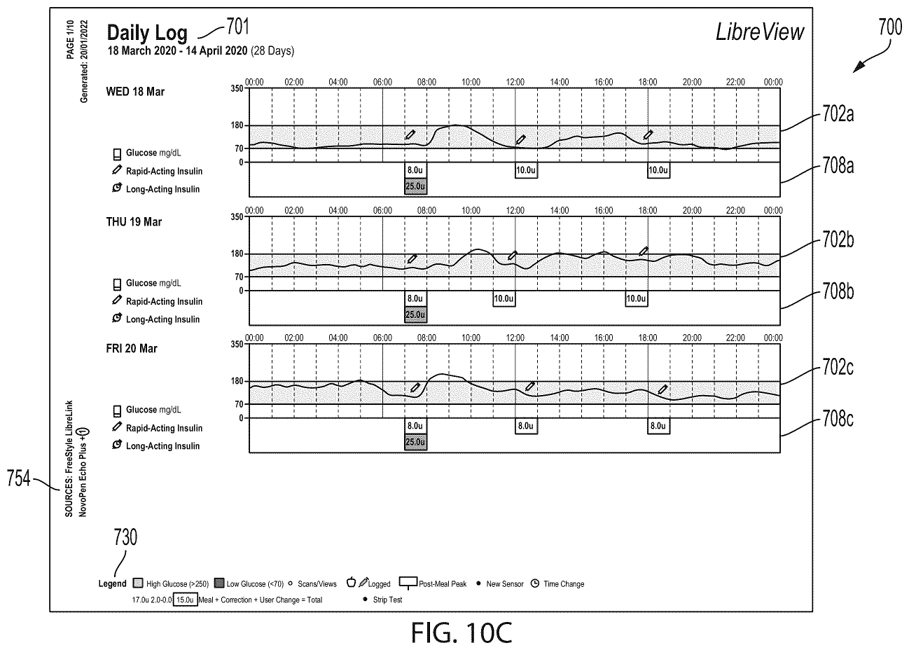

FIGS. 10A-10C depict example embodiments of another analyte monitoring system report GUI 700 including information regarding multiple types of insulin administered in a daily log report. According to one aspect of the embodiments, GUI 700 is a Daily Log report that, for each day of the time period 701, may include a glucose trend graph 702a-702c, a scans or views section 704a-704c, a carbohydrates row 706a-706c, an insulin doses administered row 708a-708c, a notes row 710a-710c, and a legend 730. The time period may be for a single week or a plurality of weeks, e.g., 7 days, 14 days (see, e.g., FIG. 10B), 21 days, 28 days (see, e.g., FIG. 10B), 32 days, 36 days, etc. FIGS. 10A-10C show examples of a first page or screen of the report 700, which contains graphs and information for the first 3 days of the time period. The glucose trend graphs 702a-702c may include the user's glucose levels over a twenty-four-hour period. In some embodiments, the glucose trend graphs 702a-702c may include sensor usage markers to indicate that a scan, a view, or both had occurred at a particular time during the twenty-four hour period. In some embodiments, the glucose level for the scan or view may be listed at the time of the scan or view in the scans or view section 704a-704c. Glucose trend graphs 702a-702c may also include logged event markers, such as logged carbohydrate intake markers and logged insulin dosage markers, as well as glucose event markers, such as low glucose event markers. Beneath the glucose trend graphs 702a-702c, the report 700 may include a plurality of rows with additional information, including a row corresponding to scans or views 704a-704c, carbohydrates 706a-706c, insulin administered 708a-708c, and notes 710a-710c. In the scans or views row 704a-704c, noteworthy glucose levels may be listed in the row in a position corresponding to the time that the glucose level was recorded. For example, a peak glucose level of an episode above the target range may be noted and color coded a first color, e.g., orange, and a low glucose level of a hypoglycemic episode may be color coded a second color, e.g., red. In the carbohydrates row 706a-706c, an amount of carbohydrates may be listed in the row in a position corresponding to the time that the carbohydrates were logged.

For the insulin administered row 708a-708c, the amounts of insulin injections may be displayed in rows under each glucose trend graph 702 for each hour time block. Each medication delivery device may appear in a separate row. For example, as seen in FIG. 10B, insulin doses from a rapid-acting pen may appear in a first row, while insulin doses from an insulin pen containing long-acting insulin may appear in another row, where the long-acting insulin doses row may appear below the rapid-acting insulin doses row. The rapid-acting insulin dose amounts may appear in white boxes with black outlines. User corrections or changes may appear before the dose amount. The long-acting insulin dose amounts may appear in dark green boxes. The sources 754 of the data may include the name of the device providing the analyte data levels and the name of the first connected insulin pen, along with a number of the additional merged glucose devices.

In some embodiments, the insulin dose amounts may have been manually entered by the user and/or determined by a dosage calculator, rather than transferred directly from a connected medication delivery device 152. The insulin dose amounts may have been entered into either the monitoring application or the reporting application. Where the insulin dose amounts were manually entered, the daily log report may note the amount of rapid-acting insulin and long-acting insulin 618 delivered, but may not list the brand name of the insulins next to their respective icons. In some embodiments, the dosing information may be automatically transferred from the connected delivery device. The sources 754 of the data may include the name of the device providing the analyte data levels and the number of the additional merged glucose devices, but may not include the names of any specific insulin pens. A row may only be displayed for a particular insulin type if dosing data for that insulin type was entered for a particular day. If there is no data for a particular day, then a row for that insulin type may not appear below the glucose trend graph 702. Beside the glucose trend graphs 702a-702c, a color-coded icon and the brand name or the type of the insulin may appear next to the insulin dose row. As with other report embodiments, different icons and different colors can be used for the rapid-acting and long-acting insulin pens. For example, the rapid acting insulin may be depicted with a light green solid syringe icon. The long-acting insulin may be depicted with a dark green, wider syringe icon. A row may only appear for a particular day when there is insulin dose data for that day. The daily log report 700 may also include a note if the user switches insulin brands in a connected pen during a particular day. A note may appear when the insulin brand was changed, which includes the old and new insulin brand names. The new insulin brand name may then be listed in the text near the glucose trend graph 702. In other embodiments, the brand name of the insulin may be included in the daily log report 700, in, e.g., the legend 730.

The notes row 710a-710c may also be included in the daily log report 700. The notes may include text that was added regarding events, e.g., exercising. The text may appear in the row in a position corresponding to the time that the notes were logged.

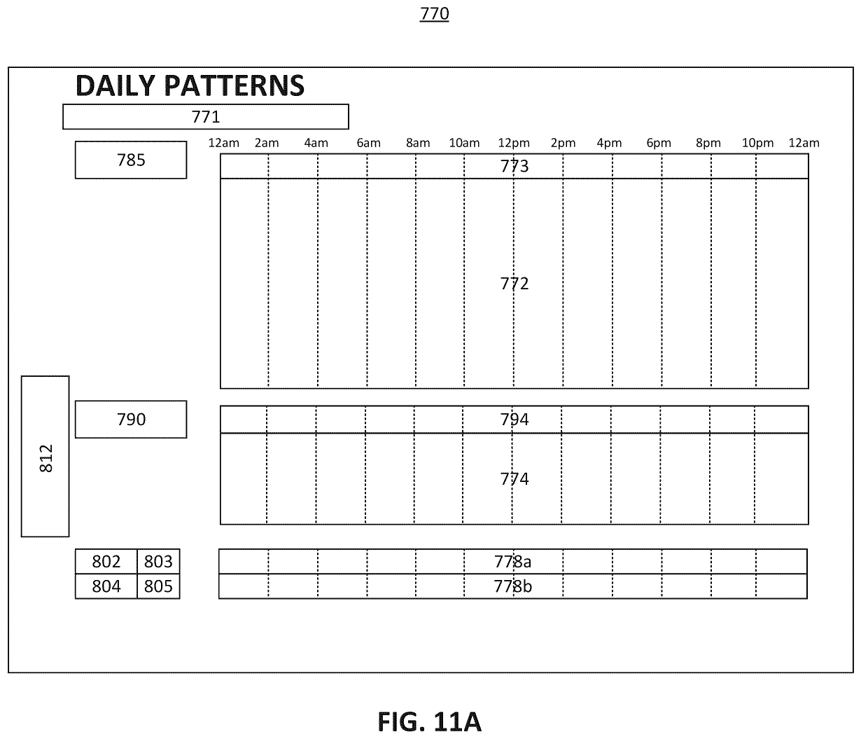

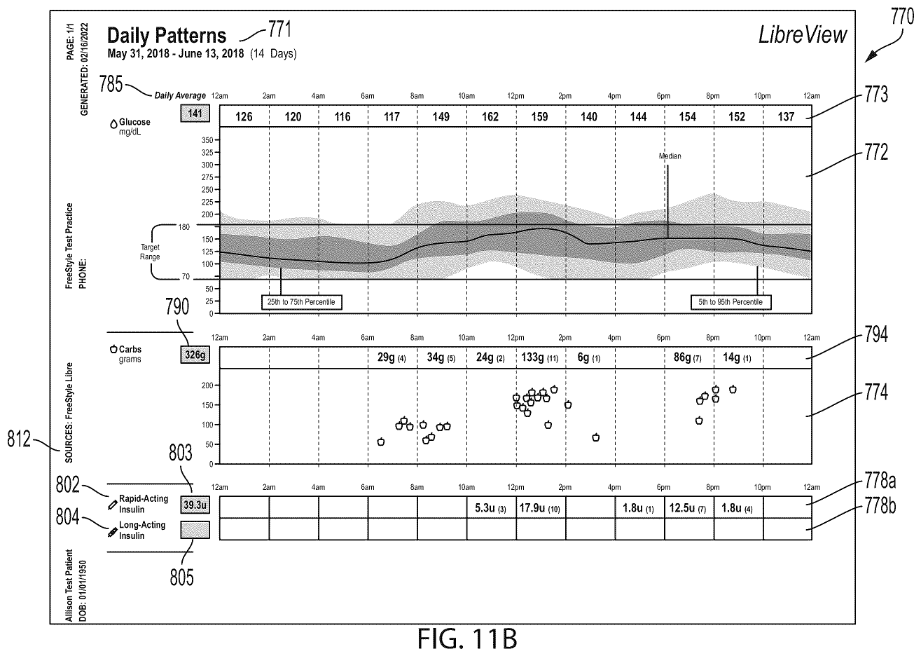

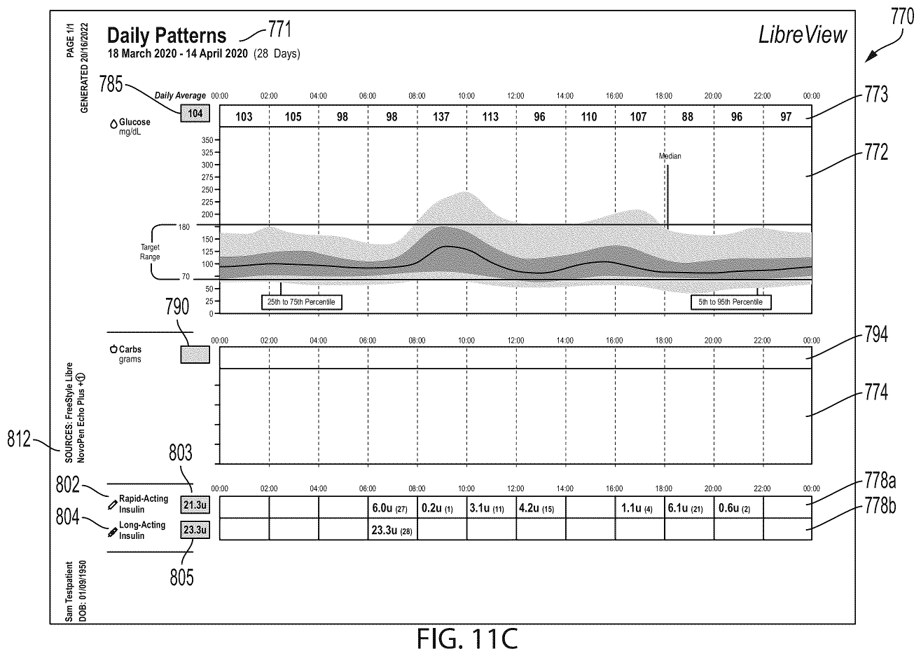

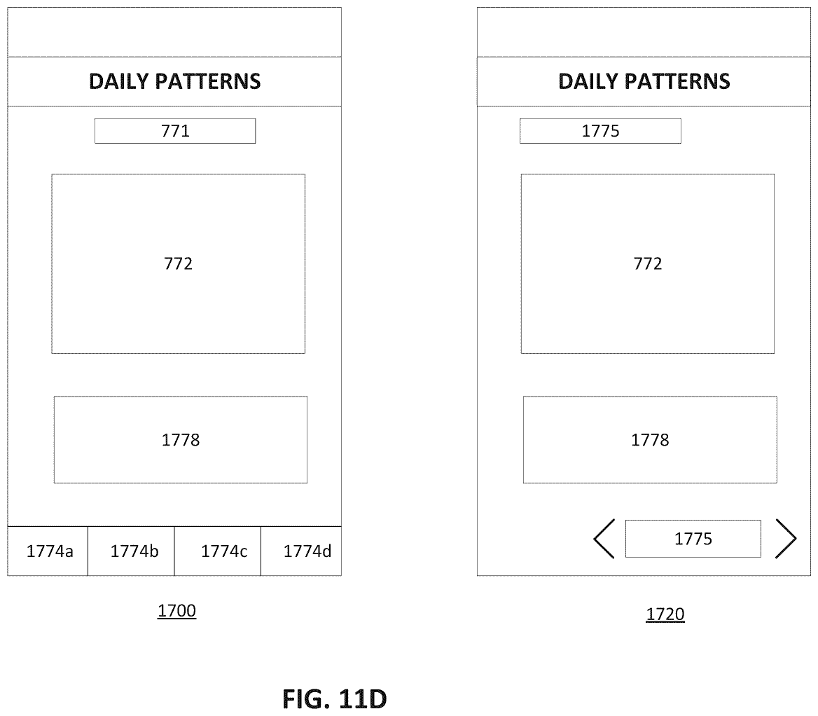

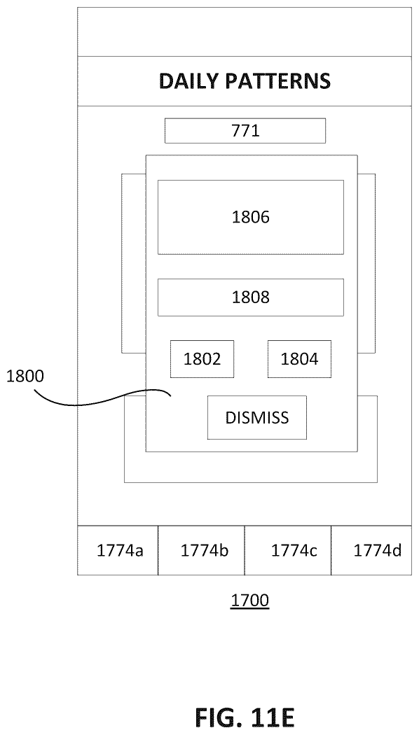

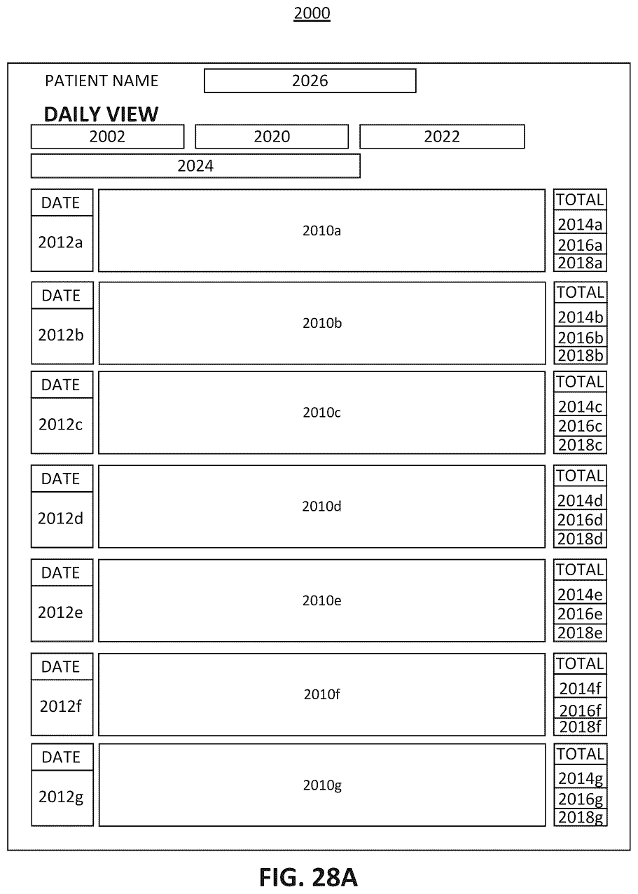

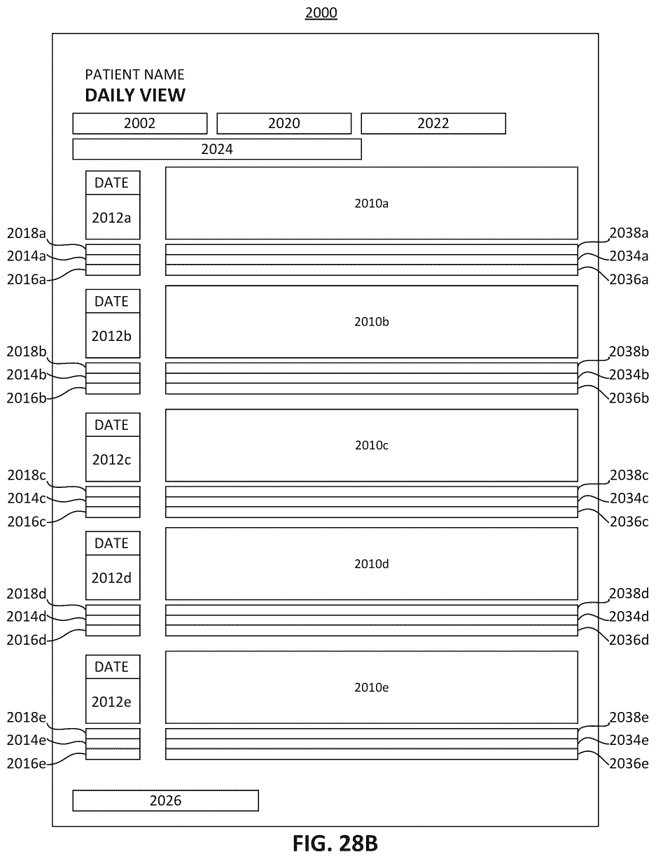

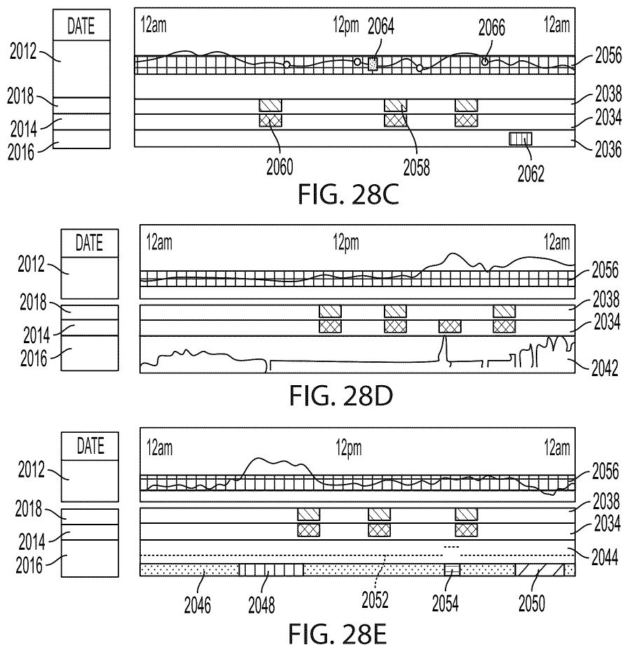

FIGS. 11A-11D depicts example embodiments of a daily patterns report GUI 770. The daily patterns report 770 includes a user's ambulatory glucose profile 772, a section reflecting the amount of carbohydrates 774 consumed in different time of day periods, and a section reflecting the dosage amounts of different types of insulin 778a-778b. The daily pattern report 770 shows a 1 day, i.e., 24-hour, time period graphical representation of glucose measurement values organized by time of day for a period of days 771—e.g., 14 days (see FIG. 11B) or 28 days (see FIG. 11C). The glucose measurement values may be displayed as individual points, or may be averaged into a gradient pattern representative of density of measurement values within particular ranges. The ambulatory glucose profile 772 may include a representation of the target glucose range for the user, as well as a median over time, represented by an average or median line, and lines representing the 10 percentile, 25 percentile, 75 percentile and 90 percentiles. The daily pattern report 770 may also have an indication of the daily average glucose level 785. Above the ambulatory glucose profile 772, a row 773 may appear where the average glucose level for each time period may be listed. Each time period may be about 2 hours, alternatively about 3 hours, alternatively about 4 hours. Noteworthy glucose levels (e.g., the highest average glucose level) may be highlighted with a different color to aid the user in easily seeing which time period had the highest glucose levels.

In addition to measured glucose levels, the daily pattern report 770 may include other average daily information, such as carbohydrates 790, rapid-acting insulin 802, and long-acting insulin 804. The section reflecting the amount of carbohydrates 774 consumed in different time of day periods may include a daily average amount of carbohydrates ingested 790 and a graph of time (x-axis) and amount of carbohydrates (y-axis (grams)), which may include icons (e.g., apples) to mark the amounts of carbohydrates consumed during different time of day periods. The total amount of carbohydrates 794 consumed in a particular time of day period may be listed on top of the graph, along with the number of meals or snacks consumed during the time period (e.g., in parentheses).

The section reflecting the dosage amounts of different types of insulin 778a-778b of the daily patterns report 770 may include a row for each type of insulin administered through a connected medication delivery device 152, e.g., a top row for rapid-acting insulin 778a and a bottom row for long-acting insulin 778b. For each type of insulin (rapid or fast-acting 802 (including “other insulin”) and long-acting 804, an icon representing the type of insulin, a daily average administered 803, 805, the average amount administered for each time of day period, and the number of entries (in parentheses) for that time of day period 810 may be reported in the insulin rows 778a-778b. The brand name of the insulin may also optionally be listed. As with other embodiments discussed herein, a light green syringe icon may be used to denote an insulin pen containing a fast-acting insulin and a dark green syringe icon may be used to denote an insulin pen containing a long-acting insulin. The sources 812 of the data may include the name of the device providing the analyte data levels and the name of the first connected insulin pen, along with a number of the additional connected device (e.g., additional insulin pens).

In some embodiments, where only a single insulin pen is connected, only a single row 778 of insulin doses may appear, along with the corresponding icon (e.g., light green syringe icon for rapid-acting insulin pen) and the brand name or type of the insulin delivered. Where only a single insulin pen is connected, the sources 812 of the data may only include the name of the device providing the analyte data levels and the name of the connected insulin pen, without any number indicating additional devices are connected.

In some embodiments, the insulin dose amounts may have been entered into either the monitoring application or the reporting application. Where the insulin dose amounts were manually entered, the daily patterns report 770 may note the amount of rapid-acting insulin and long-acting insulin delivered, but may not list the brand name of the insulins near their respective icons. In some embodiments, the dosing information may be automatically transferred from the connected delivery device. The sources 812 of the data may include the name of the device providing the analyte data levels and the number of the additional merged glucose devices, but may not include the names of any specific insulin pens. The units/day that were administered for each type of insulin and the average total daily insulin (units/day) may also be displayed. In other embodiments, the brand name of the insulin may be included in the daily pattern report 770, in, e.g., a legend.

A daily patterns report GUI 1700 may also be available on the display device 120, through the monitoring application, a reporting application, or other application. As seen in FIG. 11D, the daily patterns report GUI 1700 may include a user's ambulatory glucose profile 772, the period of days or the number of days 771 that is being displayed in the GUI 1700, an insulin summary section 1778, and alternative time periods 1774a-1774d to select and display. The ambulatory glucose profile 772 has been described in detail with respect to other embodiments and reports. The ambulatory glucose profile 772 may also indicate how many days of the time period 771 that the glucose data was available. The ambulatory glucose profile 772 may also include syringes above or below the graph to indicate the times at which the doses were administered. Alternatively, the doses may be graphed along the median line of the ambulatory glucose profile to indicate what time they were administered. The insulin summary section 1778 may include a graphical representation of the amounts of insulin dosed during the time period 771. In some embodiments, the graph may be a bar graph of time (x-axis) vs. the average number of insulin units administered. The graph may include both fast-acting insulin doses and basal doses. The insulin summary section may also indicate how many days of the time period 771 that the dosing data was available. The x-axis (time) of the graph in the insulin summary section 1778 may align with the x-axis (time) of the ambulatory glucose profile 772, with the average dose amounts for the different meal and basal doses being graphed at the average time that they were administered during the time period 771. Thus, the user may be easily able to see a correlation between their insulin doses and their glucose profile in the time periods relevant to the different doses. The GUI 1700 may also include tabs 1774a-1774b with different time periods for which the user may select to display the daily patterns report. For example, the time period tabs 1774a-1774d may be 1 day, 7 days, 14 days, 30 days, or 90 days.

Alternatively, as seen in GUI 1720 of FIG. 11D, the user may select the time period in a drop down menu 1775. In such a case, the GUI 1700 may include a date range 1775 and the user may scroll forward and backwards in time by tapping on the forward or backward carets. In some embodiments, when a user selects 1 day for the time period, a daily graph may be displayed, which shows the glucose and insulin data for the individual day selected. For the daily graph, instead of an ambulatory glucose profile, the daily graph may display the glucose profile for that day, while also highlighting the target range for the user. The glucose profile graph may also contain syringe icons above or below the graph to indicate the times at which the insulin was administered. Alternatively, the doses may be graphed along the median line of the glucose profile to indicate what time they were administered. The insulin summary section may display graphical representation of the amounts of insulin dosed during the day (as opposed to an average number of doses for a multi-day period). In some embodiments, the graph may be a bar graph of time (x-axis) vs. the number of insulin units administered. The graph may include both fast-acting insulin doses and basal doses. The x-axis (time) of the graph in the insulin summary section may align with the x-axis (time) of the glucose profile, with the dose amounts for the different meal and basal doses being graphed at the time that they were administered during the day. Thus, the bars in the bar graph may align at the same times with the syringe icons in the glucose profile graph.

As seen in FIG. 11E, if the user requests more information by, e.g., tapping on an “i” icon, a window 1800 may be displayed that includes an explanation 1806 that the daily patterns report shows the user's glucose and insulin patterns over a period of time. The window 1800 may also include the target range, e.g., 70-180 mg/dL. The user may also be able to select the type of insulin data to display—either rapid acting 1802 or long acting 1804.

FIGS. 28A-D depict example embodiments of another analyte system report GUI 2000 that includes information regarding multiple types of insulin administered in a Daily View Report GUI 2000. According to one aspect of the embodiments, the Daily View Report GUI 2000 may include, for each day of the time period 2002, a glucose profile 2010a-g, a time-in-range metric 2012a-g, a listing of the total rapid-acting insulin administered 2014a-g, a listing of the total long-acting insulin administered 2016a-g, and a listing of the total amount of carbohydrates consumed 2016a-g. The time period 2002 may be for a single week or a plurality of weeks, e.g., 7 days, 14 days, 21 days, 28 days, 32 days, 36 days, etc. FIGS. 28A-B show examples of a first page or screen of the report 2000, which contains graphs and information for the first 7 days of the time period. The Daily View Report GUI 2000 may also list the time that a sensor was active 2020 during the time period, the average number of scans or views 2022 per day during the time period, a legend 2024 of the symbols used in the report 2000, and the sources 2026 of the data used in the report 2000.

The glucose profiles 2010a-g may include the user's glucose levels over a twenty-four-hour period. In some embodiments, the glucose profiles 2010a-g may optionally include sensor usage markers (e.g., a circle) 2066 to indicate that a scan, a view, or both had occurred at a particular time during the twenty-four-hour period. In some embodiments, the glucose level for the scan or view may be listed at the time of the scan or view in the scans or view section. Each daily profile may represent a midnight to midnight period with the date displayed in the same frame as the profile. In addition to the displaying the date, each profile may also indicate the corresponding day of the week. Each profile may also contain an indication of the target glucose range (e.g., a shaded region or lines indicating the upper and lower boundaries of the targe region) to illustrate which parts of each daily profile were within the target range 2056. Portions of the graph outside of the target range 2056 may also be color-coded as a further indication of readings or analyte levels that were outside of the target range 2056. The color coding may correspond to the colors used in the Time-in-Ranges graphical representations described in other reports. For example, portions of the graph above the target range 2056 in the “high” level (e.g., 181-250 mg/dL) may be color-coded yellow. Portions of the graph below the target range 2056 in the “low” level (e.g., 54-69 mg/dL) may be color-coded red. Portions of the graph below the in the “very low” level (e.g., <54 mg/dL) may be color-coded dark red or maroon. The color-coding may include coloring the area under the curve (e.g., the area between the curve and the high threshold, low threshold, or very low threshold) a certain color or changing the portion of the graph to be a certain color, or otherwise highlighting the region with the corresponding color.

Glucose profiles 2010a-g may also optionally include logged event markers, such as logged exercise events 2064, logged carbohydrate intake markers, and logged insulin dosage markers, as well as glucose event markers, such as low glucose event markers. The logged event markers may be listed above or below the glucose graph at the time that the event was logged.

As seen in FIGS. 28B-E, in some embodiments, instead of including the numbers in the glucose profile 2010a-g, the report 2000 may include a plurality of rows with additional information beneath the glucose profiles 2010a-g, including a row corresponding to carbohydrates consumed 2038a-e, rapid-acting insulin received 2034a-e, and long-acting insulin received 2036a-e. In the carbohydrates row 2038, an amount of carbohydrates 2058 may be listed in the row 2038 in a position corresponding to the time that the carbohydrates were logged. Similarly, in the rapid-acting row 2034, the amounts of rapid-acting insulin 2060 administered may be listed under the time of administration. In the long-acting insulin rows 2036, the amounts of long-acting insulin administered 2062 may be listed under the time of administration. In some embodiments where the dosing data was obtained from a connected pen or connected pen cap, the report 2000 may include the long-acting insulin row 2036. The color-coding for these entries may be the same as the listing of carbohydrates, rapid-acting insulin, and long-acting insulin in the listing of the total amounts 2018, 2014, and 2016.

In some embodiments, the report 2000 may also or alternatively contain rows that indicate scans or views, and notes. In the scans or views row, noteworthy glucose levels may be listed in the row in a position corresponding to the time that the glucose level was recorded. For example, a peak glucose level of an episode above the target range 2056 may be noted and color coded a first color, e.g., orange, and a low glucose level of a hypoglycemic episode may be color coded a second color, e.g., red. Optionally, noteworthy glucose levels may be included in the glucose profile 2010a-g.

In some embodiments, as seen in FIG. 28D, where the basal insulin was administered using an insulin pump, the report may also or alternatively include an insulin delivery graph 2042c located below or above the glucose profile 2010 that shows when and the amount of the insulin was delivered in lieu of a long-acting insulin row 2036 showing discrete doses. The insulin delivery graph 2042c may have an x-axis of time and a y-axis of units/hr. The time axis of the insulin delivery graph 2042c and the glucose profile 2010 may be aligned. When an insulin pump is the source of basal dose information, the listing of the total long-acting insulin administered 2016c may include a name of the pump system and a percentage of time that the pump was active. FIG. 28D depicts a single day's entry from a Daily View Report 2000.

In some embodiments, as seen in FIG. 28E, where the insulin was administered using an insulin pump, the report may also or alternatively include an insulin delivery graph 2044c located below or above the glucose profile 2010 that shows how much insulin was delivered and the delivery mode of the insulin pump in lieu of a long-acting insulin row 2036 showing discrete doses. In some embodiments, the insulin graph 2044c may have an x-axis of time and a y-axis of units/hr. The insulin graph 2044c may include a trace 2052 of the insulin amount delivered and segments of time when the pump was operating on automated delivery 2046, maximum delivery 2048, or automated pause 2050, or if insulin was delivered manually 2054. When an insulin pump is the source of basal dose information, the listing of the total long-acting insulin administered 2016c may include a name of the pump system and a percentage of time that the pump was active. FIG. 28E depicts a single day's entry from a Daily View Report 2000.

The time-in-range metric 2012a-g may be a percentage of time spent in the target range 2056 for that day.

The listing of the total rapid-acting insulin administered 2014a-g for each day may be determined from data that is automatically transferred from a connected medication delivery device 152, such as a connected pen, connected pen cap, or an insulin pump. Alternatively, in some embodiments, the listing of the total rapid-acting insulin administered 2014a-g for each day may be determined from insulin doses that were manually logged by a user.

The listing of the total long-acting insulin administered 2016a-g for each day may be determined from data that is automatically transferred from a connected medication delivery device 152, such as a connected pen, connected pen cap, or an insulin pump. Alternatively, in some embodiments, the listing of the total long-acting insulin administered 2016a-g for each day may be determined from insulin doses that were manually logged by a user.

The listing of the total amount of carbohydrates consumed 2016a-g may be determined from carbohydrates logged by the user. Alternatively, the listing of the total amount of carbohydrates consumed 2016a-g may be determined from meals and snacks that are logged, where the carbohydrate content of the meals and snacks is estimated by a program.

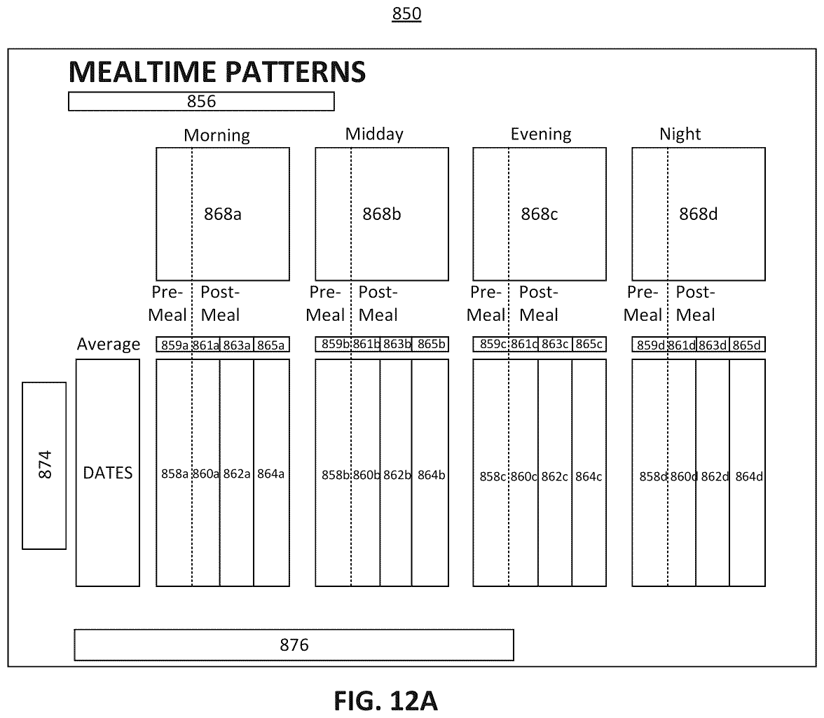

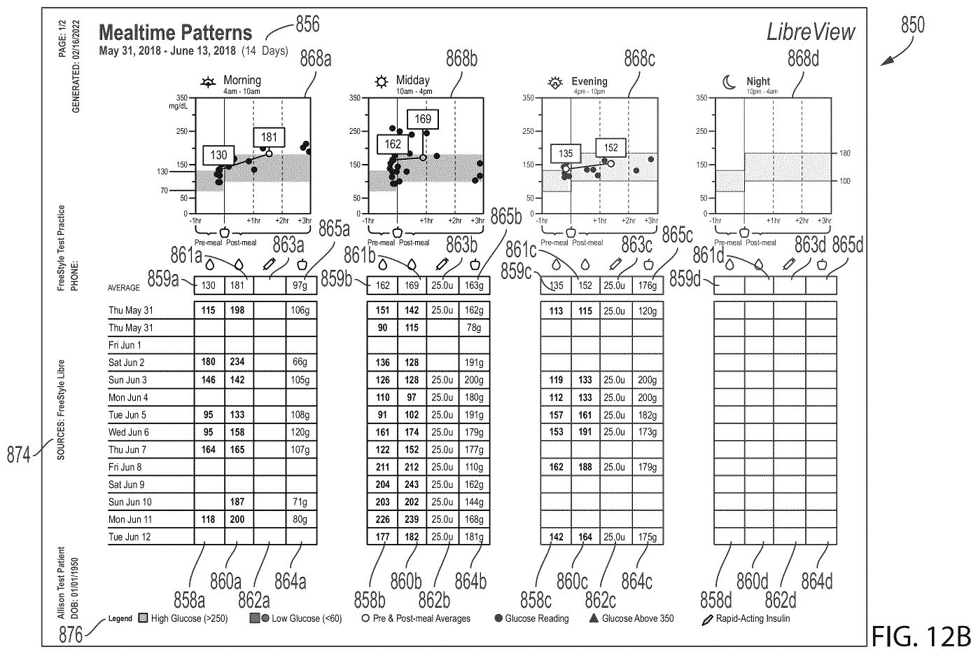

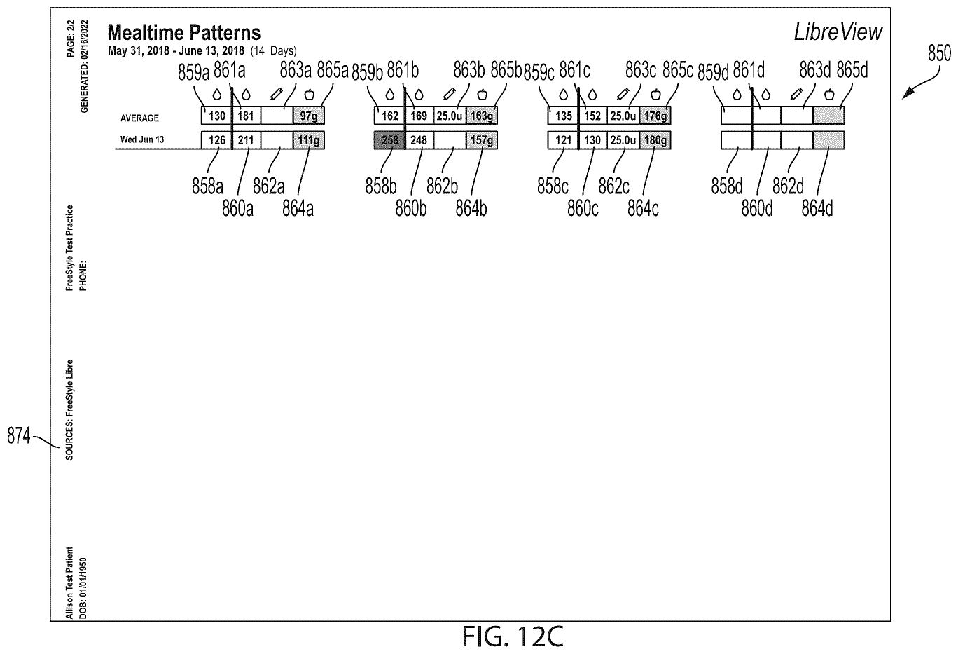

FIGS. 12A-12C depict example embodiments of a Mealtime Patterns report GUI 850. The mealtime patterns report 850 may include graphical and numerical representations of glucose level information with respect to particular times periods of the day that may be associated with meals, i.e., morning (breakfast, e.g., Gam-10 am), midday (lunch, e.g., 10 am-4 pm), evening (dinner, e.g., 4 pm-10 pm), and nighttime (bedtime, e.g., 10 pm-6 am). A vertical line may be displayed to delineate the hours before (pre-meal) 852 and after (post-meal) 854 the corresponding meal. Further, the representations may include numerical indications of the glucose levels before 858a-858d and after 860a-860d the time of the ingestion of the particular meal for each of the days within the mealtime patterns time period 856, along with the average pre-meal 859a-859d and average post-meal glucose levels 861a-861d listed above the tables for each time period. Additionally, a representation of the amount of carbohydrates 864a-864d consumed for each of the days within the mealtime patterns time period, along with the average amount of carbohydrates 865a-865d ingested for the whole time period may be reported.

The Mealtime Patterns report 850 may also contain the amount of insulin administered. An amount of insulin 862a-862d administered for each of the days within the mealtime patterns time period may be displayed in, e.g., a column 862, along with the average amount of insulin administered 863a-863d for that time-of-day period in a row above the table. An icon corresponding to the type of insulin administered (e.g., light green syringe icon for rapid-acting insulin) may also be displayed to indicate the type of insulin administered.

The sources 874 of the data, including the name of the device providing the analyte data levels and the name of the primary medication delivery device may also be displayed where the medication delivery device 152 has been connected to the IMS. The insulin brand name for the type of insulin administered may also optionally be displayed in a legend 876, which may be located at the bottom of the mealtime patterns report 850.

In some embodiments, the insulin dose amounts may have been manually entered by the user and/or determined by a dosage calculator, rather than transferred directly from a connected medication delivery device 152. The insulin dose amounts may have been entered into either the monitoring application or the reporting application. Where the insulin dose amounts were manually entered, the mealtime patterns report 850 may note the amount of rapid-acting insulin 872 delivered, but may not list the brand name in the report. In some embodiments, the dosing information may be automatically transferred from the connected delivery device. The sources 874 of the data may include the name of the device providing the analyte data levels and the number of the additional merged glucose devices, but may not include the names of any specific insulin pens. The insulin brand name for the type of insulin administered may not be displayed in the legend 876, which may be located at the bottom of the mealtime patterns report 850. Rather, the legend 876 lists the insulin as, e.g., “rapid-acting” without a brand name. In other embodiments, the brand name of the insulin may be included in the mealtime patterns report 850, in, e.g., the legend 876.

The mealtime patterns report 850 may also contain a plurality of graphs 868a-868d of the glucose levels for each of the times periods of the day, i.e., morning (breakfast, e.g., 6 am-midday (lunch, e.g., 10 am-4 pm), evening (dinner, e.g., 4 pm-10 pm), and nighttime (bedtime, e.g., 10 pm-6 am). Each graph 868a-868d may include a vertical line depicting a meal start time and may span a time frame of about 1 hour before the meal start to about 3 hours after the meal start. The graph may further include data points on the vertical line indicating the plurality of glucose levels scanned or recorded at the beginning of the meal or just before the start of the meal, which are listed in the columns 858a-858d. The average glucose level of the plurality of glucose levels scanned or recorded at the beginning of the meal or just before the start of the meal indicated at the top of the columns 858a-858d may be highlighted in each graph. Additionally, the graphs 868a-868d may include data points indicating glucose levels after the meal, e.g., at least about 2 hours after the meal start, which are listed in the columns 860a-860d. The average glucose level of the plurality of glucose levels scanned or recorded after the meal, e.g., at least about 2 hours after the meal start, indicated at the top of the column 860a-860d may also be highlighted in the graphs. The graphs 858a-858d may also highlight the target range before the meal, e.g., about 70 mg/dL to about 130 mg/dL, and the target range after the meal, e.g., about 100 mg/dL to about 180 mg/dL, so that it is readily apparent if the recorded glucose levels are in or outside the target range at a glance.

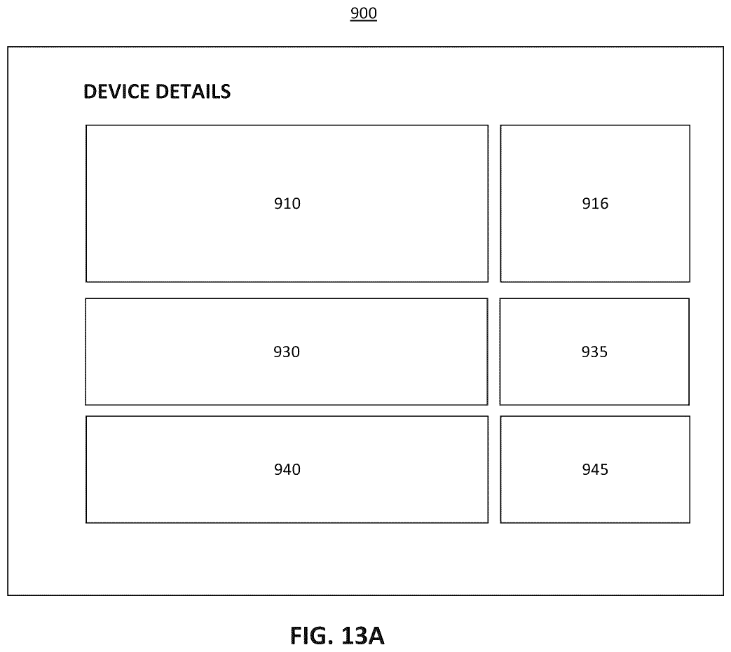

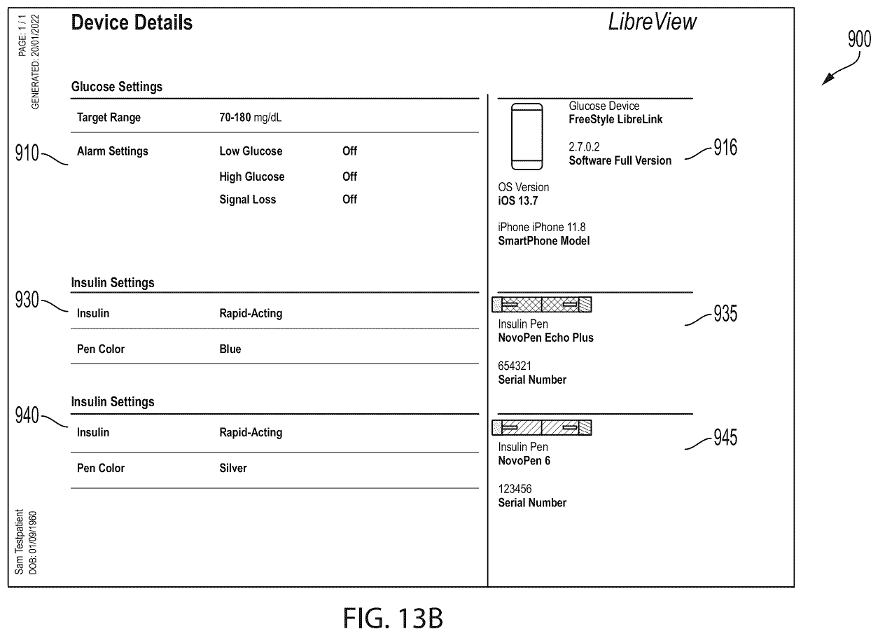

FIGS. 13A-13B depict example embodiments of Device Details reports GUI 900. The device details report 900 may include glucose settings 910 for the primary glucose device, insulin settings for a first connected insulin pen 930, and insulin settings for a second (or additional) connected insulin pen 940. The glucose settings 910 may include a target range (e.g., −180 mg/dL) and the alarm settings for low glucose (e.g., 70 mg/dL), high glucose (e.g., 240 mg/dL), and signal loss. The target range may include a low and a high threshold and be set from a monitoring application or a reader. The glucose settings 910 may optionally also include calculator settings and reminders. The Device Details report 900 may also contain details about the primary glucose device 916 that receives analyte levels or data indicative of analyte levels from the SCD 102, including the name of the device (and any icon associated with the device), the current software version on the device, the current operating system version, and the model of the smartphone on which related applications are being run. If the SCD 102 is being used in conjunction with a reader or meter, the device details 916 may include the seral number of the reader or meter.

The device details report 900 may also include details for any connected medication delivery devices 152 (e.g., connected insulin pens). The connected medication delivery devices 152 may be listed under the primary glucose device. If more than one medication delivery device is connected, then each may be displayed as its own device. For each connected medication delivery device, the insulin pen settings 930, 940 may include the insulin type, last scan (e.g., date and timestamp of last insulin value), and pen color. The insulin pen settings 930, 940 may optionally also include calculator settings, notes, and/or reminders. The device details report 900 may also contain details 935, 945 about the insulin pens near their respective settings. The details 935, 945 may include a colored icon or picture of the insulin pen, the brand name of the insulin pen or the type of insulin, and the serial number.

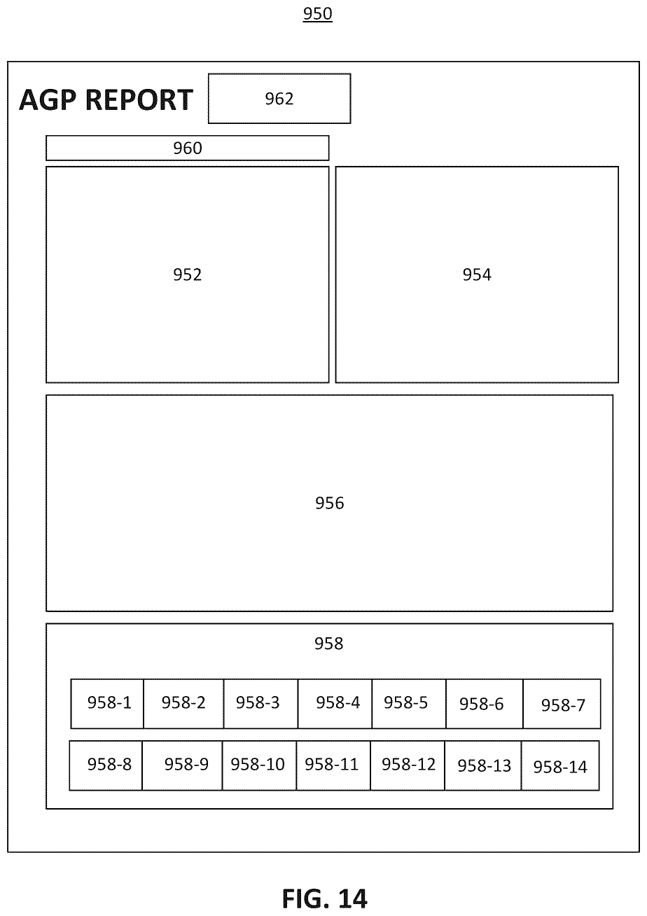

FIG. 14 depict an example embodiment of an AGP report GUI 950. The AGP report 950 may include a glucose statistics and targets section 952, a time in ranges section 954, a user's ambulatory glucose profile 956, and a daily glucose profiles section 958. The AGP report 950 shows various statistics and graphs for a period of days 960—e.g., 14 days or 28 days. The sources 962 of the information may be listed and may include the names (e.g., brand names) of the primary glucose device, the first connected pen, and the number of additional devices.

The glucose statistics and targets or glucose metrics 952 section may include any one or more of the following metrics: the days in which the statistics are reported, the amount of time the sensor was active (reported as a percentage), the amount of time (reported as percentages) that the detected analyte levels were within various ranges, the average glucose, the glucose management indicator (GMT), and the glucose variability. The amount of time in the various ranges may include the amount of time within a target range (e.g., 70-180 mg/dL), below a low threshold (e.g., below 70 mg/dL), below a lower threshold (e.g., below 54 mg/dL), above a high threshold (e.g., above 180 mg/dL), and above a higher threshold (e.g., above 250 mg/dL).

The time in ranges section 954 may include Time-in-Ranges (also referred to as Time-in-Range and/or Time-in-Target) GUIs, each of which comprise a plurality of bars or bar portions, wherein each bar or bar portion indicates an amount of time that a user's analyte level is within a predefined analyte range correlating with the bar or bar portion. In some embodiments, for example, the amount of time can be expressed as a percentage of a predefined amount of time. Time-in-Ranges GUI portion 954 may include a single bar comprising up to five bar portions including (from top to bottom): a first bar portion indicating that the user's glucose range is “Very High” or above 250 mg/dL of a predefined amount of time, a second bar portion indicating that the user's glucose range is “High” or between 180 and 250 mg/dL of the predefined amount of time, a third bar portion indicating that the user's glucose range is within a “Target Range” or between 70 and 180 mg/dL of the predefined amount of time, a fourth bar portion indicating that the user's glucose range is “Low” or between 54 and 69 mg/dL of the predefined amount of time, and a fifth bar portion indicating that the user's glucose range is “Very Low” or less than 54 mg/dL of the predefined amount of time. Time-in-Ranges GUI 954 may display text adjacent to each bar portion indicating an actual amount of time, e.g., in hours and/or minutes.

According to one aspect of the embodiment shown in FIG. 14, each bar portion of Time-in-Ranges GUI 954 may comprise a different color. In some embodiments, bar portions can be separated by dashed or dotted lines and/or interlineated with numeric markers to indicate the ranges reflected by the adjacent bar portions. In some embodiments, the time in ranges reflected by the bar portions can be further expressed as a percentage, an actual amount of time (e.g., 4 hours and 19 minutes), or both. Furthermore, those of skill in the art will recognize that the percentages of time associated with each bar portion can vary depending on the analyte data of the user. In some embodiments of Time-in-Ranges GUI 954, the Target Range can be configured by the user. In other embodiments, the Target Range of Time-in-Ranges GUI 954 is not modifiable by the user.

The AGP report 950 may also contain an AGP portion 956 similar to the AGP graph 511. The AGP graph may display the hourly 5^{th}, 25^{th}, 50^{th} (median), 75^{th}, and 95^{th} percentiles of glucose readings, presented over the “typical” day based on all days within the selected timeframe. The AGP graph may also include two horizontal lines, which indicate the boundaries of the target range defined in the glucose statistics and targets portion 952 and the Time-in-Ranges portion 954. For example, a first line may correspond to the lower boundary of the target range (e.g., 70 mg/dL) and a second line may correspond to an upper boundary of the target range (e.g., 250 mg/dL). The first and second lines may also be color-coded and correspond to the same color as the target range bar portion in the Time-in-Ranges portion 308 (e.g., green). The data or portions of the AGP within the respective concentration ranges may also be color-coded and correspond to the same color as the target range bar portions in the Time-in-Ranges portion (308). For example, data points or portions of the AGP in the target range may be colored green, data points or portions of the AGP in the high concentration range may be colored orange, data points or portions of the AGP in the high range may be colored orange, data points or portions of the AGP in the low range may be colored red, and data points or portions of the AGP in the very low range may be colored dark red or maroon. Thus, the AGP plot easily illustrates the amount of time spent in (or amount of readings falling within) the target range.

The AGP report 950 may also include a daily glucose profiles section 958. The daily glucose profiles section 958 displays a plurality of daily profiles 958-1-958-14, one for each day of the time period 960. Each daily profile may represent a midnight to midnight period with the date displayed in the same frame as the profile. In addition to the displaying the date, each profile may also indicate the corresponding day of the week. Each profile may also contain an indication of the target glucose range (e.g., a shaded region or lines indicating the upper and lower boundaries of the targe region) to illustrate which parts of each daily profile were within the target range. Portions of the graph outside of the target range may also be color-coded as a further indication of readings or analyte levels that were outside of the target range. The color coding may correspond to the colors used in the Time-in-Ranges 954 portion. For example, portions of the graph above the target range in the “high” level (e.g., 181-250 mg/dL) may be color-coded yellow. Portions of the graph below the target range in the “low” level (e.g., 54-69 mg/dL) may be color-coded red. Portions of the graph below the in the “very low” level (e.g., <54 mg/dL) may be color-coded dark red or maroon. The color-coding may include coloring the area under the curve (e.g., the area between the curve and the high threshold, low threshold, or very low threshold) a certain color or changing the portion of the graph to be a certain color, or otherwise highlighting the region with the corresponding color.

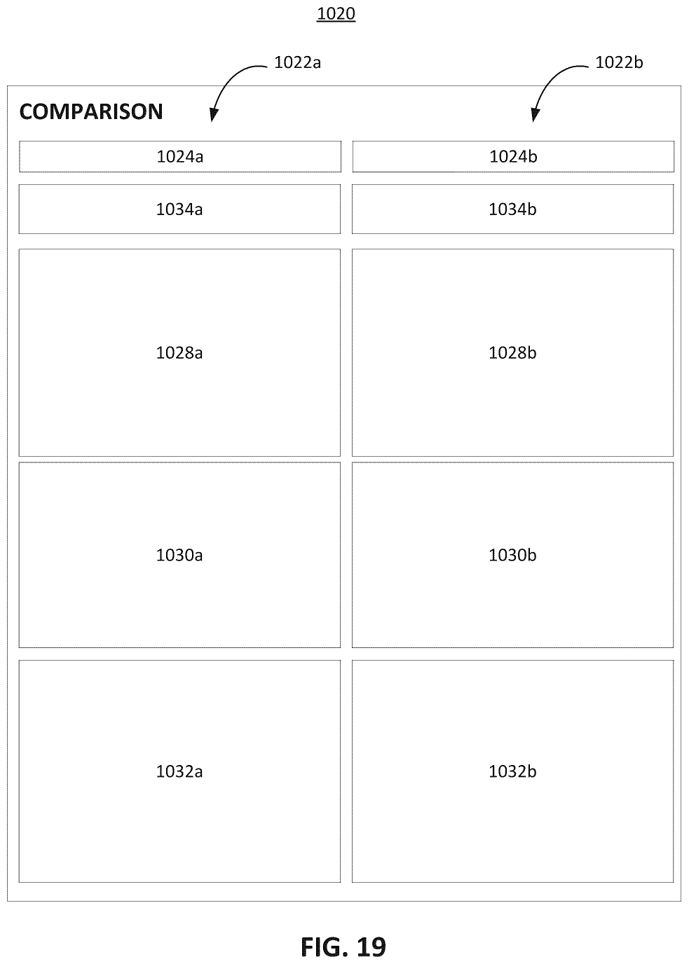

FIG. 19 depicts an example embodiment of a Comparison report GUI 1020. The Comparison report 1020 may include two sections 1022a, b that display metrics for first and second time periods 1024a, b. The metrics for the first and second time periods may be displayed side-by-side to allow for easy analysis and comparison of the different time periods. Each of the sections of the Comparison report 1020 may include a glucose metrics section 1026a,b, a time in ranges section 1028a, b, an ambulatory glucose profile section 1030a, b, and a low glucose events section 1032a, b. The Comparison report 1020 shows various statistics and graphs for two time periods—e.g., 14 days each. In some embodiments, a banner may also appear, e.g., in the header, to inform the viewer if any glucose threshold levels have been adjusted.

The Comparison report GUI 1020 may report the days in which the various metrics are reported 1024a, b, and the amount of time the sensor was active (reported as a percentage) 1034a, b.

The glucose metrics sections 1026a, b may include, for example, the average glucose, the glucose management indicator (GMI), and the glucose variability.

The time in ranges section 1028a, b may include Time-in-Ranges (also referred to as Time-in-Range and/or Time-in-Target) graphical representations, each of which comprise a plurality of bars or bar portions, wherein each bar or bar portion indicates an amount of time that a user's analyte level was within a predefined analyte range correlating with the bar or bar portion. In some embodiments, for example, the amount of time can be expressed as a percentage of a predefined amount of time. Time-in-Ranges graphs 1028a, b may each include a single bar comprising a plurality of bar portions stacked vertically. In some embodiments, the graph may include 5 bar portions: a first bar portion indicating the amount of time that the user's glucose range was “Very High” or above 250 mg/dL for a predefined amount of time, a second bar portion indicating the amount of time that the user's glucose range was “High” or between 180 and 250 mg/dL for the predefined amount of time, a third bar portion indicating the amount of time that the user's glucose range was within a “Target Range” or between 70 and 180 mg/dL for the predefined amount of time, a fourth bar portion indicating the amount of time that the user's glucose range was “Low” or between 54 and 69 mg/dL for the predefined amount of time, and a fifth bar portion indicating the amount of time that the user's glucose range was “Very Low” or less than 54 mg/dL for the predefined amount of time. Time-in-Ranges graphs 1028a, b may optionally also each display text adjacent to each bar portion indicating an actual amount of time, e.g., in hours and/or minutes. In some embodiments, the time in ranges reflected by the bar portions can be further expressed as a percentage, an actual amount of time (e.g., 4 hours and 19 minutes), or both. Furthermore, those of skill in the art will recognize that the percentages of time associated with each bar portion can vary depending on the analyte data of the user.

According to one aspect of the embodiment shown in FIG. 19, each bar portion of Time-in-Ranges graph 1028a, b may comprise a different color. In some embodiments, bar portions can be separated by dashed or dotted lines and/or interlineated with numeric markers to indicate the ranges reflected by the adjacent bar portions. In some embodiments, the “Very High” portion may be colored orange, the “High” portion may be colored yellow, the “Target Range” may be colored green, the “Low” portion may be colored red, and the “Very Low” portion may be colored dark red or maroon. This color scheme may be followed in other portions of the Comparison report 1020, such as the ambulatory glucose profile section 1030a, b and the low glucose events section 1032a, b. For example, the analyte levels in displayed in the graphs in the ambulatory glucose profile section 1030a, b and a low glucose events section 1032a, b may be color coded according to the concentration range that the analyte level falls within. For example, an analyte level of 156 mg/dL may be colored green in an ambulatory glucose profile while an analyte level of 48 mg/dL may be colored dark red or maroon in an ambulatory glucose profile or low glucose events graph. In some embodiments of Time-in-Ranges graphs 1028a, b, the Target Range can be configured by the user. In other embodiments, the Target Range of Time-in-Ranges GUI 1028a, b is not modifiable by the user.

Each section of the Comparison report 1020 may also contain an AGP portion 1030a, b similar to the AGP graph 511 displayed in FIG. 8. The AGP graph may display the hourly 5^{th}, 25^{th}, 50^{th} (median), 75^{th}, and 95^{th} percentiles of glucose readings, presented over the “typical” day based on all days within the selected timeframe. The AGP graph may also include two horizontal lines, which indicate the boundaries of the target range defined in the glucose statistics and targets portion 1026a.b and the Time-in-Ranges graph 1028a, b. For example, a first line may correspond to the lower boundary of the target range (e.g., 70 mg/dL) and a second line may correspond to an upper boundary of the target range (e.g., 250 mg/dL). The first and second lines may also be color-coded and correspond to the same color as the target range bar portion in the Time-in-Ranges portion 308 (e.g., green). The AGP graphs may adopt the same color coding as in the Time-in-Ranges GUI 1028a, b such that the glucose levels in the “Very High” range (e.g., about 250 mg/dL) may be colored orange, the glucose levels in the “High” range (e.g., between 180 mg/dL and 250 mg/dL) may be colored yellow, the glucose levels in the “Target Range” (e.g., between 70 mg/dL and 180 mg/dL) may be colored green, the glucose levels in the “Low” range (e.g., between 54 mg/dL and 70 mg/dL) may be colored red, and the glucose levels in the “Very Low” range (e.g., below 54 mg/dL) may be colored dark red or maroon. Thus, each AGP plot may easily illustrate the amount of time spent in or, the amount of readings falling within, the target range for each time period 1024a, b.

Each section of the Comparison report 1020 may also include a low glucose events section 1032a, b. The low glucose events sections 1032a, b may each include a graph of the events in which the subject's glucose levels dropped below a low threshold, e.g., 70 mg/dL and a very low threshold, e.g., 54 mg/dl. Each graph may also include a line illustrating a low threshold (e.g., 70 mg/dl) and a very low threshold (e.g., 54 mg/dl). The graph may show the glucose concentration (mg/dL) vs. time, such that the times of the low event episodes during the day are readily apparent. As mentioned previously, the graph of the glucose levels in the low glucose events section 1032a, b may adopt the same color coding as in the Time-in-Ranges GUI 1028a, b such that the glucose levels in between the low threshold and very low threshold (e.g., between 54 mg/dl and 70 mg/dl) may be colored red, and the glucose levels below the very low threshold (e.g., below 54 mg/dl) may be colored may be colored dark red or maroon. The color-coding may include coloring the area under the curve a certain color or changing the portion of the graph to be a certain color, or otherwise highlighting the region with the corresponding color. The low glucose events sections 1032a, b may also list metrics and/or statistics related to the low glucose events. In some embodiments, the low glucose events metrics sections 1032a, b may list the number of low glucose events and, optionally, the average duration of the low glucose events.

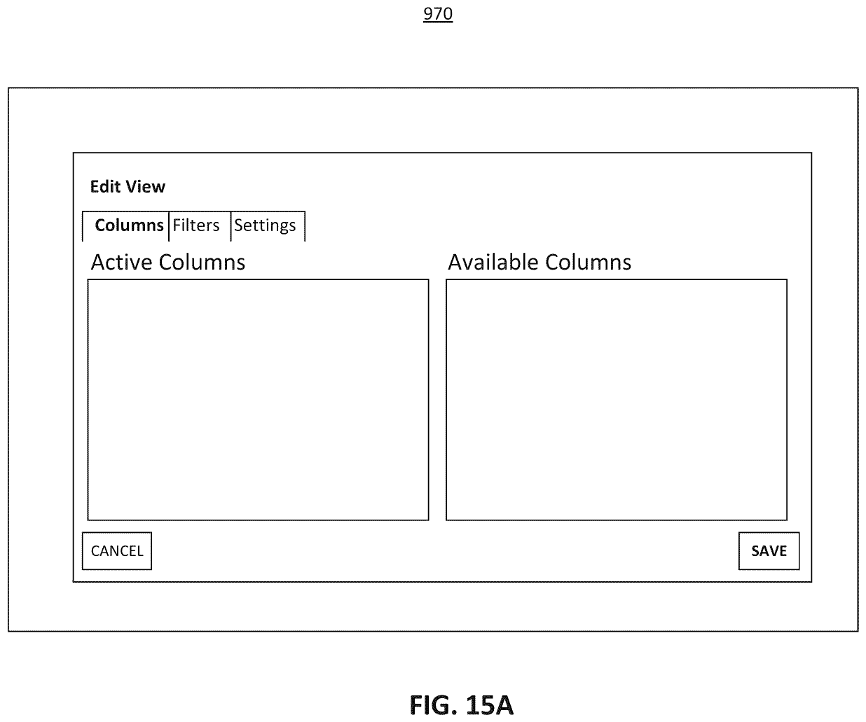

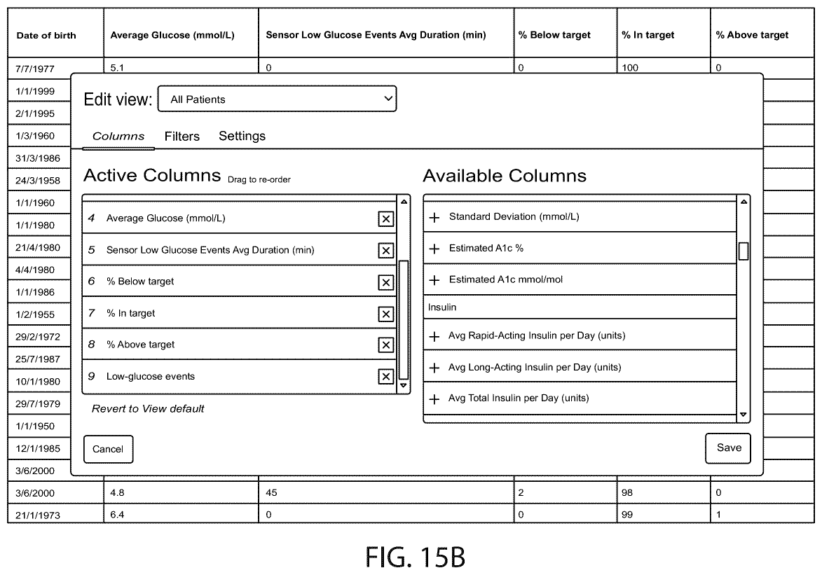

FIGS. 15A-15B depict example embodiments of a GUI associated with a patient dashboard 970. The dashboard 970 may allow a user to display merged insulin data from different sources, e.g., connected pens and manually entered insulin data. The user may select different columns to include in the patient dashboard display, including the average rapid-acting insulin administered per day (units), the average long-acting insulin administered per day (units), and the average total insulin administered per day (units). Other parameters that may be shown on the patient dashboard include average glucose (mmol/L), sensor low glucose events average duration (min), % below target, % in target, % above target, low-glucose events, standard deviation (mmol/L), estimated A1c %, and estimated A1c mmol/mol.

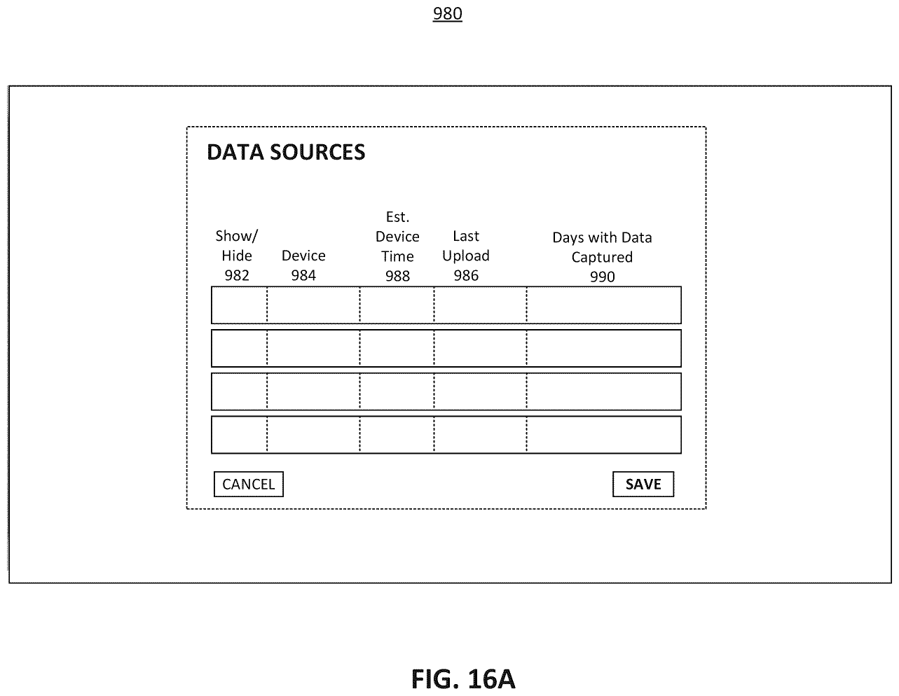

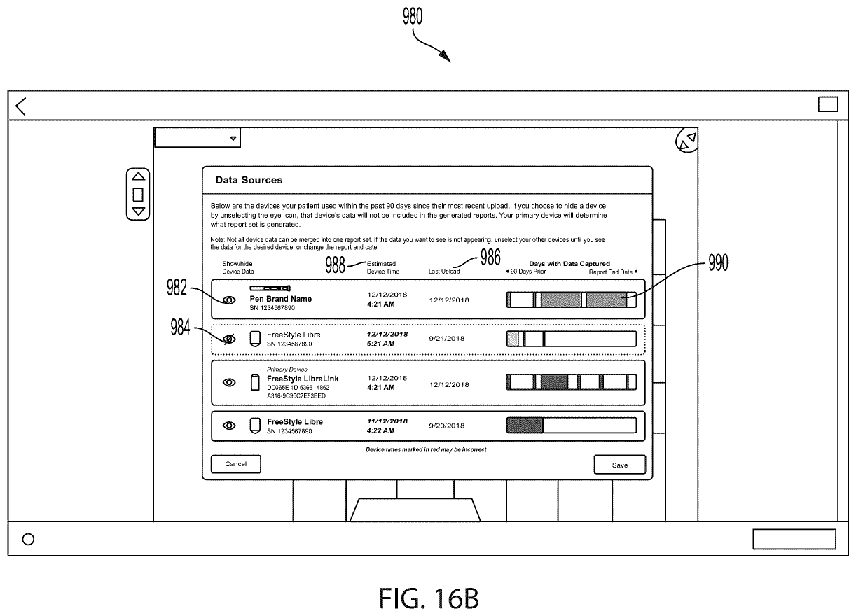

FIGS. 16A-16B depicts an exemplary embodiment of a GUI for a data sources modal with connected pens 980. In the modal 980, the user is able to select the connected pen(s) that they want to include in a report using the show/hide icon 982. A default may be set to display any connected pens with data during the reporting period. Each connected pen may appear as its own device in the modal 980. The connected devices may appear in an order according to the date of the last upload 986, which is the date of the last insulin timestamp. The listing of the devices (insulin pens) may include the insulin pen brand name, insulin pen serial number, and insulin pen image. The pen image may be positioned above the brand name. The modal 980 may also display an estimated device time 988. The estimated device time 988 may be blank if it is unknown or the same time as the device used to upload at the time of the upload. Days with insulin data captured in the last 90 days from the report end date 990 may be displayed in a different color, e.g., green, to differentiate from data related to glucose. With this GUI, an HCP or other user may determine which data sources to include in any of the reports described herein.

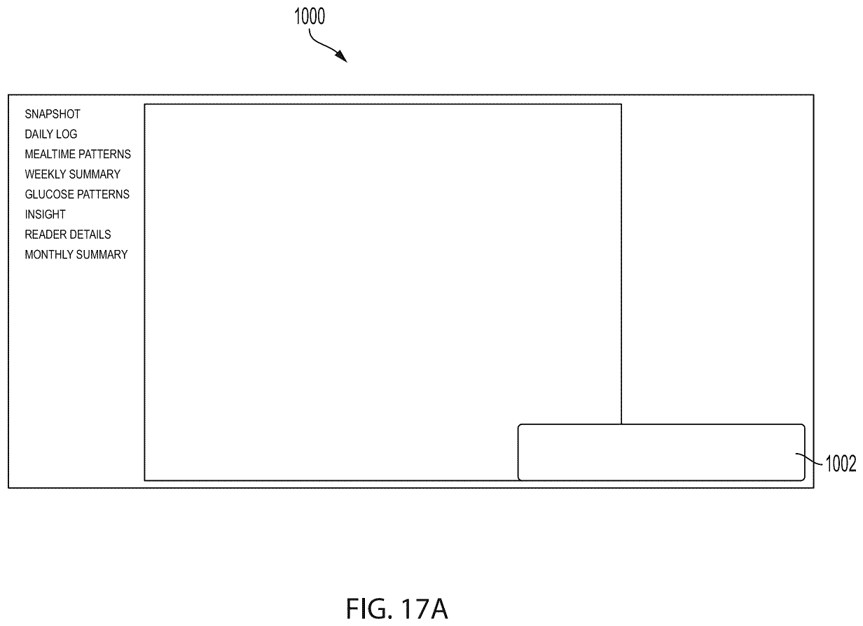

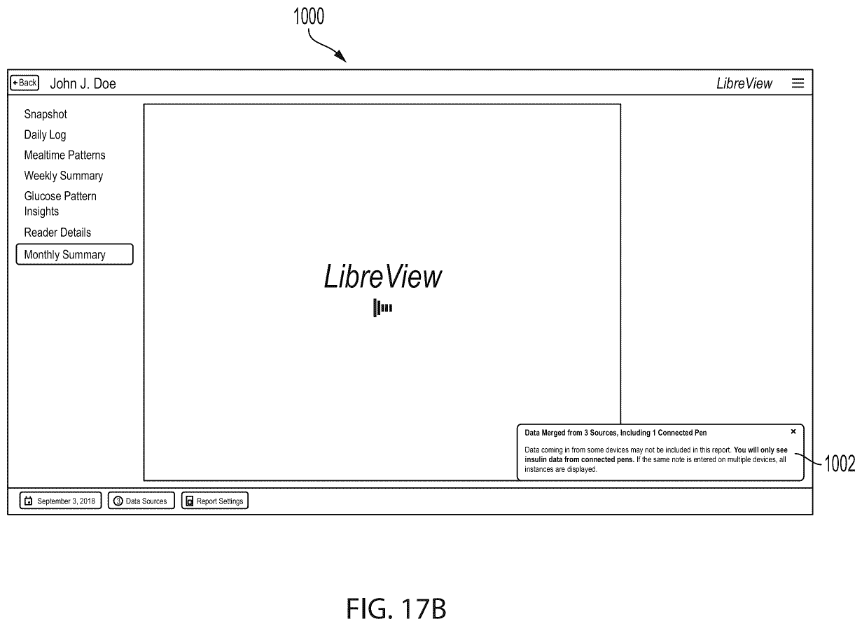

FIGS. 17A-17B depict exemplary alerts 1002, e.g., a toast alert, for reporting information regarding connected pens. The alert 1002 may be displayed as an unobtrusive non-popup box in a portion of the screen, e.g., the bottom right corner for a period of time. The alert 1002 may appear for about 1 minute, or until the user dismisses it, if the report includes insulin pen data. The alert 1002 may show the number of total sources 1004 (including both glucose and insulin sources) and the number of connected pens 1006. The alert 1002 may not be displayed if there is only merged glucose devices and no connected insulin delivery devices. The alert 1002 may indicate that the data coming in from some devices may not be included in the report. The report may only include insulin data from connected pens.

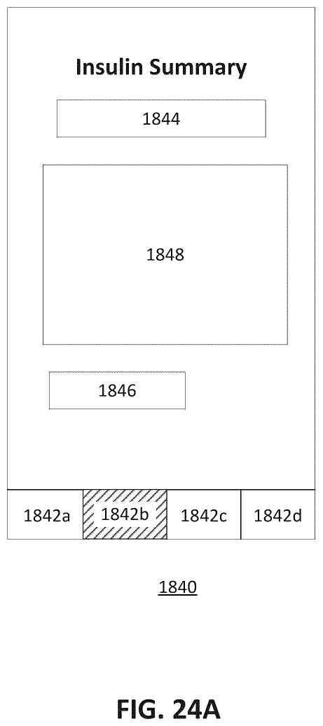

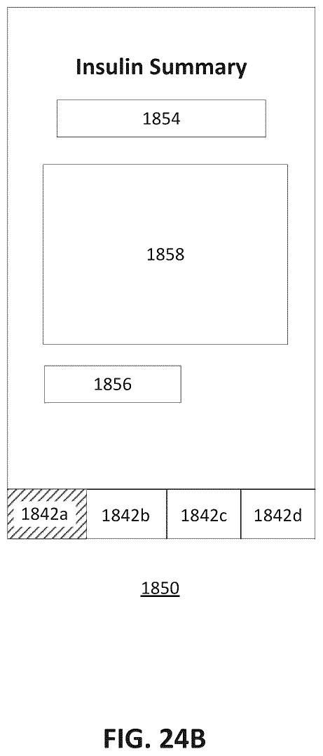

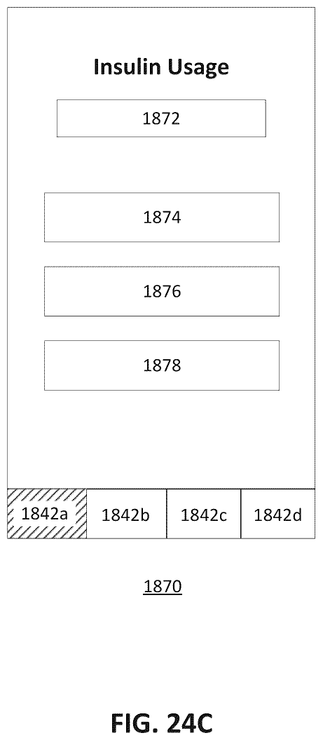

FIGS. 24A-C depict example embodiments of Insulin Summary Report GUIs 1840. Insulin Summary Report GUI 1840 may contain tabs 1842a-1842d with different time periods for which the user may select to display the daily patterns report. For example, the time period tabs 1842a-1842d may be 1 day, 7 days, 14 days, 30 days, or 90 days. The GUI 1840 may also contain the date range 1844 that is currently being displayed and a graphical representation of the total daily dose 1848, along with a legend 1846 for the graphical representation 1848. The graphical representation 1848 may be a bar graph with time on the x-axis and total units administered on the y-axis. The graphical representation may include both rapid-acting and long-acting doses. If the time period selected is 14 days 1842b, then the units of the x-axis may be single days, with each day having a bar showing the total amount of fast-acting and rapid-acting insulin administered that day. In some embodiments, the user may select an entry for a single day by tapping it and a window with details for that day (e.g., the total number of units for fast-acting and rapid-acting insulin administered that day).

In some embodiments, the user may access a daily insulin report GUI 1860 by either tapping on details window for that day or by selecting a daily insulin report through a menu. As seen in FIG. 24B, the daily insulin GUI 1850 may have the time period of 1 day 1842a selected and may display the date 1854 of the data being displayed in the graphical representation 1858. The representation 1858 may be a bar graph with time on the x-axis and total units administered on the y-axis, with each dose amount graphed at the time it was administered. The graphical representation may include both rapid-acting and long-acting doses. In the legend 1856, below each of the rapid-acting and long-acting symbols or colors, the GUI 1850 may list the total amount of each type of insulin injected that day 1854.

FIG. 24C shows an example embodiment of an insulin usage report GUI 1870, which summarizes insulin usage for a selected time period. For example, the time period tabs 1842a-1842d may be 1 day, 7 days, 14 days, 30 days, or 90 days. The GUI 1870 may display the date range 1872 for the insulin usage being displayed. For the selected date range 1872, where the date range selected is more than one day, the GUI 1870 may also display the average total daily dose amount administered 1874, the average total rapid-acting amount administered daily 1876, and the average total long-acting amount administered daily 1878

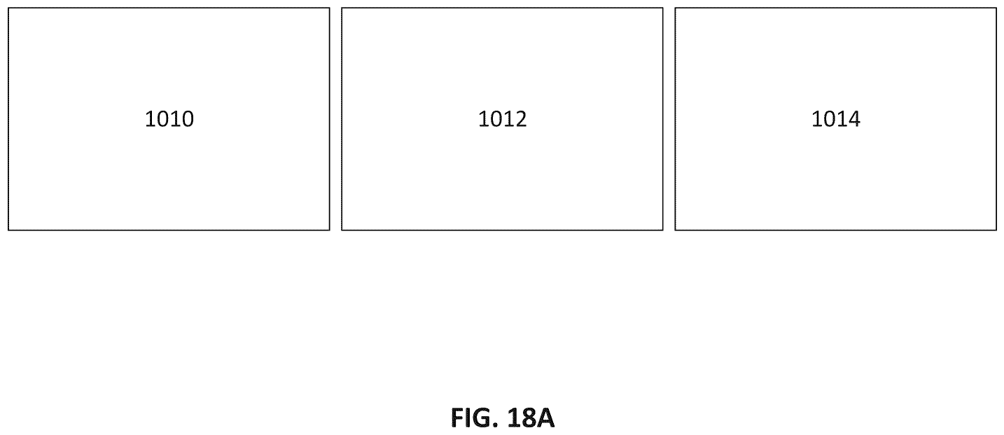

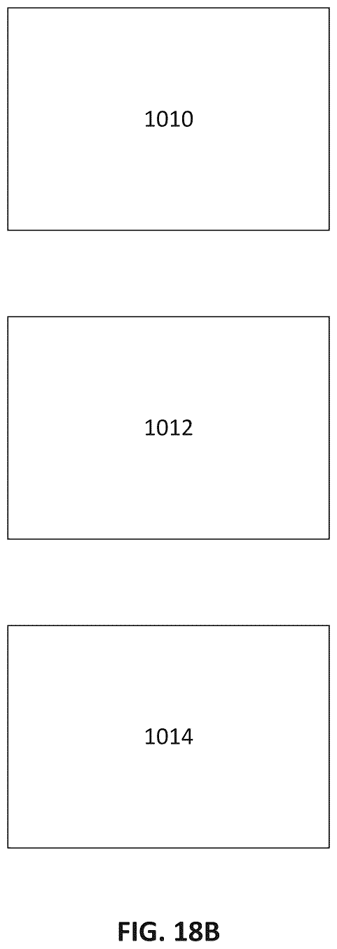

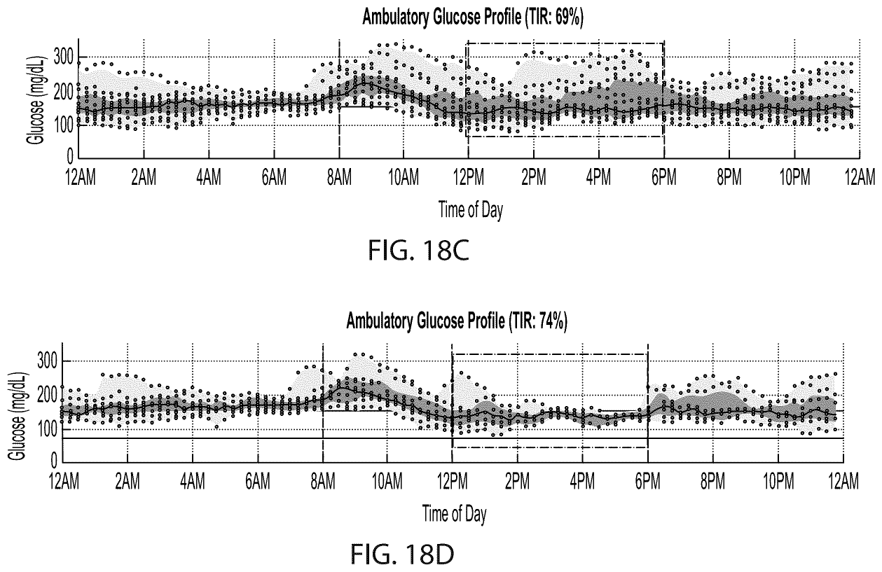

FIGS. 18A-18D depict exemplary embodiments of a GUI of a comparative profiles report. By leveraging dosing data from connected drug delivery devices and glucose data from continuous glucose monitors as inputs, in many embodiments, a GUI or report may include a plurality of glucose profiles over a selected time period. As seen in FIGS. 18A-18B, in some embodiments, the GUI or report may include two, alternatively two or more, alternatively three, alternatively three or more versions of a glucose profile 1010, 1012, 1014 over a selected time period.

In some embodiments, the glucose profile(s) 1010, 1012, 1014 may each be an ambulatory glucose profile (AGP) graph or portions of an AGP. As described with respect to exemplary FIG. 14, the AGP may display the hourly 5^{th}, 25^{th}, 50^{th} (median), 75^{th}, and 95^{th} percentiles of glucose readings, presented over the “typical” 24-hour day based on all days within the selected timeframe. Alternatively, the AGP may display other percentiles, such as the hourly 10^{th}, 25^{th}, 50^{th} (median), 75^{th}, and 95^{th} percentiles of glucose readings, presented over the “typical” 24-hour day based on all days within the selected timeframe. The AGP graph may also include two horizontal lines, which indicate the boundaries of the target range. For example, a first line may correspond to the lower boundary of the target range (e.g., 70 mg/dL) and a second line may correspond to an upper boundary of the target range (e.g., 180 mg/dL). The first and second lines may also be color-coded. The colors may correspond to the same colors used in other reports described herein, e.g., the same colors as the target range bar portion in the Time-in-Ranges portion 308 (e.g., green). Thus, the AGP graph easily illustrates the amount of time spent in (or amount of readings falling within) the target range. Other exemplary AGP graphs can be found in, e.g., US 2018/0235524, US 2014/0188400, US 2014/0350369, US 2018/0226150, all of which are expressly incorporated by reference it their entirety for all purposes.

In other embodiments, the glucose profiles may be plotted as multiple daily traces in a single graph or figure. In some embodiments, the glucose profiles may be converted into an AGP in addition to being plotted as multiple daily traces in the same graph or figure. In some embodiments, the plurality of graphs may be any type of representation of glucose data, include modal day overlays or time-series plots. These graphs may also present data, or be based on data, from other time-based analyte data or other time-based data associated with the patient, such as insulin dosing events or insulin delivery data or meal event data. Although exemplary graphs are described based on glucose data, any patient data may be represented in these exemplary formats.

The GUI or report may include a time filter that allows the user to define a time period to analyze and to display in the plurality of glucose profiles 1010, 1012, 1014. The time period may be the last 7 days, alternatively the last two weeks, alternatively the last month, alternatively the last two months, alternatively the last three months, alternatively the last 6 months, alternatively the last 9 months, alternatively the last year.

A first profile 1010 of the plurality of glucose profiles may display all of the glucose level measurements for the selected time period. See, e.g., FIG. 18C. The first profile 1010 may represent a complete set of glucose level measurements, which may include measurements taken after a glucose level-altering drug was administered and after a dose of a glucose level-altering drug was missed, or the dose information was not received by the program or application.

A second profile 1012 of the plurality of glucose profiles may display only specific data over the selected time period associated with an administered glucose-lowering medication. See, e.g., FIG. 18D. The specific data may only include glucose level measurements taken during a window of time following, or associated with, the administration of a glucose level-altering medication. The specific data associated with the administration of a glucose level-altering medication may be include glucose data or glucose level measurements taken in a window determined by a fixed time-of-day definition. The glucose data may be included from a particular window if a dose of a glucose level-altering medication is associated with that window. The window may still be associated with a dose even if the window does not occur exactly or right after the dose was administered. In some embodiments, a window may be a fixed time of day. For example, glucose levels from a fixed time window (e.g., about 7 am to about 12 pm) may be included only if a rapid-acting insulin dose was administered in that fixed time window. The length of the fixed time window may be related to the therapeutic window of the type of drug administered. For example, a basal or long-acting insulin dose may have a 24-hour fixed time window and a rapid-acting insulin may have a 5-hour fixed time window. In other embodiments, a window may be a variable time of day where a first timepoint in the variable time window is the nearest glucose value following the timestamp of the drug dose or the time of a logged dose, and the variable time window extends for a therapeutic window of the administered drug dose. For example, the therapeutic window may be about 5 hours for a rapid-acting insulin and about 24 hours for a long-acting insulin. The administration of the glucose level-altering medication may be from a connected drug delivery device. In other embodiments, the drug delivery device is not connected and a user may log a dose. The record of the medication administration may be provided by the connected drug delivery device, or by some other means such as manual entry of the medication administration event. The specific data for the second glucose profile 1012 may not include glucose level measurements during the window of time following a missed drug administration, or measurement during the window of time associated with a drug administration. The portion or tranche of glucose level measurements after a missed dose, e.g., the glucose level measurements during the window of time following a missed drug administration or alternatively, measurements from a window that is not associated with administration of a medication, may be excised from the data set including all of the glucose level measurements for the selected time period, and the remaining glucose level measurements may be used to generate the second of the plurality of glucose profiles 1012. Thus, the second of the plurality of glucose profiles 1012 may reflect the user's glycemia when the user remembers to dose. As seen when comparing FIG. 18D to FIG. 18C, the spread or variability of the data in the AGP graph for the profile in which the insulin was dosed (FIG. 18D) is much smaller than the spread or variability of the data in the AGP for the profile containing all data (including data after missed doses).

A third profile 1014 of the plurality of glucose profiles may be a display of only specific data over the selected time period when a glucose level-altering medication was not administered, e.g., by a user or a care-giver. The specific data may be displayed in the third of the plurality of glucose profiles 1014 and may only include the glucose level measurements taken during a window of time following a missed drug administration. The third profile of the plurality of glucose profiles 1014 may contain the data that was excised from the complete data set to generate the second of the plurality of glucose profiles. The length of the portion or tranche of glucose level measurements after the missed dose that is excised may depend on the type and identity of the glucose level-altering medication. The measured glucose levels displayed in the third profile 1014 may have higher glucose level measurements (e.g., a higher median) and/or higher variability as compared to the second profile and the first profile because there are no medications in the user's body to help control the glucose levels. Thus, glucose levels associated with a missed dose may result in high glucose. Moreover, the high glucose levels resulting from missed doses may mask low glucose events following administered doses when all glucose data are aggregated and plotted together. A glucose profile that displays only the data from the window of time after glucose-lowering medication doses are administered (e.g., 1012) more directly highlights the effects of those drugs on glycemia, thereby helping a trained health care professional potentially titrate a current dose regimen. A profile that highlights the glucose levels where doses are missed (e.g., 1014) may more directly highlight the effects of poor dose concordance on a subject's glycemia.

Thus, the first profile 1010, which displays the glucose levels in which no data was removed, and the third profile 1014, which displays the glucose levels measured after missed doses, may display higher glucose level measurements (e.g., a higher median) and/or higher variability as compared to the second profile 1012, which displays the glucose levels measured after the administration of glucose-lowering medication.

In some embodiments, the plurality of glucose profiles may be presented in a conjoined display, either side-by-side (e.g., three graphs on the same row as seen in FIG. 18A) or one on top of the other (e.g., three figures in the same column as seen in FIG. 18B), that may enable an HCP to identify differences between the traces easily and quickly. In some embodiments, a transparent overlay of one glucose profile on top of the other(s) to make a single figure may be displayed. In other embodiments, problematic areas may be highlighted in the glucose profiles to demonstrate how poor concordance or current dose regimens are affecting glucose outcomes. The plurality of profiles may be presented in any order or spatial arrangement.

Connected drug delivery device data may also include dose timestamps. If the connected drug delivery device data do not present a dose to the program creating the glucose profiles, then the program may assume that a regularly scheduled dose was not taken by the user, i.e., a missed dose. In the case of a missed dose, a pre-defined portion or tranche of glucose data may be removed from a data set that contains all of the glucose level measurements (which may be used to create the first glucose profile 1010), leaving only glucose data that may be associated with an administered drug dose (which may be used to create the second glucose profile 1012). This excised portion or tranche may then be used to create the third glucose profile 1014.

The length of each excised glucose data portion or tranche (or the window of time from which the data is excluded) may be dependent on the pharmacokinetic and pharmacodynamic profiles of the user's medications. For example, if a person doses a once-daily basal insulin injection on a Monday but forgets to dose on Tuesday, data from the connected drug delivery device may show a dose timestamp on Monday, but not for Tuesday. As a result, glucose data from Monday may be included in generating the second glucose profile 1012, but glucose data from Tuesday may not be included in the data displayed in the second glucose profile. The Tuesday data may, however, be used in the generation of the third glucose profile 1014. In this example, a day's worth of glucose data may be excluded because long-acting basal insulin has a pharmacodynamic glucose-lowering action time of about 24 hours. If instead a mealtime rapid acting insulin dose was missed, a smaller portion or tranche of glucose data may be excised from generation of the second glucose profile and used for generation of the third glucose profile. This is because rapid acting insulin has a pharmacodynamic glucose-lowering action time of about 6 hours.

In some embodiments, the plurality of graphs may include at least 2 graphs, alternatively at least 3 graphs, alternatively at least 4 graphs, alternatively at least 5 graphs, alternatively at least 6 graphs, alternatively at least 7 graphs, alternatively at least 8 graphs. Each of the plurality of graphs may display a different data set. In some embodiments, where a person has a treatment regimen that includes both basal insulin doses and bolus doses, 5 graphs may be generated and/or displayed. A first graph may be a display or presentation of all of the glucose measurements from a time period, a second graph may include glucose measurements taken during a window of time following, or associated with, the administration of basal insulin dose(s), a third graph may include glucose measurements taken during a window of time following, or associated with, missed administration(s) of basal insulin dose(s), a fourth graph may include glucose measurements taken during a window of time following, or associated with, the administration of bolus insulin dose(s), and a fifth graph may include glucose measurements taken during a window of time following, or associated with, missed administration(s) of bolus insulin dose(s).

The glucose level-altering medication may be a glucose lowering medication or a glucose-raising medication. The embodiments described herein relate to glucose-lowering medication, such as insulin, but the framework may be generalizable to glucose-raising medications, such as glucagon. The glucose level-lowering medication may be a type of insulin. Types of insulin include rapid-acting insulin, short-acting insulin, intermediate-acting insulin (e.g., NPH insulin), mixed insulin (e.g., premixed insulin), long-acting insulin, and ultra long-acting insulin. While the examples presented here are for insulin dosing, this methodology is generalizable to any glucose-altering drug with a known glucose-lowering time delivered from a connected drug delivery device. These may include, but are not limited to, SGLT2 inhibitors, GLP1 receptor agonists, biguanides (e.g., metformin), α-glucosidase inhibitors, thiazolidinediones, DPP4 inhibitors, and combinations thereof.

The length of the portion or tranche or the window of time from which data may be excised after a missed once-daily, long-acting, or basal insulin dose may be about 1 day, alternatively between about 20 hours and about 28 hours, alternatively between about 22 hours and about 40 hours, alternatively between about 20 hours and about 38 hours, alternatively between about 20 hours and about 36 hours.

The length of the portion or tranche or the window of time from which data may be excised after a missed rapid-acting insulin dose may be about 2 hours, alternatively about 2.5 hours, alternatively about 3 hours, alternatively about 3.5 hours, alternatively about 4 hours, alternatively about 4.5 hours, alternatively about 5.0 hours, alternatively about 5.5 hours, alternatively about 6.0 hours, alternatively about 6.5 hours, alternatively about 7.0 hours, alternatively between about 2.0 hours and about 7.0 hours, alternatively between about 3.0 hours and about 7.0 hours, alternatively between about 4.0 hours and about 7.0 hours of administration of the glucose-lowering medication. In some embodiments, the length of the portion or tranche or the window of time from which data may be excised may be defined or determined by the medication, such as by the insulin action time of a prandial insulin. In some embodiments, the length of the portion or tranche or the window of time from which data may be excised may be preset or estimated by the system. In other embodiments, the length of the portion or tranche or the window of time from which data may be excised may be manually entered.

The length of the portion or tranche or the window of time from which data may be excised after a missed intermediate-acting insulin dose may be about 12 hours, alternatively between about 8 hours and about 16 hours, alternatively between about 10 hours and about 14 hours.

The length of the portion or tranche or the window of time from which data may be excised after a missed ultra-long-acting insulin dose may be about 36 hours to about 42 hours, alternatively between about 32 hours and about 44 hours.

In some embodiments, the user may use a filter to customize a GUI or report to display different sets of data. The data may be used to illustrate different types of nonadherence. The filter may include selections to display specific data over a selected period of time when a recommended dose of a glucose level-altering medication was taken, when an under-bolused dose of a glucose level-altering medication was taken, and when an over-bolused dose of a glucose level-altering medication was taken. A graph of specific data over a selected time period when a glucose level-altering medication was under-bolused, i.e., when less than a recommended amount of the glucose level-altering medication was taken, and/or a graph of specific data over a selected time period when a glucose level-altering medication was over-bolused, i.e., when more than a recommended amount of the glucose level-altering, shown in comparison to a graph showing specific data over a selected time period when a recommended amount of a glucose-level altering medication was taken could assist an HCP in convincing a patient to follow a recommended dosing regimen, rather than altering their dose amounts.

In some embodiments, the filter may also include a selection to display specific data over a selected time period when a late meal dose, i.e., a dose was taken a period of time after a start of a meal, was taken. In some embodiments, a late meal dose or an extra meal dose may be when a dose is provided at least one hour after a meal but within three hours after a meal. In some embodiments, the filter may also include a selection to display specific data over a selected time period when an extra meal dose, i.e., an additional dose was taken during the meal, was taken. Graphs of specific data over a selected time period when a glucose level-altering medication was taken a period of time after a meal start, and/or specific data over a selected time period when an additional dose of a glucose level-altering medication was taken during a meal, may help an HCP reduce a patient's fear of hypoglycemic episodes.

In addition to the other ways described herein, nonadherence may be determined if a dose is taken outside of a preset time period. For example, nonadherence of a basal dose may be determined if a dose was missed or taken outside of a time window from a prescribed dosing time. In some embodiments, the time window may be about 30 minutes, alternatively about 1 hour, alternatively about 90 minutes after the prescribed dosing time. For bolus doses, each meal may have an associated time window. For example, an associated time window for breakfast may be about 6 am to about 11 am, an associated time window for lunch may be about 11 am to about 4 pm, an associated time window for dinner may be about 4 pm to about 10 pm. A time period associated with overnight may be 10 pm to about 6 am. Doses may be determined to be correct if a recommended dose amount was given in an associated time window. A dose may be determined to be missing if no dose was administered during an associated time window. A dose may be determined to be an extra dose if an additional dose beyond a single dose was taken during an associated time period. A dose may be determined to be non-adherent if the dose amount administered during the time period is different than the recommended dose amount. Additionally, missing and extra doses may also be determined to be non-adherent.

In other embodiments, the filter may include a selection to display specific data over a selected time period when an alarm was enabled. The alarm may be a low glucose alarm that is set to notify the user when an analyte level is below a low threshold, a high glucose alarm that is set to notify the user when an analyte level is above a high threshold, or both. The low threshold or high threshold may be set by the user or may be set by the system. The low threshold or high threshold may be set to different values for different time periods. The alarms may be enabled at different thresholds. Thus, a first graph may include a glucose profile that includes all of the analyte levels for a time period whether or not an alarm was enabled. A second graph may include a glucose profile that only includes the analyte levels for the time period when at least one alarm was enabled. A third graph may optionally be included that includes a glucose profile that only includes the analyte levels for the time period when at least one alarm was not enabled or when the at least one alarm was disabled.

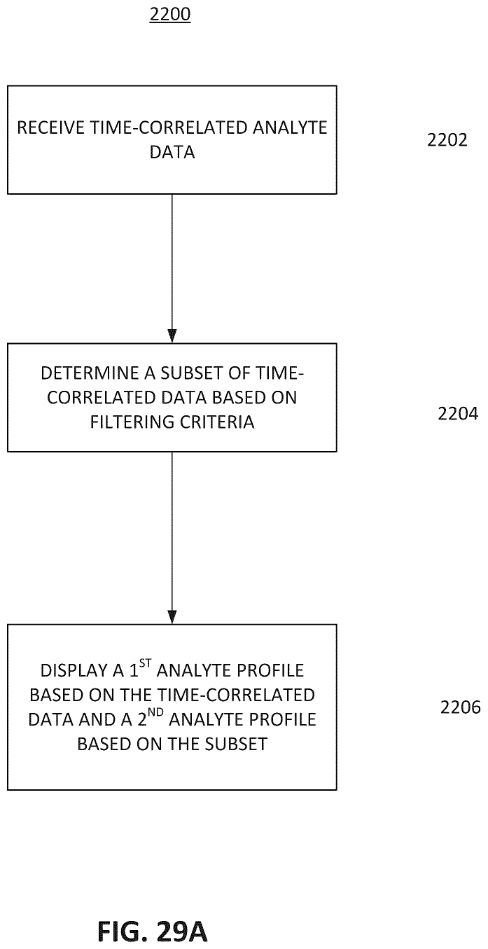

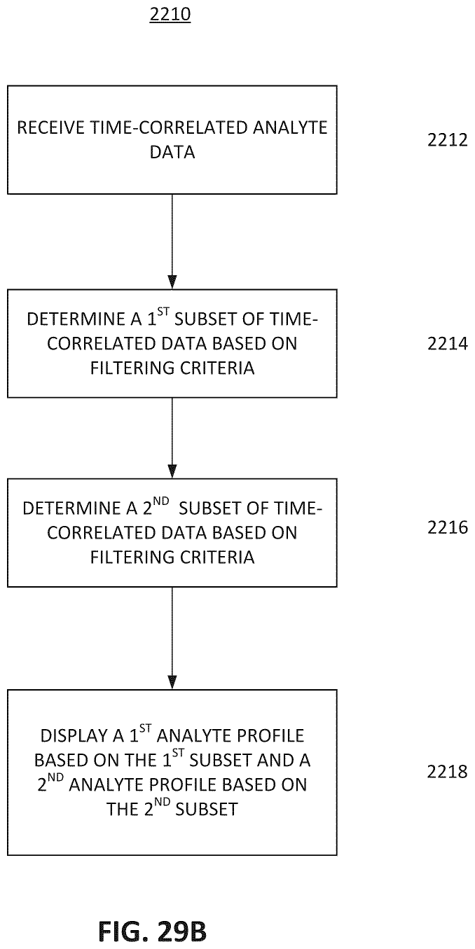

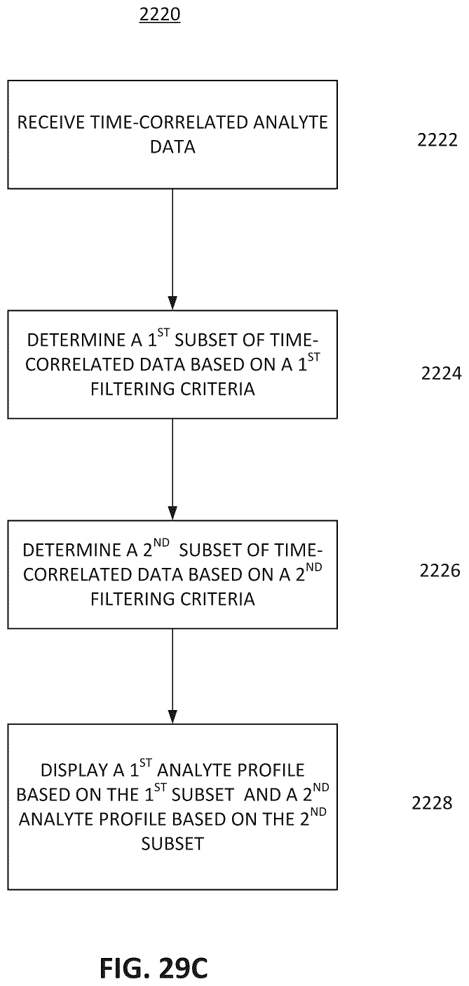

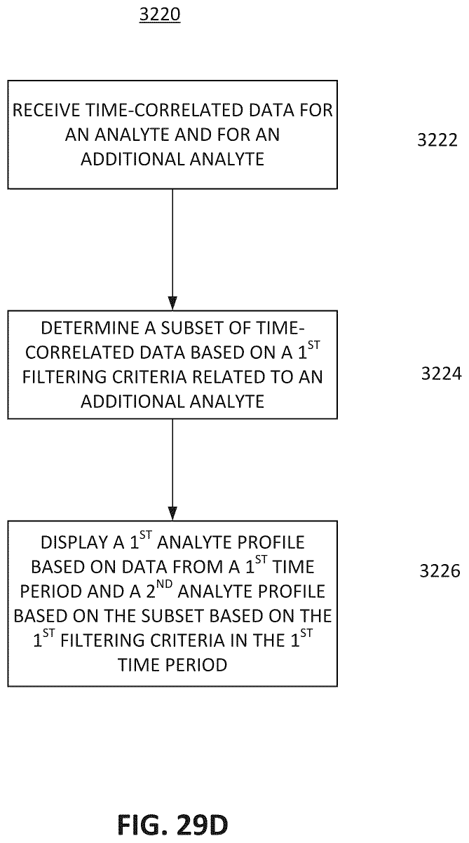

As seen in FIG. 29A, in exemplary method 2200, beginning with step 2202, the system or application may receive time-correlated analyte data or time-correlated data characterizing an analyte level. The data may be received from a server, the cloud, or from a sensor control device 102. In step 2204, the system or application may determine a subset of time correlated data based on a filtering criteria. In some embodiments, the filtering criteria may be the enablement of at least one alarm. The system may also receive time-correlated dosage data for doses of a glucose-altering medication received by the subject over a period of time and the filtering criteria may include dose concordance of the glucose altering medication. In some embodiments, the dose concordance comprises recommended doses, missed doses, under-bolused doses, over-bolused doses, late meal doses, or extra meal doses. In step 2206, the system or method may display a first analyte profile based on the time-correlated data and a second analyte profile based on the subset determined based on the filtering criteria.

In some embodiments, the filtering criteria may be based on a type of day. For example, the type of day may be a weekday, a weekend day, a work day, a vacation day, a day in which the user or person exercised, or a day in which the user or person did not exercise. In some embodiments, the filtering criteria may be based on dose adherence or dose nonadherence of the user or person. For example, the filtering criteria may be based on whether or not the user or person took a basal insulin dose or missed their basal insulin dose. In some embodiments, the filtering criteria may be based on whether or not the user or person missed at least one meal bolus dose.

In an alternative method, as seen in FIG. 29B, in exemplary method 2210, beginning with step 2212, the system or application may receive time-correlated analyte data or time-correlated data characterizing an analyte level of the subject. The data may be received from a server, the cloud, or from a sensor control device 102. In step 2214, the system or application may determine a first subset of time correlated data based on a filtering criteria. In some embodiments, the filtering criteria may be the enablement of at least one alarm. The system may also receive time-correlated dosage data for doses or dose amounts of a glucose-altering medication received by the subject over a period of time and the filtering criteria may include dose concordance of the glucose altering medication. In some embodiments, the dose concordance comprises recommended doses, missed doses, under-bolused doses, over-bolused doses, late meal doses, or extra meal doses.

In step 2216, the system may determine a second subset of time-correlated analyte data based on the filtering criteria. In some embodiments, the second subset of data may include the data that does not satisfy the filtering criteria. For example, if the filtering criteria is whether at least one alarm is enabled, the first subset of time-correlated analyte data may be the data during which the at least one alarm was enabled, and the second subset of time-correlated analyte data may be the data during which the at least one alarm was not enabled (or when the at least one alarm was disabled).

In step 2218, the system or method may display a first analyte profile based on the first subset of time-correlated analyte data and a second analyte profile based on the second subset of time-correlated analyte data based on the filtering criteria.

It will be understood that the reports and GUIs described herein may include any mix of the described graphs or profiles. Any mixture or subset (e.g., only two of the three graphs) of these graphs may also be displayed in a GUI or report. For instance, the GUI or report may include a glucose profile when the at least one alarm was enabled and a glucose profile when the at least one alarm was disabled, and may not include the glucose profile including all of the data regardless of the alarm status.

By separating and presenting glucose data based upon the presence or absence of alarms, the user and their care provider will be able to directly see their impact on glycemia. The ability to visualize glucose profiles when alarms are enabled may be particularly useful to people on therapies that may cause hypoglycemia. Comparison of the profiles with alarms enabled and without alarms enabled could show the user how enabling low glucose alarms and acting upon them can mitigate instances of hypoglycemia compared to having the low glucose alarms turned off.

In some embodiments, indications of alarm occurrences may be added to any of the glucose profiles within the relevant time windows. Alarm occurrences could be indicated by the inclusion of an icon, such as a bell or alarm clock, on the glucose profile. Alarm occurrences may include times when alarm conditions were met, regardless of whether or not alarms were enabled. The inclusion of alarm occurrences on, e.g., a glucose profile for data where alarms were not enabled may demonstrate the frequency of alarms by time of day and how the alarms were missed by the user having the alarms turned off or disabled, thereby contributing to poor glucose control.

In some embodiments, comparative glucose profiles may be presented showing alarms enabled at different thresholds. For instance, a glucose profile having a low glucose alarm threshold enabled at 70 mg/dL can be presented against a glucose profile having a low glucose alarm threshold enabled at 90 mg/dL.

In some embodiments, for any of the various filtering criteria described herein, the user may select an additional filter criteria to further customize the graphs or metrics that are displayed. For example, the user may select a first level filter to display data or a metric when a low glucose alarm has been enabled. The user may then select an additional filter, or a second level filter, related to dose concordance. For example, the user may select to display data or a metric in which the user has taken a medication. Such a representation could demonstrate that the user's current medication is leading to hypoglycemia and should be decreased. Dosing information that may be gathered from a connected device (e.g., a connected pen or pump) or from user input. In another example, the user may additionally elect to add indications of alarm occurrences (or times when alarm conditions have been satisfied) into the display, which could highlight opportunities to adjust their medication doses.

As seen in FIG. 29C, in exemplary method 2220, beginning with step 2222, the system or application may receive time-correlated analyte data or time-correlated data characterizing an analyte level of the subject. The data may be received from a server, the cloud, or from a sensor control device 102. In step 2224, the system or application may determine a first subset of time correlated data based on a first filtering criteria. In step 2226, the system or method may determine a second subset of time correlated data based on a second filtering criteria. The filtering criteria for either step may be the enablement of at least one alarm, where the different filtering criteria are different types of alarms or alarms with different thresholds. The system may also receive time-correlated dosage data for doses of a glucose-altering medication received by the subject over a period of time and the filtering criteria may include dose concordance of the glucose altering medication. In some embodiments, the dose concordance comprises recommended doses, missed doses, under-bolused doses, over-bolused doses, late meal doses, or extra meal doses. In step 2228, the system or method may display a first analyte profile based on the first subset of time-correlated analyte data and a second analyte profile based on the second subset of time-correlated analyte data based on the filtering criteria.

In an alternative embodiment, as seen in FIG. 29D, in exemplary method 3220, beginning with step 3202, the system or application may receive time-correlated data for an analyte and for an additional analyte or time-correlated data characterizing an analyte level and an additional analyte level of the subject. In some embodiments, the analyte may be glucose. In some embodiments, the additional analyte may be a ketone, a ketone body, β-hydroxybutyrate, or lactate.

In step 3224, the system or application may determine a subset of time correlated data based on a first filtering criteria related to at least one determined level of the additional analyte. In some embodiments, the first filtering criteria may be based on when the at least one determined analyte level of the additional analyte is above a threshold value. In some embodiments, if the additional analyte is ketone, a ketone body, or β-hydroxybutyrate, the threshold value may be at least 1 mmol/L, for example between about 1 mmol/L and 2 mmol/L, alternatively about 1 mmol/L, alternatively about 1.1 mmol/L, alternatively about 1.2 mmol/L, alternatively about 1.3 mmol/L, alternatively about 1.4 mmol/L, alternatively about 1.5 mmol/L, alternatively about 1.6 mmol/L, alternatively about 1.7 mmol/L, alternatively about 1.8 mmol/L, alternatively about 1.9 mmol/L, alternatively about 2.0 mmol/L. In some embodiments, if the additional analyte is lactate, the threshold value may be between about 3 mmol/L and 5 mmol/L, alternatively between about 3.5 mmol/L and 4.5 mmol/L, alternatively about 3 mmol/L, alternatively about 3.25 mmol/L, alternatively about 3.5 mmol/L, alternatively about 3.75 mmol/L, alternatively about 4 mmol/L, alternatively about 4.25 mmol/L, alternatively about 4.5 mmol/L, alternatively about 4.75 mmol/L, alternatively about 5.0 mmol/L.

In step 3226, the system or method may display a first analyte profile based on the time-correlated data and a second analyte profile based on the subset determined based on the filtering criteria. In some embodiments, the analyte displayed in the first and second analyte profiles are the same analyte, e.g., glucose.

In some embodiments, the method may optionally include the step of determining whether an activity level of the person, user, or subject is below a high activity threshold before displaying the 2^{nd} analyte profile.

For example, the user may select a first level filter to display data or a metric when a high glucose alarm has been enabled. The user may then select an additional filter, or a second level filter, related to dose concordance. If presented alongside a time-of-day distribution of alarm occurrences, this figure could highlight opportunities to increase prandial insulin doses.

In some embodiments, the various filtering criteria may be selected through a drop-down menu or a listing of options.

In some embodiments, the various filtering criteria could be used with any of the reports described herein depicting different glucose data. Moreover, the various filtering criteria could be used with any glucose report, insulin report, or meal/exercise report, or combinations thereof.

In the case of an alarm filter, the filter could be used in association with any report of remote sensing data where the alarm is associated with one of the data. In other embodiments, the data for which the alarm is associated with is included in the report. In some embodiments, the data for which the alarm is associated with is not included in the report. For example, an insulin dosing report may be filtered according to whether or not glucose alarms were enabled. The display may also include indications of the insulin doses (and optionally the amount) time-aligned with the glucose alarm occurrences (or instances when an alarm condition was satisfied).

In some embodiments, any analyte metric calculation and/or condition indication may be filtered based on a filtering criteria. For example, a report that incorporates A1c lab measurements may be filtered. In some embodiments, the user may choose to only analyze data from a certain time period (e.g., 1 month) prior to A1c values greater than a certain threshold (e.g., 6.5%, 7%, 7.5%, or 8%). Similarly, data may be filtered according to when a user viewed a detected analyte level, e.g., by accessing a GUI displaying a most recent analyte level in a mobile application or on a reader device. The user may elect to see data for a time period (e.g., about 2 hours, about 3 hours, about 4 hours, between about 1-5 hours, between about 2-5 hours, between about 2-4 hours) after the user has viewed a detected analyte level.

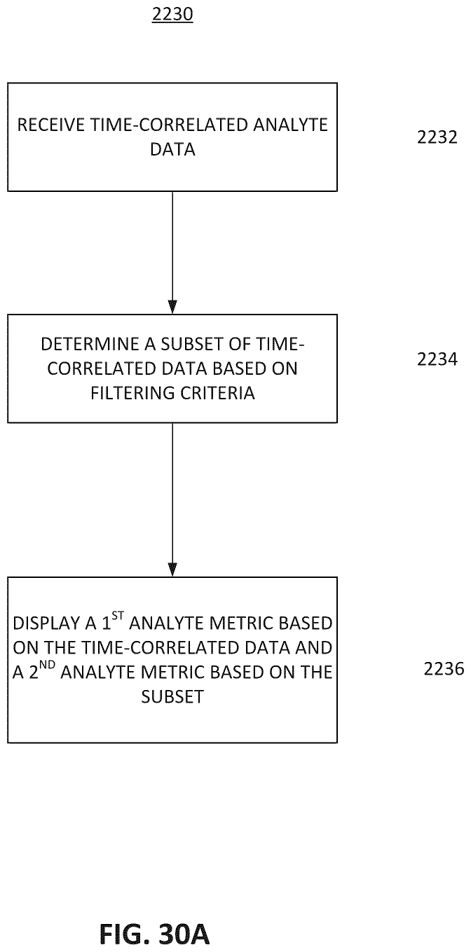

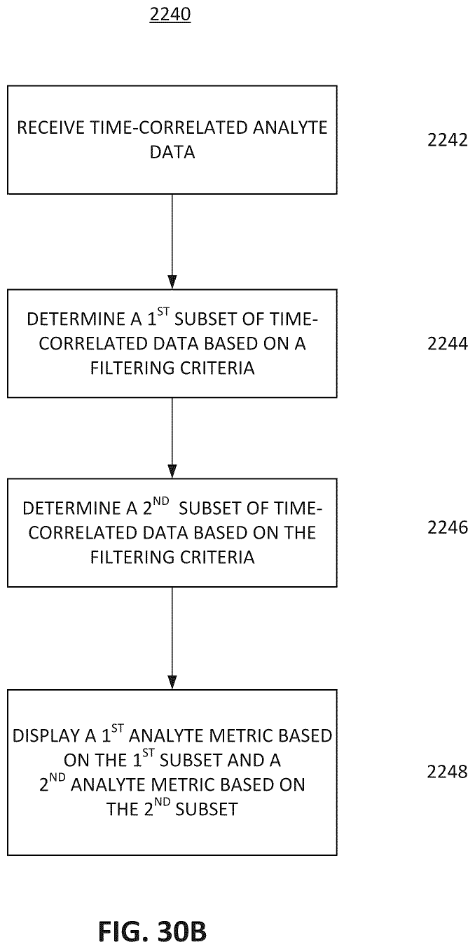

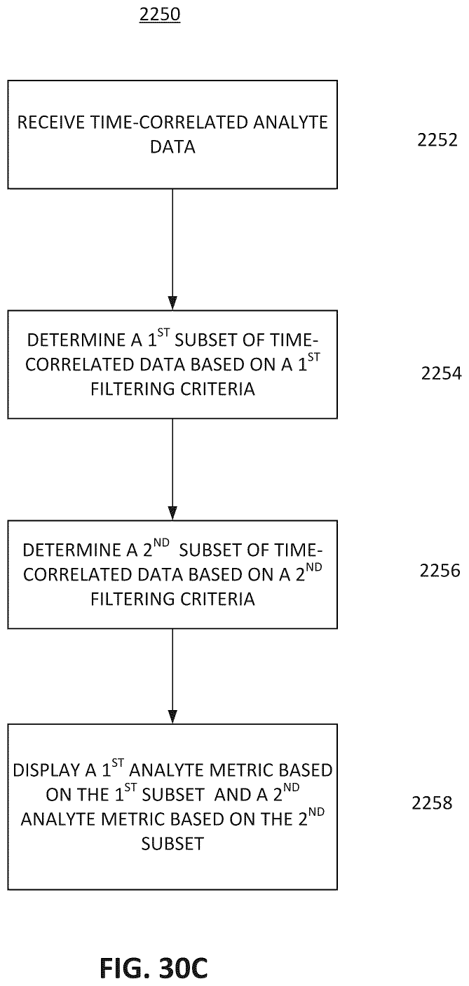

As seen in FIG. 30A, in exemplary method 2230, beginning with step 2232, the system or application may receive time-correlated analyte data or time-correlated data characterizing an analyte level of the subject. The data may be received from a server, the cloud, or from a sensor control device 102. In step 2234, the system or application may determine a subset of time correlated data based on a filtering criteria. In some embodiments, the filtering criteria may be the enablement of at least one alarm. The system may also receive dosage data for doses of a glucose-altering medication received by the subject over a period of time and the filtering criteria may include dose concordance of the glucose altering medication. In some embodiments, the dose concordance comprises recommended doses, missed doses, under-bolused doses, over-bolused doses, late meal doses, or extra meal doses. In step 2236, the system or method may display a first analyte metric based on the time-correlated data and a second analyte metric based on the subset determined based on the filtering criteria.

In an alternative method, as seen in FIG. 30B, in exemplary method 2240, beginning with step 2242, the system or application may receive time-correlated analyte data or time-correlated data characterizing an analyte level of the subject. The data may be received from a server, the cloud, or from a sensor control device 102. In step 2244, the system or application may determine a first subset of time correlated data based on a filtering criteria. In some embodiments, the filtering criteria may be the enablement of at least one alarm. The system may also receive dosage data for doses of a glucose-altering medication received by the subject over a period of time and the filtering criteria may include dose concordance of the glucose altering medication. In some embodiments, the dose concordance comprises recommended doses, missed doses, under-bolused doses, over-bolused doses, late meal doses, or extra meal doses.

In step 2246, the system may determine a second subset of time-correlated analyte data based on the filtering criteria. In some embodiments, the second subset of data may include the data that does not satisfy the filtering criteria. For example, if the filtering criteria is whether at least one alarm is enabled, the first subset of time-correlated analyte data may be the data during which the at least one alarm was enabled, and the second subset of time-correlated analyte data may be the data during which the at least one alarm was not enabled (or when the at least one alarm was disabled).

In step 2248, the system or method may display a first analyte metric based on the first subset of time-correlated analyte data and a second analyte metric based on the second subset of time-correlated analyte data based on the filtering criteria.

As seen in FIG. 30C, in exemplary method 2250, beginning with step 2252, the system or application may receive time-correlated analyte data or time-correlated data characterizing an analyte level of the subject. The data may be received from a server, the cloud, or from a sensor control device 102. In step 2254, the system or application may determine a first subset of time correlated data based on a first filtering criteria. In step 2256, the system or method may determine a second subset of time correlated data based on a second filtering criteria. The filtering criteria for either step may be the enablement of at least one alarm, where the different filtering criteria are different types of alarms or alarms with different thresholds. The system may also receive dosage data for doses of a glucose-altering medication received by the subject over a period of time and the filtering criteria may include dose concordance of the glucose altering medication. In some embodiments, the dose concordance comprises recommended doses, missed doses, under-bolused doses, over-bolused doses, late meal doses, or extra meal doses. In step 2258, the system or method may display a first analyte metric based on the first subset of time-correlated analyte data and a second analyte metric based on the second subset of time-correlated analyte data based on the filtering criteria.

The analyte metric calculation that may be filtered may include time in range metrics. The time in range metrics may include a plurality of ranges. As described elsewhere, the time in range metrics may be displayed in a graph, such as a bar graph, a histogram, a pie chart, and other known graphs. In some embodiments, Time-in-Ranges GUI portion 954 may include a single bar comprising up to five bar portions including (from top to bottom): a first bar portion indicating that the user's glucose range is “Very High” or above 250 mg/dL of a predefined amount of time, a second bar portion indicating that the user's glucose range is “High” or between 180 and 250 mg/dL of the predefined amount of time, a third bar portion indicating that the user's glucose range is within a “Target Range” or between 70 and 180 mg/dL of the predefined amount of time, a fourth bar portion indicating that the user's glucose range is “Low” or between 54 and 69 mg/dL of the predefined amount of time, and a fifth bar portion indicating that the user's glucose range is “Very Low” or less than 54 mg/dL of the predefined amount of time. Time-in-Ranges GUI 954 may display text adjacent to each bar portion indicating an actual amount of time, e.g., in hours and/or minutes.

In some embodiments, the different filtered glucose profiles or metrics may be displayed on the same graph. In other embodiments, the different filtered glucose profiles or metrics may be displayed on different graphs that are displayed on a single GUI or in a single report.

Meals may be identified or logged by a subject and a meal start time may be determined from the manual entry. Alternatively, meal start times may be estimated by many methods. Exemplary methods are described in U.S. application Ser. No. 16/944,736 and U.S. application Ser. No. 17/591,229; see also Harvey, R. A. et al. “Design of the Glucose Rate Increase Detector—A Meal Detection Module for the Health Monitoring System,” J Diabetes Sci Techol. 2014 March; 8(2): 307-320, which is hereby expressly incorporated by reference in its entirety for all purposes. A late dose may be determined if a dose is determined to be taken a period of time after a meal start time, e.g., about 30 minutes, alternatively about 45 minutes, alternatively about 60 minutes, alternatively about 90 minutes after an estimated meal start. In other embodiments, a meal may be determined to have occurred if glucose levels from a CGM are >70 mg/dl and there was a >70 mg/dl rise within two hours. In such a case, a late meal bolus dose may be defined when glucose levels from a CGM increased >50 mg/dl from baseline prior to the insulin dose. A missed meal bolus dose may be defined when no insulin dose was taken within two hours before the start of the rise in glucose levels. In some embodiments, a missed meal dose may be defined by an 80 mg/dl glucose increase over ≤2 hours not preceded within 1 hour by an insulin dose.

In some embodiments, the GUI or report may also include at least some analyte metrics related to each profile of the plurality of glucose profiles. The at least some analyte metrics may include, but are not limited to, an average or median analyte level for the time period, a standard deviation (SD), a CV ([(SD of glucose)/(mean glucose)]×100), a display of a time-in-range for the time period, a GMI index for the time period, a count of a number of high and/or low excursions or their duration below a high and/or low threshold, respectively, a count of a number of very high and/or very low excursions or their duration below a very high and/or very low threshold, respectively, a plurality of dose indicators corresponding to doses of the medication administered, as described with respect to other reports and embodiments in this specification. In some embodiments, pattern analysis of the glucose profile may be based on various inclusions or exclusions. For example, a “low” pattern may be determined for the windows that are associated with medication administration, but not for windows that are not associated with medication delivery. Dose guidance or delivery may be based on these patterns determine only from windows associated with medication administration. In some embodiments, recommendations may be made based on the types of pattern(s) determined or detected. Further details of pattern analysis are described in WO 2021/026004, which is hereby expressly incorporated by reference in its entirety for all purposes.

The methods described herein are not limited to metrics or plots to be displayed but may also be used in processes, such as automatic insulin (or other medication) delivery processes, insulin (or other medication) dose guidance systems and therapy guidance systems. In some embodiments, data used as input or feedback in these systems may include or exclude different glucose (or analyte) readings if associated with missed medication doses as described here. For example, a therapy guidance system may provide therapy change recommendations based on glucose data that excludes glucose data associated with missed medication doses. In other embodiments, an automated insulin delivery system may utilize an adaptive model; the adaptive processing may be based on glucose data excluding glucose data where medication doses are missed.

In other embodiments, a plurality of graphs may be presented in a comparative profiles report. By leveraging dosing data of medications from, e.g., connected drug delivery devices, and analyte data from continuous analyte monitors as inputs, in many embodiments, a GUI or report may include a plurality of analyte profiles over a selected time period. As seen in FIGS. 18A-18B, in some embodiments, the GUI or report may include two, alternatively two or more, alternatively three, alternatively four, alternatively five, alternatively six, alternatively six or more versions of analyte profiles over a selected time period. The analytes may be, but are not limited to, acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The analyte profiles may be any of the different types of graphs that were described with respect to other embodiments, such as the glucose profiles.

In some embodiments, the plurality of graphs may include at least 2 graphs, alternatively at least 3 graphs, alternatively at least 4 graphs, alternatively at least 5 graphs, alternatively at least 6 graphs, alternatively at least 7 graphs, alternatively at least 8 graphs. Each of the plurality of graphs may display a different data set. In some embodiments, where a person is taking multiple medications at a time, a graph may be a display or presentation of all of the data from a time period, and two graphs may be displayed for each type of medication taken. For each medication, a first graph may include analyte measurements taken during a window of time following, or associated with, the administration of the medicine and a second graph may include analyte measurements taken during a window of time following, or associated with, missed administration(s) of the medicine. Thus, for a person taking n medications, a plurality of graphs that include 2n+1 graphs may be presented. As explained earlier with respect to other embodiments, the window of time following, or associated with, the administration of the medicine may be determined by the duration of action of the medication(s) being administered.

### Detecting if Medication is Taken or not

Determination of whether a medication is delivered into the patient may be readily determined using a connected medication delivery device. For medication delivery devices (such as a syringe) that are not connected, the system may use another means of determining if a medication is delivered. The system may provide a UI in which the patient can log whenever they take a medication. In addition, the system may include a predefined time of day window when the patient usually takes the medication, and if a medication log is not made in this time window, then the system may prompt the patient to confirm whether or not they took the medication. For oral medications, connected pill boxes or similar devices may be used to determine if the medication was taken. The system may provide a combination of connected device and UI means, where if the connected device did not indicate that a medication was taken, then the system may prompt the patient to confirm that they did not (or did) take the medication.

When a medication is taken later than the typical or prescribed time of day window, the glucose data associated with that time of day may be excluded. Similarly, glucose data associated with the time of day where the dose is taken late may also be excluded. The glucose data associated with these time-of-day periods may be included in some form of data analysis; for instance, for the purpose of showing the effect on glucose metrics when doses are taken late. In this case, the glucose during time-of-day periods associated with doses taken late may be included in the metric calculation only and may be compared to a metric calculated using glucose data from time-of-day periods only where doses are taken on time.

### Feedback Mechanisms on the Medication Delivery Device

Feedback Related to Recommended Dose

In some embodiments, a medication dosing calculation module in the DSS system may be linked with the interface or display of the MDD 152. As explained elsewhere, the MDD 152 may be a smart pen, a pen cap, or a pump that delivers a glucose level altering medication such as insulin. The DSS system may receive a recommended medication dose from the medication dosing calculation module of the DSS system. The recommended dose may be communicated to the MDD 152. When a dose is entered or selected on the MDD 152 by a user, the MDD 152 may provide feedback to the user as to whether the entered dose on the MDD 152 is the same, lower, or higher than the recommended dose from the medication dosing calculation module.

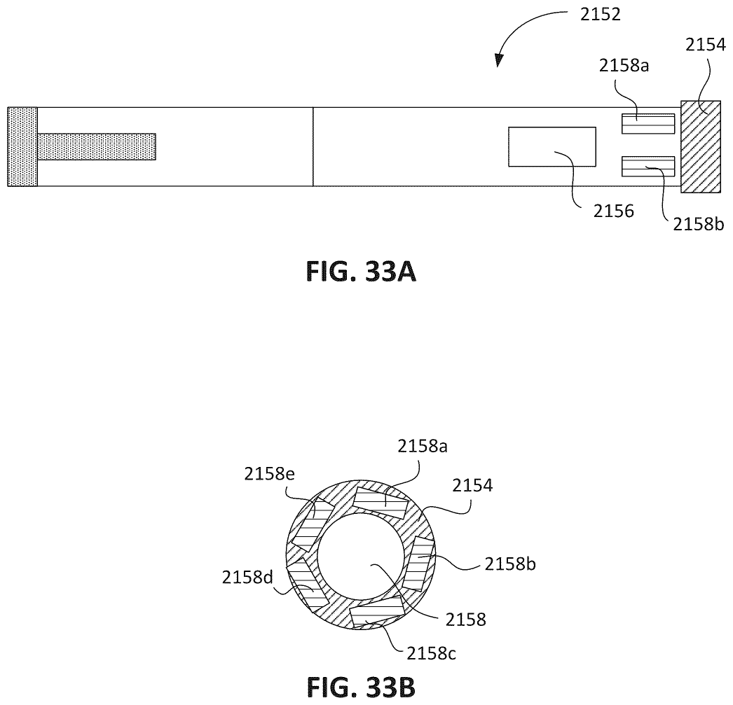

In some embodiments, the feedback provided may be visual feedback. For example, where the MDD 152 is a connected insulin pen 2152, as seen in FIGS. 33A-33D, the dose may be entered by the user using a rotatable dial or knob 2154, and the dose entered using the dial or knob 2154 may be shown in a display 2156 and/or 2158 to indicate the amount chosen for the manual injection. The background color of the display 2156 and/or 2158 may be a different color depending on the dose entered. For example, when the amount entered is the same as the recommended dose, the background color may be a first color, e.g., green. If the amount entered is higher than the recommended dose, the background color may be a second color, e.g., red. If the amount entered is lower than the recommended dose, the background color may be a third color, e.g., gray.

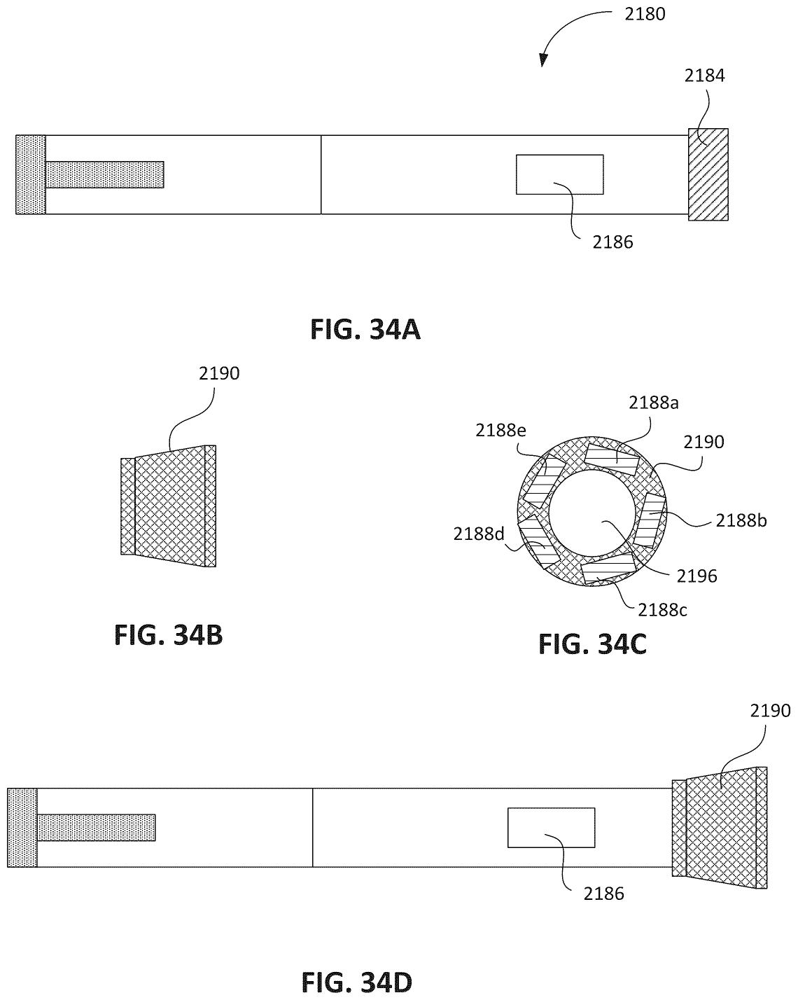

In some embodiments, the MDD 152 may be a smart button configured to fit over the actuator of the delivery pen or a pen cap configured to attach to the pen, e.g., attaches to the needle end, body, or dosing selector (e.g., dial). As seen in FIGS. 34A-34D, the medication pen 2180, which may be a single use medication delivery pen, may include a display 2186 and a dosing selector such as a rotatable dial 2184 to select the dose. A pen cap or smart button 2190 may fit over the dosing selector 2184 or other part of the pen, such as the needle end or body, and snap together with the pen 2180. After the pen cap or smart button 2190 is paired with a reader device, the pen cap or smart button 2190 may track the dose, store the dosing data, and transmit it to the reader device. Medication may be delivered by pushing straight down on the pen cap or smart button 2190 to inject the dose. The pen cap or smart button 2190 may also contain a display 2196 that can display the recommended dose and/or the dose administered. The background color of the display 2196 may be a different color depending on the dose entered. For example, when the amount entered is the same as the recommended dose, the background color may be a first color, e.g., green. If the amount entered is higher than the recommended dose, the background color may be a second color, e.g., red. If the amount entered is lower than the recommended dose, the background color may be a third color, e.g., gray.

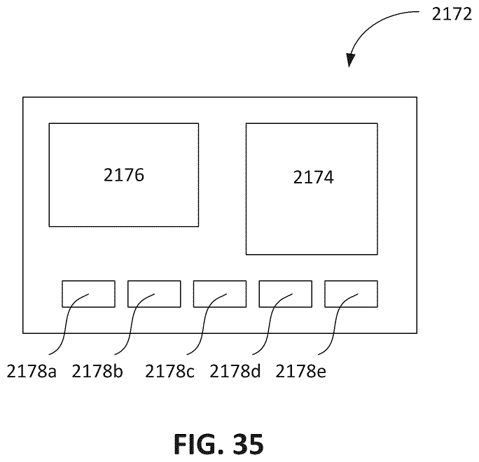

Similarly, if the MDD 152 is an insulin pump 2172, as seen in FIG. 35, the pump may include at least one button or a keypad 2174 used to enter the dose amount. The dose amount entered may be shown on a display 2176. Similar to the connected pen example, the display 2176 may have a first background color, e.g., green, if the dose entered is the same as the recommended dose. The display may have a different color, e.g., red, background if the dose entered is higher than the recommended dose. The display may be yet a different color, e.g., gray, if the dose entered is lower than the recommended dose.

In some embodiments, the feedback provided may be auditory feedback. For example, if the dose entered is the same as the recommended dose, the MDD 152 may emit a first sound, e.g., a single short beep or chirp. If the dose entered is higher than the recommended dose, the MDD 152 may emit a second sound, e.g., a long beep. If the dose entered is lower than the recommended dose, the MDD 152 may emit a third sound, e.g., a series of short beeps.

In some embodiments, the feedback provided may be tactile feedback. For example, if the dose entered is the same as the recommended dose, the MDD 152 may not vibrate. If the dose entered is higher than the recommended dose, the MDD 152 may vibrate for a first period of time. If the dose entered is lower than the recommended dose, the MDD 152 may vibrate for a second period of time, which is different than the first period of time. In some embodiments, if the dose entered is the same as the recommended dose, the MDD 152 may vibrate for a third period of time, which is different from the first and second periods of time. Alternatively, the MDD 152 may emit different vibration patterns depending on the relationship between the dose entered and the recommended dose.

In some embodiments, the medication dosing calculation module may determine a recommended dosing range with an upper and lower limit. Alternatively, the medication dosing calculation module may recommend a nominal value within a recommended dosing range. The above-referenced visual, auditory, and tactile feedback mechanisms described may be used by comparing the entered dose amount to the recommended dose range to determine if the entered dose amount is within the recommended dose range, greater than the upper limit of the recommended dose range, or lower than the lower limit of the recommended dose range.

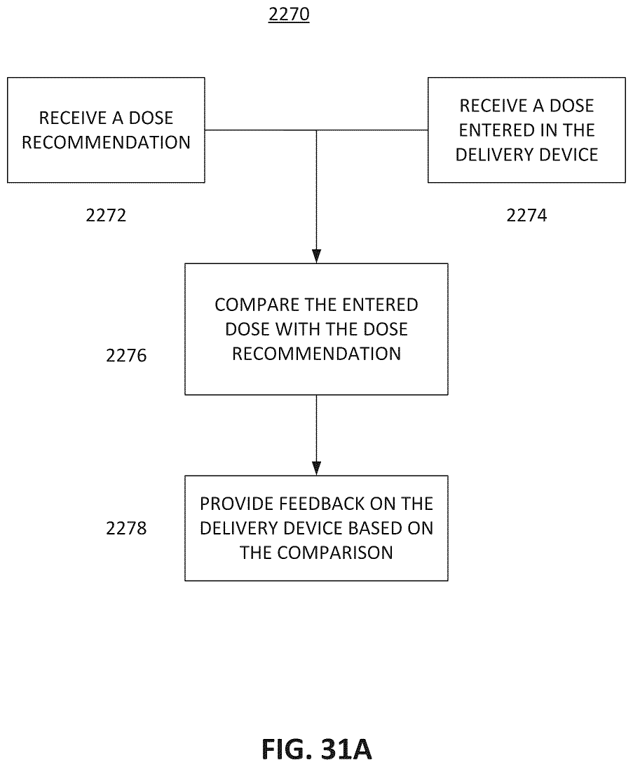

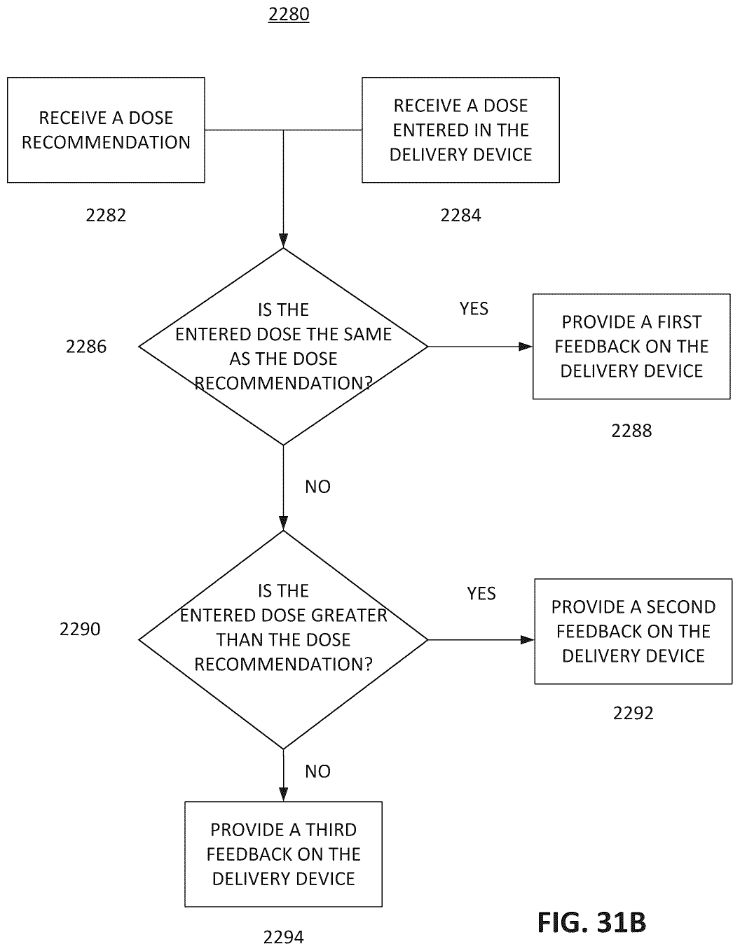

As seen in FIG. 31A, in exemplary method 2270, beginning with steps 2272 and 2274, one or more processors of an MDD 152 may receive a dose recommendation (step 2272) and a dose entered by the user (step 2274). In step 2276, the one or more processors may compare the entered dose with the dose recommendation. In step 2278, the one or more processors may provide feedback on the delivery device based on the comparison.

As described elsewhere, the comparison may be whether the entered dose is the same, greater than, or less than the dose recommendation.

As seen in FIG. 31B, in exemplary method 2280, beginning with steps 2282 and 2284, one or more processors of an MDD 152 may receive a dose recommendation (step 2282) and a dose entered by the user (step 2284). In step 2286, the one or more processors may determine if the entered dose is the same as the dose recommendation. If it the entered dose is the same as the dose recommendation, then in step 2288, a first type of feedback may be provided on the MDD 152. In different embodiments, the feedback may be visual, auditory, or tactile, as described in relation to other embodiments.

If the entered dose is not the same as or equal to the dose recommendation, then in step 2290, the one or more processors may determine if the entered dose is greater than the dose recommendation. If the entered dose is greater than the dose recommendation, in step 2292, a second type of feedback may be provided on the MDD 152. In different embodiments, the feedback may be visual, auditory, or tactile, as described in relation to other embodiments. Alternatively, in step 2290, the one or more processors may determine if the entered dose is less than the dose recommendation.

If the entered dose is not greater than (i.e., less than) the dose recommendation, then in step 2294, a third type of feedback may be provided on the MDD 152. In different embodiments, the feedback may be visual, auditory, or tactile, as described in relation to other embodiments.

The steps in any of the methods described herein are optional and may be excluded from the method or done in a different order than what appears in the figures. For example, the method may only determine if the entered dose is the same as the dose recommendation. If it is the same as the dose recommendation, then the one or more processors may provide feedback on the delivery device. If the entered dose is different (i.e., lower or higher) than the dose recommendation, in some embodiments, the one or more processors may not provide feedback on the delivery device. In another example, the method may only determine if the entered dose is different (e.g., higher) than the dose recommendation and not determine if the entered dose is equal to the dose recommendation.

As described above, the feedback may be visual (e.g., different color backgrounds), auditory (e.g., different sounds), or tactile (e.g., different vibrations). The feedback provided based on a comparison of the entered dose amount to the dose recommendation amount may be different depending on whether the entered dose amount is the same, greater than, or less than the dose recommendation amount.

Safeguards Against Higher than Dose Recommendations

Haptic Feedback on Medication Delivery Device

In another embodiment, as a safeguard to prevent higher or lower than recommended dosing, the MDD 152 may include haptic feedback when a user is entering a dose higher or lower than the dose recommendation. For example, in the case of a connected insulin pen, if the user is entering a dose higher than the dose recommendation using a rotatable dial or knob 2154, as seen in FIGS. 33A-33B, the dial or knob 2154 may be configured to set itself by default to the dose recommendation amount or the nominal recommended value within a recommended dosing range. Alternatively or in addition to, the dial or knob 2154 may be configured to have increased resistance to rotating, as compared to rotating or dialing the knob 2154 to the dose recommendation, if the dose being entered is above the dose recommendation. The dial or knob 2154 may alternatively be configured to bounce back to the dose recommendation if it is dialed to a dose above the dose recommendation. For example, the dial or knob 2154 may be resiliently biased to the dose recommendation.

In another example, the MDD 152 may be an insulin pump that includes at least one button for entering the dose to be administered. The at least one button may be configured to have an increased resistance to a downward motion (e.g., harder to push down or select) when a dose higher or lower than the dose recommendation is being entered. For example, as seen in FIG. 35, the insulin pump 2172 may include “+” and “−” buttons or a numeric keypad 2174 that allow the user to enter the dose amount to be delivered. The buttons or keypad may be configured to have increased resistance to a downward motion if the dose being entered is out of range.

As seen in FIG. 32A, in exemplary method 3000, beginning with steps 3002, one or more processors of a MDD 152 may receive a dose recommendation. In step 3004, the one or more processors may also determine a dose being entered using a dosing selector of the MDD 152. In step 3006, the one or more processors may determine if the dose being entered is different than the dose recommendation, i.e., if the entered dose is above or below than the dose recommendation. In step 3008, if the dose being entered is different than the dose recommendation, the one or more processors may provide haptic feedback on the MDD 152.

As described elsewhere, the haptic feedback may be increased resistance, bounce back, rebound, and other known haptic feedback mechanisms.

Lockout when Higher than Dose Recommendations are Entered

In another embodiment, as another safeguard to prevent higher or lower than recommended dosing, the MDD 152 may be configured to mechanically lock the MDD 152 to prevent any medication from being delivered to the user. In some embodiments, the MDD 152 may only be locked if a difference between the dose being entered, and the dose recommendation is greater than a predetermined number of units. The predetermined number of units may be about 1 unit, alternatively about 2 units, alternatively about 3 units, alternatively about 4 units.

In some embodiments, the MDD 152 may be locked by mechanically locking the dosing selector. For example, if the MDD 152 is a connected pen 2152, the rotatable dial 2154 may be mechanically locked such that it may no longer rotate. Alternatively, the MDD 152 may be locked by mechanically locking or disengaging the injector.

In some embodiments, the entered dose amount may be communicated to the reader device and one or more processors of the reader device may display a notice or alert regarding the higher dose entered and may require the user to confirm the higher dose on the GUI of the DSS module.

As seen in FIG. 32B, in exemplary method 3020, beginning with step 3022, one or more processors of a MDD 152 may receive a dose recommendation. In step 3024, the one or more processors of the MDD 152 may also determine a dose being entered using a dosing selector of the MDD 152. In step 3026, the one or more processors may determine if the dose being entered is different than the dose recommendation, i.e., if the entered dose is above or below the dose recommendation. In step 3028, if the dose being entered is different than the dose recommendation, the one or more processors may lock the MDD 152 to prevent it from delivering medication.

Adjust Alerts when Higher than Dose Recommendation Given

In some embodiments, if the user sets and/or delivers an insulin dose that is different than the dose recommendation or outside of the recommended range, then the DSS system may adjust an alert parameter. For example, if the dose delivered by or entered on the MDD 152 is higher than the dose recommendation by a first predetermined amount or lower than the dose recommendation by a second predetermined amount, then the DSS system may adjust the urgency or nature of low and/or high glucose alerts for a period of time after the insulin dose delivery. Examples of adjusting the urgency of the alert include increasing a low glucose threshold, decreasing a high glucose threshold, or increasing a prediction horizon (the period of time during which a predicted glucose level is determined) of the impending low or high glucose alerts. Examples of adjusting the nature of the alert include decreasing an alert snooze period, increasing a volume of the alert, prolonging the enunciation of an audible alert, or requiring additional acknowledgement to dismiss the alert.

As seen in FIG. 32C, in exemplary method 3040, one or more processors of a reader device may determine a recommended dose in step 3042. The one or more processors may also receive a dose delivered by or a dose entered on the MDD 152 in step 3044. The one or more processors may then determine if the dose delivered is different than the dose recommendation in step 3046. In step 3048, if the dose delivered or entered is different than the dose recommendation, then the one or more processors may adjust an alert parameter.

In some embodiments, a low glucose threshold may be increased to a higher concentration. Alternatively or in addition to, a high glucose threshold may be decreased to a lower concentration. Alternatively or in addition to, the time period during which an alert is predicted may be increased by, e.g., at least about 20 minutes, alternatively at least about 30 minutes, alternatively at least about 40 minutes, alternatively at least about 50 minutes, alternatively at least about 60 minutes. In some embodiments, a snooze period is decreased to a shorter time period. For example, the snooze period may be decreased by at least 3 minutes, for example the snooze period may be decreased by about 15 minutes, alternatively by about 10 minutes, alternatively by about 5 minutes, alternatively by about 3 minutes. In some embodiments, a volume of an audible alert may be increased. In some embodiments, the DSS may enable a requirement that the user acknowledge an alert.

Safeguards when Late Dosing

Timeout Feature on Medication Delivery Device

The DSS may also include safeguards against a late dose, e.g., a dose administered after a start of a meal. In some embodiments, the MDD 152 may include a timeout feature such that after a predetermined amount of time has passed since the determination of the dose recommendation, the MDD 152 may be disabled such that no medication dose may be delivered. For example, the dosing selector of the MDD 152 may be set to a zero dose amount and/or may be locked. The MDD 152 may also be disabled from delivering any medication until a new dose recommendation is determined and communicated to the MDD 152.

As seen in FIG. 32D, in exemplary method 3060, in step 3062, one or more processors of the MDD 152 may receive a dose recommendation at a first time, T1, from the medication dosing calculation module. In step 3064, one or more processors of the MDD 152 may receive dose data for a dose entered on the MDD 152 at a second time, T2. In step 3066, the one or more processors of the MDD 152 may determine if the difference between T2 and T1 is greater than a predetermined amount of time, e.g., at least about 20 minutes, alternatively at least about 30 minutes, alternatively at least about 40 minutes, alternatively at least about 50 minutes, alternatively at least about 60 minutes. If the difference between T2 and T1 is greater than the predetermined amount of time, then in step 3068, the one or more processors may change a configuration of the mediation delivery device. For example, the dosing selector of the MDD 152 may be changed to a locked configuration. Alternatively or in addition to, the dosing selector may be set to a zero dose amount if the MDD 152 is an insulin pump.

In another embodiment, as seen in FIG. 32E in exemplary method 3070, in step 3072, one or more processors of the MDD 152 may receive a dose recommendation at time T1 from the medication dosing calculation module. In step 3074, the one or more processors of the MDD 152 may determine if a dose has been administered during a predetermined time since the dose recommendation was determined at time T1. If no dose has been administered, then in step 3076, the one or more processors of the MDD 152 may change a configuration of the MDD 152. For example, the dosing selector of the MDD 152 may be changed to a locked configuration. Alternatively or in addition to, the dosing selector may be set to a zero dose amount.

Updating Display on the Medication Delivery Device

An additional safeguard for late dosing may include the medication dosing calculation module taking into account the latency or time delay of the current dose relative the recommended dosing time. The medication dosing calculation module may calculate a new dose recommendation. This new dose recommendation may then be communicated to the MDD 152, which may be displayed on the delivery device.

As seen in FIG. 32F, in exemplary method 3080, in step 3082, one or more processors of the reader device may determine a recommended dose at a first time, T1. In some embodiments, the medication dosing calculation module may determine the dose recommendation. In step 3084, the one or more processors of the reader device may determine if a dose has been administered during a predetermined time since the dose recommendation was determined at the first time T1. If no dose has been administered, then in step 3086, the one or more processors of the reader device may determine a new dose recommendation. In step 3088, the new dose recommendation may be communicated to the MDD 152. In some embodiments, the MDD 152 may display the new dose recommendation. In some embodiments, the reader device may display the new dose recommendation.

In some embodiments, the predetermined period of time may be at least about 20 minutes, alternatively at least about 30 minutes, alternatively at least about 40 minutes, alternatively at least about 50 minutes, alternatively at least about 60 minutes.

In another embodiment, the new dose recommendation is a periodically updated function of the first dose recommendation at fixed time intervals since the previous recommendation. As seen in FIG. 32G, in exemplary method 3090, in step 3092, one or more processors of the reader device may determine a first dose recommendation. In some embodiments, the medication dosing calculation module may determine the dose recommendation. In step 3095, the one or more processors may determine if the dose recommendation is below a threshold value. If the first dose recommendation is below a threshold such as zero units, then in some embodiments, the process stops providing the dose recommendation (step 3093). Otherwise, in step 3095, the one or more processors may wait for a first time period (such as 20 minutes) to elapse (step 3091). Afterwards, in step 3094, the one or more processors of reader device may determine whether a dose has been delivered. If so, then the process stops providing the dose recommendation (step 3093). Otherwise, in step 3096, the process determines a new dose recommendation by accounting for factors including the time elapsed since the previous time period. In step 3098, if the dose recommendation is below a threshold value, then the process stops providing the dose recommendation (step 3093). Otherwise, the one or more processors will wait for a second time period (such as 5 minutes) to elapse (step 3099). Afterwards, in step 3097, the one or more processors of reader device may determine whether a dose has been delivered. If so, then the process stops providing the dose recommendation (step 3093). Otherwise, the process returns to step 3096. In some embodiment, an additional timeout may be provided such that if the time elapsed since the first dose recommendation exceeds a third time period (such as 5 hours), then the process stops providing the dose recommendation.

### Other Safety Features

Lockout when Zero Dose Allowed

The DSS may include other safety features. In some embodiments, when the dose recommendation amount is determined to be zero (0), the MDD 152 may lock the MDD 152 such that no medication may be delivered until further action is taken or until a non-zero dose recommendation is received. The medication dosing calculation module may determine that the dose recommendation should be zero based on several factors, including insulin on board, and/or the risk of hypoglycemia exceeding a threshold. Exemplary methods of determining the risk of hypoglycemia may be found in U.S. Patent Publ. No. 2014/0088392, U.S. Patent Publ. No. 2014/0187887, U.S. Patent Publ. No. 2014/0188400, and U.S. Patent Publ. No. 2014/0350369, all of which are hereby expressly incorporated by reference in their entireties for all purposes.

As seen in FIG. 32H, in exemplary method 3100, in step 3102, the MDD 152 may receive a dose recommendation from the reader device. One or more processors of the MDD 152 may determine if the dose recommendation is equal to a dosage of zero (0) in step 3104. In step 3106, the one or more processors of the MDD 152 may lock the MDD 152, thereby preventing the delivery of any medication from the device until further action is taken or until a non-zero dose recommendation is received.

Actions on Pen Trigger GUIs on the Reader Device

In some embodiments, a set of actions on the MDD 152 may trigger the application module on the reader device to display a particular user interface or a dialog menu. For example, the set of actions can include when the user enters a dose that is several increments higher during a first predetermined time period. Alternatively, the set of actions can include when the user enters a dose (proposed dose amount) that is higher or lower than the dose recommendation in a second predetermined amount of time. In response to the set of actions, a user interface such as a dialog menu may be displayed on the reader device that may present possible dose guidance options (e.g., additional dose guidance). In some embodiments, depending on the time of day and prior patterns of the user, the dialog menu for a meal dose advice or a post-meal correction dose may be presented. In some embodiments, a basal dose guidance may not be displayed if the user pattern and that particular time of day was not within a period of time (e.g., 1 hour) of previous basal doses taken by the user.

As seen in FIG. 32I, in exemplary method 3120, in step 3122, one or more processors of the reader device may determine a recommended dose. In step 3124, the reader device may receive dose data for a dose being entered using a dosing selector on a MDD 152. In step 3126, the one or more processors of the reader device may determine if the entered dose is different than the dose recommendation. For example, the entered dose may be several units higher or lower than the dose recommendation. In step 3128, one or more processors of the reader device may cause the display of a GUI related to the dose recommendation. For example, the GUI may include suggested dose guidance options or may include a warning about under or over-dosing. In some embodiments, the dose guidance may be a suggested meal dose based on the time of day the dose is being entered. In some embodiments, the dose guidance may be a suggested correction dose based on the time of day the dose is being entered. In some embodiments, the dose guidance may be a suggested basal dose based on the time of day the dose is being entered.

### Shortcuts on the Medication Delivery Device

In some embodiments, the MDD 152 may include a single shortcut button or a plurality of shortcut buttons 2158a-e, in FIGS. 33A-33B, 2188a-e in FIG. 34C, and 2178a-e in FIG. 35 for specific dose recommendations. For example, the MDD 152 may include a shortcut button for each of breakfast, lunch, dinner, basal, snack, and/or correction dose. When the button is depressed or selected, one or more processors of the MDD 152 may retrieve the corresponding recommended medication dose amount determined by the medication dosing calculation module and the dose recommendation may be displayed.

As seen in FIG. 32J, in exemplary method 3140 in step 3142, the MDD 152 may receive a dose recommendation for at least one meal type. In step 3144, one or more processors of the MDD 152 may assign the dose recommendation for the at least one meal type to one of a plurality of buttons on the MDD 152. In response to the user selecting one of the plurality of buttons (step 3146), the MDD 152 may display the dose recommendation (step 3148). More specifically, in response to the user selecting the button corresponding to the dose recommendation (step 3146), the MDD 152 may display the dose recommendation (step 3148). In an optional step, the MDD 152 may also set the dose amount to the dose recommendation displayed and associated with the selected button.

In other methods, the MDD 152 may receive a plurality of dose recommendations for different meal types. One or more processors of the MDD 152 may assign the each of the recommend doses for the different meal types to a different button on the MDD 152. In response to the user selecting one of the plurality of buttons, the MDD 152 may display the dose recommendation for that corresponding meal type. In an optional step, the MDD 152 may also set the dose amount to the dose recommendation displayed and associated with the selected button.

The steps in any of the methods described herein are optional and may be excluded from the method or done in a different order than what appears in the figures.

Systems, devices and methods are provided for incorporating a medication delivery device into an integrated management system. The integrated management system may be an integrated diabetes management system and may include a glucose monitor, a connected insulin pen, and software. The integrated management system may produce a plurality of reports that may include data related to analyte levels (e.g., glucose levels) and medication delivered (e.g., insulin delivered). The medication delivery device may also provide feedback to the user.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, an analyte monitoring system includes a medication delivery device configured to deliver an amount of a medication to the subject, the medication delivery device comprising: a display; a dosing selector; first wireless communications circuitry configured to receive dose recommendations from a reader device; and first one or more processors coupled to a first memory; a reader device, comprising: a display; second wireless communication circuitry configured to receive time-correlated data characterizing an analyte level of the subject and time-correlated dosage data of the medication received by the subject over a period of time; and second one or more processors coupled to a second memory, the second memory storing instructions that, when executed by the second one or more processors, cause the second one or more processors to: determine a dose recommendation of the medication based on at least calculated analyte levels, the calculated analyte levels based on the time-correlated data characterizing the analyte level of the subject, communicate the dose recommendation of the medication to the medication delivery device, wherein instructions stored in the first memory, when executed, cause the first one or more processors to provide feedback based on a comparison between the dose recommendation and a dose entered using the dosing selector.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of medication received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the feedback comprises visual feedback.

In some embodiments, the feedback comprises haptic feedback.

In some embodiments, the feedback comprises tactile feedback.

In some embodiments, the medication delivery device is a pen. In some embodiments, the medication delivery device further comprises a pen cap. In some embodiments, the display is on the pen cap.

In some embodiments, the medication delivery device further comprises a smart button. In some embodiments, the display is on the smart button.

In some embodiments, the dosing selector is a rotatable knob.

In some embodiments, the medication delivery device is a pump. In some embodiments, the dosing selector is at least one button.

In some embodiments, the display of the medication delivery device has a background color, and wherein if the dose entered using the dosing selector is equal to the dose recommendation of the medication, the feedback comprises the background color of the display of the medication delivery device being a first color. In some embodiments, the first color is green.

In some embodiments, if the dose entered using the dosing selector is greater than the dose recommendation of the medication, the feedback comprises the background color of the display of the medication delivery device being a second color. In some embodiments, the second color is red.

In some embodiments, if the dose entered using the dosing selector is less than the dose recommendation of the medication, the feedback comprises the background color of the display of the medication delivery device being a third color. In some embodiments, wherein the third color is gray.

In some embodiments, if the dose entered using the dosing selector is equal to the dose recommendation of the medication, the feedback comprises a first pattern, wherein if the dose entered using the dosing selector is greater than the dose recommendation of the medication, the feedback comprises a second pattern, and wherein if the dose entered using the dosing selector is lower than the dose recommendation of the medication, the feedback comprises a third pattern. In some embodiments, the first, second, and third patterns are different vibration patterns. In some embodiments, the first, second, and third patterns are different auditory patterns. In some embodiments, the first, second, and third patterns are different light patterns. In some embodiments, the light patterns comprise blinking patterns of light.

In some embodiments, the dose recommendation is a single dose amount.

In some embodiments, the dose recommendation comprises a dose recommendation range comprising an upper and lower limit, and wherein if the dose entered using the dosing selector is within the upper and lower limit of the dose recommendation range, or equal to either the upper or lower limit, the feedback comprises the background color of the display of the medication delivery device being a first color. In some embodiments, if the dose entered using the dosing selector is greater than the dose recommendation of the medication, the feedback comprises the background color of the display of the medication delivery device being a second color. In some embodiments, if the dose entered using the dosing selector is less than the dose recommendation of the medication, the feedback comprises the background color of the display of the medication delivery device being a third color.

In some embodiments, the system further includes a sensor control device comprising an analyte sensor, wherein at least a portion of the analyte sensor is configured to be in fluid contact with a bodily fluid of a subject.

In many embodiments, a method includes the steps of: receiving, by one or more processors of a medication delivery device, a dose recommendation; receiving, by the one or more processors of a medication delivery device, dosage data for a dose entered on the medication delivery device; comparing the dose entered to the dose recommendation; and providing feedback on the medication delivery device based on the comparison.

In some embodiments, the comparing step comprises determining if the entered dose is equal to the dose recommendation.

In some embodiments, in response to a determination that the entered dose is equal to the dose recommendation, a first feedback is provided on the medication delivery device. In some embodiments, the first feedback is a first color background of a display of the medication delivery device.

In some embodiments, the comparing step comprises determining if the entered dose is greater than the dose recommendation. In some embodiments, in response to a determination that the entered dose is greater than the dose recommendation, a second feedback is provided on the medication delivery device, wherein the second feedback is different than the first feedback.

In some embodiments, the comparing step comprises determining if the entered dose is greater than the dose recommendation. In some embodiments, in response to a determination that the entered dose is greater than the dose recommendation, a second feedback is provide on the medication delivery device, wherein the second feedback is different than the first feedback.

In some embodiments, the comparing step comprises determining if the entered dose is less than the dose recommendation. In some embodiments, in response to a determination that the entered dose is less than the dose recommendation, a third feedback is provide on the medication delivery device.

In some embodiments, the feedback is visual, auditory, or tactile.

In many embodiments, an analyte monitoring system includes a medication delivery device configured to deliver an amount of a medication to the subject, the medication delivery device comprising: a display; a dosing selector; first wireless communications circuitry configured to receive dose recommendations from a reader device; and first one or more processors coupled to a first memory; a reader device, comprising: a display; second wireless communication circuitry configured to receive time-correlated data characterizing an analyte level of the subject and doses of the medication received by the subject over a period of time; and second one or more processors coupled to a second memory, the second memory storing instructions that, when executed by the second one or more processors, cause the one or more processors to: determine a dose recommendation of the medication based on at least calculated analyte levels, the calculated analyte levels based on the time-correlated data characterizing the analyte of the subject, wherein instructions stored in the first memory, when executed, cause the first one or more processors to provide haptic feedback through the dosing selector based on a comparison between the dose recommendation and a dose entered using the dosing selector.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the haptic feedback comprises an increased resistance when the dosing selector is set to a dose higher than the dose recommendation.

In some embodiments, the haptic feedback comprises an increased resistance when the dosing selector is set to a dose lower than the dose recommendation.

In some embodiments, the dosing selector is a rotatable knob that is configured to turn in a first direction to increase a dose amount, and wherein the haptic feedback comprises a bias to turn in a second direction opposite the first direction when the dosing selector is turned to a dose higher, or optionally lower, than the dose recommendation.

In some embodiments, the medication delivery device is a pen.

In some embodiments, the medication delivery device is a pump.

In some embodiments, the dosing selector is at least one button, and optionally wherein the at least one button is configured to have an increased resistance to a downward motion when a dose higher than the dose recommendation is selected.

In some embodiments, the system also includes a sensor control device comprising an analyte sensor, wherein at least a portion of the analyte sensor is configured to be in fluid contact with a bodily fluid of a subject.

In many embodiments, a method includes the steps of: receiving, by one or more processors of a medication delivery device, a dose recommendation; receiving, by one or more processors of a medication delivery device, a dose being entered on the medication delivery device; comparing the dose being entered to the dose recommendation; and providing haptic feedback on the medication delivery device if the dose being entered is different than the dose recommendation.

In some embodiments, the haptic feedback comprises increased resistance of a dosing selector.

In some embodiments, the dose being entered is entered using a dosing selector, and wherein the dosing selector is configured to be set by default to the dose recommendation.

In some embodiments, the dose being entered is entered using a dosing selector. In some embodiments, the dosing selector is a rotatable dial. In some embodiments, the rotatable dial is configured to bounce back to a setting of the dose recommendation. For example, the rotatable dial may be resiliently biased to the setting of the dose recommendation. In some embodiments, the rotatable dial is configured to have a resistance when rotating to an amount above the dose recommendation.

In many embodiments, an analyte monitoring system includes: a medication delivery device configured to deliver an amount of a medication to the subject, the medication delivery device comprising: a display; a dosing selector; first wireless communications circuitry configured to receive dose recommendations from a reader device; and first one or more processors coupled to a first memory; a reader device, comprising: a display; second wireless communication circuitry configured to receive time-correlated data characterizing an analyte level of the subject and dosage data for doses of the medication received by the subject over a period of time; and second one or more processors coupled to a second memory, the second memory storing instructions that, when executed by the second one or more processors, cause the second one or more processors to: determine a dose recommendation of the medication based on at least calculated analyte levels, the calculated analyte levels based on the time-correlated data characterizing the analyte level of the subject, wherein instructions stored in the first memory, when executed, cause the first one or more processors to lock the medication delivery device to prevent delivery of a medication when the dosing selector is set to a dose that is different than the dose recommendation.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, instructions stored in the first memory, when executed, cause the first one or more processors to lock the dosing selector when the dosing selector is set to a dose that is different than the dose recommendation.

In some embodiments, instructions stored in the first memory, when executed, cause the first one or more processors to lock the medication delivery device when the dosing selector is set to a dose that is a predetermined number of units higher than the dose recommendation.

In some embodiments, the predetermined number of units is about 2 units.

In some embodiments, the second instructions further cause the one or more processors of the reader device to display a GUI comprising a notification of the dose that is a predetermined number of units greater than the dose recommendation.

In some embodiments, the second instructions further cause the second one or more processors of the reader device to request confirmation from the subject regarding the dose that is a predetermined number of units greater than the dose recommendation.

In some embodiments, the medication delivery device is a pen, and wherein the dosing selector is a rotatable knob.

In some embodiments, the medication delivery device is a pump, and wherein the dosing selector is at least one button.

In some embodiments, the system further includes a sensor control device comprising an analyte sensor, wherein at least a portion of the analyte sensor is configured to be in fluid contact with a bodily fluid of a subject.

In many embodiments, a method includes the steps of: receiving, by one or more processors of a medication delivery device, a dose recommendation; receiving, by one or more processors of a medication delivery device, a dose being entered on the medication delivery device; comparing the dose being entered to the dose recommendation; and locking the medication delivery device to prevent delivery of a medication if the dose being entered is above the dose recommendation.

In some embodiments, the step of locking the medication delivery device comprises locking the dosing selector.

In some embodiments, the medication deliver device is locked when the dosing selector is set to a dose that is a predetermined number of units higher than the dose recommendation. In some embodiments, the predetermined number of units is about 2 units.

In some embodiments, the medication delivery device is a pen, and wherein the dosing selector is a rotatable knob.

In some embodiments, the medication delivery device is a pump, and wherein the dosing selector is at least one button.

In many embodiments, an analyte monitoring system includes: a medication delivery device configured to deliver an amount of a medication to the subject, the medication delivery device comprising: a display; a dosing selector; first wireless communications circuitry configured to receive dose recommendations from a reader device; and first one or more processors coupled to a first memory; a reader device, comprising: a display; second wireless communication circuitry configured to receive time-correlated data characterizing an analyte level of the subject and dosage data for doses of the medication received by the subject over a period of time; and second one or more processors coupled to a second memory, the second memory storing instructions that, when executed by the second one or more processors, cause the second one or more processors to: determine a dose recommendation of the medication based on at least calculated analyte levels and the doses of the medication received by the subject over the period of time, the calculated analyte levels based on the time-correlated data characterizing the analyte level of the subject, and communicate the dose recommendation to the medication delivery device, wherein instructions stored in the first memory, when executed, cause the first one or more processors to lock the medication delivery device to prevent delivery of a medication when the dose recommendation is equal to zero.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, instructions stored in the first memory, when executed, cause the first one or more processors to lock the dosing selector when the dose recommendation is equal to zero.

In some embodiments, the dose recommendation is determined to be zero based on an insulin-on-board determination.

In some embodiments, the dose recommendation is determined to be zero based on a determination of a risk of hypoglycemia based on the at least calculated analyte levels based on the time-correlated data characterizing the analyte level of the subject and the doses of the medication received by the subject over the period of time.

In some embodiments, the medication delivery device is a pen, and optionally wherein the dosing selector is a rotatable knob. In some embodiments, the pen further comprises a delivery button and a plunger mechanically coupled to the delivery button, wherein when the dose recommendation is zero, the delivery button decouples from the plunger.

In some embodiments, the medication delivery device is a pump, and wherein the dosing selector is at least one button.

In some embodiments, the system further includes a sensor control device comprising an analyte sensor, wherein at least a portion of the analyte sensor is configured to be in fluid contact with a bodily fluid of a subject.

In many embodiments, a method includes the steps of: receiving, by one or more processors of a medication delivery device, a dose recommendation; determining if the dose recommendation is equal to zero; and locking the medication delivery device to prevent delivery of a medication if the dose recommendation is determined to be a dosage of zero. The locking of the medication delivery device may comprise decoupling of parts of the medication delivery device required for delivery of medication from the medication delivery device.

In some embodiments, the step of locking the medication delivery device comprises locking the dosing selector.

In some embodiments, the dose recommendation is determined to be zero based on an insulin-on-board determination.

In some embodiments, the dose recommendation is determined to be zero based on a determination of a risk of hypoglycemia based on the at least calculated analyte levels and the doses of the medication received by the subject over the period of time. The calculated analyte levels may also be based on the time-correlated data characterizing the analyte of the subject.

In some embodiments, the medication delivery device is a pen, and/or wherein the dosing selector is a rotatable knob.

In some embodiments, the pen further comprises a delivery button and a plunger mechanically coupled to the delivery button, wherein step of locking the medication delivery device comprises decoupling the delivery button from the plunger.

In many embodiments, an analyte monitoring system includes: a medication delivery device configured to deliver an amount of a medication to the subject, the medication delivery device comprising: a display; a dosing selector; and first wireless communications circuitry configured to receive dose recommendations from a reader device and to communicate doses of the medication received by the subject; and a reader device, comprising: a display; second wireless communication circuitry configured to receive time-correlated data characterizing an analyte level of the subject and dosage data for the doses of the medication received by the subject from the medication delivery device; and one or more processors coupled to a memory, the memory storing instructions that, when executed by the one or more processors, cause the one or more processors to: determine a dose recommendation of the medication based on at least calculated analyte levels, the calculated analyte levels based on the time-correlated data characterizing the analyte level of the subject, compare the dose recommendation of the medication with a corresponding amount of the medication delivered by the medication delivery device, and adjust an alert parameter if the corresponding amount of medication delivered is different than the dose recommendation. The corresponding amount of the medication delivered may be the amount of the dose of the medication delivered at the same or similar time as the time corresponding to the dose recommendation.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the alert parameter is a low glucose threshold, and wherein the low glucose threshold is increased to a higher concentration when the amount of medication delivered is higher than the dose recommendation.

In some embodiments, the alert parameter is a high glucose threshold, and wherein the high glucose threshold is decreased to a lower concentration when the amount of medication delivered is lower than the dose recommendation.

In some embodiments, the alert parameter is a time period during which an alert is predicted, and wherein the time period during which an alert is predicted is increased when the corresponding amount of medication delivered is different than the dose recommendation. In some embodiments, the time period during which an alert is predicted is increased by at least about 20 minutes when the corresponding amount of medication delivered is different than the dose recommendation.

In some embodiments, the alert parameter is a snooze period, and wherein the snooze period is decreased to a shorter time period when the corresponding amount of medication delivered is different than the dose recommendation.

In some embodiments, the alert parameter is a volume of an audible alert, and wherein the volume is increased when the corresponding amount of medication delivered is different than the dose recommendation.

In some embodiments, the alert parameter is a duration of an audible alert, and wherein the duration is increased when the corresponding amount of medication delivered is different than the dose recommendation.

In some embodiments, the alert parameter is a requirement of an acknowledgement of the alert, and wherein the requirement of the acknowledgement of the alert is enabled when the corresponding amount of medication delivered is different than the dose recommendation.

In some embodiments, the instructions, when executed, further cause the one or more processors to display additional information regarding the amount of medication delivered.

In some embodiments, the alert parameter is a tactile parameter, and wherein the alert parameter is adjusted to cause the reader device to vibrate if the corresponding amount of medication delivered is higher than the dose recommendation. In some embodiments, the alert parameter is a tactile parameter, and wherein the alert parameter is adjusted to cause the reader device to vibrate if the corresponding amount of medication delivered is lower than the dose recommendation.

In some embodiments, the alert parameter is an audible parameter, and wherein the alert parameter is adjusted to cause the reader device to emit a sound if the corresponding amount of medication delivered is higher than the dose recommendation. In some embodiments, the alert parameter is an audible parameter, and wherein the alert parameter is adjusted to cause the reader device to emit a sound if the corresponding amount of medication delivered is lower than the dose recommendation.

In some embodiments, the system further includes a sensor control device comprising an analyte sensor, wherein at least a portion of the analyte sensor is configured to be in fluid contact with a bodily fluid of a subject.

In many embodiments, a method includes the steps of: determining, by one or more processors of a reader device, a dose recommendation; receiving, by one or more processors of a reader device, dosage data for a dose entered or delivered by a medication delivery device; comparing the dose entered or delivered to the dose recommendation; and adjusting, by one or more processors of a reader device, an alert parameter if the dose entered or delivered is different than the dose recommendation.

In some embodiments, the alert parameter is a low glucose threshold, and wherein the low glucose threshold is increased to a higher concentration when the amount of medication delivered is higher than the dose recommendation.

In some embodiments, the alert parameter is a high glucose threshold, and wherein the high glucose threshold is decreased to a lower concentration when the amount of medication delivered is lower than the dose recommendation.

In some embodiments, the alert parameter is a time period during which an alert is predicted, and wherein the time period during which an alert is predicted is increased when the corresponding amount of medication delivered is different than the dose recommendation. In some embodiments, the time period during which an alert is predicted is increased by at least about 20 minutes when the corresponding amount of medication delivered is different than the dose recommendation.

In some embodiments, the alert parameter is a snooze period, and wherein the snooze period is decreased to a shorter time period when the corresponding amount of medication delivered is different than the dose recommendation. The snooze period may be decreased by, for example, at least 5 minutes when the corresponding amount of medication delivered is different than the dose recommendation.

In some embodiments, the alert parameter is a volume of an audible alert, and wherein the volume is increased when the corresponding amount of medication delivered is different than the dose recommendation.

In some embodiments, the alert parameter is a duration of an audible alert, and wherein the duration is increased when the corresponding amount of medication delivered is different than the dose recommendation.

In some embodiments, the alert parameter is a requirement of an acknowledgement of the alert, and wherein the requirement of the acknowledgement of the alert is enabled when the corresponding amount of medication delivered is different than the dose recommendation.

In some embodiments, the method further includes the step of: displaying additional information regarding the amount of medication delivered.

In some embodiments, the alert parameter is a tactile parameter, and wherein the alert parameter is adjusted to cause the reader device to vibrate if the corresponding amount of medication delivered is higher than the dose recommendation.

In some embodiments, the alert parameter is an audible parameter, and wherein the alert parameter is adjusted to cause the reader device to emit a sound if the corresponding amount of medication delivered is higher than the dose recommendation.

In many embodiments, an analyte monitoring system includes: a medication delivery device configured to deliver an amount of a medication to the subject, the medication delivery device comprising: a display; a dosing selector; and first wireless communications circuitry configured to communicate proposed dose amounts of the medication entered using the dosing selector from the medication delivery device before delivery of the doses of the medication entered; the reader device, comprising: a display; second wireless communication circuitry configured to receive time-correlated data characterizing an analyte level of the subject and the communicated proposed dose amounts of the medication entered using the dosing selector from the medication delivery device before delivery of the doses of the medication entered; and one or more processors coupled to a memory, the memory storing instructions that, when executed by the one or more processors, cause the one or more processors to: determine a dose recommendation of the medication based on at least calculated analyte levels, the calculated analyte levels based on the time-correlated data characterizing the analyte level of the subject, compare the dose recommendation of the medication with a proposed dose amount of the medication entered using the dosing selector, and cause a graphic user interface to be displayed on the display, the graphic user interface comprising additional dose guidance if the proposed dose amount of the medication is different than the dose recommendation of the medication.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the proposed dose amount of the medication is greater than the dose recommendation.

In some embodiments, the proposed dose amount of the medication is smaller than the dose recommendation.

In some embodiments, the graphic user interface is displayed if the proposed dose amount of the medication is entered within a predetermined amount of time of the determination of the dose recommendation.

In some embodiments, the graphic user interface is displayed if the proposed dose amount of the medication is entered after a predetermined amount of time of the determination of the dose recommendation.

In some embodiments, the additional dose guidance relates to a time of day that the proposed dose amount of the medication is entered.

In some embodiments, the system further includes a sensor control device comprising an analyte sensor, wherein at least a portion of the analyte sensor is configured to be in fluid contact with a bodily fluid of a subject.

In many embodiments, a method includes the steps of: determining, by one or more processors of a reader device, a dose recommendation (i.e. a recommended dose); receiving, by one or more processors of a reader device, a dose being entered on a medication delivery device; comparing the dose being entered to the dose recommendation; and displaying a user interface related to the dose recommendation if the dose entered is different than the dose recommendation. The dose recommendation may be a recommendation as to the amount and/or timing of a dose of medication. The dose recommendation may be referred to throughout the specification as dose recommendation.

In some embodiments, the user interface is displayed if the dose being entered is within a predetermined amount of time of the determination of the dose recommendation.

In some embodiments, the user interface is displayed if the dose being entered is after a predetermined amount of time of the determination of the dose recommendation.

In some embodiments, the user interface comprises additional dose guidance related to a time of day corresponding to the dose being entered on the medication delivery device.

In many embodiments, an analyte monitoring system includes: a medication delivery device configured to deliver an amount of a medication to the subject, the medication delivery device comprising: a display; at least one button; first wireless communications circuitry configured to receive dose recommendations from a reader device; and first one or more processors coupled to a first memory; a reader device, comprising: a display; second wireless communication circuitry configured to receive time-correlated data characterizing an analyte level of the subject and dosage data for doses of the medication received by the subject over a period of time from the medication delivery device; and second one or more processors coupled to a second memory, the second memory storing instructions that, when executed by the second one or more processors, cause the second one or more processors to: determine a dose recommendation of the medication for at least one meal type based on at least calculated analyte levels, the calculated analyte levels based on the time-correlated data characterizing the analyte level of the subject, and communicate the dose recommendation of the medication for at least one meal type to the medication delivery device, wherein instructions stored in the first memory, when executed, cause the first one or more processors of the medication delivery device to display the dose recommendation of the medication on the display of the medication delivery device for the at least one meal type when the at least one button is selected.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the second instructions, when executed, cause the second one or more processors to determine a dose recommendation for each of a plurality of meal types, and wherein the at least one button comprises a plurality of buttons, wherein each meal type corresponds to a respective button of the plurality of buttons, wherein the first memory of the medication delivery device causes the first one or more processors of the medication delivery device to display a dose recommendation for one of the plurality of meal types in response to a selection of the respective button of the plurality of buttons. Each meal type may correspond, for example, may be assigned to, a corresponding button. The dose recommendation for one meal type may be displayed in response to selection of the corresponding button. In some embodiments, the plurality of meal types comprises breakfast, lunch, dinner, and snack.

In some embodiments, the second instructions, when executed, cause the second one or more processors to determine a dose recommendation for a basal dose, and wherein the at least one button comprises a plurality of buttons, wherein the dose recommendation for the basal dose corresponds to a respective button of the plurality of buttons, wherein the first memory of the medication delivery device causes the first one or more processors of the medication delivery device to display the dose recommendation for the basal dose in response to a selection of the respective button of the plurality of buttons.

In some embodiments, the second instructions, when executed, cause the second one or more processors to determine a dose recommendation for a correction dose, and wherein the at least one button comprises a plurality of buttons, wherein the recommended dose for the correction dose corresponds to a respective button of the plurality of buttons, wherein the first memory of the medication delivery device causes the first one or more processors of the medication delivery device to display the dose recommendation for the correction dose in response to a selection of the respective button of the plurality of buttons.

In some embodiments, the system further includes a sensor control device comprising an analyte sensor, wherein at least a portion of the analyte sensor is configured to be in fluid contact with a bodily fluid of a subject.

In many embodiments, a method includes the steps of: receiving, by one or more processors of a medication delivery device, a dose recommendation for at least one meal type; assigning, by the one or more processors of the medication delivery device, the dose recommendation for the at least one meal type to one of a plurality of buttons on the medication delivery device; and displaying the dose recommendation on the medication delivery device in response to the selection of the one of the plurality of buttons on the medication delivery device.

In some embodiments, the method further includes the step of: setting, by the one or more processors of the medication delivery device, a dose selector of the medication delivery device to the dose recommendation.

In some embodiments, the receiving step comprises receiving, by one or more processors of a medication delivery device, a dose recommendation for each of a plurality of meal types. In some embodiments, the plurality of meal types comprises at least two of breakfast, lunch, dinner, and snack.

In some embodiments, the method further includes the steps of: receiving, by the one or more processors of the medication delivery device, a dose recommendation for a basal dose; assigning, by the one or more processors of the medication delivery device, the dose recommendation for the basal dose to one of a plurality of buttons on the medication delivery device.

In some embodiments, the method further includes the steps of: receiving, by the one or more processors of the medication delivery device, a dose recommendation for a correction dose; assigning, by the one or more processors of the medication delivery device, the dose recommendation for the correction dose to one of a plurality of buttons on the medication delivery device.

In many embodiments, an analyte monitoring includes: a medication delivery device configured to deliver an amount of a medication to the subject, the medication delivery device comprising: a display: a dosing selector; first wireless communications circuitry configured to receive dose recommendations from a reader device; and first one or more processors coupled to a first memory; a reader device, comprising: a display; second wireless communication circuitry configured to receive time-correlated data characterizing an analyte level of the subject and dosage data for doses of the medication received by the subject over a period of time; and second one or more processors coupled to a second memory, the second memory storing instructions that, when executed by the second one or more processors, cause the second one or more processors to: determine a dose recommendation of the medication based on at least calculated analyte levels, the calculated analyte levels based on the time-correlated data characterizing the analyte level of the subject and the doses of the medication received by the subject over the period of time, wherein instructions in the first memory, when executed, cause the first one or more processors to change a configuration of the medication delivery device to prevent the medication delivery device from delivering the medication when a predetermined time since the dose recommendation of the medication was determined has elapsed.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the dosing selector is changed to a locked configuration after the predetermined time has elapsed. In the locked configuration, the dosing selector cannot be adjusted to a different dose by a user.

In some embodiments, the dosing selector is changed to a dosage of zero after the predetermined time has elapsed.

In some embodiments, the system further includes a sensor control device comprising an analyte sensor, wherein at least a portion of the analyte sensor is configured to be in fluid contact with a bodily fluid of a subject.

In many embodiments, a method includes the steps of: receiving, by one or more processors of a medication delivery device, dosage data for a dose recommendation at a first time; receiving, by one or more processors of a medication delivery device, dosage data for a dose being entered on the medication delivery device at a second time; determining, by one or more processors of a medication delivery device, if the difference between the second time and the first time is greater than a predetermined amount of time; and changing, by one or more processors of a medication delivery device, a configuration of the medication delivery device if the difference between the second time and the first time is greater than a predetermined amount of time.

In some embodiments, a dosing selector of the medication delivery device is changed to a locked configuration after the predetermined time has elapsed.

In some embodiments, a dosing selector of the medication delivery device is changed to zero after the predetermined time has elapsed.

In many embodiments, an analyte monitoring system includes: a medication delivery device configured to deliver an amount of a medication to the subject, the medication delivery device comprising: a display: a dosing selector; first wireless communications circuitry configured to receive a dose recommendation from a reader device at a first time, T1; and first one or more processors coupled to a first memory, the first memory storing instructions that, when executed by the first one or more processors, cause the first one or more processors to: determine if a dose has been delivered during a predetermined period of time since first time, T1, a reader device, comprising: a display; second wireless communication circuitry configured to receive time-correlated data characterizing an analyte level of the subject and dosage data for doses of the medication received by the subject over a period of time; and second one or more processors coupled to a second memory, the second memory storing instructions that, when executed by the second one or more processors, cause the second one or more processors to: determine a recommended dose of the medication based on at least calculated analyte levels and the doses of the medication received by the subject over the period of time, the calculated analyte levels based on the time-correlated data characterizing the analyte level of the subject, wherein instruction stored in the first memory, when executed, cause the first one or more processors to change a configuration of the medication delivery device to prevent the medication delivery device from delivering the medication if it is determined that the dose has not been delivered during the predetermined period of time since the first time, T1.

In some embodiments, the dosing selector is changed to a locked configuration after the predetermined period of time since the first time, T1 has elapsed.

In some embodiments, the dosing selector is changed to a dosage of zero after the predetermined period of time since the first time, T1 has elapsed.

In some embodiments, the system further includes a sensor control device comprising an analyte sensor, wherein at least a portion of the analyte sensor is configured to be in fluid contact with a bodily fluid of a subject.

In many embodiments, a method includes the steps of: receiving, by one or more processors of a medication delivery device, dosage data for a dose recommendation at a first time; determining, by one or more processors of the medication delivery device, if a dose has been delivered during a predetermined period of time since a first time, T1; and changing a configuration of the medication delivery device if the it is determined that no dose has been delivered during the predetermined period of time since the first time, T1.

In some embodiments, the step of changing the configuration of the medication delivery device comprises changing a dosing selector of the medication delivery device to a locked configuration.

In some embodiments, the step of changing the configuration of the medication delivery device comprises changing a dosing selector of the medication delivery device to a dosage of zero.

In many embodiments, an analyte monitoring system includes: a medication delivery device configured to deliver an amount of a medication to the subject, the medication delivery device comprising: a display; a dosing selector; and wireless communications circuitry configured to receive dose recommendations from a reader device and to communicate doses administered to the reader device; a reader device, comprising: a display; wireless communication circuitry configured to receive time-correlated data characterizing an analyte level of the subject and dosage data for doses of the medication received by the subject over a period of time from the medication delivery device; and one or more processors coupled to a memory, the memory storing instructions that, when executed by the one or more processors, cause the one or more processors to: determine a dose recommendation of the medication based on at least calculated analyte levels, the calculated analyte levels based on the time-correlated data characterizing the analyte level of the subject at a first time, determine if a dose has been administered during a predetermined period of time since the dose recommendation was determined at the first time, determine a new dose recommendation of the medication based on at least calculated analyte levels based on the time-correlated data characterizing the analyte level of the subject at a second time; and communicate the new dose recommendation to the medication delivery device.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the medication delivery device further comprises one or more processors coupled to an additional memory, the additional memory storing instructions that, when executed by the one or more processors, cause the dose recommendation to be displayed on the display of the medication delivery device.

In some embodiments, the new dose recommendation is lower than the dose recommendation.

In some embodiments, the system further includes a sensor control device comprising an analyte sensor, wherein at least a portion of the analyte sensor is configured to be in fluid contact with a bodily fluid of a subject.

In many embodiments, a method includes the steps of: determining, by one or more processors of a reader device, a dose recommendation at a first time, T1; receiving, by one or more processors of a reader device, if a dose has been delivered during a predetermined period of time since the first time, T1; determining, by one or more processors of a reader device, a new dose recommendation if it is determined that no dose has been delivered during the predetermined period of time since time T1; and communicating the new dose recommendation to a medication delivery device.

In some embodiments, the method further includes the step of: displaying the new dose recommendation on the medication delivery device.

In some embodiments, the method further includes the step of: displaying the new dose recommendation on the reader device.

In some embodiments, the predetermined period time is at least about 20 minutes.

In some embodiments, the new dose recommendation is lower than the dose recommendation.

In many embodiments, an analyte monitoring system comprising: a medication delivery device configured to deliver an amount of a medication to the subject, the medication delivery device comprising: a display; a dosing selector; and wireless communications circuitry configured to receive dose recommendations from a reader device and to communicate doses administered to the reader device; a reader device, comprising: a display; wireless communication circuitry configured to receive time-correlated data characterizing an analyte level of the subject and receive dosage data for doses of the medication received by the subject over a period of time from the medication delivery device; and one or more processors coupled to a memory, the memory storing instructions that, when executed by the one or more processors, cause the one or more processors to: determine a first dose recommendation of the medication based on at least calculated analyte levels based on the time-correlated data characterizing the analyte level of the subject at a first time, determine if the first dose recommendation is below a threshold value, determine if a dose has been administered during a predetermined period of time since the first time if it is determined that the first dose recommendation is not below a threshold value, determine a second dose recommendation of the medication at a second time based on at least calculated analyte levels, the calculated analyte levels based on the time-correlated data characterizing the analyte level of the subject if it is determined that the dose has not been administered within the predetermined time period.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the instructions further cause the one or more processors to: determine if the second dose recommendation is below a threshold value, determine if the dose has been administered during a second predetermined period of time since the second time if it is determined that the dose is not below the threshold value, and revise the second dose recommendation at a third time based on at least calculated analyte levels based on the time-correlated data characterizing the analyte level of the subject if it is determined that the dose has not been administered within the second predetermined time period.

In some embodiments, the system further includes a sensor control device comprising an analyte sensor, wherein at least a portion of the analyte sensor is configured to be in fluid contact with a bodily fluid of a subject.

In many embodiments, a method includes the steps of; determining, by one or more processors of a reader device, a first dose recommendation of the medication based on at least calculated analyte levels, the calculated analyte levels based on the time-correlated data characterizing the analyte level of the subject at a first time, determining, by the one or more processors of the reader device, if the first dose recommendation is below a threshold value, determining, by the one or more processors of the reader device, if a dose has been administered during a predetermined period of time since the first time if it is determined that the first recommended dose is not below a threshold value, and determining, by the one or more processors of the reader device, a second dose recommendation of the medication at a second time based on at least calculated analyte levels based on the time-correlated data characterizing the analyte level of the subject if it is determined that the dose has not been administered within the predetermined time period.

In some embodiments, the methods further includes the steps of: determining, by the one or more processors of the reader device, if the second dose recommendation is below the threshold value, determining, by the one or more processors of the reader device, if the dose has been administered during a second predetermined period of time since the second time if it is determined that the second dose recommendation is not below the threshold value, revising, by the one or more processors of the reader device, the second dose recommendation at a third time based on at least calculated analyte levels based on the time-correlated data characterizing the analyte level of the subject if it is determined that the dose has not been administered within the second predetermined time period.

In many embodiments, a system for displaying metrics relating to a subject includes: wireless communications circuitry configured to receive time-correlated data characterizing an analyte level of the subject; a display configured to visually present information; a memory; and one or more processors coupled with the wireless communications circuitry, the display, and the memory, wherein the memory stores instructions and time-correlated data characterizing an analyte level of the subject, wherein the instructions, when executed by the one or more processors, cause the system to: determine a subset of time-correlated data based on a filtering criteria selected by the subject, wherein the filtering criteria comprises enablement of at least one alarm; and display a first glucose profile and a second glucose profile on a single graphical subject interface, wherein the first glucose profile displays glucose levels associated with the time-correlated data over a first time period, and wherein the second glucose profile displays the subset of the time-correlated data over the first time period. Enablement of the alarm may be referred to herein as output or activation of the alarm.

In some embodiments, the at least one alarm is a low glucose alarm. In some embodiments, the low glucose alarm has a threshold of about 70 mg/dL.

In some embodiments, the at least one alarm is a high glucose alarm. In some embodiments, the high glucose alarm has a threshold of about 180 mg/dL.

In some embodiments, the instructions, when executed by the one or more processor, further cause the system to: display occurrences of the at least one alarm in the first time period on at least one of the first and second glucose profiles.

In some embodiments, the wireless communications circuitry is further configured to receive dosage data for doses of a medication received by the subject over a period of time, and wherein the memory further stores doses of a glucose level-altering medication received by the subject over a period of time, wherein the instructions, when executed by the one or more processor, further cause the system to: display instances of doses of the medication received in the first time period on at least one of the first and second glucose profiles.

In some embodiments, the instructions, when executed by the one or more processor, further cause the system to: determine a second subset of time-correlated data based on the filtering criteria selected by the subject, wherein the second subset of time-correlated data comprises time-correlated data when the at least one alarm is disabled, and display a third glucose profile on the single graphical subject interface, wherein the third glucose profile displays the second subset of time-correlated data over the first time period. In such an embodiment, the first subset may comprise time-correlated data when the at least one alarm is enabled. Disablement of the alarm may involve silencing or turning off the alarm. In some embodiments, the instructions, when executed by the one or more processor, further cause the system to: display occurrences of the at least one alarm in the first time period on at least one of the first, second, and third glucose profiles. In some embodiments, the wireless communications circuitry is further configured to receive doses of a medication received by the subject over a period of time, and wherein the memory further stores doses of a glucose level-altering medication received by the subject over a period of time, wherein the instructions, when executed by the one or more processor, further cause the system to: display instances of doses of the medication received in the first time period on at least one of the first, second, and third glucose profiles. In some embodiments, the instances of doses the medication received in the first time period are displayed on the second glucose profile.

In some embodiments, the wireless communications circuitry is further configured to receive dosage data for doses of a medication received by the subject over a period of time, and wherein the memory further stores doses of a glucose level-altering medication received by the subject over a period of time, wherein the instructions cause the one or more processors to determine at least one dose recommendation, and wherein the filtering criteria further comprises dose concordance of the glucose altering medication. In some embodiments, the wireless communications circuitry is further configured to receive dosage data for a dose regimen for the subject, and wherein the at least one dose recommendation is at least partly determined from the dose regimen. In some embodiments, the dose concordance comprises missed doses, under-bolused doses with reference to the at least one dose recommendation, over-bolused doses with reference to the at least one dose recommendation, late meal doses, or extra meal doses.

In some embodiments, the first glucose profile and the second glucose profile each comprise a trace of a median glucose level. In some embodiments, the first glucose profile and the second glucose profile each further comprise an upper trace of an hourly 95^{th} percentile of glucose levels and a lower trace of an hourly 5^{th} percentile of glucose levels. In some embodiments, the first glucose profile and the second glucose profile each further comprise a second upper trace of an hourly 75^{th} percentile of glucose levels and a second lower trace of an hourly 25^{th} percentile of glucose levels.

In some embodiments, the first glucose profile and the second glucose profile each further comprise data indicative of an hourly 75^{th} percentile of glucose levels and a second lower trace of an hourly 15^{th} percentile of glucose levels.

In some embodiments, the first glucose profile and the second glucose profile each comprise a plurality of traces, wherein a trace of the plurality of traces corresponds to a day of the first period of time, and wherein the plurality of traces are displayed in an overlay pattern.

In some embodiments, the instructions, when executed by the one or more processor, further cause the system to: determine a first analyte metric based on the time-correlated data for the first time period and a second analyte metric based on the subset of the time correlated data for the first time period; and display the first analyte metric and the second analyte metric on the single graphical subject interface.

In some embodiments, the instructions, when executed by the one or more processor, further cause the system to: determine at least one first analyte statistic based on the time-correlated data for the first time period and at least one second analyte statistic based on the subset of the time correlated data for the first time period; and display the at least one first analyte statistic and the at least one second analyte statistic on the single graphical subject interface. In some embodiments, the at least one first analyte statistic comprises a first glucose management indicator, a first average glucose, a first standard deviation, or combinations thereof, and/or wherein the at least one second analyte statistic comprises a second glucose management indicator, a second average glucose, a second standard deviation, or combinations thereof.

In some embodiments, the instructions, when executed by the one or more processor, further cause the system to: determine a first hypoglycemia risk based on the time-correlated data for the first time period and a second hypoglycemia risk based on the subset of the time correlated data for the first time period; and display the first and the second hypoglycemia risk on the single graphical subject interface.

In many embodiments, a method includes the steps of: receiving time-correlated data characterizing an analyte level of a subject; determining a subset of time-correlated data based on a filtering criteria selected by the subject, wherein the filtering criteria comprises enablement of at least one alarm; and displaying a first glucose profile and a second glucose profile on a single graphical subject interface, wherein the first glucose profile displays glucose levels associated with the time-correlated data over a first time period, and wherein the second glucose profile displays the subset of the time-correlated data over the first time period.

In some embodiments, the at least one alarm is a low glucose alarm. In some embodiments, the low glucose alarm has a threshold of about 70 mg/dL.

In some embodiments, the at least one alarm is a high glucose alarm. In some embodiments, the high glucose alarm has a threshold of about 180 mg/dL.

In some embodiments, the method further includes the step of: displaying occurrences of the at least one alarm in the first time period on at least one of the first and second glucose profiles.

In some embodiments, the method further includes the steps of: receiving dosage data for doses of a medication received by the subject over a period of time; storing dosage data for doses of a glucose level-altering medication received by the subject over a period of time; and displaying instances of doses of the medication received in the first time period on at least one of the first and second glucose profiles.

In some embodiments, the method further includes the steps of: determining a second subset of time-correlated data based on the filtering criteria selected by the subject, wherein the second subset of time-correlated data comprises time-correlated data when the at least one alarm is disabled, and displaying a third glucose profile on the single graphical subject interface, wherein the third glucose profile displays the second subset of time-correlated data over the first time period.

In some embodiments, the method further includes the step of: displaying occurrences of the at least one alarm in the first time period on at least one of the first, second, and third glucose profiles.

In some embodiments, the method further includes the steps of: receiving dosage data for doses of a medication received by the subject over a period of time; storing dosage data for doses of a glucose level-altering medication received by the subject over a period of time; and displaying instances of doses of the medication received in the first time period on at least one of the first, second, and third glucose profiles. In some embodiments, the instances of doses the subject received in the first time period are displayed on the second glucose profile.

In some embodiments, the wireless communications circuitry is further configured to receive dosage data for doses of a medication received by the subject over a period of time, and wherein the memory further stores doses of a glucose level-altering medication received by the subject over a period of time, and wherein the filtering criteria further comprises dose concordance. In some embodiments, the dose concordance comprises dose recommendations, missed doses, under-bolused doses, over-bolused doses, late meal doses, or extra meal doses.

In some embodiments, the first glucose profile and the second glucose profile each comprise a trace of a median glucose level. In some embodiments, the first glucose profile and the second glucose profile each further comprise an upper trace of an hourly 95^{th} percentile of glucose levels and a lower trace of a 5^{th} percentile of glucose levels. In some embodiments, the first glucose profile and the second glucose profile each further comprise a second upper trace of an hourly 75^{th} percentile of glucose levels and a second lower trace of an hourly 25^{th} percentile of glucose levels. In some embodiments, the first glucose profile and the second glucose profile each further comprise data indicative of an hourly 75^{th} percentile of glucose levels and a second lower trace of an hourly 15^{th} percentile of glucose levels.

In some embodiments, the first glucose profile and the second glucose profile each comprise a plurality of traces, wherein a trace of the plurality of traces corresponds to a day of the first period of time, and wherein the plurality of traces are displayed in an overlay pattern.

In some embodiments, the method further includes the steps of: determining a first analyte metric based on the time-correlated data for the first time period and a second analyte metric based on the subset of the time correlated data for the first time period; and displaying the first analyte metric and the second analyte metric on the single graphical subject interface.

In some embodiments, the method further includes the steps of: determining at least one first analyte statistic based on the time-correlated data for the first time period and at least one second analyte statistic based on the subset of the time correlated data for the first time period; and displaying the at least one first analyte statistic and the at least one second analyte statistic on the single graphical subject interface. In some embodiments, the at least one first analyte statistic comprises a first glucose management indicator, a first average glucose, a first standard deviation, or combinations thereof, and/or wherein the at least one second analyte statistic comprises a second glucose management indicator, a second average glucose, a second standard deviation, or combinations thereof.

In some embodiments, the method further includes the steps of: determining a first hypoglycemia risk based on the time-correlated data for the first time period and a second hypoglycemia risk based on the subset of the time correlated data for the first time period; and displaying the first and the second hypoglycemia risk on the single graphical subject interface.

In many embodiments, a system for displaying metrics relating to a subject including: wireless communications circuitry configured to receive time-correlated data characterizing an analyte level of the subject; a display configured to visually present information; a memory; and one or more processors coupled with the wireless communications circuitry, the display, and the memory, wherein the memory stores instructions and time-correlated data characterizing an analyte level of the subject, wherein the instructions, when executed by the one or more processors, cause the system to: determine a subset of time-correlated data based on a filtering criteria selected by the subject; determine a first analyte metric based on the time-correlated data for a first time period and a second analyte metric based on the subset of the time correlated data for the first time period; and display the first analyte metric and the second analyte metric on a single graphical subject interface.

In some embodiments, the first analyte metric is an amount of time that analyte levels determined from the time-correlated data were within at least one concentration range during the first time period, and the second analyte metric is an amount of time that analyte levels determined from the subset of time-correlated data were within the at least one concentration range during the first time period. In some embodiments, the at least one concentration range comprises a target concentration range.

The dosing selector referred to throughout the application may also be referred to as a dosage selector. The dosing selector is configured to receive input indicating a dose of medication to be administered. The dose of medication input via the dosing selector may be input and/or chosen by a user. Time-correlated data characterizing the analyte of the subject may be data that represents/reflects the analyte concentration for the subject over time. The dosage data may be amounts and timings of doses of mediation received by the subject over the period of time. The dosage data for doses of medication received by the subject may be simply referred to throughout the specification as doses of the medication received by the subject. The comparison between the dose recommendation to the dose entered using the dosing selector may involve comparing the amount of the dose recommendation to the amount of the dose entered using the dosing selector. The dose recommendation may correspond to a similar time or the same time as the time of the dose entered using the dosing selector. The first memory and the second memory referred to herein are part of the analyte monitoring system. The first memory may be part of the medication delivery device. The second memory may be part of the reader device.

In some embodiments, the at least one concentration range comprises a plurality of concentration ranges. In some embodiments, the plurality of concentration ranges comprises a low glucose concentration range, a target concentration range, and a high glucose concentration range. In some embodiments, the plurality of concentration ranges comprises a very low glucose concentration range, a low glucose concentration range, a target concentration range, a high glucose concentration range, and a very high glucose concentration range.

In some embodiments, the filtering criteria comprises enablement of at least one alarm.

In some embodiments, the instructions, when executed by the one or more processor, further cause the system to: determine a second subset of time-correlated data based on the filtering criteria selected by the subject, wherein the second subset of time-correlated data comprises time-correlated data when the at least one alarm is disabled, and display a third analyte metric on the single graphical subject interface, wherein the third glucose profile displays the second subset of time-correlated data over the first time period.

In some embodiments, the instructions, when executed by the one or more processor, further cause the system to: determine an analyte level based on the time-correlated data; and display the analyte level on an additional graphical user interface; wherein the filtering criteria comprises the subject viewing the analyte level on the additional graphical user interface within a period of time. In some embodiments, the period of time is about 3 hours.

In some embodiments, the filtering criteria comprises time-correlated data corresponding to an A1c value greater than a threshold. In some embodiments, the threshold is about 7%.

In some embodiments, the instructions, when executed by the one or more processor, further cause the system to: display a first glucose profile and a second glucose profile on the single graphical subject interface, wherein the first glucose profile displays glucose levels associated with the time-correlated data over the first time period, and wherein the second glucose profile displays the subset of the time-correlated data over the first time period. In some embodiments, the first glucose profile and the second glucose profile each comprise a trace of a median glucose level. In some embodiments, the first glucose profile and the second glucose profile each further comprise an upper trace of an hourly 95^{th} percentile of glucose levels and a lower trace of a 5^{th} percentile of glucose levels. In some embodiments, the first glucose profile and the second glucose profile each further comprise a second upper trace of an hourly 75^{th} percentile of glucose levels and a second lower trace of an hourly 25^{th} percentile of glucose levels. In some embodiments, the first glucose profile and the second glucose profile each further comprise data indicative of an hourly 75^{th} percentile of glucose levels and a second lower trace of an hourly 15^{th} percentile of glucose levels. In some embodiments, the first glucose profile and the second glucose profile each comprise a plurality of traces, wherein a trace of the plurality of traces corresponds to a day of the first period of time, and wherein the plurality of traces are displayed in an overlay pattern.

In many embodiments, a method for displaying metrics relating to a subject includes the steps of: receiving time-correlated data characterizing an analyte level of a subject; determining a subset of time-correlated data based on a filtering criteria selected by the subject; determining a first analyte metric based on the time-correlated data for a first time period and a second analyte metric based on the subset of the time correlated data for the first time period; and displaying the first analyte metric and the second analyte metric on a single graphical subject interface.

In some embodiments, the first analyte metric is an amount of time that analyte levels determined from the time-correlated data were within at least one concentration range during the first time period, and the second analyte metric is an amount of time that analyte levels determined from the subset of time-correlated data were within the at least one concentration range during the first time period. In some embodiments, the at least one concentration range comprises a target concentration range. In some embodiments, the at least one concentration range comprises a plurality of concentration ranges. In some embodiments, the plurality of concentration ranges comprises a low glucose concentration range, a target concentration range, and a high glucose concentration range. In some embodiments, the plurality of concentration ranges comprises a very low glucose concentration range, a low glucose concentration range, a target concentration range, a high glucose concentration range, and a very high glucose concentration range.

In some embodiments, the filtering criteria comprises enablement of at least one alarm.

In some embodiments, the method further includes the steps of: determining an analyte level based on the time-correlated data; and displaying the analyte level on an additional graphical user interface, wherein the filtering criteria comprises the subject viewing the analyte level on the additional graphical user interface within a period of time.

In some embodiments, the period of time is about 3 hours.

In some embodiments, the filtering criteria comprises time-correlated data corresponding to an A1c value greater than a threshold.

In some embodiments, the threshold is about 7%.

In some embodiments, the instructions, when executed by the one or more processor, further cause the system to: display a first glucose profile and a second glucose profile on the single graphical subject interface, wherein the first glucose profile displays glucose levels associated with the time-correlated data over the first time period, and wherein the second glucose profile displays the subset of the time-correlated data over the first time period. In some embodiments, the first glucose profile and the second glucose profile each comprise a trace of a median glucose level. In some embodiments, the first glucose profile and the second glucose profile each further comprise an upper trace of an hourly 95^{th} percentile of glucose levels and a lower trace of a 5^{th} percentile of glucose levels. In some embodiments, the first glucose profile and the second glucose profile each further comprise a second upper trace of an hourly 75^{th} percentile of glucose levels and a second lower trace of an hourly 25^{th} percentile of glucose levels.

In some embodiments, the first glucose profile and the second glucose profile each further comprise data indicative of an hourly 75^{th} percentile of glucose levels and a second lower trace of an hourly 15^{th} percentile of glucose levels.

In some embodiments, the first glucose profile and the second glucose profile each comprise a plurality of traces, wherein a trace of the plurality of traces corresponds to a day of the first period of time, and wherein the plurality of traces are displayed in an overlay pattern.

In many embodiments, a system for displaying metrics relating to a subject, the system comprising: wireless communications circuitry configured to receive time-correlated data characterizing glucose of the subject and time-correlated data characterizing an additional analyte of the subject; a display configured to visually present information; and a memory; and one or more processors coupled with the wireless communications circuitry, the display, the memory, wherein the memory stores instructions and time-correlated data characterizing an analyte level of the subject, wherein the instructions, when executed by the one or more processors, cause the system to: determine a subset of time-correlated data characterizing glucose based at least on a first filtering criteria related to at least one determined level of the additional analyte; and display a first glucose profile and a second glucose profile on a single graphical subject interface, wherein the first glucose profile displays glucose levels associated with the time-correlated data over a first time period, and wherein the second glucose profile displays the subset of the time-correlated data characterizing glucose based at least on the first filtering criteria over the first time period. The additional analyte is an analyte of the subject that is different from glucose.

In some embodiments, the first filtering criteria comprises the at least one determined analyte level of the additional analyte is above a threshold value.

In some embodiments, the additional analyte is a ketone or ketone body. In some embodiments, the additional analyte is β-hydroxybutyrate. In some embodiments, the additional analyte is lactate.

In some embodiments, the wireless communications circuitry is further configured to receive data characterizing activity of the subject, and wherein the first filtering criteria further comprises a determined activity level of the subject is below a high activity threshold.

In many embodiments, a method for displaying metrics relating to a subject includes the steps of: receiving time-correlated data characterizing an analyte level of a subject; determining a subset of time-correlated data characterizing glucose based at least on a first filtering criteria related to at least one determined level of the additional analyte; and displaying a first glucose profile and a second glucose profile on a single graphical subject interface, wherein the first glucose profile displays glucose levels associated with the time-correlated data over a first time period, and wherein the second glucose profile displays the subset of the time-correlated data characterizing glucose based at least on the first filtering criteria over the first time period.

In some embodiments, the first filtering criteria comprises the at least one determined analyte level of the additional analyte is above a threshold value.

In some embodiments, the additional analyte is a ketone or ketone body. In some embodiments, the additional analyte is β-hydroxybutyrate. In some embodiments, the additional analyte is lactate.

In some embodiments, the wireless communications circuitry is further configured to receive data characterizing activity of the subject, and wherein the first filtering criteria further comprises a determined activity level of the subject is below a high activity threshold.

In many embodiments, a system for displaying metrics relating to a subject, the system comprising: wireless communications circuitry configured to receive time-correlated data characterizing glucose of the subject; a display configured to visually present information; and a memory, and one or more processors coupled with the wireless communications circuitry, the display, and the memory, wherein the memory stores instructions and time-correlated data characterizing an analyte level of the subject, wherein the instructions, when executed by the one or more processors, cause the system to: determine a subset of time-correlated data characterizing glucose based at least on a first filtering criteria; and display a first glucose profile and a second glucose profile on a single graphical subject interface, wherein the first glucose profile displays glucose levels associated with the time-correlated data over a time period, and wherein the second glucose profile displays the subset of the time-correlated data characterizing glucose based at least on the first filtering criteria over the time period.

In some embodiments, the first filtering criteria comprises a type of day.

In some embodiments, the type of day is a weekday. In some embodiments, the type of day is a weekend day. In some embodiments, the type of day is a work day of the subject. In some embodiments, the type of day is a vacation day of the subject.

In some embodiments, the type of day is a day in which the subject exercised. In some embodiments, the type of day is a day in which the subject did not exercise.

In some embodiments, the type of day is a day in which the subject missed a basal insulin dose. In some embodiments, the type of day is a day in which the subject received at least one basal insulin dose.

In some embodiments, type of day is a day in which the subject missed at least one bolus insulin dose.

In some embodiments, the wireless communications circuitry is further configured to receive data characterizing activity of the subject.

In many embodiments, a method for displaying metrics relating to a subject includes the steps of: receiving time-correlated data characterizing an analyte level of a subject; determining a subset of time-correlated data characterizing glucose based at least on a first filtering criteria; and displaying a first glucose profile and a second glucose profile on a single graphical subject interface, wherein the first glucose profile displays glucose levels associated with the time-correlated data over a time period, and wherein the second glucose profile displays the subset of the time-correlated data characterizing glucose based at least on the first filtering criteria over the time period.

In some embodiments, the first filtering criteria comprises a type of day.

In some embodiments, the type of day is a weekday. In some embodiments, the type of day is a weekend day. In some embodiments, the type of day is a work day of the subject. In some embodiments, the type of day is a vacation day of the subject.

In some embodiments, the type of day is a day in which the subject exercised. In some embodiments, the type of day is a day in which the subject did not exercise.

In some embodiments, the type of day is a day in which the subject missed a basal insulin dose. In some embodiments, the type of day is a day in which the subject received at least one basal insulin dose.

In some embodiments, type of day is a day in which the subject missed at least one bolus insulin dose.

In some embodiments, the wireless communications circuitry is further configured to receive data characterizing activity of the subject.

### Clauses

Exemplary embodiments are set out in the following numbered clauses.

Clause 1. An analyte monitoring system, comprising:
a medication delivery device configured to deliver an amount of a medication to the subject, the medication delivery device comprising:
   a display;
   a dosing selector;
   first wireless communications circuitry configured to receive dose recommendations from a reader device;
   and
   first one or more processors coupled to a first memory;
a reader device, comprising:
   a display;
   second wireless communication circuitry configured to receive time-correlated data characterizing an analyte level of the subject and time-correlated dosage data of the medication received by the subject over a period of time;
   and
   second one or more processors coupled to a second memory, the second memory storing instructions that, when executed by the second one or more processors, cause the second one or more processors to:
      determine a dose recommendation of the medication based on at least calculated analyte levels, the calculated analyte levels based on the time-correlated data characterizing the analyte level of the subject,
      communicate the dose recommendation of the medication to the medication delivery device,
wherein instructions stored in the first memory, when executed, cause the first one or more processors to provide feedback based on a comparison between the dose recommendation and a dose entered using the dosing selector.

Clause 2. The system of clause 1, wherein the feedback comprises visual feedback.

Clause 3. The system of any of clauses 1-2, wherein the feedback comprises haptic feedback.

Clause 4. The system of any of clauses 1-3, wherein the feedback comprises tactile feedback.

Clause 5. The system of any of clauses 1-4, wherein the medication delivery device is a pen.

Clause 6. The system of any of clauses 1-5, wherein the medication delivery device further comprises a pen cap.

Clause 7. The system of any of clauses 1-6, wherein the display is on the pen cap.

Clause 8. The system of any of clauses 1-7, wherein the medication delivery device further comprises a smart button.

Clause 9. The system of any of clauses 1-8, wherein the display is on the smart button.

Clause 10. The system of any of clauses 1-9, wherein the dosing selector is a rotatable knob.

Clause 11. The system of any of clauses 1-10, wherein the medication delivery device is a pump.

Clause 12. The system of any of clauses 1-11, wherein the dosing selector is at least one button.

Clause 13. The system of any of clauses 1-12, wherein the display of the medication delivery device has a background color, and wherein if the dose entered using the dosing selector is equal to the dose recommendation of the medication, the feedback comprises the background color of the display of the medication delivery device being a first color.

Clause 14. The system of any of clauses 1-13, wherein the first color is green.

Clause 15. The system of any of clauses 1-14, wherein if the dose entered using the dosing selector is greater than the dose recommendation of the medication, the feedback comprises the background color of the display of the medication delivery device being a second color.

Clause 16. The system of any of clauses 1-15, wherein the second color is red.

Clause 17. The system of any of clauses 1-16, wherein if the dose entered using the dosing selector is less than the dose recommendation of the medication, the feedback comprises the background color of the display of the medication delivery device being a third color.

Clause 18. The system of any of clauses 1-17, wherein the third color is gray.

Clause 19. The system of any of clauses 1-18, wherein if the dose entered using the dosing selector is equal to the recommended dose of the medication, the feedback comprises a first pattern, wherein if the dose entered using the dosing selector is greater than the dose recommendation of the medication, the feedback comprises a second pattern, and wherein if the dose entered using the dosing selector is lower than the dose recommendation of the medication, the feedback comprises a third pattern.

Clause 20. The system of any of clauses 1-19, wherein the first, second, and third patterns are different vibration patterns.

Clause 21. The system of any of clauses 1-20, wherein the first, second, and third patterns are different auditory patterns.

Clause 22. The system of any of clauses 1-21, wherein the first, second, and third patterns are different light patterns.

Clause 23. The system of any of clauses 1-22, wherein the light patterns comprise blinking patterns of light.

Clause 24. The system of any of clauses 1-23, wherein the dose recommendation is a single dose amount.

Clause 25. The system of any of clauses 1-24, wherein the dose recommendation comprises a recommended dose range comprising an upper and lower limit, and wherein if the dose entered using the dosing selector is within the upper and lower limit of the recommended dose range, or equal to either the upper or lower limit, the feedback comprises the background color of the display of the medication delivery device being a first color.

Clause 26. The system of any of clauses 1-25, wherein if the dose entered using the dosing selector is greater than the dose recommendation of the medication, the feedback comprises the background color of the display of the medication delivery device being a second color.

Clause 27. The system of any of clauses 1-26, wherein if the dose entered using the dosing selector is less than the dose recommendation of the medication, the feedback comprises the background color of the display of the medication delivery device being a third color.

Clause 28. The system of any of clauses 1-27, further comprising a sensor control device comprising an analyte sensor, wherein at least a portion of the analyte sensor is configured to be in fluid contact with a bodily fluid of a subject.

Clause 29. A method, comprising the steps of:
receiving, by one or more processors of a medication delivery device, a dose recommendation;
receiving, by the one or more processors of a medication delivery device, dosage data for a dose entered on the medication delivery device;
comparing the dose entered to the dose recommendation; and
providing feedback on the medication delivery device based on the comparison.

Clause 30. The method of clause 29, wherein the comparing step comprises determining if the entered dose is equal to the dose recommendation.

Clause 31. The method of any of clauses 29-30, wherein, in response to a determination that the entered dose is equal to the dose recommendation, a first feedback is provided on the medication delivery device.

Clause 32. The method of any of clauses 29-31, wherein the first feedback is a first color background of a display of the medication delivery device.

Clause 33. The method of any of clauses 29-32, wherein the comparing step comprises determining if the entered dose is greater than the dose recommendation.

Clause 34. The method of any of clauses 29-33, wherein, in response to a determination that the entered dose is greater than the dose recommendation, a second feedback is provided on the medication delivery device, wherein the second feedback is different than the first feedback.

Clause 35. The method of any of clauses 29-34, wherein the comparing step comprises determining if the entered dose is greater than the dose recommendation.

Clause 36. The method of any of clauses 29-35, wherein, in response to a determination that the entered dose is greater than the dose recommendation, a second feedback is provide on the medication delivery device, wherein the second feedback is different than the first feedback.

Clause 37. The method of any of clauses 29-36, wherein the comparing step comprises determining if the entered dose is less than the dose recommendation.

Clause 38. The method of any of clauses 29-37, wherein, in response to a determination that the entered dose is less than the dose recommendation, a third feedback is provide on the medication delivery device.

Clause 39. The method of any of clauses 29-38, wherein the feedback is visual, auditory, or tactile.

Clause 40. An analyte monitoring system, comprising:
a medication delivery device configured to deliver an amount of a medication to the subject, the medication delivery device comprising:
   a display;
   a dosing selector;
   first wireless communications circuitry configured to receive dose recommendations from a reader device; and
   first one or more processors coupled to a first memory;
a reader device, comprising:
   a display;
   second wireless communication circuitry configured to receive time-correlated data characterizing an analyte level of the subject and dosage data for doses of the medication received by the subject over a period of time;
   and
   second one or more processors coupled to a second memory, the second memory storing instructions that, when executed by the second one or more processors, cause the one or more processors to:
      determine a dose recommendation of the medication based on at least calculated analyte levels, the calculated analyte levels based on the time-correlated data characterizing the analyte level of the subject,
wherein instructions stored in the first memory, when executed, cause the first one or more processors to provide haptic feedback through the dosing selector based on a comparison between the dose recommendation and a dose entered using the dosing selector.

Clause 41. The system of clause 40, wherein the haptic feedback comprises an increased resistance when the dosing selector is set to a dose higher than the dose recommendation.

Clause 42. The system of any of clauses 40-41, wherein the haptic feedback comprises an increased resistance when the dosing selector is set to a dose lower than the dose recommendation.

Clause 43. The system of any of clauses 40-42, wherein the dosing selector is a rotatable knob that is configured to turn in a first direction to increase a dose amount, and wherein the haptic feedback comprises a bias to turn in a second direction opposite the first direction when the dosing selector is turned to a dose higher than the dose recommendation.

Clause 44. The system of any of clauses 40-43, wherein the medication delivery device is a pen.

Clause 45. The system of any of clauses 40-44, wherein the medication delivery device is a pump.

Clause 46. The system of any of clauses 40-45, wherein the dosing selector is at least one button, and wherein the at least one button is configured to have an increased resistance to a downward motion when a dose higher than the dose recommendation is selected.

Clause 47. The system of any of clauses 40-46, further comprising a sensor control device comprising an analyte sensor, wherein at least a portion of the analyte sensor is configured to be in fluid contact with a bodily fluid of a subject.

Clause 48. A method, comprising the steps of:
receiving, by one or more processors of a medication delivery device, a dose recommendation;
receiving, by one or more processors of a medication delivery device, a dose being entered on the medication delivery device;
comparing the dose being entered to the dose recommendation; and
providing haptic feedback on the medication delivery device if the dose being entered is different than the dose recommendation.

Clause 49. The method of clause 48, wherein the haptic feedback comprises increased resistance of a dosing selector.

Clause 50. The method of any of clauses 48-49, wherein the dose being entered is entered using a dosing selector, and wherein the dosing selector is configured to be set by default to the dose recommendation.

Clause 51. The method of any of clauses 48-50, wherein the dose being entered is entered using a dosing selector.

Clause 52. The method of any of clauses 48-51, wherein the dosing selector is a rotatable dial.

Clause 53. The method of any of clauses 48-52, wherein the rotatable dial is configured to bounce back to a setting of the dose recommendation.

Clause 54. The method of any of clauses 48-53, wherein the rotatable dial is configured to have a resistance when rotating to an amount above the dose recommendation.

Clause 55. An analyte monitoring system, comprising:
a medication delivery device configured to deliver an amount of a medication to the subject, the medication delivery device comprising:
   a display;
   a dosing selector;
   first wireless communications circuitry configured to receive dose recommendations from a reader device; and
   first one or more processors coupled to a first memory;
a reader device, comprising:
   a display;
   second wireless communication circuitry configured to receive time-correlated data characterizing an analyte level of the subject and dosage data for doses of the medication received by the subject over a period of time; and
   second one or more processors coupled to a second memory, the second memory storing instructions that, when executed by the second one or more processors, cause the second one or more processors to:
      determine a dose recommendation of the medication based on at least calculated analyte levels, the calculated analyte levels based on the time-correlated data characterizing the analyte level of the subject,
wherein instructions stored in the first memory, when executed, cause the first one or more processors to lock the medication delivery device to prevent delivery of a medication when the dosing selector is set to a dose that is different than the dose recommendation.

Clause 56. The system of clause 55, wherein instructions stored in the first memory, when executed, cause the first one or more processors to lock the dosing selector when the dosing selector is set to a dose that is different than the dose recommendation.

Clause 57. The system of any of clauses 55-56, wherein instructions stored in the first memory, when executed, cause the first one or more processors to lock the medication delivery device when the dosing selector is set to a dose that is a predetermined number of units higher than the dose recommendation.

Clause 58. The system of any of clauses 55-57, wherein the predetermined number of units is about 2 units.

Clause 59. The system of any of clauses 55-58, wherein the second instructions further cause the one or more processors of the reader device to display a GUI comprising a notification of the dose that is a predetermined number of units greater than the dose recommendation.

Clause 60. The system of any of clauses 55-59, wherein the second instructions further cause the second one or more processors of the reader device to request confirmation from the subject regarding the dose that is a predetermined number of units greater than the dose recommendation.

Clause 61. The system of any of clauses 55-60, wherein the medication delivery device is a pen, and wherein the dosing selector is a rotatable knob.

Clause 62. The system of any of clauses 55-61, wherein the medication delivery device is a pump, and wherein the dosing selector is at least one button.

Clause 63. The system of any of clauses 55-62, further comprising a sensor control device comprising an analyte sensor, wherein at least a portion of the analyte sensor is configured to be in fluid contact with a bodily fluid of a subject.

Clause 64. A method, comprising the steps of:
receiving, by one or more processors of a medication delivery device, a dose recommendation;
receiving, by one or more processors of a medication delivery device, a dose being entered on the medication delivery device;
comparing the dose being entered to the dose recommendation; and
locking the medication delivery device to prevent delivery of a medication if the dose being entered is above the dose recommendation.

Clause 65. The method of clause 64, wherein the step of locking the medication delivery device comprises locking the dosing selector.

Clause 66. The method of any of clauses 64-65, wherein the medication deliver device is locked when the dosing selector is set to a dose that is a predetermined number of units higher than the dose recommendation.

Clause 67. The method of any of clauses 64-66, wherein the predetermined number of units is about 2 units.

Clause 68. The method of any of clauses 64-67, wherein the medication delivery device is a pen, and wherein the dosing selector is a rotatable knob.

Clause 69. The method of any of clauses 64-68, wherein the medication delivery device is a pump, and wherein the dosing selector is at least one button.

Clause 70. An analyte monitoring system, comprising:
a medication delivery device configured to deliver an amount of a medication to the subject, the medication delivery device comprising:
   a display;
   a dosing selector;
   first wireless communications circuitry configured to receive dose recommendations from a reader device; and
   first one or more processors coupled to a first memory;
a reader device, comprising:
   a display;
   second wireless communication circuitry configured to receive time-correlated data characterizing an analyte level of the subject and dosage data for doses of the medication received by the subject over a period of time; and
   second one or more processors coupled to a second memory, the second memory storing instructions that, when executed by the second one or more processors, cause the second one or more processors to:
      determine a dose recommendation of the medication based on at least calculated analyte levels and the doses of the medication received by the subject over the period of time, the calculated analyte levels based on the time-correlated data characterizing the analyte level of the subject, and
      communicate the recommended dose to the medication delivery device,
wherein instructions stored in the first memory, when executed, cause the first one or more processors to lock the medication delivery device to prevent delivery of a medication when the dose recommendation is equal to zero.

Clause 71. The system of clause 70, wherein instructions stored in the first memory, when executed, cause the first one or more processors to lock the dosing selector when the dose recommendation is equal to zero.

Clause 72. The system of any of clauses 70-71, wherein the dose recommendation is determined to be zero based on an insulin-on-board determination.

Clause 73. The system of any of clauses 70-72, wherein the dose recommendation is determined to be zero based on a determination of a risk of hypoglycemia based on the at least calculated analyte levels based on the time-correlated data characterizing the analyte level of the subject and the doses of the medication received by the subject over the period of time.

Clause 74. The system of any of clauses 70-73, wherein the medication delivery device is a pen, and wherein the dosing selector is a rotatable knob.

Clause 75. The system of any of clauses 70-74, wherein the pen further comprises a delivery button and a plunger mechanically coupled to the delivery button, wherein when the dose recommendation is zero, the delivery button decouples from the plunger.

Clause 76. The system of any of clauses 70-75, wherein the medication delivery device is a pump, and wherein the dosing selector is at least one button.

Clause 77. The system of any of clauses 70-76, further comprising a sensor control device comprising an analyte sensor, wherein at least a portion of the analyte sensor is configured to be in fluid contact with a bodily fluid of a subject.

Clause 78. A method, comprising the steps of:
receiving, by one or more processors of a medication delivery device, a dose recommendation;
determining if the dose recommendation is equal to zero; and
locking the medication delivery device to prevent delivery of a medication if the dose recommendation is determined to be a dosage of zero.

Clause 79. The method of clause 78, wherein the step of locking the medication delivery device comprises locking the dosing selector.

Clause 80. The method of any of clauses 78-79, wherein the dose recommendation is determined to be a dosage of zero based on an insulin-on-board determination.

Clause 81. The method of any of clauses 78-80, wherein the dose recommendation is determined to be zero based on a determination of a risk of hypoglycemia based on the at least calculated analyte levels based on the time-correlated data characterizing the analyte level of the subject and dose data for the doses of the medication received by the subject over the period of time.

Clause 82. The method of any of clauses 78-81, wherein the medication delivery device is a pen, and wherein the dosing selector is a rotatable knob.

Clause 83. The method of any of clauses 78-82, wherein the pen further comprises a delivery button and a plunger mechanically coupled to the delivery button, wherein step of locking the medication delivery device comprises decoupling the delivery button from the plunger.

Clause 84. An analyte monitoring system, comprising:
a medication delivery device configured to deliver an amount of a medication to the subject, the medication delivery device comprising:
   a display;
   a dosing selector; and
   first wireless communications circuitry configured to receive dose recommendations from a reader device and to communicate doses of the medication received by the subject; and
a reader device, comprising:
   a display;
   second wireless communication circuitry configured to receive time-correlated data characterizing an analyte level of the subject and dosage data for the doses of the medication received by the subject from the medication delivery device; and
   one or more processors coupled to a memory, the memory storing instructions that, when executed by the one or more processors, cause the one or more processors to:
      determine a dose recommendation of the medication based on at least calculated analyte levels, the calculated analyte levels based on the time-correlated data characterizing the analyte level of the subject,
      compare the dose recommendation of the medication with a corresponding amount of the medication delivered by the medication delivery device, and
      adjust an alert parameter if the corresponding amount of medication delivered is different than the dose recommendation.

Clause 85. The system of clause 84, wherein the alert parameter is a low glucose threshold, and wherein the low glucose threshold is increased to a higher concentration when the amount of medication delivered is higher than the dose recommendation.

Clause 86. The system of any of clauses 84-85, wherein the alert parameter is a high glucose threshold, and wherein the high glucose threshold is decreased to a lower concentration when the amount of medication delivered is lower than the dose recommendation.

Clause 87. The system of any of clauses 84-86, wherein the alert parameter is a time period during which an alert is predicted, and wherein the time period during which an alert is predicted is increased when the corresponding amount of medication delivered is different than the recommended dose.

Clause 88. The system of any of clauses 84-87, wherein the time period during which an alert is predicted is increased by at least about 20 minutes when the corresponding amount of medication delivered is different than the recommended dose.

Clause 89. The system of any of clauses 84-88, wherein the alert parameter is a snooze period, and wherein the snooze period is decreased to a shorter time period when the corresponding amount of medication delivered is different than the recommended dose.

Clause 90. The system of any of clauses 84-89, wherein the alert parameter is a volume of an audible alert, and wherein the volume is increased when the corresponding amount of medication delivered is different than the recommended dose.

Clause 91. The system of any of clauses 84-90, wherein the alert parameter is a duration of an audible alert, and wherein the duration is increased when the corresponding amount of medication delivered is different than the recommended dose.

Clause 92. The system of any of clauses 84-91, wherein the alert parameter is a requirement of an acknowledgement of the alert, and wherein the requirement of the acknowledgement of the alert is enabled when the corresponding amount of medication delivered is different than the recommended dose.

Clause 93. The system of any of clauses 84-92, wherein the instructions, when executed, further cause the one or more processors to display additional information regarding the amount of medication delivered.

Clause 94. The system of any of clauses 84-93, wherein the alert parameter is a tactile parameter, and wherein the alert parameter is adjusted to cause the reader device to vibrate if the corresponding amount of medication delivered is higher than the dose recommendation.

Clause 95. The system of any of clauses 84-94, wherein the alert parameter is an audible parameter, and wherein the alert parameter is adjusted to cause the reader device to emit a sound if the corresponding amount of medication delivered is higher than the dose recommendation.

Clause 96. The system of any of clauses 84-95, further comprising a sensor control device comprising an analyte sensor, wherein at least a portion of the analyte sensor is configured to be in fluid contact with a bodily fluid of a subject.

Clause 97. A method, comprising the steps of:
determining, by one or more processors of a reader device, a dose recommendation;
receiving, by one or more processors of a reader device, dosage data for a dose entered or delivered by a medication delivery device;
comparing the dose entered or delivered to the dose recommendation; and
adjusting, by one or more processors of a reader device, an alert parameter if the dose entered or delivered is different than the dose recommendation.

Clause 98. The method of clause 97, wherein the alert parameter is a low glucose threshold, and wherein the low glucose threshold is increased to a higher concentration when the amount of medication delivered is higher than the dose recommendation.

Clause 99. The method of any of clauses 97-98, wherein the alert parameter is a high glucose threshold, and wherein the high glucose threshold is decreased to a lower concentration when the amount of medication delivered is lower than the dose recommendation.

Clause 100. The method of any of clauses 97-99, wherein the alert parameter is a time period during which an alert is predicted, and wherein the time period during which an alert is predicted is increased when the corresponding amount of medication delivered is different than the recommended dose.

Clause 101. The method of any of clauses 97-100, wherein the time period during which an alert is predicted is increased by at least about 20 minutes when the corresponding amount of medication delivered is different than the recommended dose.

Clause 102. The method of any of clauses 97-101, wherein the alert parameter is a snooze period, and wherein the snooze period is decreased to a shorter time period when the corresponding amount of medication delivered is different than the recommended dose.

Clause 103. The method of any of clauses 97-102, wherein the alert parameter is a volume of an audible alert, and wherein the volume is increased when the corresponding amount of medication delivered is different than the recommended dose.

Clause 104. The method of any of clauses 97-103, wherein the alert parameter is a duration of an audible alert, and wherein the duration is increased when the corresponding amount of medication delivered is different than the recommended dose.

Clause 105. The method of any of clauses 97-104, wherein the alert parameter is a requirement of an acknowledgement of the alert, and wherein the requirement of the acknowledgement of the alert is enabled when the corresponding amount of medication delivered is different than the recommended dose.

Clause 106. The method of any of clauses 97-105, further comprising the step of displaying additional information regarding the amount of medication delivered.

Clause 107. The method of any of clauses 97-106, wherein the alert parameter is a tactile parameter, and wherein the alert parameter is adjusted to cause the reader device to vibrate if the corresponding amount of medication delivered is higher than the recommended dose.

Clause 108. The method of any of clauses 97-107, wherein the alert parameter is an audible parameter, and wherein the alert parameter is adjusted to cause the reader device to emit a sound if the corresponding amount of medication delivered is higher than the recommended dose.

Clause 109. An analyte monitoring system, comprising:
a medication delivery device configured to deliver an amount of a medication to the subject, the medication delivery device comprising:
   a display;
   a dosing selector; and
   first wireless communications circuitry configured to communicate proposed dose amounts of the medication entered using the dosing selector from the medication delivery device before delivery of the doses of the medication entered;
the reader device, comprising:
   a display;
   second wireless communication circuitry configured to receive time-correlated data characterizing an analyte level of the subject and the communicated proposed dose amounts of the medication entered using the dosing selector from the medication delivery device before delivery of the doses of the medication entered; and
   one or more processors coupled to a memory, the memory storing instructions that, when executed by the one or more processors, cause the one or more processors to:
      determine a dose recommendation of the medication based on at least calculated analyte levels, the calculated analyte levels based on the time-correlated data characterizing the analyte level of the subject,
      compare the dose recommendation of the medication with a proposed dose amount of the medication entered using the dosing selector, and
      cause a graphic user interface to be displayed on the display, the graphic user interface comprising additional dose guidance if the proposed dose amount of the medication is different than the dose recommendation of the medication.

Clause 110. The system of clause 109, wherein the proposed dose amount of the medication is greater than the dose recommendation.

Clause 111. The system of any of clauses 109-110, wherein the proposed dose amount of the medication is smaller than the dose recommendation.

Clause 112. The system of any of clauses 109-111, wherein the graphic user interface is displayed if the proposed dose amount of the medication is entered within a predetermined amount of time of the determination of the dose recommendation.

Clause 113. The system of any of clauses 109-112, wherein the graphic user interface is displayed if the proposed dose amount of the medication is entered after a predetermined amount of time of the determination of the dose recommendation.

Clause 114. The system of any of clauses 109-113, wherein the additional dose guidance relates to a time of day that the proposed dose amount of the medication is entered.

Clause 115. The system of any of clauses 109-114, further comprising a sensor control device comprising an analyte sensor, wherein at least a portion of the analyte sensor is configured to be in fluid contact with a bodily fluid of a subject.

Clause 116. A method, comprising the steps of:
determining, by one or more processors of a reader device, a dose recommendation;
receiving, by one or more processors of a reader device, a dose being entered on a medication delivery device;
comparing the dose being entered to the recommended dose; and
displaying a user interface related to the dose recommendation if the dose entered is different than the recommended dose.

Clause 117. The method of clause 116, wherein the user interface is displayed if the dose being entered is within a predetermined amount of time of the determination of the dose recommendation.

Clause 118. The method of any of clauses 116-117, wherein the user interface is displayed if the dose being entered is after a predetermined amount of time of the determination of the dose recommendation.

Clause 119. The method of any of clauses 116-118, wherein the user interface comprises additional dose guidance related to a time of day corresponding to the dose being entered on the medication delivery device.

Clause 120. An analyte monitoring system, comprising:
a medication delivery device configured to deliver an amount of a medication to the subject, the medication delivery device comprising:
   a display;
   at least one button;
   first wireless communications circuitry configured to receive dose recommendations from a reader device; and
   first one or more processors coupled to a first memory;
a reader device, comprising:
   a display;
   second wireless communication circuitry configured to receive time-correlated data characterizing an analyte level of the subject and dosage data for doses of the medication received by the subject over a period of time from the medication delivery device;
   and
   second one or more processors coupled to a second memory, the second memory storing instructions that, when executed by the second one or more processors, cause the second one or more processors to:
      determine a dose recommendation of the medication for at least one meal type based on at least calculated analyte levels, the calculated analyte levels based on the time-correlated data characterizing the analyte level of the subject, and
      communicate the dose recommendation of the medication for at least one meal type to the medication delivery device,
wherein instructions stored in the first memory, when executed, cause the first one or more processors of the medication delivery device to display the dose recommendation of the medication on the display of the medication delivery device for the at least one meal type when the at least one button is selected.

Clause 121. The system of clause 120, wherein the second instructions, when executed, cause the second one or more processors to determine a dose recommendation for each of a plurality of meal types, and wherein the at least one button comprises a plurality of buttons, wherein each meal type corresponds to a respective button of the plurality of buttons, wherein the first memory of the medication delivery device causes the first one or more processors of the medication delivery device to display a dose recommendation for one of the plurality of meal types in response to a selection of the respective button of the plurality of buttons.

Clause 122. The system of any of clauses 120-121, wherein the plurality of meal types comprises breakfast, lunch, dinner, and snack.

Clause 123. The system of any of clauses 120-122, wherein the second instructions, when executed, cause the second one or more processors to determine a dose recommendation for a basal dose, and wherein the at least one button comprises a plurality of buttons, wherein the dose recommendation for the basal dose corresponds to a respective button of the plurality of buttons, wherein the first memory of the medication delivery device causes the first one or more processors of the medication delivery device to display the dose recommendation for the basal dose in response to a selection of the respective button of the plurality of buttons.

Clause 124. The system of any of clauses 120-123, wherein the second instructions, when executed, cause the second one or more processors to determine a dose recommendation for a correction dose, and wherein the at least one button comprises a plurality of buttons, wherein the recommended dose for the correction dose corresponds to a respective button of the plurality of buttons, wherein the first memory of the medication delivery device causes the first one or more processors of the medication delivery device to display the dose recommendation for the correction dose in response to a selection of the respective button of the plurality of buttons.

Clause 125. The system of any of clauses 120-124, further comprising a sensor control device comprising an analyte sensor, wherein at least a portion of the analyte sensor is configured to be in fluid contact with a bodily fluid of a subject.

Clause 126. A method, comprising the steps of:
receiving, by one or more processors of a medication delivery device, a dose recommendation for at least one meal type;
assigning, by the one or more processors of the medication delivery device, the dose recommendation for the at least one meal type to one of a plurality of buttons on the medication delivery device; and
displaying the dose recommendation on the medication delivery device in response to the selection of the one of the plurality of buttons on the medication delivery device.

Clause 127. The method of clause 126, further comprising the step of setting, by the one or more processors of the medication delivery device, a dose selector of the medication delivery device to the dose recommendation.

Clause 128. The method of any of clauses 126-127, wherein the receiving step comprises receiving, by one or more processors of a medication delivery device, a dose recommendation for each of a plurality of meal types.

Clause 129. The method of any of clauses 126-128, wherein the plurality of meal types comprises at least two of breakfast, lunch, dinner, and snack.

Clause 130. The method of any of clauses 126-129, further comprising the steps of:
receiving, by the one or more processors of the medication delivery device, a dose recommendation for a basal dose;
assigning, by the one or more processors of the medication delivery device, the dose recommendation for the basal dose to one of a plurality of buttons on the medication delivery device.

Clause 131. The method of any of clauses 126-130, further comprising the steps of:
receiving, by the one or more processors of the medication delivery device, a dose recommendation for a correction dose;
assigning, by the one or more processors of the medication delivery device, the dose recommendation for the correction dose to one of a plurality of buttons on the medication delivery device.

Clause 132. An analyte monitoring system, comprising:
a medication delivery device configured to deliver an amount of a medication to the subject, the medication delivery device comprising:
   a display:
   a dosing selector;
   first wireless communications circuitry configured to receive dose recommendations from a reader device; and
   first one or more processors coupled to a first memory;
a reader device, comprising:
   a display;
   second wireless communication circuitry configured to receive time-correlated data characterizing an analyte level of the subject and dosage data for doses of the medication received by the subject over a period of time; and
   second one or more processors coupled to a second memory, the second memory storing instructions that, when executed by the second one or more processors, cause the second one or more processors to:
      determine a dose recommendation of the medication based on at least calculated analyte levels, the calculated analyte levels based on the time-correlated data characterizing the analyte level of the subject and the doses of the medication received by the subject over the period of time,
wherein instructions in the first memory, when executed, cause the first one or more processors to change a configuration of the medication delivery device to prevent the medication delivery device from delivering the medication when a predetermined time since the dose recommendation of the medication was determined has elapsed.

Clause 133. The system of clause 132, wherein the dosing selector is changed to a locked configuration after the predetermined time has elapsed.

Clause 134. The system of any of clauses 132-133, wherein the dosing selector is changed to zero after the predetermined time has elapsed.

Clause 135. The system of any of clauses 132-134, further comprising a sensor control device comprising an analyte sensor, wherein at least a portion of the analyte sensor is configured to be in fluid contact with a bodily fluid of a subject.

Clause 136. A method, comprising the steps of:
receiving, by one or more processors of a medication delivery device, dosage data for a dose recommendation at a first time;
receiving, by one or more processors of a medication delivery device, dosage data for a dose being entered on the medication delivery device at a second time;
determining, by one or more processors of a medication delivery device, if the difference between the second time and the first time is greater than a predetermined amount of time; and
changing, by one or more processors of a medication delivery device, a configuration of the medication delivery device if the difference between the second time and the first time is greater than a predetermined amount of time.

Clause 137. The method of clause 136, wherein a dosing selector of the medication delivery device is changed to a locked configuration after the predetermined time has elapsed.

Clause 138. The method of any of clauses 136-137, wherein a dosing selector of the medication delivery device is changed to zero after the predetermined time has elapsed.

Clause 139. An analyte monitoring system, comprising:
a medication delivery device configured to deliver an amount of a medication to the subject, the medication delivery device comprising:
   a display:
   a dosing selector;
   first wireless communications circuitry configured to receive a dose recommendation from a reader device at a first time, T1; and
   first one or more processors coupled to a first memory, the first memory storing instructions that, when executed by the first one or more processors, cause the first one or more processors to:
      determine if a dose has been delivered during a predetermined period of time since the first time, T1,
a reader device, comprising:
   a display;
   second wireless communication circuitry configured to receive time-correlated data characterizing an analyte level of the subject and dosage data for doses of the medication received by the subject over a period of time; and
   second one or more processors coupled to a second memory, the second memory storing instructions that, when executed by the second one or more processors, cause the second one or more processors to:
      determine a recommended dose of the medication based on at least calculated analyte levels and the doses of the medication received by the subject over the period of time, the calculated analyte levels based on the time-correlated data characterizing the analyte level of the subject,
wherein instruction stored in the first memory, when executed, cause the first one or more processors to change a configuration of the medication delivery device to prevent the medication delivery device from delivering the medication if it is determined that the dose has not been delivered during the predetermined period of time since the first time, T1.

Clause 140. The system of clause 139, wherein the dosing selector is changed to a locked configuration after the predetermined period of time since the first time, T1 has elapsed.

Clause 141. The system of any of clauses 139-140, wherein the dosing selector is changed to a dosage of zero after the predetermined period of time since the first time, T1 has elapsed.

Clause 142. The system of any of clauses 139-141, further comprising a sensor control device comprising an analyte sensor, wherein at least a portion of the analyte sensor is configured to be in fluid contact with a bodily fluid of a subject.

Clause 143. A method, comprising the steps of:
receiving, by one or more processors of a medication delivery device, dosage data for a recommended dose at a first time;
determining, by one or more processors of the medication delivery device, if a dose has been delivered during a predetermined period of time since a first time, T1; and
changing a configuration of the medication delivery device if the it is determined that no dose has been delivered during the predetermined period of time since the first time, T1.

Clause 144. The method of clause 143, wherein the step of changing the configuration of the medication delivery device comprises changing a dosing selector of the medication delivery device to a locked configuration.

Clause 145. The method of any of clauses 143-144, wherein the step of changing the configuration of the medication delivery device comprises changing a dosing selector of the medication delivery device to a dosage of zero.

Clause 146. An analyte monitoring system, comprising:
a medication delivery device configured to deliver an amount of a medication to the subject, the medication delivery device comprising:
   a display;
   a dosing selector; and
   wireless communications circuitry configured to receive dose recommendations from a reader device and to communicate doses administered to the reader device;
a reader device, comprising:
   a display;
   wireless communication circuitry configured to receive time-correlated data characterizing an analyte level of the subject and dosage data for doses of the medication received by the subject over a period of time from the medication delivery device; and
   one or more processors coupled to a memory, the memory storing instructions that, when executed by the one or more processors, cause the one or more processors to:
      determine a dose recommendation of the medication based on at least calculated analyte levels, the calculated analyte levels based on the time-correlated data characterizing the analyte level of the subject at a first time,
      determine if a dose has been administered during a predetermined period of time since the dose recommendation was determined at the first time,
      determine a new dose recommendation of the medication based on at least calculated analyte levels based on the time-correlated data characterizing the analyte level of the subject at a second time; and
      communicate the new dose recommendation to the medication delivery device.

Clause 147. The system of clause 146, wherein the medication delivery device further comprises one or more processors coupled to an additional memory, the additional memory storing instructions that, when executed by the one or more processors, cause the dose recommendation to be displayed on the display of the medication delivery device.

Clause 148. The system of any of clauses 146-147, wherein the new recommended dose is lower than the dose recommendation.

Clause 149. The system of any of clauses 146-148, further comprising a sensor control device comprising an analyte sensor, wherein at least a portion of the analyte sensor is configured to be in fluid contact with a bodily fluid of a subject.

Clause 150. A method, comprising the steps of
determining, by one or more processors of a reader device, a dose recommendation at a first time, T1;
receiving, by one or more processors of a reader device, if a dose has been delivered during a predetermined period of time since the first time, T1;
determining, by one or more processors of a reader device, a new dose recommendation if it is determined that no dose has been delivered during the predetermined period of time since time T1; and
communicating the new dose recommendation to a medication delivery device.

Clause 151. The method of clause 150, further comprising the step of displaying the new dose recommendation on the medication delivery device.

Clause 152. The method of any of clauses 150-151, further comprising the step of displaying the new dose recommendation on the reader device.

Clause 153. The method of any of clauses 150-152, wherein the predetermined period time is at least about 20 minutes.

Clause 154. The method of any of clauses 150-153, wherein the new dose recommendation is lower than the dose recommendation.

Clause 155. An analyte monitoring system, comprising:
a medication delivery device configured to deliver an amount of a medication to the subject, the medication delivery device comprising:
   a display;
   a dosing selector; and
   wireless communications circuitry configured to receive dose recommendations from a reader device and to communicate doses administered to the reader device;
a reader device, comprising:
   a display;
   wireless communication circuitry configured to receive time-correlated data characterizing an analyte level of the subject and receive dosage data for doses of the medication received by the subject over a period of time from the medication delivery device;
   and
   one or more processors coupled to a memory, the memory storing instructions that, when executed by the one or more processors, cause the one or more processors to:
      determine a first recommended dose of the medication based on at least calculated analyte levels based on the time-correlated data characterizing the analyte level of the subject at a first time,
      determine if the first recommended dose is below a threshold value,
      determine if a dose has been administered during a predetermined period of time since the first time if it is determined that the first recommended dose is not below a threshold value,
      determine a second recommended dose of the medication at a second time based on at least calculated analyte levels, the calculated analyte levels based on the time-correlated data characterizing the analyte level of the subject if it is determined that the dose has not been administered within the predetermined time period

Clause 156. The system of clause 155, wherein the instructions further cause the one or more processors to:
determine if the second recommended dose is below a threshold value,
determine if the dose has been administered during a second predetermined period of time since the second time if it is determined that the dose is not below the threshold value, and
revise the second recommended dose at a third time based on at least calculated analyte levels based on the time-correlated data characterizing the analyte level of the subject if it is determined that the dose has not been administered within the second predetermined time period.

Clause 157. The system of any of clauses 155-156, further comprising a sensor control device comprising an analyte sensor, wherein at least a portion of the analyte sensor is configured to be in fluid contact with a bodily fluid of a subject.

Clause 158. A method, comprising the steps of
determining, by one or more processors of a reader device, a first recommended dose of the medication based on at least calculated analyte levels, the calculated analyte levels based on the time-correlated data characterizing the analyte level of the subject at a first time,
determining, by the one or more processors of the reader device, if the first recommended dose is below a threshold value,
determining, by the one or more processors of the reader device, if a dose has been administered during a predetermined period of time since the first time if it is determined that the first recommended dose is not below a threshold value, and
determining, by the one or more processors of the reader device, a second recommended dose of the medication at a second time based on at least calculated analyte levels based on the time-correlated data characterizing the analyte level of the subject if it is determined that the dose has not been administered within the predetermined time period.

Clause 159. The method of clause 158, further comprising the steps of:
determining, by the one or more processors of the reader device, if the second recommended dose is below the threshold value,
determining, by the one or more processors of the reader device, if the dose has been administered during a second predetermined period of time since the second time if it is determined that the second recommended dose is not below the threshold value, and
revising, by the one or more processors of the reader device, the second recommended dose at a third time based on at least calculated analyte levels based on the time-correlated data characterizing the analyte level of the subject if it is determined that the dose has not been administered within the second predetermined time period.

Clause 160. A system for displaying metrics relating to a subject, the system comprising:
wireless communications circuitry configured to receive time-correlated data characterizing an analyte level of the subject;
a display configured to visually present information;
a memory; and one or more processors coupled with the wireless communications circuitry, the display, and the memory, wherein the memory stores instructions and time-correlated data characterizing an analyte level of the subject, wherein the instructions, when executed by the one or more processors, cause the system to:
   determine a subset of time-correlated data based on a filtering criteria selected by the subject, wherein the filtering criteria comprises enablement of at least one alarm; and
   display a first glucose profile and a second glucose profile on a single graphical subject interface, wherein the first glucose profile displays glucose levels associated with the time-correlated data over a first time period, and wherein the second glucose profile displays the subset of the time-correlated data over the first time period.

Clause 161. The system of clause 160, wherein the at least one alarm is a low glucose alarm.

Clause 162. The system of any of clauses 160-161, wherein the low glucose alarm has a threshold of about 70 mg/dL.

Clause 163. The system of any of clauses 160-162, wherein the at least one alarm is a high glucose alarm.

Clause 164. The system of any of clauses 160-163, wherein the high glucose alarm has a threshold of about 180 mg/dL.

Clause 165. The system of any of clauses 160-164, wherein the instructions, when executed by the one or more processor, further cause the system to:
display occurrences of the at least one alarm in the first time period on at least one of the first and second glucose profiles.

Clause 166. The system of any of clauses 160-165, wherein the wireless communications circuitry is further configured to receive dosage data for doses of a medication received by the subject over a period of time, and wherein the memory further stores doses of a glucose level-altering medication received by the subject over a period of time,
wherein the instructions, when executed by the one or more processor, further cause the system to:
   display instances of doses of the medication received in the first time period on at least one of the first and second glucose profiles.

Clause 167. The system of any of clauses 160-166, wherein the instructions, when executed by the one or more processor, further cause the system to:
determine a second subset of time-correlated data based on the filtering criteria selected by the subject, wherein the second subset of time-correlated data comprises time-correlated data when the at least one alarm is disabled, and
display a third glucose profile on the single graphical subject interface, wherein the third glucose profile displays the second subset of time-correlated data over the first time period.

Clause 168. The system of any of clauses 160-167, wherein the instructions, when executed by the one or more processor, further cause the system to:
display occurrences of the at least one alarm in the first time period on at least one of the first, second, and third glucose profiles.

Clause 169. The system of any of clauses 160-168, wherein the wireless communications circuitry is further configured to receive dosage data for doses of a medication received by the subject over a period of time, and wherein the memory further stores doses of a glucose level-altering medication received by the subject over a period of time,
wherein the instructions, when executed by the one or more processor, further cause the system to:

display instances of doses of the medication received in the first time period on at least one of the first, second, and third glucose profiles.

Clause 170. The system of any of clauses 160-169, wherein the instances of doses the medication received in the first time period are displayed on the second glucose profile.

Clause 171. The system of any of clauses 160-170, wherein the wireless communications circuitry is further configured to receive doses of a medication received by the subject over a period of time, and wherein the memory further stores doses of a glucose level-altering medication received by the subject over a period of time, wherein the instructions cause the one or more processors to determine at least one recommended dose, and wherein the filtering criteria further comprises dose concordance of the glucose altering medication.

Clause 172. The system of any of clauses 160-171, wherein the wireless communications circuitry is further configured to receive dosage data for a dose regimen for the subject, and wherein the at least one recommended dose is at least partly determined from the dose regimen.

Clause 173. The system of any of clauses 160-172, wherein the dose concordance comprises missed doses, under-bolused doses with reference to the at least one recommended dose, over-bolused doses with reference to the at least one recommended dose, late meal doses, or extra meal doses.

Clause 174. The system of any of clauses 160-173, wherein the first glucose profile and the second glucose profile each comprise a trace of a median glucose level.

Clause 175. The system of any of clauses 160-174, wherein the first glucose profile and the second glucose profile each further comprise an upper trace of an hourly 95^{th} percentile of glucose levels and a lower trace of an hourly 5^{th} percentile of glucose levels.

Clause 176. The system of any of clauses 160-175, wherein the first glucose profile and the second glucose profile each further comprise a second upper trace of an hourly 75^{th} percentile of glucose levels and a second lower trace of an hourly 25^{th} percentile of glucose levels.

Clause 177. The system of any of clauses 160-176, wherein the first glucose profile and the second glucose profile each further comprise data indicative of an hourly 75^{th} percentile of glucose levels and a second lower trace of an hourly 15^{th} percentile of glucose levels.

Clause 178. The system of any of clauses 160-177, wherein the first glucose profile and the second glucose profile each comprise a plurality of traces, wherein a trace of the plurality of traces corresponds to a day of the first period of time, and wherein the plurality of traces are displayed in an overlay pattern.

Clause 179. The system of any of clauses 160-178, wherein the instructions, when executed by the one or more processor, further cause the system to:
determine a first analyte metric based on the time-correlated data for the first time period and a second analyte metric based on the subset of the time correlated data for the first time period; and
display the first analyte metric and the second analyte metric on the single graphical subject interface.

Clause 180. The system of any of clauses 160-179, wherein the instructions, when executed by the one or more processor, further cause the system to:
determine at least one first analyte statistic based on the time-correlated data for the first time period and at least one second analyte statistic based on the subset of the time correlated data for the first time period; and
display the at least one first analyte statistic and the at least one second analyte statistic on the single graphical subject interface.

Clause 181. The system of any of clauses 160-180, wherein the at least one first analyte statistic comprises a first glucose management indicator, a first average glucose, a first standard deviation, or combinations thereof, and/or wherein the at least one second analyte statistic comprises a second glucose management indicator, a second average glucose, a second standard deviation, or combinations thereof.

Clause 182. The system of any of clauses 160-181, wherein the instructions, when executed by the one or more processor, further cause the system to:
determine a first hypoglycemia risk based on the time-correlated data for the first time period and a second hypoglycemia risk based on the subset of the time correlated data for the first time period; and
display the first and the second hypoglycemia risk on the single graphical subject interface.

Clause 183. A method for displaying metrics relating to a subject, the method comprising:
receiving time-correlated data characterizing an analyte level of a subject;
determining a subset of time-correlated data based on a filtering criteria selected by the subject, wherein the filtering criteria comprises enablement of at least one alarm; and
displaying a first glucose profile and a second glucose profile on a single graphical subject interface, wherein the first glucose profile displays glucose levels associated with the time-correlated data over a first time period, and wherein the second glucose profile displays the subset of the time-correlated data over the first time period.

Clause 184. The method of clause 183, wherein the at least one alarm is a low glucose alarm.

Clause 185. The method of any of clauses 183-184, wherein the low glucose alarm has a threshold of about 70 mg/dL.

Clause 186. The method of any of clauses 183-185, wherein the at least one alarm is a high glucose alarm.

Clause 187. The method of any of clauses 183-186, wherein the high glucose alarm has a threshold of about 180 mg/dL.

Clause 188. The method of any of clauses 183-187, further comprising the step of: displaying occurrences of the at least one alarm in the first time period on at least one of the first and second glucose profiles.

Clause 189. The method of any of clauses 183-188, further comprising the step of: receiving dosage data for doses of a medication received by the subject over a period of time;
storing dosage data for doses of a glucose level-altering medication received by the subject over a period of time; and
displaying instances of doses of the medication received in the first time period on at least one of the first and second glucose profiles.

Clause 190. The method of any of clauses 183-189, further comprising the step of:
determining a second subset of time-correlated data based on the filtering criteria selected by the subject, wherein the second subset of time-correlated data comprises time-correlated data when the at least one alarm is disabled, and
displaying a third glucose profile on the single graphical subject interface, wherein the third glucose profile displays the second subset of time-correlated data over the first time period.

Clause 191. The method of any of clauses 183-190, further comprising the step of: displaying occurrences of the at least one alarm in the first time period on at least one of the first, second, and third glucose profiles.

Clause 192. The method of any of clauses 183-191, further comprising the step of:
receiving dosage data for doses of a medication received by the subject over a period of time;
storing dosage data for doses of a glucose level-altering medication received by the subject over a period of time; and
displaying instances of doses of the medication received in the first time period on at least one of the first, second, and third glucose profiles.

Clause 193. The method of any of clauses 183-192, wherein the instances of doses the subject received in the first time period are displayed on the second glucose profile.

Clause 194. The method of any of clauses 183-193, wherein the wireless communications circuitry is further configured to receive dosage data for doses of a medication received by the subject over a period of time, and wherein the memory further stores doses of a glucose level-altering medication received by the subject over a period of time, and wherein the filtering criteria further comprises dose concordance.

Clause 195. The method of any of clauses 183-194, wherein the dose concordance comprises recommended doses, missed doses, under-bolused doses, over-bolused doses, late meal doses, or extra meal doses.

Clause 196. The method of any of clauses 183-195, wherein the first glucose profile and the second glucose profile each comprise a trace of a median glucose level.

Clause 197. The method of any of clauses 183-196, wherein the first glucose profile and the second glucose profile each further comprise an upper trace of an hourly 95^{th} percentile of glucose levels and a lower trace of a 5^{th} percentile of glucose levels.

Clause 198. The method of any of clauses 183-197, wherein the first glucose profile and the second glucose profile each further comprise a second upper trace of an hourly 75^{th} percentile of glucose levels and a second lower trace of an hourly 25^{th} percentile of glucose levels.

Clause 199. The method of any of clauses 183-198, wherein the first glucose profile and the second glucose profile each further comprise data indicative of an hourly 75^{th} percentile of glucose levels and a second lower trace of an hourly 15^{th} percentile of glucose levels.

Clause 200. The method of any of clauses 183-199, wherein the first glucose profile and the second glucose profile each comprise a plurality of traces, wherein a trace of the plurality of traces corresponds to a day of the first period of time, and wherein the plurality of traces are displayed in an overlay pattern.

Clause 201. The method of any of clauses 183-200, further comprising the steps of: determining a first analyte metric based on the time-correlated data for the first time period and a second analyte metric based on the subset of the time correlated data for the first time period; and
displaying the first analyte metric and the second analyte metric on the single graphical subject interface.

Clause 202. The method of any of clauses 183-201, further comprising the steps of:
determining at least one first analyte statistic based on the time-correlated data for the first time period and at least one second analyte statistic based on the subset of the time correlated data for the first time period; and
displaying the at least one first analyte statistic and the at least one second analyte statistic on the single graphical subject interface.

Clause 203. The method of any of clauses 183-202, wherein the at least one first analyte statistic comprises a first glucose management indicator, a first average glucose, a first standard deviation, or combinations thereof, and/or wherein the at least one second analyte statistic comprises a second glucose management indicator, a second average glucose, a second standard deviation, or combinations thereof.

Clause 204. The method of any of clauses 183-203, further comprising the steps of:
determining a first hypoglycemia risk based on the time-correlated data for the first time period and a second hypoglycemia risk based on the subset of the time correlated data for the first time period; and
displaying the first and the second hypoglycemia risk on the single graphical subject interface.

Clause 205. A system for displaying metrics relating to a subject, the system comprising:
wireless communications circuitry configured to receive time-correlated data characterizing an analyte level of the subject;
a display configured to visually present information;
a memory; and one or more processors coupled with the wireless communications circuitry, the display, and the memory, wherein the memory stores instructions and time-correlated data characterizing an analyte level of the subject, wherein the instructions, when executed by the one or more processors, cause the system to:
   determine a subset of time-correlated data based on a filtering criteria selected by the subject;
   determine a first analyte metric based on the time-correlated data for a first time period and a second analyte metric based on the subset of the time correlated data for the first time period; and
   display the first analyte metric and the second analyte metric on a single graphical subject interface.

Clause 206. The system of clause 205, wherein the first analyte metric is an amount of time that analyte levels determined from the time-correlated data were within at least one concentration range during the first time period, and the second analyte metric is an amount of time that analyte levels determined from the subset of time-correlated data were within the at least one concentration range during the first time period.

Clause 207. The system of any of clauses 205-206, wherein the at least one concentration range comprises a target concentration range.

Clause 208. The system of any of clauses 205-207, wherein the at least one concentration range comprises a plurality of concentration ranges.

Clause 209. The system of any of clauses 205-208, wherein the plurality of concentration ranges comprises a low glucose concentration range, a target concentration range, and a high glucose concentration range.

Clause 210. The system of any of clauses 205-209, wherein the plurality of concentration ranges comprises a very low glucose concentration range, a low glucose concentration range, a target concentration range, a high glucose concentration range, and a very high glucose concentration range.

Clause 211. The system of any of clauses 205-210, wherein the filtering criteria comprises enablement of at least one alarm.

Clause 212. The system of any of clauses 205-211, wherein the instructions, when executed by the one or more processor, further cause the system to:
determine a second subset of time-correlated data based on the filtering criteria selected by the subject, wherein the second subset of time-correlated data comprises time-correlated data when the at least one alarm is disabled, and
display a third analyte metric on the single graphical subject interface, wherein the third glucose profile displays the second subset of time-correlated data over the first time period.

Clause 213. The system of any of clauses 205-212, wherein the instructions, when executed by the one or more processor, further cause the system to:
determine an analyte level based on the time-correlated data; and
display the analyte level on an additional graphical user interface;
wherein the filtering criteria comprises the subject viewing the analyte level on the additional graphical user interface within a period of time.

Clause 214. The system of any of clauses 205-213, wherein the period of time is about 3 hours.

Clause 215. The system of any of clauses 205-214, wherein the filtering criteria comprises time-correlated data corresponding to an A1c value greater than a threshold.

Clause 216. The system of any of clauses 205-215, wherein the threshold is about 7%.

Clause 217. The system of any of clauses 205-216, wherein the instructions, when executed by the one or more processor, further cause the system to:

display a first glucose profile and a second glucose profile on the single graphical subject interface, wherein the first glucose profile displays glucose levels associated with the time-correlated data over the first time period, and wherein the second glucose profile displays the subset of the time-correlated data over the first time period.

Clause 218. The system of any of clauses 205-217, wherein the first glucose profile and the second glucose profile each comprise a trace of a median glucose level.

Clause 219. The system of any of clauses 205-218, wherein the first glucose profile and the second glucose profile each further comprise an upper trace of an hourly 95^{th} percentile of glucose levels and a lower trace of a 5^{th} percentile of glucose levels.

Clause 220. The system of any of clauses 205-219, wherein the first glucose profile and the second glucose profile each further comprise a second upper trace of an hourly 75^{th} percentile of glucose levels and a second lower trace of an hourly 25^{th} percentile of glucose levels.

Clause 221. The system of any of clauses 205-220, wherein the first glucose profile and the second glucose profile each further comprise data indicative of an hourly 75^{th} percentile of glucose levels and a second lower trace of an hourly 15^{th} percentile of glucose levels.

Clause 222. The system of any of clauses 205-221, wherein the first glucose profile and the second glucose profile each comprise a plurality of traces, wherein a trace of the plurality of traces corresponds to a day of the first period of time, and wherein the plurality of traces are displayed in an overlay pattern.

Clause 223. A method for displaying metrics relating to a subject, the method comprising:
receiving time-correlated data characterizing an analyte level of a subject;
determining a subset of time-correlated data based on a filtering criteria selected by the subject;
determining a first analyte metric based on the time-correlated data for a first time period and a second analyte metric based on the subset of the time correlated data for the first time period; and
displaying the first analyte metric and the second analyte metric on a single graphical subject interface.

Clause 224. The method of clause 223, wherein the first analyte metric is an amount of time that analyte levels determined from the time-correlated data were within at least one concentration range during the first time period, and the second analyte metric is an amount of time that analyte levels determined from the subset of time-correlated data were within the at least one concentration range during the first time period.

Clause 225. The method of any of clauses 223-224, wherein the at least one concentration range comprises a target concentration range.

Clause 226. The method of any of clauses 223-225, wherein the at least one concentration range comprises a plurality of concentration ranges.

Clause 227. The method of any of clauses 223-226, wherein the plurality of concentration ranges comprises a low glucose concentration range, a target concentration range, and a high glucose concentration range.

Clause 228. The method of any of clauses 223-227, wherein the plurality of concentration ranges comprises a very low glucose concentration range, a low glucose concentration range, a target concentration range, a high glucose concentration range, and a very high glucose concentration range.

Clause 229. The method of any of clauses 223-228, wherein the filtering criteria comprises enablement of at least one alarm.

Clause 230. The method of any of clauses 223-229, further comprising the steps of: determining a second subset of time-correlated data based on the filtering criteria selected by the subject, wherein the second subset of time-correlated data comprises time-correlated data when the at least one alarm is disabled, and displaying a third analyte metric on the single graphical subject interface, wherein the third glucose profile displays the second subset of time-correlated data over the first time period.

Clause 231. The method of any of clauses 223-230, further comprising the steps of: determining an analyte level based on the time-correlated data; and displaying the analyte level on an additional graphical user interface, wherein the filtering criteria comprises the subject viewing the analyte level on the additional graphical user interface within a period of time.

Clause 232. The method of any of clauses 223-231, wherein the period of time is about 3 hours.

Clause 233. The method of any of clauses 223-232, wherein the filtering criteria comprises time-correlated data corresponding to an A1c value greater than a threshold.

Clause 234. The method of any of clauses 223-233, wherein the threshold is about 7%.

Clause 235. The method of any of clauses 223-234, wherein the instructions, when executed by the one or more processor, further cause the system to:

display a first glucose profile and a second glucose profile on the single graphical subject interface, wherein the first glucose profile displays glucose levels associated with the time-correlated data over the first time period, and wherein the second glucose profile displays the subset of the time-correlated data over the first time period.

Clause 236. The method of any of clauses 223-235, wherein the first glucose profile and the second glucose profile each comprise a trace of a median glucose level.

Clause 237. The method of any of clauses 223-236, wherein the first glucose profile and the second glucose profile each further comprise an upper trace of an hourly 95^{th} percentile of glucose levels and a lower trace of a 5^{th} percentile of glucose levels.

Clause 238. The method of any of clauses 223-237, wherein the first glucose profile and the second glucose profile each further comprise a second upper trace of an hourly 75^{th} percentile of glucose levels and a second lower trace of an hourly 25^{th} percentile of glucose levels.

Clause 239. The method of any of clauses 223-238, wherein the first glucose profile and the second glucose profile each further comprise data indicative of an hourly 75^{th} percentile of glucose levels and a second lower trace of an hourly 15^{th} percentile of glucose levels.

Clause 240. The method of any of clauses 223-239, wherein the first glucose profile and the second glucose profile each comprise a plurality of traces, wherein a trace of the plurality of traces corresponds to a day of the first period of time, and wherein the plurality of traces are displayed in an overlay pattern.

Clause 241. A system for displaying metrics relating to a subject, the system comprising:
wireless communications circuitry configured to receive time-correlated data characterizing glucose of the subject and time-correlated data characterizing an additional analyte of the subject;
a display configured to visually present information; and
a memory; and one or more processors coupled with the wireless communications circuitry, the display, the memory, wherein the memory stores instructions and time-correlated data characterizing an analyte level of the subject, wherein the instructions, when executed by the one or more processors, cause the system to:
   determine a subset of time-correlated data characterizing glucose based at least on a first filtering criteria related to at least one determined level of the additional analyte; and
   display a first glucose profile and a second glucose profile on a single graphical subject interface, wherein the first glucose profile displays glucose levels associated with the time-correlated data over a first time period, and wherein the second glucose profile displays the subset of the time-correlated data characterizing glucose based at least on the first filtering criteria over the first time period.

Clause 242. The system of clause 241, wherein the first filtering criteria comprises the at least one determined analyte level of the additional analyte is above a threshold value.

Clause 243. The system of any of clauses 241-242, wherein the additional analyte is a ketone or ketone body.

Clause 244. The system of any of clauses 241-243, wherein the additional analyte is β-hydroxybutyrate.

Clause 245. The system of any of clauses 241-244, wherein the additional analyte is lactate.

Clause 246. The system of any of clauses 241-245, wherein the wireless communications circuitry is further configured to receive data characterizing activity of the subject, and wherein the first filtering criteria further comprises a determined activity level of the subject is below a high activity threshold.

Clause 247. A method for displaying metrics relating to a subject, comprising the steps of:
receiving time-correlated data characterizing an analyte level of a subject;
determining a subset of time-correlated data characterizing glucose based at least on a first filtering criteria related to at least one determined level of the additional analyte; and
displaying a first glucose profile and a second glucose profile on a single graphical subject interface, wherein the first glucose profile displays glucose levels associated with the time-correlated data over a first time period, and wherein the second glucose profile displays the subset of the time-correlated data characterizing glucose based at least on the first filtering criteria over the first time period.

Clause 248. The method of clause 247, wherein the first filtering criteria comprises the at least one determined analyte level of the additional analyte is above a threshold value.

Clause 249. The method of any of clauses 247-248, wherein the additional analyte is a ketone or ketone body.

Clause 250. The method of any of clauses 247-249, wherein the additional analyte is β-hydroxybutyrate.

Clause 251. The method of any of clauses 247-250, wherein the additional analyte is lactate.

Clause 252. The method of any of clauses 247-251, wherein the wireless communications circuitry is further configured to receive data characterizing activity of the subject, and wherein the first filtering criteria further comprises a determined activity level of the subject is below a high activity threshold.

Clause 253. A system for displaying metrics relating to a subject, the system comprising:
wireless communications circuitry configured to receive time-correlated data characterizing glucose of the subject;
a display configured to visually present information; and
a memory, and one or more processors coupled with the wireless communications circuitry, the display, and the memory, wherein the memory stores instructions and time-correlated data characterizing an analyte level of the subject, wherein the instructions, when executed by the one or more processors, cause the system to:
   determine a subset of time-correlated data characterizing glucose based at least on a first filtering criteria; and
   display a first glucose profile and a second glucose profile on a single graphical subject interface, wherein the first glucose profile displays glucose levels associated with the time-correlated data over a time period, and wherein the second glucose profile displays the subset of the time-correlated data characterizing glucose based at least on the first filtering criteria over the time period.

Clause 254. The system of clause 253, wherein the first filtering criteria comprises a type of day.

Clause 255. The system of any of clauses 253-254, wherein the type of day is a weekday.

Clause 256. The system of any of clauses 253-255, wherein the type of day is a weekend day.

Clause 257. The system of any of clauses 253-256, wherein the type of day is a work day of the subject.

Clause 258. The system of any of clauses 253-257, wherein the type of day is a vacation day of the subject.

Clause 259. The system of any of clauses 253-258, wherein the type of day is a day in which the subject exercised.

Clause 260. The system of any of clauses 253-259, wherein the type of day is a day in which the subject did not exercise.

Clause 261. The system of any of clauses 253-260, wherein the type of day is a day in which the subject missed a basal insulin dose.

Clause 262. The system of any of clauses 253-261, wherein the type of day is a day in which the subject received at least one basal insulin dose.

Clause 263. The system of any of clauses 253-262, wherein the type of day is a day in which the subject missed at least one bolus insulin dose.

Clause 264. The system of any of clauses 253-263, wherein the wireless communications circuitry is further configured to receive data characterizing activity of the subject.

Clause 265. A method for displaying metrics relating to a subject, comprising the steps of:
receiving time-correlated data characterizing an analyte level of a subject;
determining a subset of time-correlated data characterizing glucose based at least on a first filtering criteria; and
displaying a first glucose profile and a second glucose profile on a single graphical subject interface, wherein the first glucose profile displays glucose levels associated with the time-correlated data over a time period, and wherein the second glucose profile displays the subset of the time-correlated data characterizing glucose based at least on the first filtering criteria over the time period.

Clause 266. The method of clause 265, the first filtering criteria comprises a type of day.

Clause 267. The method of any of clauses 265-266, the type of day is a weekday.

Clause 268. The method of any of clauses 265-267, the type of day is a weekend day.

Clause 269. The method of any of clauses 265-268, the type of day is a work day of the subject.

Clause 270. The method of any of clauses 265-269, the type of day is a vacation day of the subject.

Clause 271. The method of any of clauses 265-270, the type of day is a day in which the subject exercised.

Clause 272. The method of any of clauses 265-271, the type of day is a day in which the subject did not exercise.

Clause 273. The method of any of clauses 265-272, the type of day is a day in which the subject missed a basal insulin dose.

Clause 274. The method of any of clauses 265-273, the type of day is a day in which the subject received at least one basal insulin dose.

Clause 275. The method of any of clauses 265-274, the type of day is a day in which the subject missed at least one bolus insulin dose.

Clause 276. The method of any of clauses 265-275, wherein the wireless communications circuitry is further configured to receive data characterizing activity of the subject.

1-55. (canceled)

56. A system for displaying metrics relating to a subject, the system comprising:
wireless communications circuitry configured to receive time-correlated data characterizing an analyte level of the subject;
a display configured to visually present information;
a memory; and one or more processors coupled with the wireless communications circuitry, the display, and the memory, wherein the memory stores instructions and time-correlated data characterizing an analyte level of the subject, wherein the instructions, when executed by the one or more processors, cause the system to:
determine a subset of time-correlated data based on a filtering criteria selected by the subject, wherein the filtering criteria comprises enablement of at least one alarm; and
display a first glucose profile and a second glucose profile on a single graphical subject interface, wherein the first glucose profile displays glucose levels associated with the time-correlated data over a first time period, and wherein the second glucose profile displays the subset of the time-correlated data over the first time period.

57. The system of claim 56, wherein the at least one alarm is a low glucose alarm.

58. The system of claim 57, wherein the low glucose alarm has a threshold of about 70 mg/dL.

59. The system of claim 56, wherein the at least one alarm is a high glucose alarm.

60. The system of claim 59, wherein the high glucose alarm has a threshold of about 180 mg/dL.

61. The system of claim 56, wherein the instructions, when executed by the one or more processor, further cause the system to:
display occurrences of the at least one alarm in the first time period on at least one of the first and second glucose profiles.

62. The system of claim 56, wherein the wireless communications circuitry is further configured to receive dosage data for doses of a medication received by the subject over a period of time, and wherein the memory further stores doses of a glucose level-altering medication received by the subject over a period of time,
wherein the instructions, when executed by the one or more processor, further cause the system to:
display instances of doses of the medication received in the first time period on at least one of the first and second glucose profiles.

63. The system of claim 56, wherein the instructions, when executed by the one or more processor, further cause the system to:
determine a second subset of time-correlated data based on the filtering criteria selected by the subject, wherein the second subset of time-correlated data comprises time-correlated data when the at least one alarm is disabled, and
display a third glucose profile on the single graphical subject interface, wherein the third glucose profile displays the second subset of time-correlated data over the first time period.

64. The system of claim 63, wherein the instructions, when executed by the one or more processor, further cause the system to:
display occurrences of the at least one alarm in the first time period on at least one of the first, second, and third glucose profiles.

65. The system of claim 64, wherein the wireless communications circuitry is further configured to receive dosage data for doses of a medication received by the subject over a period of time, and wherein the memory further stores doses of a glucose level-altering medication received by the subject over a period of time,
wherein the instructions, when executed by the one or more processor, further cause the system to:
display instances of doses of the medication received in the first time period on at least one of the first, second, and third glucose profiles.

66. The system of claim 65, wherein the instances of doses the medication received in the first time period are displayed on the second glucose profile.

67. The system of claim 56, wherein the wireless communications circuitry is further configured to receive doses of a medication received by the subject over a period of time, and wherein the memory further stores doses of a glucose level-altering medication received by the subject over a period of time, wherein the instructions cause the one or more processors to determine at least one recommended dose, and wherein the filtering criteria further comprises dose concordance of the glucose altering medication.

68. The system of claim 67, wherein the wireless communications circuitry is further configured to receive dosage data for a dose regimen for the subject, and wherein the at least one recommended dose is at least partly determined from the dose regimen.

69. The system of claim 67, wherein the dose concordance comprises missed doses, under-bolused doses with reference to the at least one recommended dose, over-bolused doses with reference to the at least one recommended dose, late meal doses, or extra meal doses.

70. The system of claim 56, wherein the first glucose profile and the second glucose profile each comprise a trace of a median glucose level.

71. The system of claim 70, wherein the first glucose profile and the second glucose profile each further comprise an upper trace of an hourly 95^{th} percentile of glucose levels and a lower trace of an hourly 5^{th} percentile of glucose levels.

72. The system of claim 71, wherein the first glucose profile and the second glucose profile each further comprise a second upper trace of an hourly 75^{th} percentile of glucose levels and a second lower trace of an hourly 25^{th} percentile of glucose levels.

73. The system of claim 70, wherein the first glucose profile and the second glucose profile each further comprise data indicative of an hourly 75^{th} percentile of glucose levels and a second lower trace of an hourly 15^{th} percentile of glucose levels.

74. The system of claim 56, wherein the first glucose profile and the second glucose profile each comprise a plurality of traces, wherein a trace of the plurality of traces corresponds to a day of the first period of time, and wherein the plurality of traces are displayed in an overlay pattern.

75. The system of claim 56, wherein the instructions, when executed by the one or more processor, further cause the system to:
determine a first analyte metric based on the time-correlated data for the first time period and a second analyte metric based on the subset of the time correlated data for the first time period; and
display the first analyte metric and the second analyte metric on the single graphical subject interface.

76. The system of claim 56, wherein the instructions, when executed by the one or more processor, further cause the system to:
determine at least one first analyte statistic based on the time-correlated data for the first time period and at least one second analyte statistic based on the subset of the time correlated data for the first time period; and
display the at least one first analyte statistic and the at least one second analyte statistic on the single graphical subject interface.

77. The system of claim 76, wherein the at least one first analyte statistic comprises a first glucose management indicator, a first average glucose, a first standard deviation, or combinations thereof, and/or wherein the at least one second analyte statistic comprises a second glucose management indicator, a second average glucose, a second standard deviation, or combinations thereof.

78. The system of claim 56, wherein the instructions, when executed by the one or more processor, further cause the system to:
determine a first hypoglycemia risk based on the time-correlated data for the first time period and a second hypoglycemia risk based on the subset of the time correlated data for the first time period; and
display the first and the second hypoglycemia risk on the single graphical subject interface.

79-138. (canceled)
